(19) United States
(12) Patent Application Publication
(10) Pub. No.: US 2021/0052621 A1
(43) Pub. Date: Feb. 25, 2021

(54) METHODS FOR TREATING FLAVIVIRUSES AND ZIKA INFECTIONS

(71) Applicant: THE REGENTS OF THE UNIVERSITY OF CALIFORNIA, Oakland, CA (US)

(72) Inventor: Tariq M. Rana, San Diego, CA (US)

(21) Appl. No.: 16/478,119

(22) PCT Filed: Jan. 17, 2018
(86) PCT No.: PCT/US18/14108

§ 371 Date: Jul. 15, 2019

Related U.S. Application Data
(60) Provisional application No. 62/447,290, filed on Jan. 17, 2017.

(51) Int. Cl. A61K 31/7072; A61P 31/14; A61K 31/472; A61K 38/16; A61K 38/45; A61K 38/48

(52) U.S. Cl. CPC A61K 31/7072; A61P 31/14; A61K 38/482; A61K 38/162; A61K 38/45; A61K 31/472

(57) ABSTRACT

There are provided, inter alia, methods of treatment of Zika virus infection.

### CROSS-REFERENCES TO RELATED APPLICATIONS

This application claims the benefit of U.S. Provisional Patent Application No. 62/447,290 filed Jan. 17, 2017, which is incorporated herein by reference in its entirety.

### BACKGROUND OF THE INVENTION

Human infection with ZIKA virus (ZIKV), a mosquito-borne flavivirus, has spread rapidly since the 2015 outbreak in Brazil, and the World Health Organization declared ZIKV infection an International Public Health Emergency in 2016. ZIKV was discovered in 1947 and, although it had previously caused only sporadic disease in Africa and Asia, more recent outbreaks occurred in Micronesia in 2007 and in French Polynesia in 2013. ZIKV infection has been identified as the etiological agent of severe neurological defects, including microcephaly during fetal development and neuronal injury associated with Guillain-Barré syndrome in adults. New modes of viral transmission, including maternal-fetal and sexual transmission have been reported. ZIKV can infect human skin explants, peripheral blood mononuclear cells, human neuroprogenitor cells, and human cerebral organoids. In mouse models, ZIKV may be neurotropic.

ZIKV and other members of the Flaviviridae family, such as dengue (DENV), West Nile (WNV), yellow fever (YFV), and Japanese encephalopathy (JEV), are positive (+) single-stranded RNA viruses. The ZIKV genome encodes a single polyprotein precursor that is cleaved by viral and host proteases to produce three structural and seven nonstructural proteins. Although our understanding of the molecular mechanisms involved in ZIKV infection of human cells has increased dramatically in the past few years, key determinants of ZIKV pathogenicity, such as cell-type specificity, mode of entry, and host factors essential for replication, are still largely unknown. In particular, there is a large gap in our understanding of the genetic and epigenetic regulatory mechanisms governing the viral life cycle and viral interactions with host cells.

Solutions to this and other problems in the art are provided. Specifically, we discovered a number of FDA approved drugs that can be used to treat ZIKV infections and possibly other flaviviruses including Dengue, West Nile, JEV, and HCV. Specifically, we performed cell-based screens using libraries of compounds containing antiviral drugs and other available potential antiviral like compounds. We have accordingly identified FDA approved drugs that inhibit ZIKV in vitro and in vivo. These drugs are well tolerated in many cell lines including stem cells and mice and potently inhibit ZIKV infection.

### BRIEF SUMMARY OF THE INVENTION

In a first aspect, there is provided a method of treating a Zika viral infection. The method includes administering to a subject in need thereof an effective amount of a compound as set forth in any of FIG. 1A, 1B, 2, 3, 6, 8, or 9.

In another aspect, there is provided a method of treating a Zika viral infection. The method includes administering to a subject in need thereof an effective amount of an NS5 polymerase inhibitor.

In another aspect, there is provided a method of treating a Zika viral infection. The method includes administering to a subject in need thereof an effective amount of an HIV protease inhibitor.

In another aspect, there is provided a method of treating a Zika viral infection. The method includes administering to a subject in need thereof an effective amount of a calcium channel blocker.

In another aspect, there is provided a method of treating a Zika viral infection. The method includes administering to a subject in need thereof a combined effective amount of a therapeutic composition including an NS5 polymerase inhibitor and a HIV protease inhibitor.

In another aspect, there is provided a method of treating a Zika viral infection. The method includes administering to a subject in need thereof an effective amount of a protein or a gene encoding the protein. In embodiments, the protein is a ZIKV non-structural (NS) protein. In embodiments, the ZIKV non-structural protein is NS5. In embodiments, the protein is NS5 RNA polymerase. In embodiment, the ZIKV non-structural protein is NS2B-NS3. In embodiments, the protein is NS2B-NS3 protease.

### BRIEF DESCRIPTION OF THE DRAWINGS

FIGS. 1A-1B. FIG. 1A: Antiviral activity of approved drugs/clinical molecules library was tested against Zika virus in human 293 T cells at the MOI of 5. Cells were pretreated with drugs (10 μM) for 1 hour and infected with ZIKV (MOI=5). After 24 hours of infection, mRNA was isolated and the levels of cellular ZIKV was determined by qRT-PCR. FIG. 1B: Methyl transferase inhibitors potently reduced ZIKV replication. Methods were as described in FIG. 1A.

FIGS. 2A-2D. Dose dependent inhibition of Zika virus in 293 T cells by selected antiviral molecules (at the MOI of 5 using different concentration of drugs, ICso was calculated). FIG. 2A—Lopinavir. FIG. 2B—Daclatasvir. FIG. 2C—Oxyclozanide. FIG. 2D—Rafoxanide.

FIGS. 3A-3B. Synergistic activity of lopinavir and ritonavir. 293 T cells were pretreated with indicated drugs and after 1 hour, the cells were infected with ZIKV at the MOI of 5. After 24 hours, proteins were analyzed by western blotting (FIG. 3A) and mRNA was isolated and the levels of cellular ZIKV was determined by qRT-PCR (FIG. 3B).

FIG. 4. Experimental outline to determine the drug efficacy for ZIKV inhibition in vivo.

FIGS. 5A-5D. Protease inhibitors reduced ZIKV in animals (brain—FIG. 5A; blood—FIG. 5B; testes—FIG. 5C; spleen—FIG. 5D) both as prophylaxis and therapeutic regimens.

FIG. 6. Screening of additional compounds for ZIKA levels using Vero cells (in 10 μM concentration). Methods of screening were as described herein. Viral RNA released was analyzed by qRT-PCR for ZIKA. Compounds (left to right): control, berberine, fluoxetine, formoterol, genistine, U0126, dibucaine, pirlindole, suramin, mycophenolic acid, polyhydroxyalkanoates, (PHA) and niclosamide.

FIG. 7. Screening of additional compounds for ZIKA levels using Vero cells (in 10 μM concentration) as described for FIG. 6. Compounds (left to right): clomiphene, amphotericin B, and toremifene.

FIG. 8. Screening of additional compounds for ZIKA levels using Vero cells (in 10 μM concentration) as described for FIG. 6. Compounds providing hits: ganciclovir, procaine hydrochloride, zidovudine, aciclovir, drostanolone propionate, dapivirine (TMC120), tilorone hydrochloride (2,7-bis[2-(diethylamino)ethoxy]-9H-fluoren-9-one, HCl), and Docosanol.

FIGS. 9A-9K. ZIKA inhibition by various drugs in 293 cells at 20 μM, visualized by immune staining. Drugs: mock (FIG. 9A), DMSO (FIG. 9B), PHA-690509 (FIG. 9C), formoterol (FIG. 9D), fluoxetine (FIG. 9E), genistein (FIG. 9F), U0126 (FIG. 9G), berberine (FIG. 9H), dibucaine (FIG. 9I), pirlindole (FIG. 9J), and suramin (FIG. 9K). Left panels: DAPI; right panels: ZIKV.

FIGS. 10A-10E. Variable ZIKV Infection and Replication Levels in Different Cell Types. Immunohistochemistry of ZIKV gene expression in microglial (FIG. 10A), fibroblast (BJ) (FIG. 10B), kidney (293FT) (FIG. 10C), and macrophage (THP-1) (FIG. 10D) cell lines at 24 h post-infection. Cells were stained with anti-flavivirus envelope protein (ZIKVE), and nuclei were visualized with Hoechst 33258. FIG. 10E: ZIKV replication assessed by one-step RT-qPCR analysis of viral supernatants at the indicated times post-infection. Data are presented as the mean±SEM of n=3. *p<0.05, **p<0.01, ***p<0.001; ns, not significant by two-way ANOVA.

FIGS. 11A-11E. Circos plots of transcriptional changes in microglia (FIG. 11A), BJ (FIG. 11B), 293FT (FIG. 11C), and THP-1 derived macrophages (FIG. 11D) cells at 24 h post-infection. The outer circles represent the expression levels of the transcripts before infection. The inner circles represent the differentially expressed genes, with the size of the lines indicating the fold change in expression. Genes upregulated and downregulated by ZIKV infection are shown, respectively. (FIG. 11E) Circos plot showing differentially expressed genes in all four cell types. The number of differentially expressed genes and the magnitude of the expression change are inversely correlated with ZIKV expression. Upregulated genes are in red and downregulated genes are in blue. Outer to inner circles: THP-1, 293FT, BJ, and microglia.

FIG. 12A-12E. Cell Type-Specific Differences in Steady-State Expression of Viral Response Genes Reveal Potential Antiviral Targets. (FIG. 12A) Hierarchical clustering of genes associated with “response to virus” in microglia, BJ, 293FT, and THP-1 derived macrophages before infection (top) and the fold change in expression of the same genes at 24 h post-ZIKV infection (bottom). Left scale bar: 0:10 represents gene expression log 2(RPKM+1) for all cell times. Right scale bar: −2:2 represents fold change in gene expressed between mock treated and ZIKV infected cells. Also see Table 1. (FIG. 12B) Pre-infection expression levels of genes associated with “response to virus,” showing high expression in THP-1 derived macrophages compared with microglia, BJ, and 293T cells. (FIG. 12C) Hierarchical clustering of genes associated with “cell surface” (top) and their associated fold change in expression following ZIKV infection (bottom). (FIG. 12D) Differential gene expression of cell surface proteins and receptors involved in cell activation, immune response, and cell surface signaling in THP-1 derived macrophages. (FIG. 12E) Gene ontology analysis of cell surface genes highly expressed specifically in THP-1 and lowly expressed in other cell lines.

FIGS. 13A-13G. Analysis of Differentially Expressed Genes Post-ZIKV Infection Identifies Key Pathways Exploited by ZIKV. (FIG. 13A) Hierarchical clustering of differentially expressed genes following ZIKV infection, showing cell type-specific transcriptional changes. Genes displayed have fold changes of >1.4 and p<0.05 in at least one cell type. The vertical bar to the right indicates the differentially expressed gene clusters described by color in the text. (FIG. 13B) Interactome of differentially expressed genes across all cell types. (FIG. 13C) Gene ontology analysis of “red cluster” genes (vertical bar in A), which are mostly upregulated in microglia and THP-1 derived macrophages and downregulated in 293FT and BJ cells. (FIG. 13D) Gene ontology analysis of “yellow cluster” genes, which are mostly downregulated in microglia and THP-1 derived macrophages and upregulated in 293FT and BJ cells. (FIG. 13E) Analysis of “green cluster” genes, which are upregulated only in THP-1, showing enrichment in genes that modulate the adaptive immune response and complement cascades. (FIG. 13F) Analysis of “magenta cluster” genes, which are upregulated in 293FT and THP-1 derived macrophages and downregulated in microglia and BJ cells, showing enrichment in genes that regulate viral receptor activity, endothelial cells, and angiogenesis. (FIG. 13G) Analysis of “blue cluster” genes, which are upregulated in microglia cells and downregulated in BJ, 293T, and THP-1 derived macrophages, showing enrichment in genes that regulate cellular and metabolic processes.

FIGS. 14A-14D. Nucleoside Metabolic Inhibitors Attenuate ZIKV Replication. (FIG. 14A) ZIKV replication assessed by RT-qPCR analysis 48 hours post-infection in mock, ZIKV only, ZIKV plus 1 μM floxuridine or ZIKV plus 10 μM floxuridine treated microglia. Data are presented as the mean±SEM of n=3. *p<0.05, **p<0.01 by t-test. (FIG. 14B) Immunohistochemistry of ZIKV gene expression in mock, ZIKV only, ZIKV plus 1 μM floxuridine (FUDX1) or ZIKV plus 10 μM floxuridine (FUDX10) treated microglia. Cells were stained with anti-flavivirus envelope protein (ZIKVE), and nuclei were visualized with Hoechst 33258. (FIG. 14C) ZIKV replication assessed by RT-qPCR analysis 48 hours post-infection in mock, ZIKV only, ZIKV plus fluorouracil or ZIKV plus 10 μM fluorouracil treated microglia. Data are presented as the mean±SEM of n=3. *p<0.05, **p<0.01 by t-test. (FIG. 14D) Immunohistochemistry of ZIKV gene expression in mock, ZIKV only, ZIKV plus 1 μM fluorouracil (FU1) or ZIKV plus 10 μM fluorouracil (FU10) treated microglia. Cells were stained with anti-flavivirus envelope protein (ZIKVE), and nuclei were visualized with Hoechst 33258.

FIGS. 15A-15E. Amplification of MR766 ZIKV in Vero and BHK Cells (related to FIGS. 10A-10E). (FIG. 15A). Immunohistochemical staining for ZIKV expression in Vero and BHK cells. (FIG. 15B) RT-qPCR analysis of Vero, BHK, microglia, BJ, 293T, and THP-1 cell supernatants. (FIG. 15C-15E) RT-qPCR analysis of innate immune response genes TLR3, IRF3, APOBEC in microglia, BJ and THP-1 derived macrophages, respectively.

FIGS. 16A-16F. Analysis of Viral Response and Immune-Related Genes Reveals Potential Antiviral Targets (related to FIGS. 12A-12E). (FIG. 16A) Heat map of genes highly expressed in microglia, BJ, and 293T cells, but not in THP-1 derived macrophages, from the analysis of “response to virus”-associated genes in FIG. 12A. Left scale bar: 0:10 represents gene expression log 2(RPKM+1) for all cell times. Right scale bar: −2:2 represents fold change in gene expressed between mock treated and ZIKV infected cells. (FIG. 16B-16D) Hierarchical clustering of differentially expressed genes related to “modulation by virus of host gene expression” (FIG. 16B), “viral transcription” (FIG. 16C), and “viral release from host cell” (FIG. 16D) between mock-infected cells (left) and their associated fold change in expression after ZIKV infection (right). (FIG. 16E) Scatterplot of immune response genes showing higher expression of immune-related genes in THP-1 derived macrophages compared with microglia cells. (FIG. 16F) Hierarchical clustering of differentially expressed genes related to “immune response” in mock-infected cells (top) and their associated fold change in expression after ZIKV infection (bottom). Also see Table 3.

FIGS. 17A-17D. Gene ontology analysis of differentially expressed genes in microglia (FIG. 17A), BJ (FIG. 17B), 293FT (FIG. 17C), and THP-1 (FIG. 17D) cells (related to FIGS. 13A-13G).

### DETAILED DESCRIPTION OF THE INVENTION

### Definitions

The following definitions are provided to facilitate understanding of certain terms used frequently herein and are not meant to limit the scope of the present disclosure. The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include di- and multivalent radicals, having the number of carbon atoms designated (i.e., C₁-C₁₀ means one to ten carbons). Alkyl is not cyclized. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds (e.g. alkene, alkyne). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., selected from the group consisting of O, N, P, Si, and S), and wherein the nitrogen and sulfur atoms may optionally be oxidized, and the nitrogen heteroatom may optionally be quaternized. Heteroalkyl is not cyclized. The heteroatom(s) (e.g., O, N, P, S, and Si) may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Cycloalkyl and heterocycloalkyl are non-aromatic. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

The term “oxo,” as used herein, means an oxygen that is double bonded to a carbon atom.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, monophosphate (or derivatives thereof), diphosphate (or derivatives thereof), triphosphate (or derivatives thereof), in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, monophosphate (or derivatives thereof), diphosphate (or derivatives thereof), triphosphate (or derivatives thereof), in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:

(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), or triphosphate (or derivatives thereof), and
(B) alkyl, heteroalkyl, cycloalkyl, heterocycloalkyl, aryl, heteroaryl, monophosphate (or derivatives thereof), diphosphate (or derivatives thereof), or triphosphate (or derivatives thereof), substituted with at least one substituent selected from:
(i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), or triphosphate (or derivatives thereof), and
(ii) alkyl, heteroalkyl, cycloalkyl, heterocycloalkyl, aryl, heteroaryl, monophosphate (or derivatives thereof), diphosphate (or derivatives thereof), or triphosphate (or derivatives thereof), substituted with at least one substituent selected from:
   (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), or triphosphate (or derivatives thereof), and
   (b) alkyl, heteroalkyl, cycloalkyl, heterocycloalkyl, aryl, heteroaryl, monophosphate (or derivatives thereof), diphosphate (or derivatives thereof), or triphosphate (or derivatives thereof), substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), and triphosphate (or derivatives thereof).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in embodiments each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In embodiments, the compound is a chemical species set forth herein.

Certain complexes and compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus, a Roman alphabetic symbol may be used to distinguish each appearance of that particular R group. For example, where multiple R¹³ substituents are present, each R¹³ substituent may be distinguished as R^{13A}, R^{13B}, R^{13C}, etc., wherein each of R^{13A}, R^{13B}, R^{13C}, R^{13D}, etc. is defined within the scope of the definition of R¹³ and optionally differently.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

“Analog,” or “analogue” are used in accordance with plain ordinary meaning within Chemistry and Biology and refer to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analogue is a compound that is similar or comparable in function and appearance but not in structure or origin to a reference compound.

The term “pharmaceutically acceptable salts” is meant to include salts of active compounds that are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds disclosed herein contain both basic and acidic functionalities that allow the compounds to be converted into either base or acid addition salts.

Thus, the compounds disclosed herein may exist as salts, such as with pharmaceutically acceptable acids. The compounds disclosed herein include such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, there are provided compounds which are in a prodrug form. Prodrugs of the compounds described herein include those compounds that readily undergo chemical or enzymatic changes under physiological conditions to provide the compounds disclosed herein. Additionally, prodrugs can be converted to the compounds disclosed herein by chemical or biochemical methods in an ex vivo environment. For example, prodrugs can be slowly converted to the compounds disclosed herein when placed in a transdermal patch reservoir with a suitable enzyme or chemical reagent.

Certain compounds disclosed herein can exist in unsolvated forms as well as solvated forms, including hydrated forms. In general, the solvated forms are equivalent to unsolvated forms and are encompassed within the scope disclosed herein. Certain compounds disclosed herein may exist in multiple crystalline or amorphous forms. In general, all physical forms are equivalent for the uses disclosed herein and are intended to be within the scope of the compounds and methods disclosed herein.

As used herein, the term “salt” refers to acid or base salts of the compounds used in the methods disclosed herein. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “treating”, or “treatment” refer to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being. The treatment or amelioration of symptoms can be based on objective or subjective parameters, including the results of a physical examination, neuropsychiatric exams, and/or a psychiatric evaluation. The term “treating” and conjugations thereof, include prevention of an injury, pathology, condition, or disease.

An “effective amount” is an amount sufficient to accomplish a stated purpose (e.g., achieve the effect for which it is administered, treat a disease, reduce enzyme activity, increase enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the methods disclosed herein should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In embodiments, a control is the measurement of the activity of a protein in the absence of a compound as described herein (including embodiments and examples).

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein or enzyme. Contacting may include allowing a compound described herein to interact with a protein or enzyme that is involved in a signaling pathway.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As defined herein, the terms “activation”, “activate”, “activating” and the like in reference to a protein-activator (e.g. agonist) interaction means positively affecting (e.g. increasing) the activity or function of the relative to the activity or function of the protein in the absence of the activator (e.g. composition described herein). Thus, in embodiments, activation may include, at least in part, partially or totally increasing stimulation, increasing or enabling activation, or activating, sensitizing, or up-regulating signal transduction or enzymatic activity or the amount of a protein decreased in a disease. The amount of activation may be 10%, 20%, 30%, 40%, 50%, 60%, 70%, 80%, 90%, 100% or more in comparison to a control in the absence of the agonist. In embodiments, the activation is 1.5-fold, 2-fold, 3-fold, 4-fold, 5-fold, 10-fold, or more than the expression or activity in the absence of the agonist.

As defined herein, the terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In embodiments inhibition refers to reduction of a disease or symptoms of disease. Thus, in embodiments, inhibition includes, at least in part, partially or totally blocking stimulation, decreasing, preventing, or delaying activation, or inactivating, desensitizing, or down-regulating signal transduction or enzymatic activity or the amount of a protein. The amount of inhibition may be 10%, 20%, 30%, 40%, 50%, 60%, 70%, 80%, 90%, 100% or less in comparison to a control in the absence of the antagonist. In embodiments, the inhibition is 1.5-fold, 2-fold, 3-fold, 4-fold, 5-fold, 10-fold, or more than the expression or activity in the absence of the antagonist.

As defined herein, the terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

As defined herein, the term “modulator” refers to a composition that increases or decreases the level of a target molecule or the function of a target molecule or the physical state of the target of the molecule.

As defined herein, the term “modulate” is used in accordance with its plain ordinary meaning and refers to the act of changing or varying one or more properties. “Modulation” refers to the process of changing or varying one or more properties. For example, a modulator of a target protein changes by increasing or decreasing a property or function of the target molecule or the amount of the target molecule. A modulator of a disease decreases a symptom, cause, or characteristic of the targeted disease.

As defined herein, “selective” or “selectivity” or the like of a compound refers to the compound's ability to discriminate between molecular targets. “Specific”, “specifically”, “specificity”, or the like of a compound refers to the compound's ability to cause a particular action, such as inhibition, to a particular molecular target with minimal or no action to other proteins in the cell.

As defined herein, “pharmaceutically acceptable excipient” and “pharmaceutically acceptable carrier” refer to a substance that aids the administration of an active agent to and absorption by a subject and can be included in the compositions disclosed herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds disclosed herein. One of skill in the art will recognize that other pharmaceutical excipients are useful in the compositions and methods disclosed herein.

As defined herein, the term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The compositions disclosed herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions disclosed herein may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions disclosed herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions disclosed herein can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions disclosed herein into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions can also be delivered as nanoparticles.

As defined herein, the terms “acceptable,” “effective,” or “sufficient” when used to describe the selection of any components, ranges, dose forms, etc. disclosed herein intend that said component, range, dose form, etc. is suitable for the disclosed purpose.

As used herein, an “effective amount” is an amount sufficient to accomplish a stated purpose (e.g. achieve the effect for which it is administered, treat a disease (e.g., targeted by Zika virus, Dengue virus, West Nile virus, etc.), reduce receptor signalling activity, reduce one or more symptoms of a disease or condition). An example of an “effective amount” is an amount sufficient to contribute to the treatment, prevention, or reduction of a symptom or symptoms of a disease (e.g., targeted by Zika virus, Dengue virus, West Nile virus, etc.)), which could also be referred to as a “therapeutically effective amount.” A “reduction” of a symptom or symptoms (and grammatical equivalents of this phrase) means decreasing of the severity or frequency of the symptom(s), or elimination of the symptom(s). Guidance can be found in the literature for appropriate dosages for given classes of pharmaceutical products. For example, for the given parameter, a therapeutically effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). An “effective amount” refers to an antiviral drug or a composition of an antiviral drug in an amount that is sufficient to reduce the amount and/or kill virus (Zika virus, Dengue virus, West Nile virus, etc.). The virus is contacted with an amount of the antiviral drug or composition thereof (an NS5 polymerase inhibitor or HIV protease inhibitor, or combinations thereof) effective to reduce and/or kill virus. When used herein in reference to administration to a subject in need thereof, the terms “amount effective” or “effective amount” mean an amount of an NS5 polymerase inhibitor or HIV protease inhibitor, or combinations thereof which treat a viral infection. An effective amount can be administered in one or more administrations, applications or dosages. Such delivery is dependent on a number of variables including the time period which the individual dosage unit is to be used, the bioavailability of the composition, the route of administration, etc. It is understood, however, that specific amounts of an NS5 polymerase inhibitor or HIV protease inhibitor, or combinations thereof for any particular subject depends upon a variety of factors including the activity of the specific agent employed, the age, body weight, general health, sex, and diet of the subject, the time of administration, the rate of excretion, the composition combination, severity of the particular cancer being treated and form of administration.

In certain embodiments, the NS5 polymerase inhibitor and HIV protease inhibitor are administered in a combined synergistic amount. A “combined synergistic amount” as used herein refers to the sum of a first amount (e.g., an amount of an NS5 polymerase inhibitor) and a second amount (e.g., an amount of an HIV protease inhibitor) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the NS5 polymerase inhibitor when used separately from the HIV protease inhibitor. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the HIV protease inhibitor when used separately from the NS5 polymerase inhibitor.

The synergistic effect may be a disease-treating effect such as a disease triggered by Zika virus, Dengue virus, West Nile virus, etc.

The NS5 polymerase inhibitor and the HIV protease inhibitor may be administered in combination either simultaneously (e.g., as a mixture), separately but simultaneously (e.g., via separate intravenous lines or separate tablets) or sequentially (e.g., one agent is administered first followed by administration of the second agent). Thus, the term combination is used to refer to concomitant, simultaneous or sequential administration of the NS5 polymerase inhibitor and the HIV protease inhibitor.

In embodiments, the NS5 polymerase inhibitor and the HIV protease inhibitor are administered simultaneously or sequentially. In embodiments, the NS5 polymerase inhibitor and the HIV protease inhibitor are administered simultaneously. In embodiments, the NS5 polymerase inhibitor and the HIV protease inhibitor are administered sequentially. During the course of treatment the NS5 polymerase inhibitor and the HIV protease inhibitor may at times be administered sequentially and at other times be administered simultaneously.

Pharmaceutical compositions may include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds disclosed herein. Adjustment and manipulation of established dosages (e.g., frequency and duration) are well within the ability of those skilled in the art.

The compounds described herein can be used in combination with one another, with other active drugs known to be useful in treating a disease or with adjunctive agents that may not be effective alone, but may contribute to the efficacy of the active agent. Thus, the compounds described herein may be co-administered with one another or with other active drugs known to be useful in treating a disease.

By “co-administer” it is meant that a compound described herein is administered at the same time, just prior to, or just after the administration of one or more additional therapies. The compounds described herein can be administered alone or can be co-administered to the patient. Co-administration is meant to include simultaneous or sequential administration of the compound individually or in combination (more than one compound or agent). Thus, the preparations can also be combined, when desired, with other active substances.

Co-administration includes administering one active agent within 0.5, 1, 2, 4, 6, 8, 10, 12, 16, 20, or 24 hours of a second active agent. Also contemplated herein, are embodiments, where co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. In other embodiments, the active agents can be formulated separately. The active and/or adjunctive agents may be linked or conjugated to one another.

The term “associated” or “associated with” in the context of a substance or substance activity or function associated with a disease means that the disease is caused by (in whole or in part), a symptom of the disease is caused by (in whole or in part) the substance or substance activity or function, or a side-effect of the compound (e.g., toxicity) is caused by (in whole or in part) the substance or substance activity or function.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals.

“Chemotherapeutic” or “chemotherapeutic agent” is used in accordance with its plain ordinary meaning and refers to a chemical composition or compound having antineoplastic properties or the ability to inhibit the growth or proliferation of cells.

“Disease” or “condition” refer to a state of being or health status of a patient or subject capable of being treated with a compound, pharmaceutical composition, or method provided herein. In embodiments, the disease or condition is ZIKA infection.

The term “ZIKA infection,” “Zika fever” or the like refer, in the usual and customary sense, to a viral infection due to the Zika virus (ZIKV), a member of the Flaviviridae family. The Zika virus is typically enveloped and icosahedral, having a nonsegmented, single-stranded, 10 kilobase positive-sense RNA genome.

The term “nucleic acid” refers to deoxyribonucleotides (DNA) or ribonucleotides (RNA) and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

An “antisense nucleic acid” as referred to herein is a nucleic acid (e.g. DNA or RNA molecule) that is complementary to at least a portion of a specific target nucleic acid (e.g. an mRNA translatable into a protein) and is capable of reducing transcription of the target nucleic acid (e.g. mRNA from DNA) or reducing the translation of the target nucleic acid (e.g. mRNA) or altering transcript splicing (e.g. single stranded morpholino oligo). See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbonemodified nucleotides. Antisense nucleic acids include, for example, siRNA, mircoRNA and the like.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when present in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In other embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

As used herein, “viral (ZIKA) non-structural (NS) protein” refers to a protein encoded by virus (e.g., ZIKA virus) that is an RNA-binding protein that plays an integral role in virus replication.

As used herein, “ZIKV non-structural protein 5 (NS5)” refers to an RNA-dependent RNA polymerase that plays an essential role in viral replication in the infected host cytoplasm and contributes integrally to pathogenesis by localizing in the host cell nucleus. NS5 is comprised of two domains. The N-terminal domain binds GTP and can perform two biochemically distinct methylation reactions required for RNA cap formation. The C-terminal domain contains RNA-dependent RNA polymerase activity. ZIKV NS5 is recognized with high affinity by the host cell importin α/β1 heterodimer, thus representing a validated target for compounds capable of blocking that interaction.

As used herein, “ZIKV NS5 polymerase inhibitors” refer to compounds capable of preventing viral replication by selectively binding to NS5 polymerase and blocking its interaction with the host cell importin α/β1 heterodimer. The compounds shown to exhibit an inhibitory activity against ZIKA NS5 polymerase include, but are not limited to, beclabuvir, dasabuvir, deleobuvir, filibuvir, setrobuvir, radalbuvir, sofosbuvir, N-(4-hydroxyphenyl) retinamide (4-HPR), 2,1-benzothiazine-2,2-dioxide, or chloroquine (Q).

As used herein, “ZIKV non-structural protein NS2B-NS3 (NS2B-NS3)” is an HIV protease, which consists of the NS2B cofactor and the NS3 protease domain, both of which are essential for cleavage of the ZIKV polyprotein precursor and generation of fully functional viral proteins.

As used herein, “ZIKV NS2B-NS3 HIV protease inhibitors” refer to compounds capable of preventing viral replication by selectively binding to NS2B-NS3 HIV protease and blocking proteolytic cleavage of protein precursors that are necessary for production of infectious viral particles. The compounds shown to exhibit an inhibitory activity against ZIKA NS2B-NS3 HIV protease include, but are not limited to, amprenavir, atazanavir, darunavir, fosamprenavir, indinavir, lopinavir, nelfinavir, ritonavir, saquinavir, tipranavir, asunaprevir, boceprevir, grazoprevir, paritaprevir, simeprevir, or telaprevir.

As used herein, “calcium channel” refers to an ion channel which shows selective permeability to calcium ions. Voltage-gated or voltage-dependent calcium channels (VDCCs) is a group of voltage-gated ion channels found in the membranes of excitable (e.g., muscle, glial cells, neurons, etc.) with a permeability to the calcium ion. Activation of particular VDCCs allows calcium ions to rush into the cell, which, depending on the cell type, results in activation of calcium-sensitive potassium channels, muscular contraction, excitation of neurons, up-regulation of gene expression, or release of hormones or neurotransmitters. Ligand-gated ion channels (LICs, LGIC), also commonly referred as ionotropic receptors, are a group of transmembrane ion-channel proteins which open to allow ions such as Na⁺, K⁺, Ca²⁺, and/or Cl⁻ to pass through the membrane in response to the binding of a chemical messenger (i.e. a ligand), such as a neurotransmitter.

As used herein, “calcium channel blockers” (CCB) refer to compounds that are capable of disrupting the movement of calcium through calcium channels. Calcium channel blockers are used as antihypertensive drugs, i.e., as medications to decrease blood pressure in patients with hypertension. CCBs are particularly effective against large vessel stiffness, one of the common causes of elevated systolic blood pressure in elderly patients. Calcium channel blockers are also frequently used to alter heart rate, to prevent cerebral vasospasm, and to reduce chest pain caused by angina pectoris. Calcium channel blockers can be in short-acting and long-acting forms. Short-acting calcium channel blockers work quickly, but their effects lasts for a few hours. Long-acting medications are slowly released to provide a longer lasting effect. Available calcium channel blockers include, but are not limited to, Amlodipine, Aranidipine, Azelnidipine, Barnidipine Benidipine, Clevidipine, Efonidipine, Felodipine, Isradipine, Lacidipine, Lercanidipine, Manidipine, Nicardipine, Nifedipine, Nilvadipine, Nimodipine, Nisoldipine, Nitrendipine, Pranidipine, Diltiazem, and Verapamil.

### Methods

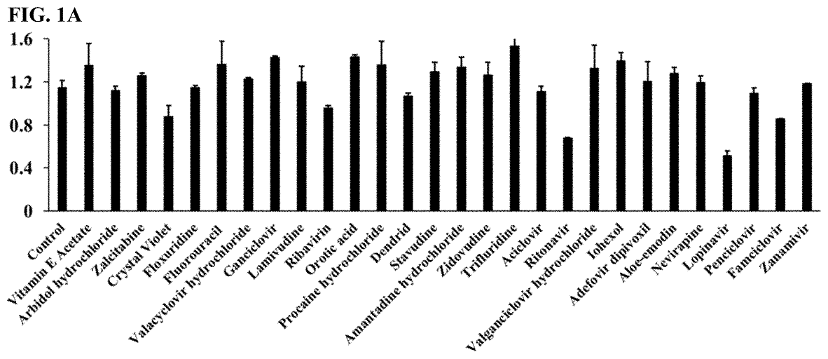

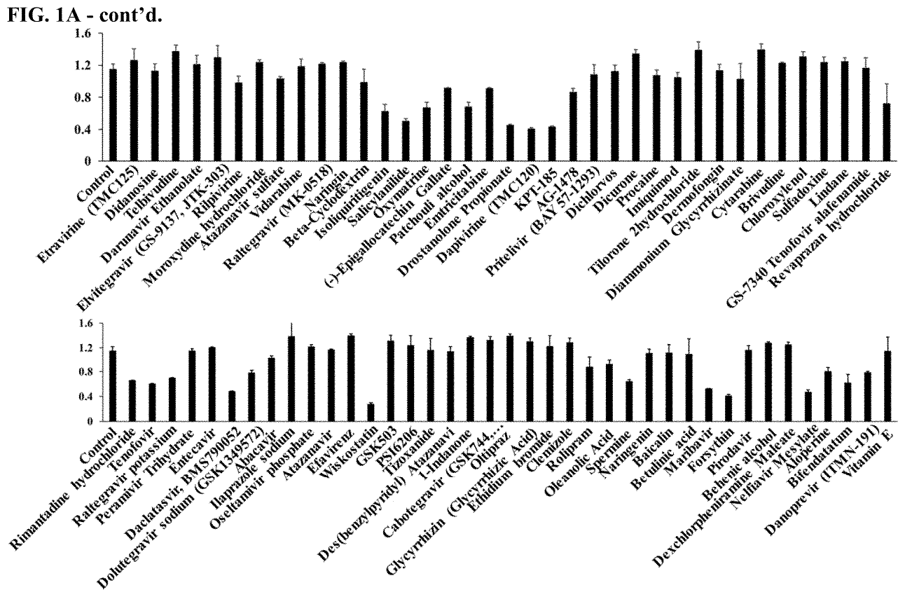

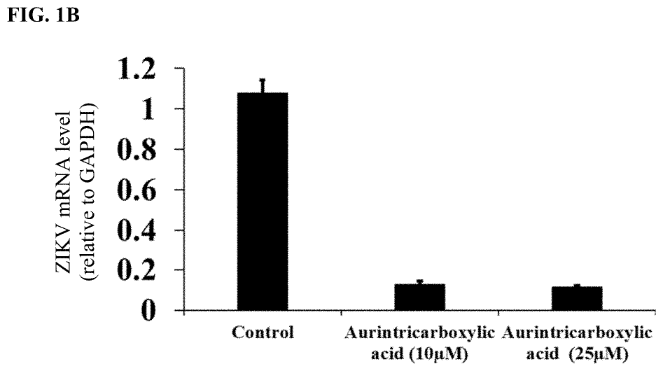

In a first aspect, there is provided a method of treating a Zika viral infection. The method includes administering to a subject in need thereof an effective amount of a compound as set forth in any of FIG. 1A1B, 2A-2D, 3A-3B, 6, 8, or 9A-9K.

In embodiments, the compound is Ganciclovir, Procaine hydrochloride, Zidovudine, Acyclovir, Drostanolone Propionate, Dapivirine (TMC120), Tilorone hydrochloride, Docosanol, Suramin, Clomiphene, Amphotericin B, Toremifene, Mycophenolic acid, Fluoxetine, Niclosamide, or polyhydroxyalkanoate (PHA). In the embodiments below, the compounds are identified by their IUPAC chemical names.

In embodiments, the compound is Ganciclovir (also referred to herein as 9-(1,3-dihydroxy-2-propoxymethyl)guanine), CAS Number 824 10-32-0.

In embodiments, the compound is Procaine hydrochloride (also referred to herein as 4-amino-, 2-(diethylamino) ethyl ester monohydrochloride), CAS Number: 51-05-8.

In embodiments, the compound is Zidovudine (also referred to herein as 3′-deoxy-3′-azido-thymidine or 1-[(2R,4S,5S)-4-Azido-5-(hydroxymethyl)oxolan-2-yl]-5-methylpyrimidine-2,4-dione), CAS Number 30516-87-1.

In embodiments, the compound is Acyclovir (also referred to herein as 2-Amino-1,9-dihydro-9-((2-hydroxyethoxy)methyl)-3H-purin-6-one), CAS Number 59277-89-3.

In embodiments, the compound is Drostanolone Propionate (also referred to herein as (2R,5S,8R,9S,10S,13S,14S,17S)-17-hydroxy-2,10,13-trimethyl-1,2,4,5,6,7,8,9,11,12,14,15,16,17-tetradecahydrocyclopenta[a]phenanthren-3-one), CAS Number 58-19-5.

In embodiments, the compound is Dapivirine (also referred to herein as 4-{[4-(mesitylamino)-2-pyrimidinyl]amino}benzonitrile, CAS Number 244767-67-7.

In embodiments, the compound is Tilorone hydrochloride (also referred to herein as 2,7-Bis(2-diethylaminoethoxy)fluoren-9-one hydrochloride); CAS Number 27591-69-1.

In embodiments, the compound is Docosanol having CAS number 661-19-8.

In embodiments, the compound is Suramin (also referred to herein as 8,8′-{Carbonylbis[imino-3,1-phenylenecarbonylimino(4-methyl-3,1-phenylene)carbonylimino]}di(1,3,5-naphthalenetrisulfonic acid), CAS Number 145-63-1.

In embodiments, the compound is Clomiphene (also referred to herein as (E,Z)-2-(4-(2-chloro-1,2-diphenylethenyl)phenoxy)-N,N-diethylethanamine), CAS Number 911-45-5.

In embodiments, the compound is Amphotericin B (also referred to herein as (1R,3S,5R,6R,9R,11R,15S,16R,17R,18S,19E,21E,23E,25E,27E,29E,31E,33R,35S,36R,37S)-33-[(3-amino-3,6-dideoxy-β-D-mannopyranosyl)oxy]-1,3,5,6,9,11,17,37-octahydroxy-15,16,18-trimethyl-13-oxo-14,39-dioxabicyclo [33.3.1]nonatriaconta-19,21,23,25,27,29,31-heptaene-36-carboxylic acid); CAS Number 1397-89-3.

In embodiments, the compound is Toremifene (also referred to herein as 2-[4-[(1Z)-4-chloro-1,2-diphenyl-but-1-en-1-yl]phenoxy]-N,N-dimethylethanamine), CAS number 89778-26-7.

In embodiments, the compound is Mycophenolic acid (also referred to herein as (4E)-6-(4-Hydroxy-6-methoxy-7-methyl-3-oxo-1,3-dihydro-2-benzofuran-5-yl)-4-methylhex-4-enoic acid), CAS Number 24280-93-1.

In embodiments, the compound is Fluoxetine (also referred to herein as N-methyl-3-phenyl-3-[4-(trifluoromethyl)phenoxy]propan-1-amine), CAS Number 54910-89-3.

In embodiments, the compound is Niclosamide (also referred to herein as 5-Chloro-N-(2-chloro-4-nitrophenyl)-2-hydroxybenzamide), CAS Number 50-65-7.

In embodiments, the compound is polyhydroxyalkanoate (PHA).

In another aspect, there is provided a method of treating a Zika viral infection. The method comprises administering to a subject in need thereof an effective amount of an NS5 polymerase inhibitor.

In embodiments, the NS5 polymerase inhibitor is beclabuvir, dasabuvir, deleobuvir, filibuvir, setrobuvir, radalbuvir, or sofosbuvir.

In embodiments, the NS5 polymerase inhibitor is beclabuvir (also referred to herein as (1aR,12bS)-8-Cyclohexyl-N-(dimethylsulfamoyl)-11-methoxy-1a-{[(1R,5S)-3-methyl-3,8-diazabicyclo[3.2.1]oct-8-yl]carbonyl}-1,1a,2,12b-tetrahydrocyclopropa[d]indolo[2,1-a][2]benzazepine-5-carboxamide), CAS Number 958002-33-0.

In embodiments, the NS5 polymerase inhibitor is dasabuvir (also referred to herein as N-{6-[5-(2,4-Dioxo-3,4-dihydro-1(2H)-pyrimidinyl)-2-methoxy-3-(2-methyl-2-propanyl)phenyl]-2-naphthyl}methanesulfonamide), CAS Number 1132935-63-7.

In embodiments, the NS5 polymerase inhibitor is deleobuvir (also referred to herein as (2E)-3-(2-{1-[2-(5-Bromopyrimidin-2-yl)-3-cyclopentyl-1-methyl-1H-indole-6-carboxamido]cyclobutyl 1-1-methyl-1H-benzimidazol-6-yl)prop-2-enoic acid), CAS Number 863884-77-9.

In embodiments, the NS5 polymerase inhibitor is filibuvir (also referred to herein as (2R)-2-cyclopentyl-2-[2-(2,6-diethylpyridin-4-yl)ethyl]-5-[(5,7-dimethyl-[1,2,4]triazolo[1,5-a]pyrimidin-2-yl)methyl]-4-hydroxy-3H-pyran-6-one), CAS Number 877130-28-4.

In embodiments, the NS5 polymerase inhibitor is setrobuvir (also referred to herein as N-(3-1(4aR,5S,8R,8aS)-1-[(4-fluorophenyl)methyl]-4-hydroxy-2-oxo-1,2,4a,5,6,7,8,8a-octahydro-5,8-methanoquinolin-3-yl}-1,1-dioxo-1,4-dihydro-1λ⁶,2,4-benzothiadiazin-7-yl)methanesulfonamide), CAS Number 1071517-39-9.

In embodiments, the NS5 polymerase inhibitor is radalbuvir (also referred to herein as 5-(3,3-Dimethylbut-1-yn-1-yl)-3-{(1R)—N-[(1s,4s)-4-hydroxy-4-(1[(3S)-oxolan-3-yl]oxy}methyl)cyclohexyl]-4-methylcyclohex-3-ene-1-carboxamido}thiophene-2-carboxylic acid), CAS Number 1314795-11-3.

In embodiments, the NS5 polymerase inhibitor is sofosbuvir (also referred to herein as Isopropyl (2S)-2-[[[(2R,3R,4R,5R)-5-(2,4-dioxopyrimidin-1-yl)-4-fluoro-3-hydroxy-4-methyl-tetrahydrofuran-2-yl]methoxy-phenoxy-phosphoryl]amino]propanoate), CAS Number 1190307-88-0.

In another aspect, there is provided a method of treating a Zika viral infection. The method comprises administering to a subject in need thereof an effective amount of an HIV protease inhibitor.

In embodiments, the HIV protease inhibitor is amprenavir, atazanavir, darunavir, fosamprenavir, indinavir, lopinavir, nelfinavir, ritonavir, saquinavir, tipranavir, asunaprevir, boceprevir, grazoprevir, paritaprevir, simeprevir, or telaprevir.

In embodiments, the HIV protease inhibitor is amprenavir (also referred to herein as (3S)-oxolan-3-yl N-[(2S,3R)-3-hydroxy-4-[N-(2-methylpropyl)(4-aminobenzene)sulfonamido]-1-phenylbutan-2-yl]carbamate), CAS Number 161814-49-9.

In embodiments, the HIV protease inhibitor is atazanavir (also referred to herein as methyl N-[(1S)-1-{[(2S,3S)-3-hydroxy-4-[(2S)-2-[(methoxycarbonyl)amino]-3,3-dimethyl-N′-1-[4-(pyridin-2-yl)phenyl]methyl}butanehydrazido]-1-phenylbutan-2-yl]carbamoyl}-2,2-dimethylpropyl]carbamate), CAS Number 198904-31-3.

In embodiments, the HIV protease inhibitor is darunavir (also referred to herein as [(1R,5S,6R)-2,8-dioxabicyclo[3.3.0]oct-6-yl]N-[(2S,3R)-4-[(4-aminophenyl)sulfonyl-(2-methylpropyl)amino]-3-hydroxy-1-phenyl-butan-2-yl]carbamate), CAS Number 206361-99-1.

In embodiments, the HIV protease inhibitor is fosamprenavir (also referred to herein as [(1R,3S)-1-[N-(2-methylpropyl)(4-aminobenzene)sulfonamido]-3-(1[(3S)-oxolan-3-yloxy]carbonyl}amino)-4-phenylbutan-2-yl]oxy}phosphonic acid), CAS Number 226700-81-8.

In embodiments, the HIV protease inhibitor is indinavir (also referred to herein as (2S)-1-[(2S,4R)-4-benzyl-2-hydroxy-4-1[(1S,2R)-2-hydroxy-2,3-dihydro-1H-inden-1-yl]carbamoyl}butyl]-N-tert-butyl-4-(pyridin-3-ylmethyl)piperazine-2-carboxamide), CAS Number 150378-17-9.

In embodiments, the HIV protease inhibitor is lopinavir (also referred to herein as (2S)—N-[(2S,4S,5S)-5-[2-(2,6-dimethylphenoxy)acetamido]-4-hydroxy-1,6-diphenylhexan-2-yl]-3-methyl-2-(2-oxo-1,3-diazinan-1-yl)butanamide), CAS Number 192725-17-0.

In embodiments, the HIV protease inhibitor is nelfinavir (also referred to herein as (3S,4aS,8aS)—N-tert-butyl-2-[(2R,3R)-2-hydroxy-3-[(3-hydroxy-2-methylphenyl)formamido]-4-(phenylsulfanyl)butyl]-decahydroisoquinoline-3-carboxamide), CAS Number 159989-64-7.

In embodiments, the HIV protease inhibitor is ritonavir (also referred to herein as 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate), CAS Number 155213-67-5.

In embodiments, the HIV protease inhibitor is saquinavir (also referred to herein as (2S)—N-[(2S,3R)-4-[(3S)-3-(tert-butylcarbamoyl)-decahydroisoquinolin-2-yl]-3-hydroxy-1-phenylbutan-2-yl]-2-(quinolin-2-ylformamido)butanediamide), CAS Number 127779-20-8.

In embodiments, the HIV protease inhibitor is tipranavir (also referred to herein as N-{3-[(1R)-1-[(2R)-6-hydroxy-4-oxo-2-(2-phenylethyl)-2-propyl-3,4-dihydro-2H-pyran-5-yl]propyl]phenyl}-5-(trifluoromethyl)pyridine-2-sulfonamide), CAS Number 17-44-84-41-4.

In embodiments, the HIV protease inhibitor is asunaprevir (also referred to herein as 3-Methyl-N-{[(2-methyl-2-propanyl)oxy]carbonyl}-L-valyl-(4R)-4-[(7-chloro-4-methoxy-1-isoquinolinyl)oxy]-N-{(1R,2S)-1-[(cyclopropylsulfonyl)carbamoyl]-2-vinylcyclopropyl}-L-prolinamide), CAS Number 630420-16-5.

In embodiments, the HIV protease inhibitor is boceprevir (also referred to herein as (1R,5S)—N-[3-Amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexane-2(S)-carboxamide), CAS Number 394730-60-0.

In embodiments, the HIV protease inhibitor is grazoprevir (also referred to herein as (1R,18R,20R,24S,27S)—N-1(1R,2S)-1-[(cyclopropylsulfonyl)carbamoyl]-2-vinylcyclopropyl}-7-methoxy-24-(2-methyl-2-propanyl)-22,25-dioxo-2,21-dioxa-4,11,23,26-tetraazapentacyclo[24.2.1.03,12.05,10.0 18,20]nonacosa-3,5,7,9,11-pentaene-27-carboxamide), CAS Number 1350514-68-9.

In embodiments, the HIV protease inhibitor is paritaprevir (also referred to herein as (2R,6S,12Z,13 aS,14aR,16aS)—N-(Cyclopropylsulfonyl)-6-1[(5-methyl-2-pyrazinyl)carbonyl]amino}-5,16-dioxo-2-(6-phenanthridinyloxy)-1,2,3,6,7,8,9,10,11,13a,14,15,16,16a-tetradecahydrocyclopropa[e]pyrrolo[1,2-a][1,4]diazacyclopentadecine-14a(5H)-carboxamide), CAS Number 1216941-48-8.

In embodiments, the HIV protease inhibitor is simeprevir (also referred to herein as (2R,3aR,10Z,11aS,12aR,14aR)—N-(Cyclopropylsulfonyl)-2-{[2-(4-isopropyl-1,3-thiazol-2-yl)-7-methoxy-8-methyl-4-quinolinyl]oxy}-5-methyl-4,14-dioxo-2,3,3a,4,5,6,7,8,9,11a,12,13,14,14a-tetradecahydrocyclopenta[c]cyclopropa[g][1,6]diazacyclotetradecine-12a(1H)-carboxamide), CAS Number 923604-59-5.

In embodiments, the HIV protease inhibitor is telaprevir (also referred to herein as (1S,3aR,6aS)-2-[(2S)-2-[[(2S)-2-Cyclohexyl-2-(pyrazine-2-carbonylamino)acetyl]amino]-3,3-dimethylbutanoyl]-N-[(3S)-1-(cyclopropylamino)-1,2-dioxohexan-3-yl]-3,3a,4,5,6,6a-hexahydro-1H-cyclopenta[c]pyrrole-1-carboxamide), CAS Number 402957-28-2.

In another aspect, there is provided a method of treating a Zika viral infection. The method comprises administering to a subject in need thereof an effective amount of a protein or a gene encoding the protein.

In embodiments, the protein is a ZIKV non-structural (NS) protein. In embodiments, the ZIKV non-structural protein is NS5. In embodiments, the protein is NS5 RNA polymerase. In embodiment, the ZIKV non-structural protein is NS2B-NS3. In embodiments, the protein is NS2B-NS3 protease.

In another aspect, there is provided a method of treating a Zika viral infection. The method includes administering to a subject in need thereof an effective amount of a calcium channel blocker.

In embodiments, the calcium channel blocker is manidipine, cilnidipine, or benidipine.

In another aspect, there is provided a method of treating a Zika viral infection. The method includes administering to a subject in need thereof an effective amount of a combination therapeutic composition including an NS5 polymerase inhibitor and a HIV protease inhibitor. The term “combination therapeutic composition” or the like refers, in the usual and customary sense, to administration of a plurality of pharmaceutically acceptable compounds or agents, each optionally including a pharmaceutically acceptable excipient. The plurality of pharmaceutically acceptable compounds or agents can be administered in a single dosage. The plurality of pharmaceutically acceptable compounds or agents can be administered in a multi-dose regimen. The plurality of pharmaceutically acceptable compounds or agents can be co-administered with each other.

In embodiments, the combination therapeutic composition includes Suramin, Ganciclovir, Procaine hydrochloride, Zidovudine, Acyclovir, Drostanolone Propionate, Dapivirine (TMC120), Tilorone hydrochloride, Docosanol, clomiphene, amphotericin B, toremifene, mycophenolic acid, fluoxetine, niclosamide, and/or polyhydroxyalkanoates (PHA).

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1. Screening Studies

As shown in FIG. 1A, antiviral activity of approved drugs/clinical molecules library was tested against Zika virus in human 293 T cells at the MOI of 5. Cells were pretreated with drugs (10 uM) for 1 hour and infected with ZIKV (MOI=5). After 24 hours of infection, mRNA was isolated and the levels of cellular ZIKV was determined by qRT-PCR.

As shown in FIG. 1B, methyl transferase inhibitors potently reduced ZIKV replication. Methods were as described in FIG. 1A.

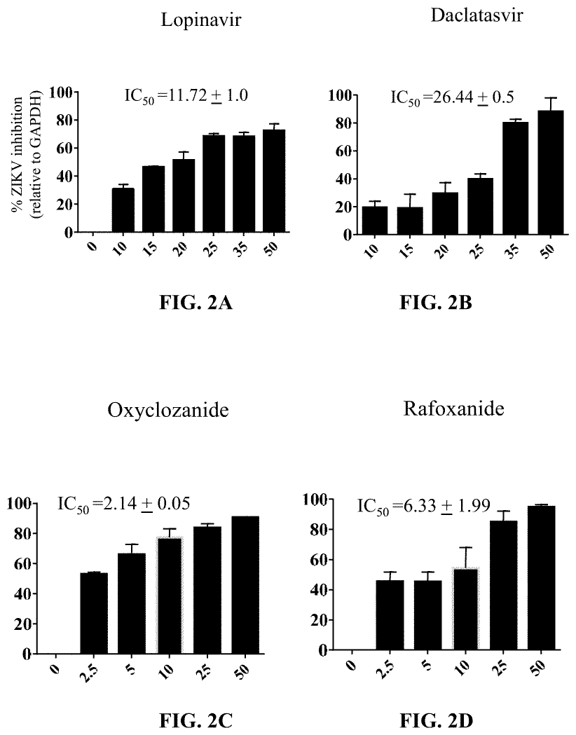

As shown in FIGS. 2A-2D, there is observed a dose dependent inhibition of selected antiviral molecules. Conditions: Zika virus in 293 T cells at the MOI of 5 using different concentration of drugs. IC₅₀ was calculated.

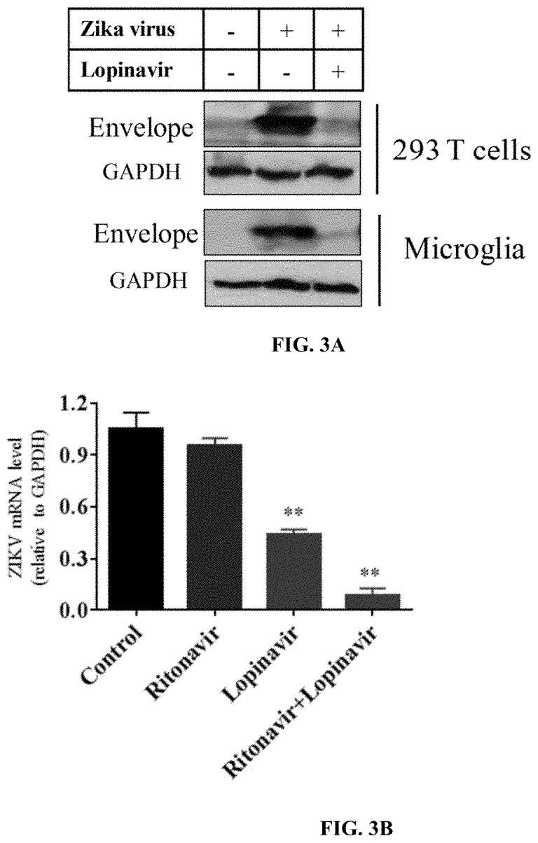

As shown in FIGS. 3A-3B, there is observed synergistic activity of lopinavir and ritonavir. 293 T cells were pretreated with indicated drugs and after 1 hour, the cells were infected with ZIKV at the MOI of 5. After 24 hours, proteins were analyzed by western blotting (FIG. 3A) and mRNA was isolated and the levels of cellular ZIKV was determined by qRT-PCR (FIG. 3B).

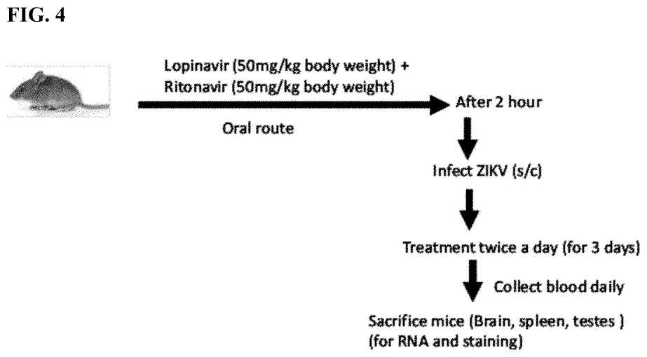

FIG. 4 outlines an experimental protocol to determine the drug efficacy for ZIKV inhibition in vivo.

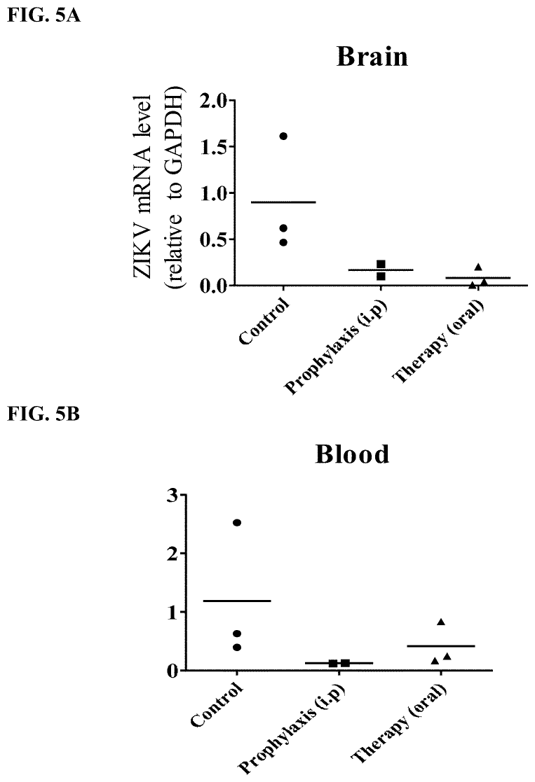

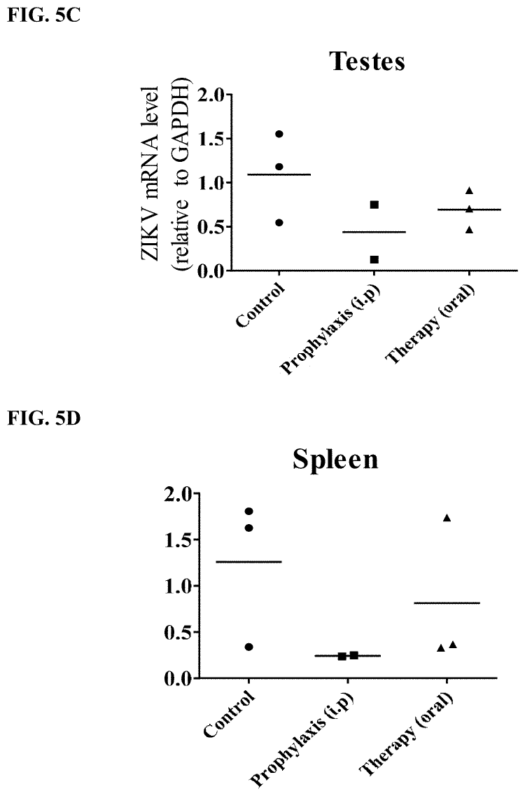

FIGS. 5A-5D depict results demonstrating ZIKV protease inhibition in animal tissues (i.e., brain, blood, testes, spleen), in prophylaxis (i.p) and therapeutic (oral) regimens.

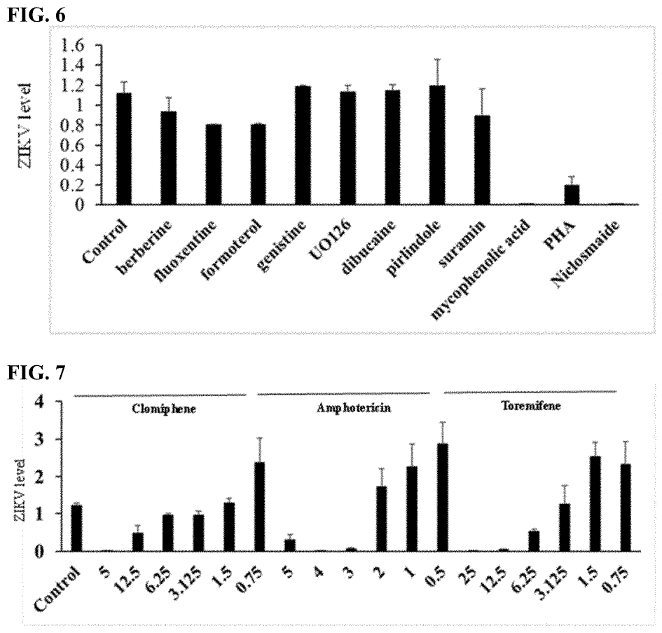

FIG. 6 depicts ZIKV levels as determined by qRT-PCR for ZIKA in Vero cells at 10 μM concentration (left to right) of control, berberine, fluoxentine, formoterol, genistine, U0126, dibucaine, pirlindole, suramin, mycophenolic acid, PHA, and niclosamide.

FIG. 7 depicts dose response studies on ZIKV levels for clomiphene, amphotericin B, and toremifene.

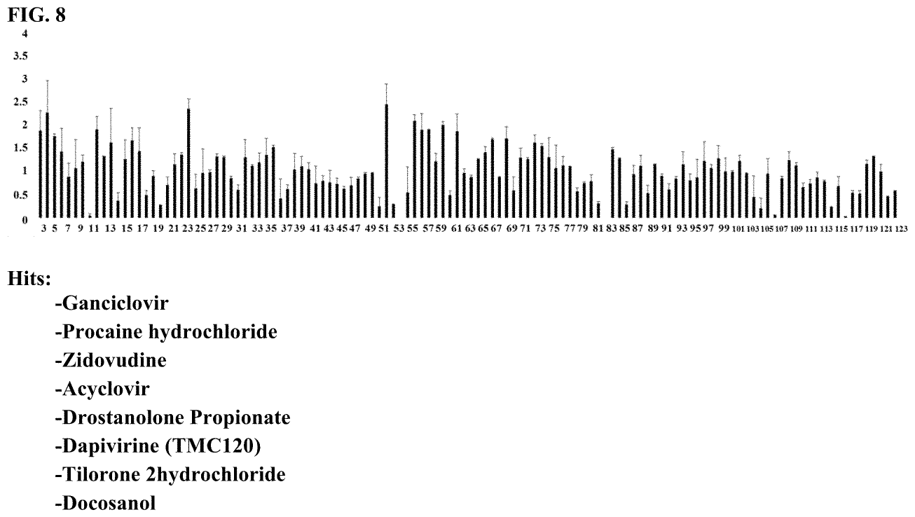

FIG. 8 depicts ZIKV levels after administration of various agents. The term “Hits” refers to agent which reduce ZIKV load, including Ganciclovir, Procaine hydrochloride. Zidovudine. Acyclovir. Drostanolone Propionate, Dapivirine (TMC120), Tilorone hydrochloride, and Docosanol.

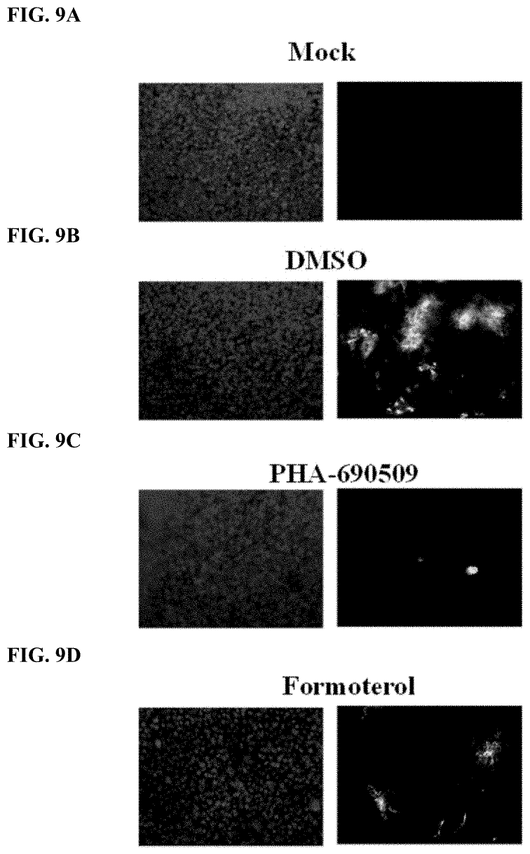

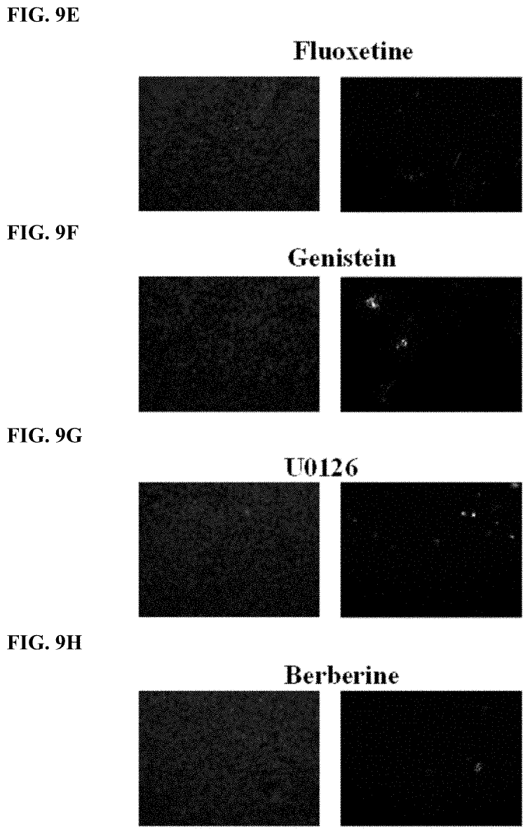

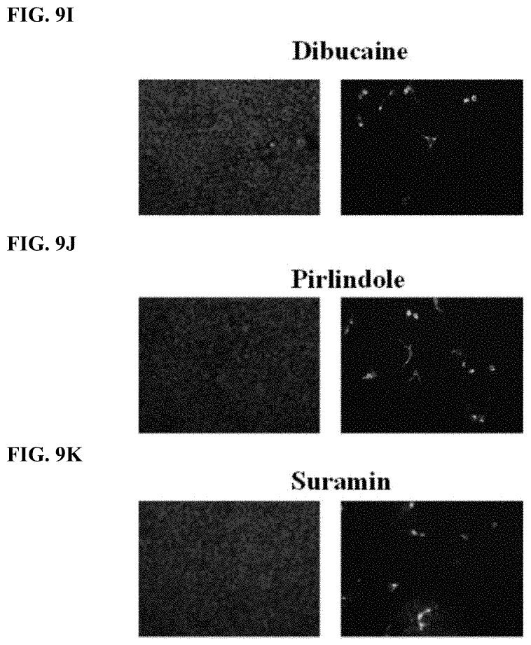

FIGS. 9A-9K depict ZIKA inhibition by various drugs as visualized by immunostaining. Agents in order (top to bottom): mock, DMSO, PHA-690509, formoterol, fluoxetine, genistein, U0126, berberine, dibucaine, pirlindole, and suramin.

### Example 2. Zika Virus Infection Reprograms Global Transcription of Host Cells to Allow Sustained Infection

Abstract. Zika virus (ZIKV) is an emerging virus causally linked to neurological disorders, including congenital microcephaly and Guillain-Barré syndrome. There are currently no targeted therapies for ZIKV infection. To identify novel antiviral targets and to elucidate the mechanisms by which ZIKV exploits the host cell machinery to support sustained replication, we analyzed the transcriptomic landscape of human microglia, fibroblast, embryonic kidney, and monocyte-derived macrophage cell lines before and after ZIKV infection. The four cell types differed in their susceptibility to ZIKV infection, consistent with differences in their expression of viral response genes before infection. Clustering and network analyses of genes differentially expressed after ZIKV infection revealed changes related to the adaptive immune system, angiogenesis, and host metabolic processes that are conducive to sustained viral production. Genes related to the adaptive immune response were downregulated in microglia cells, suggesting that ZIKV effectively evades the immune response after reaching the central nervous system. Like other viruses, ZIKV diverts host cell resources and reprograms the metabolic machinery to support RNA metabolism, ATP production, and glycolysis. Consistent with these transcriptomic analyses, nucleoside metabolic inhibitors abrogated ZIKV replication in microglia cells.

Introduction

Zika virus (ZIKV) is an emerging arbovirus of the Flaviviridae family [1,2], which includes West Nile (WNV), yellow fever, Chikungunya, dengue, and Japanese encephalitis viruses [2]. These viruses cause mosquito-borne diseases transmitted by the Aedes genus [2]. ZIKV may also be transmitted sexually and vertically [3,4]. ZIKV was first discovered more than 60 years ago in samples taken from a sentinel rhesus monkey in the Zika forest of Uganda, and has since been isolated from mosquitoes and humans [5,6]. Various epidemiological studies have revealed a worldwide spread of ZIKV to geographic areas ranging from Asia and the Pacific to, most recently, the Americas [1]. The rapid spread of ZIKV from Asia to the Americas has affected more than 30 countries. Due to its sporadic nature and mild symptoms, ZIKV infection was initially ignored. Approximately 80% of ZIKV infections are asymptomatic, and the most common symptoms include fever, arthralgia, rash, myalgia, edema, vomiting, and non-purulent conjunctivitis [7]. However, ZIKV infection in pregnant women has been linked to the increasing incidence of congenital microcephaly and other disorders such as placental insufficiency, fetal growth retardation, and fetal death. Emerging evidence suggests that ZIKV causes mild symptoms in non-pregnant individuals, but it has also been associated with neurological abnormalities and Guillain-Barré syndrome [8-11].

Female Aedes mosquitoes act as vectors to transmit ZIKV through the skin of the mammalian host, which is followed by infection of permissive cells through specific receptors. Current reports indicate that dermal fibroblasts, dendritic cells, neural progenitor cells, and epidermal keratinocytes are permissive to ZIKV infection while placental trophoblasts are resistant due to constitutive release of type III interferon [12-16]. Interferon knockout mouse models have also shown susceptibility to ZIKV infection [17-19]. However, the pathogenesis of ZIKV infection remains poorly understood. In this study, we analyzed transcriptomic changes induced by ZIKV infection in four human cell lines (microglia, fibroblast, macrophage, and human embryonic kidney cells) to identify genes that could be developed as potential therapeutic targets and to provide insight into the interaction between ZIKV and the host cell.

Results.

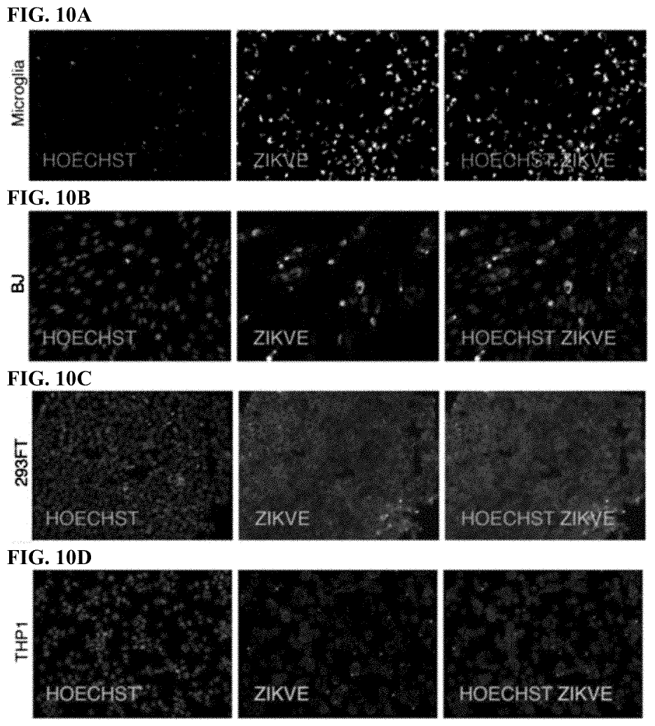

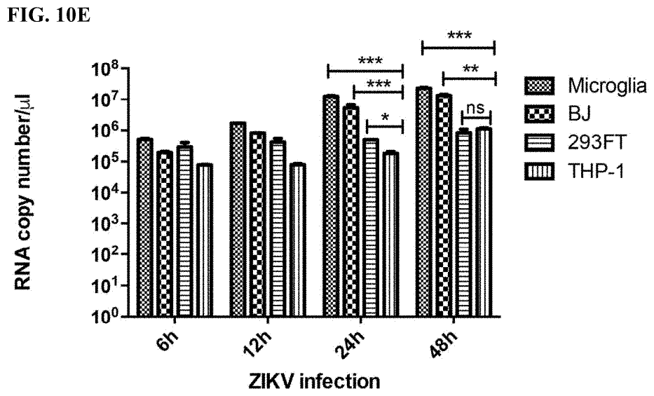

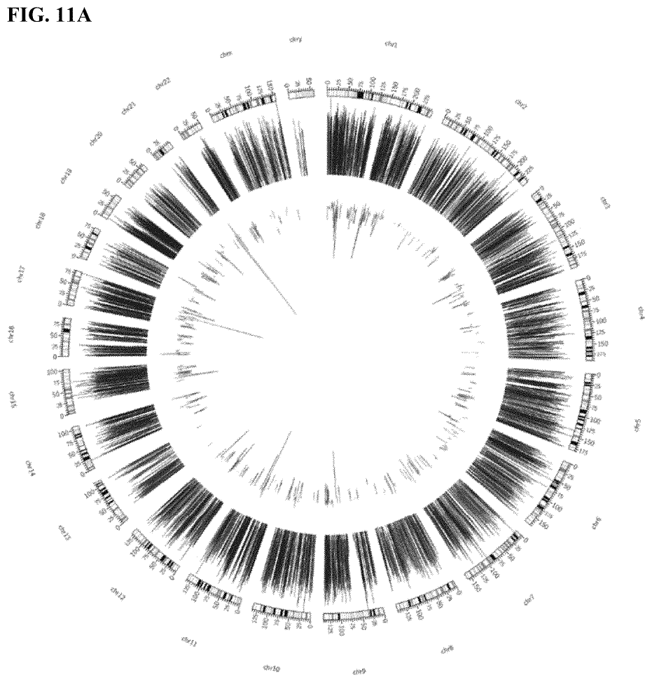

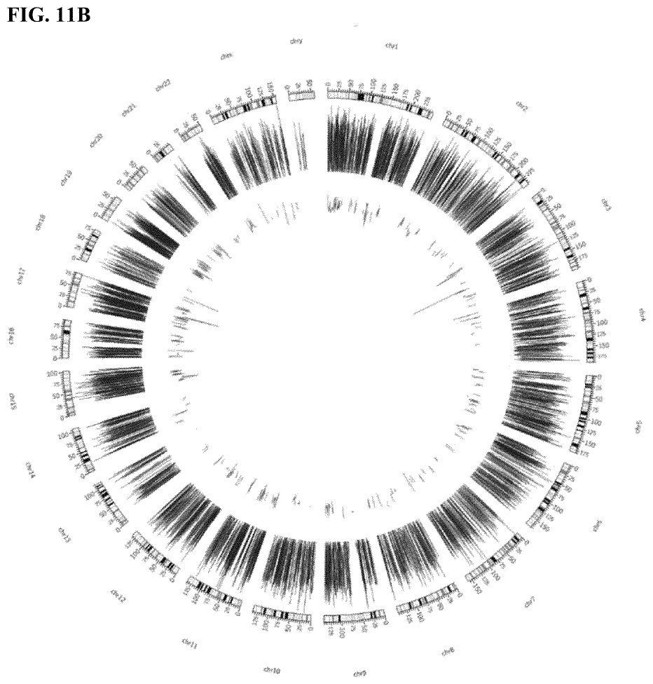

Cell Type-Specific ZIKV Replication and Infection. To analyze factors contributing to ZIKV pathogenesis, we selected four human cell lines, microglia, BJ (foreskin fibroblast), 293FT (embryonic kidney), and THP-1 derived macrophages (monocyte-derived macrophage), and inoculated them with ZIKV produced in Vero and BHK cells at a multiplicity of infection of 1 (FIGS. S1A-S1E). The choice of cell lines was driven by a desire to understand different aspects of ZIKV pathogenesis. Microglia cells model the resident macrophages in the brain and provide information on how they may contribute to neuroinflammation and other ZIKV-associated neurodegenerative disorders, such as Guillain-Barré syndrome. BJ cells were selected to model dermal infection, the primary route of mosquito-driven ZIKV infection. The 293FT human embryonic kidney cell line is well known for robust lentivirus production and may provide an ideal host for ZIKV replication as well. Lastly, THP-1 macrophages provide a model of the effect of ZIKV infection on a host cell critical for the immune response. ZIKV expression was assessed by immunofluorescent staining of cells with an antibody against the ZIKVE flavivirus envelope protein at 24 h post-infection (hpi). ZIKV infection was most marked in microglia cells, followed by BJ, 293FT, and THP-1 derived macrophages (FIGS. 10A-10D). These findings were confirmed by one-step RT-qPCR of viral RNA in the cell supernatants, which showed statistically significant differences in the levels of viral transcripts between microglia and THP-1 derived macrophages at 24 and 48 hpi (FIG. 10E). This marked difference in ZIKV susceptibility is notable because both cell types are macrophages; microglia cells are microglial and THP-1 derived macrophages are monocyte-derived.

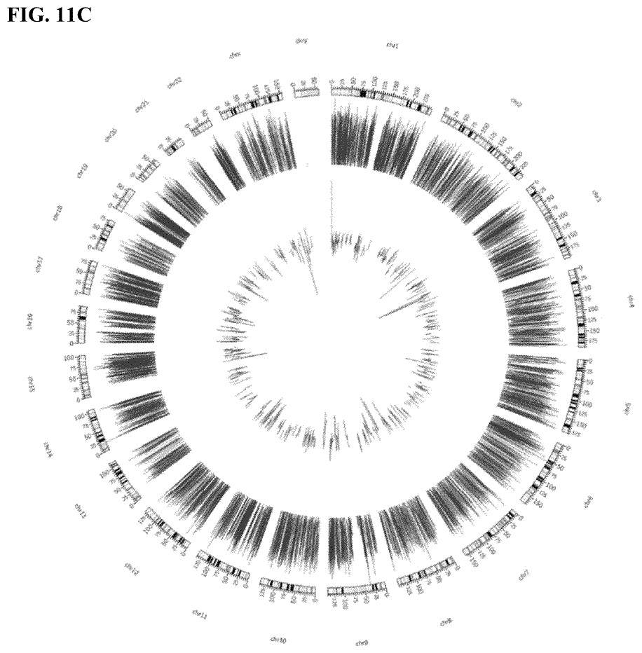

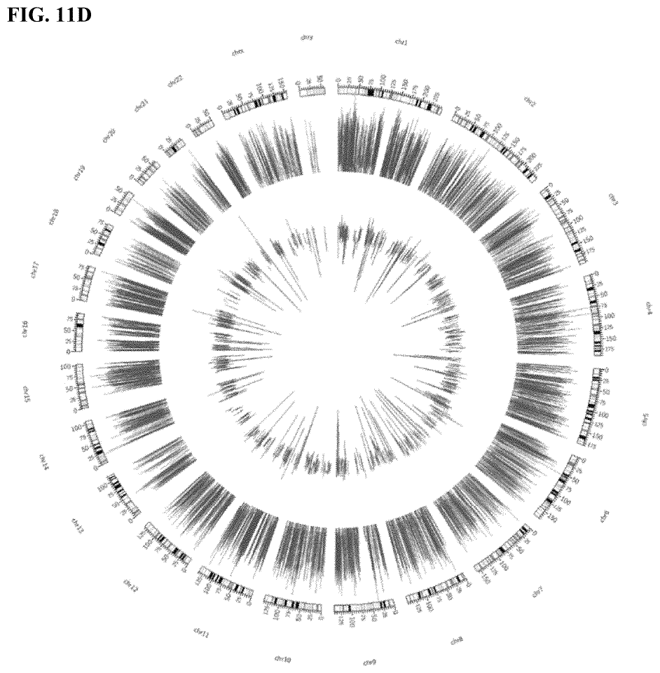

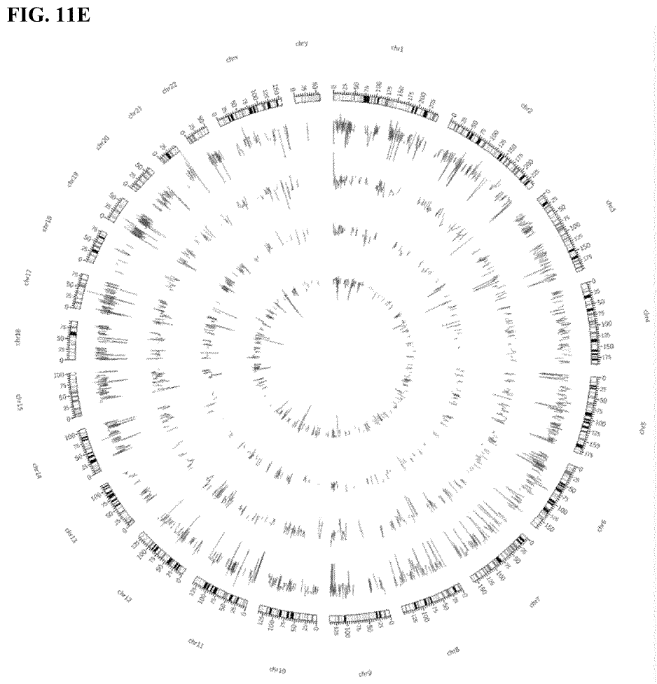

We next analyzed changes in the transcriptomic landscapes of the four cell types after ZIKV infection to identify potential key regulators responsible for the cell type-specific differences in ZIKV replication. For this, total RNA was extracted from mock- and ZIKV-infected cell lines at 24 hpi and analyzed by Illumina NextSeq 500. We elected to examine gene expression at 24 hpi to assess the effects of ZIKV infection immediately following the early innate immune response (FIGS. S1E-S1E). FIGS. 11A-11E show the data presented in Circos plots, in which the fold change for differentially expressed genes (shown in inner circles) are plotted in contrast with the overall gene expression level (in outer circles) for microglia, BJ, 293FT and THP-1 derived macrophages (FIGS. 11A-11D, respectively). Interestingly, the level of ZIKV expression and the impact of ZIKV infection on the transcriptome were inversely correlated. Thus, microglia cells showed the fewest changes in gene expression following infection (FIG. 11A), followed by BJ (FIG. 11B), 293FT (FIG. 11C), and THP-1 derived macrophages (FIG. 11D). Furthermore, the magnitude of the gene expression changes also differed between the cell types, with the differentially expressed genes in THP-1 derived macrophages displaying the biggest differences between mock- and ZIKV-infected cells (FIG. 11E).

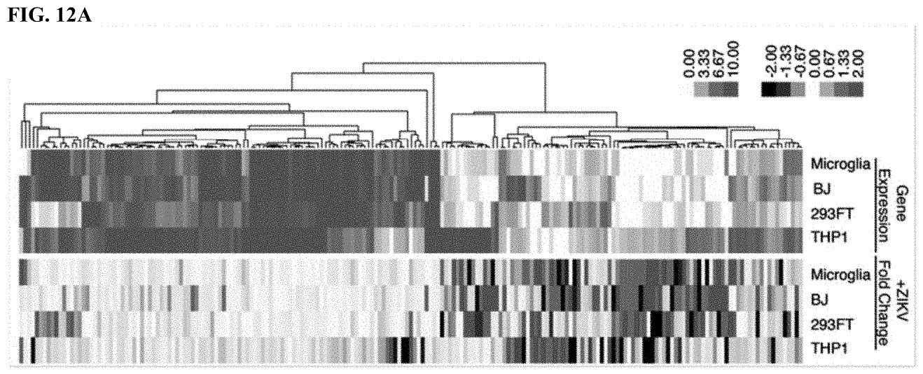

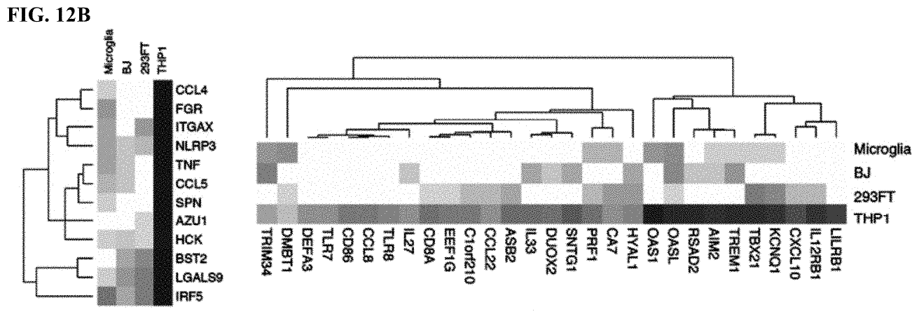

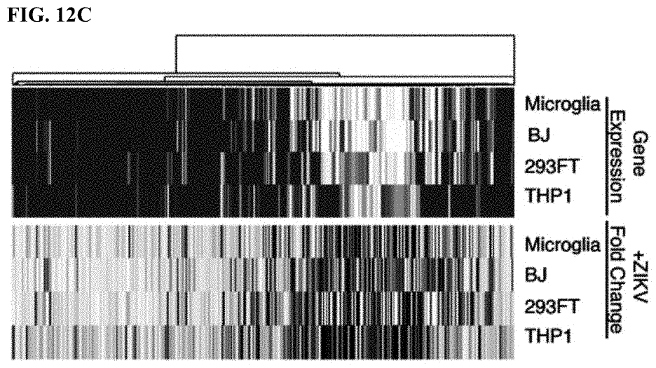

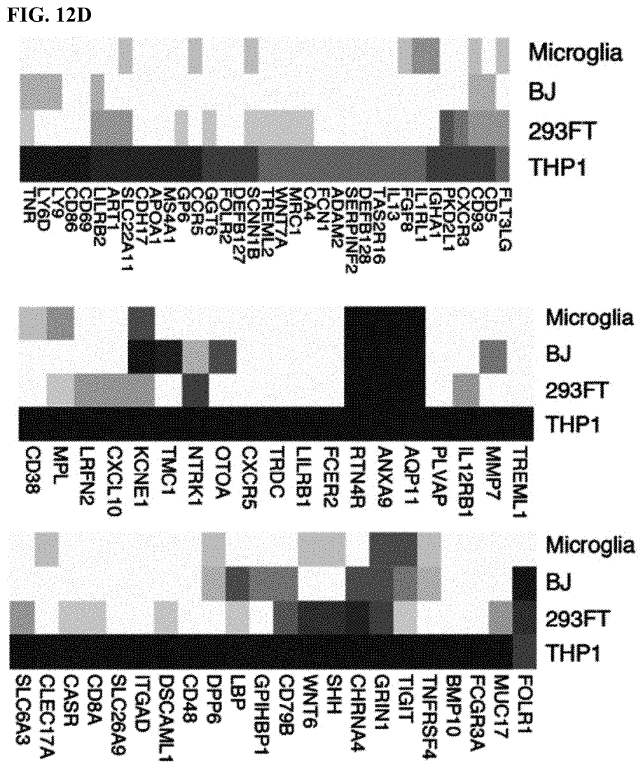

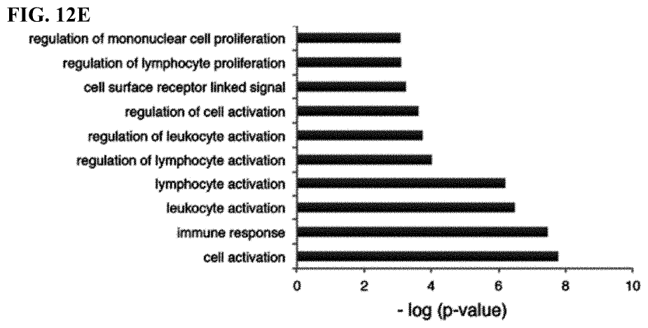

Analysis of Viral Response Genes Identifies Potential Cell Type-Specific Regulators. To identify genes that may account for the different levels of ZIKV expression in the four cell lines, we analyzed the expression of endogenous viral response genes before infection. We hypothesized that the initial ZIKV viral entry and replication mechanisms might be dependent on the expression levels of specific host genes. Thus, genes from the human GRCh38.p5 database in Ensembl associated with the following gene ontology terms were analyzed in the mock-infected cells: “response to virus,” “modulation of virus host gene expression,” “viral transcription,” and “viral release from host cell” (FIGS. 12A-12B, FIGS. S1A-S2D, and Table 1).

Consistent with our hypothesis, THP-1 derived macrophages, which were the most resistant to ZIKV infection, expressed the greatest number of viral response genes and immune-related genes, while microglia cells express the fewest (FIGS. S2E-S2F, Table 3). Genes that were uniquely or highly expressed in THP-1 derived macrophages but showed low expression in microglia were analyzed as potential antiviral ZIKV factors. Interestingly, expression of immune regulatory molecules, including CCL3, CCL4, CCL5, TNF, IRF5, CXCL10, OAS1, TLR7, TLR8, and IL27 was highest in THP-1 derived macrophages, indicating that they are primed to mount a vigorous defense as part of the initial innate immune response to ZIKV and to elicit greater transcriptional changes in response to ZIKV infection (FIGS. 11E and 12B). Comparison of genes differentially expressed in the four cell types before infection revealed high expression of CD4 in THP-1 derived macrophages and low expression of CHMP4C (charged multivesicular body protein 4C). CHMP4C is a component of the ESCRT-III family responsible for surface receptor degradation and viral budding [20]. Further mechanistic studies will be necessary to determine which of these viral response and immune-related genes might be vital for regulating ZIKV expression.

We also examined the differential expression of cell surface receptor genes in the four cell types prior to ZIKV infection to identify potential antiviral receptors that may confer greater ZIKV resistance on THP-1 derived macrophages. Heat map clustering of cell surface receptor genes identified several genes that were expressed highly in THP-1 compared with the other cell types, including CD86, LY6D, CXCL10, CD48, and IL12RB1 (FIGS. 12C-12D, Table 2). Gene ontology analysis of these and other genes showing selectively high expression in THP-1 derived macrophages revealed an enrichment of genes involved in cell activation, immune response, and cell receptor signaling (FIG. 12E). These analyses of the steady-state expression of endogenous genes in the four cell lines before ZIKV infection have identified a number antiviral genes highly expressed in THP-1 derived macrophages, suggesting they could be exploited to therapeutic effect.

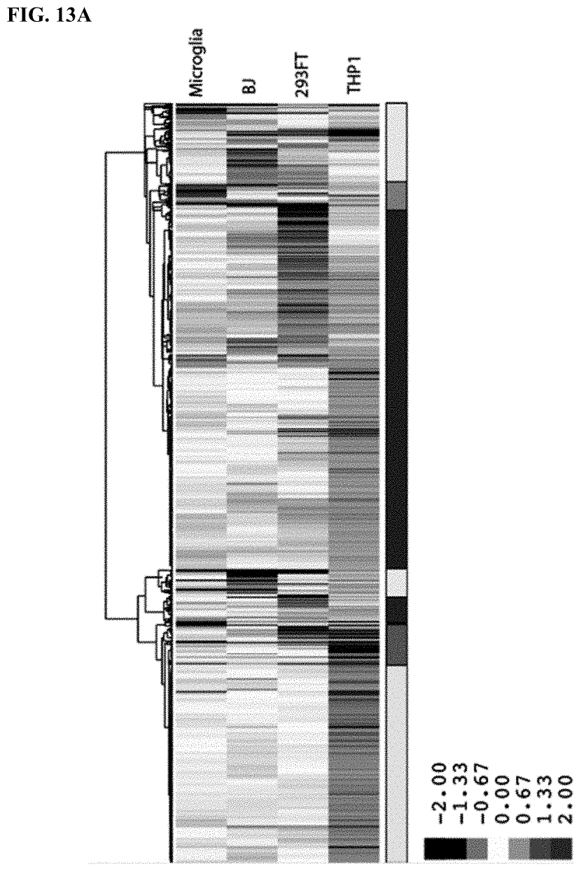

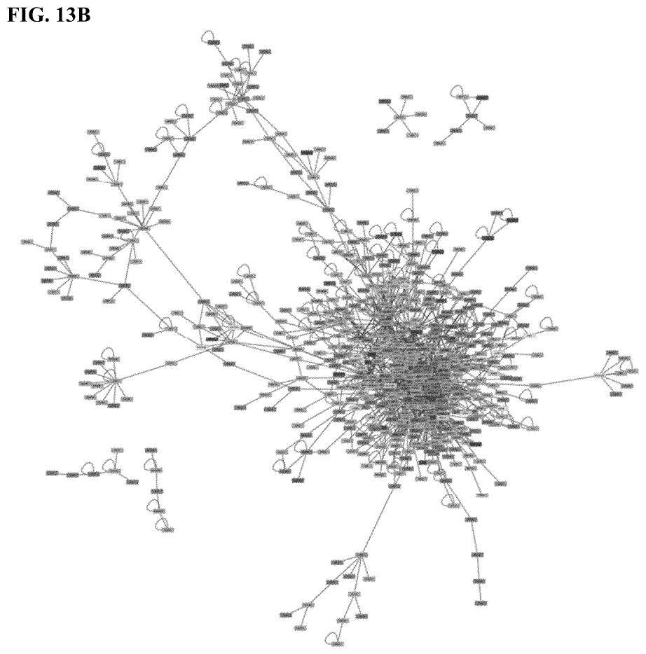

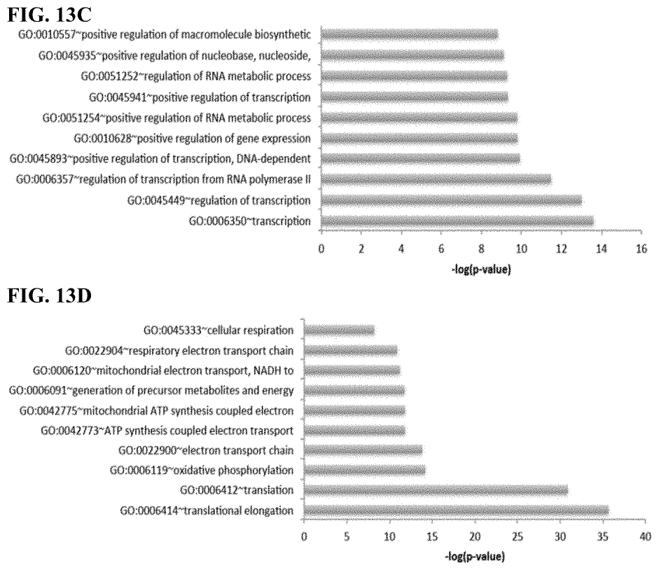

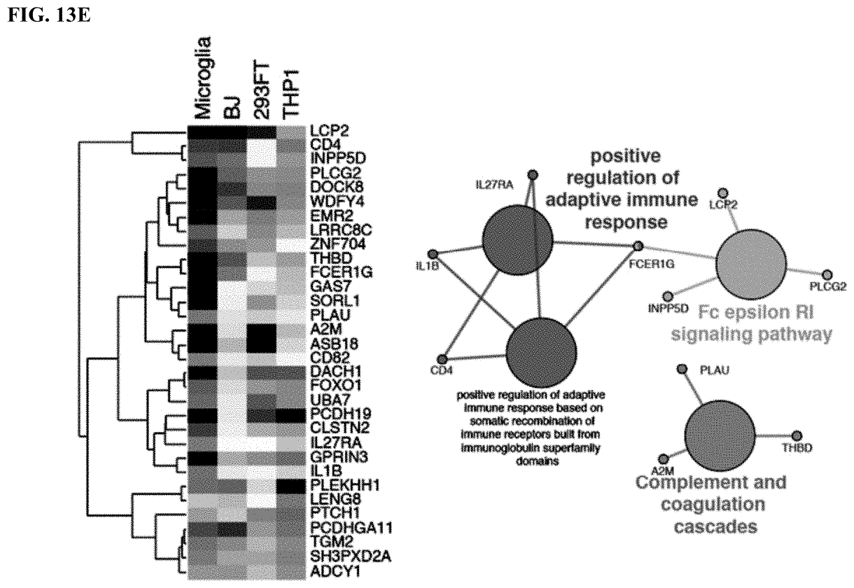

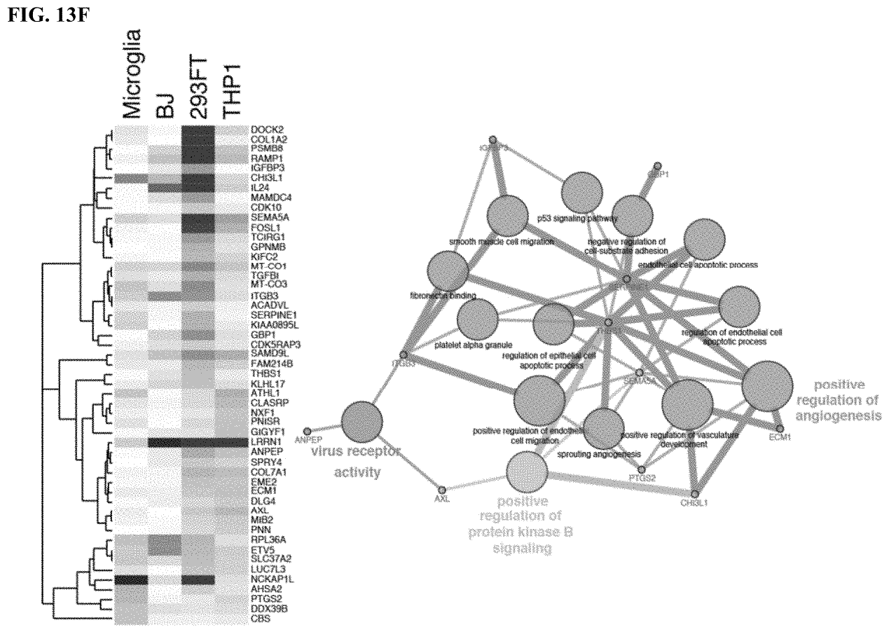

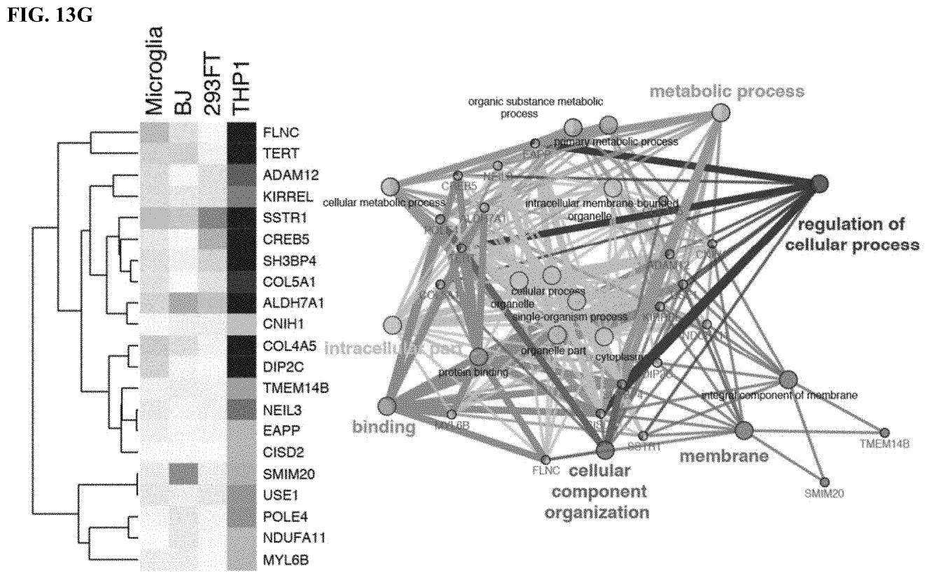

ZIKV Infection Modulates the Metabolic and Transcriptional Landscape. To determine the effects of ZIKV infection on the host transcriptome, we analyzed differentially expressed genes between mock- and ZIKV-infected cell lines at 24 hpi. As mentioned above, the total number of differentially expressed genes in ZIKV-infected cells was inversely correlated with the level of ZIKV infection, with the least and most marked changes occurring in CHME-1 and THP-1 derived macrophages, respectively (FIGS. 10A-10E and 11E). In-depth analysis of the differentially expressed genes revealed that ZIKV infection elicited a similar pattern of gene expression changes between BJ and 293FT cells and between microglia and THP-1 derived macrophages, both macrophage cell types; however, the fold change in expression is much greater in THP-1 derived macrophages (FIG. 13A, Table 4). Predictive network analyses suggested that a significant portion of the differentially expressed genes following ZIKV infection interact to regulate common pathways such as including mRNA processing, metabolic processes, RNA processing, and cellular component disassembly (FIG. 13B and FIGS. S3A-S3D).

Further clustering analysis highlighted the distinct patterns of gene expression changes in the ZIKV-infected cells (right vertical bar in FIG. 13A). Analysis of genes that were predominantly upregulated in microglia and THP-1 macrophages and downregulated in BJ and 293FT cells (red on the vertical bar in FIG. 13A) indicated that they are associated with transcriptional regulators, RNA metabolic processes, and macromolecule biosynthesis (FIG. 13C). Genes upregulated in THP-1 macrophages and downregulated in all other cell lines (green bar) included IL1B, CD4, IL27RA, and FCER1G which act as positive regulators of the adaptive immune response, Fc epsilon RI signaling pathway and complement and coagulation cascades (FIGS. 13A and 13E). Interestingly, these genes are downregulated in microglia, BJ, and 293FT. These findings help to explain why THP-1 derived macrophages, which have the highest expression of innate immune genes in the uninfected state and the greatest upregulation of adaptive immune response genes post-infection, have the lowest level of ZIKV expression.

The cluster of genes downregulated by ZIKV infection in microglia and BJ but upregulated in 293FT and THP-1 derived macrophages (bar, FIG. 13A) included several key genes that regulate virus receptor activity, protein kinase B activity, and angiogenesis (FIG. 13F). A notable gene in this cluster is the viral entry receptor AXL, which has previously been associated with ZIKV pathogenesis [12,21]. In contrast, the gene cluster downregulated in the macrophage lines (bar in FIG. 13A), and particularly in THP-1 derived macrophages, are strongly associated with translational elongation and various cellular metabolic processes (FIG. 13D). Finally, genes differentially expressed in ZIKV-infected microglia and THP-1 derived macrophages (bar in FIG. 13A) were related to metabolic processes (FIG. 13G). Viruses are obligate parasites that exploit the host's metabolic processes to reproduce [22-24]. Understanding the mechanisms by which ZIKV alters the host cell metabolism may thus provide additional novel therapeutic targets. Further examination of the roles of metabolic control genes such as TERT, ALDH7A1, CREB5, EAPP, and NDUFA11 as they relate to ZIKV infection may provide further insights into ZIKV pathogenesis.

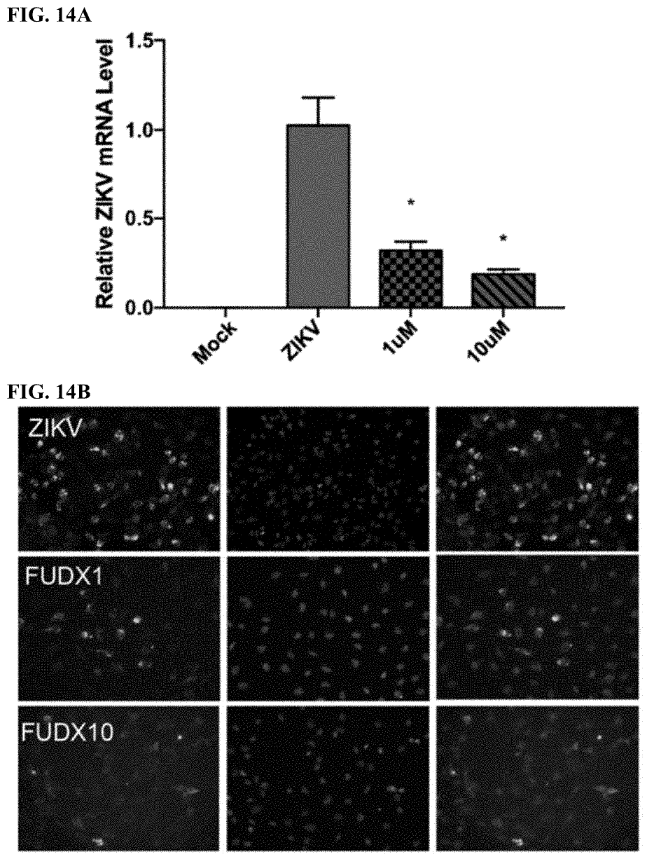

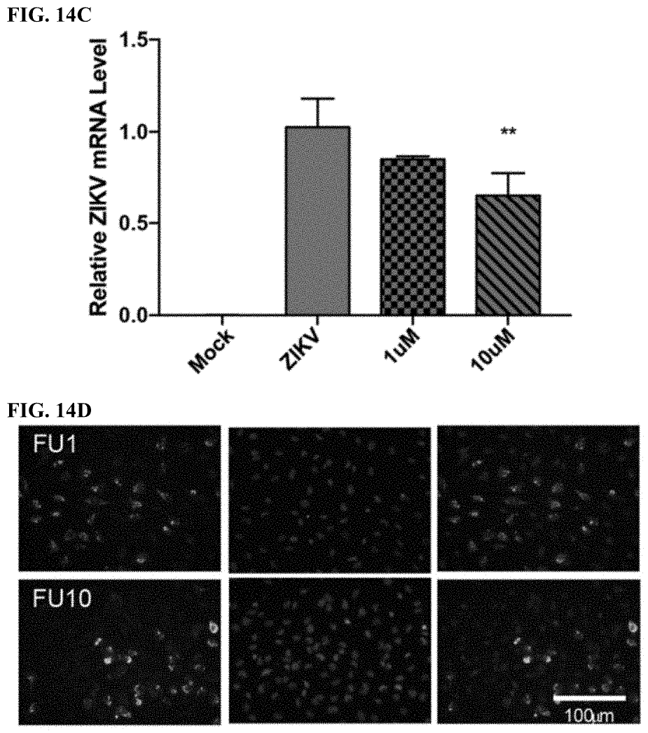

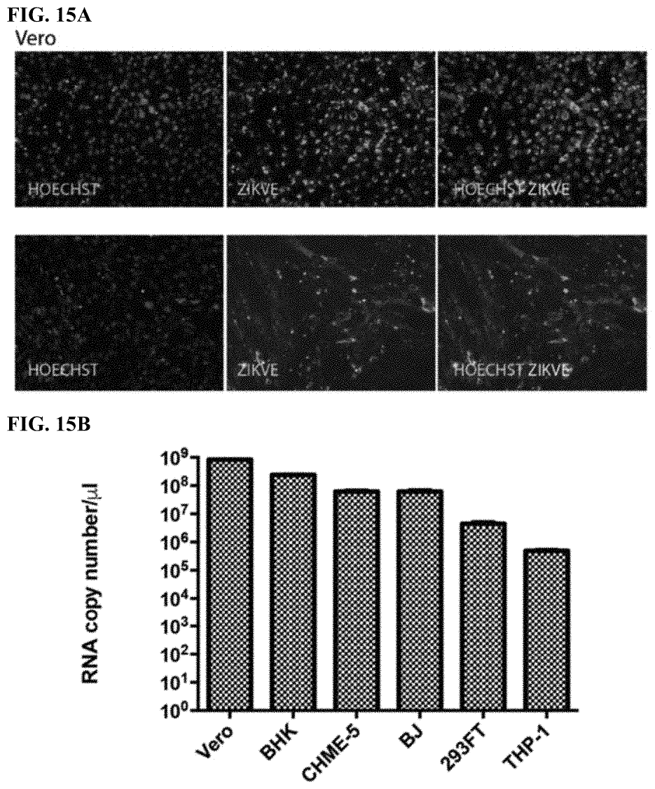

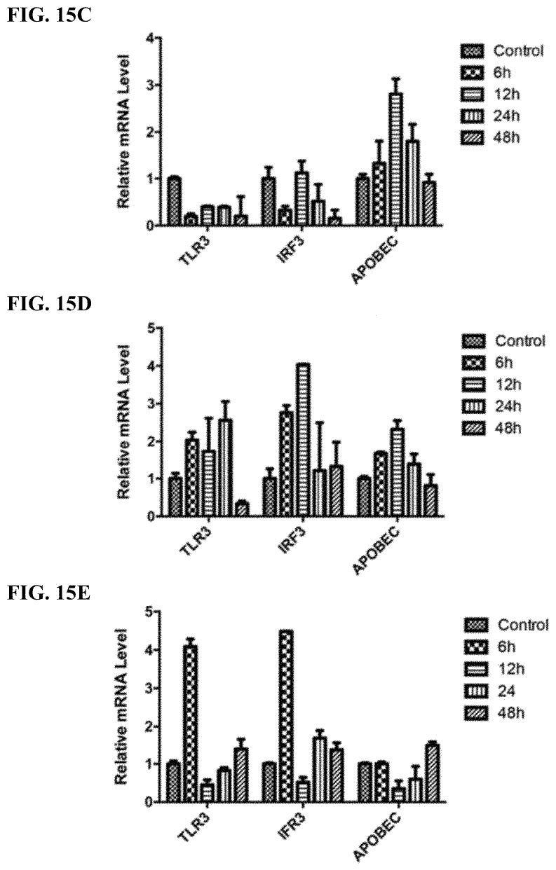

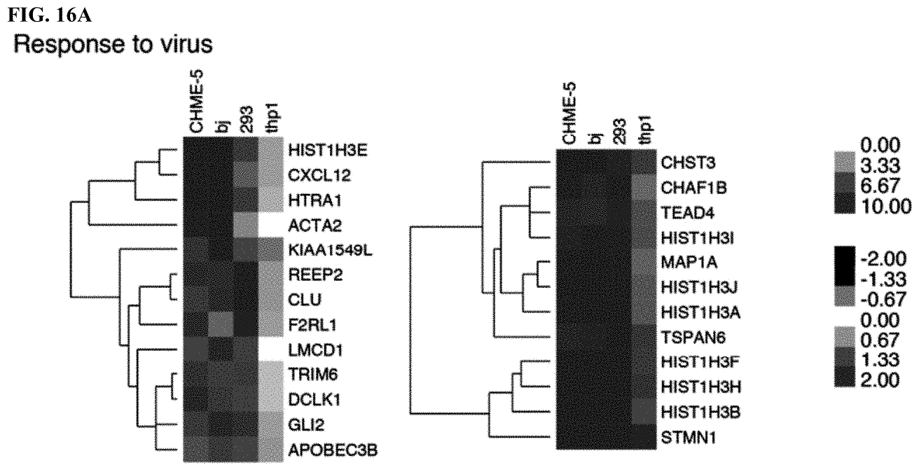

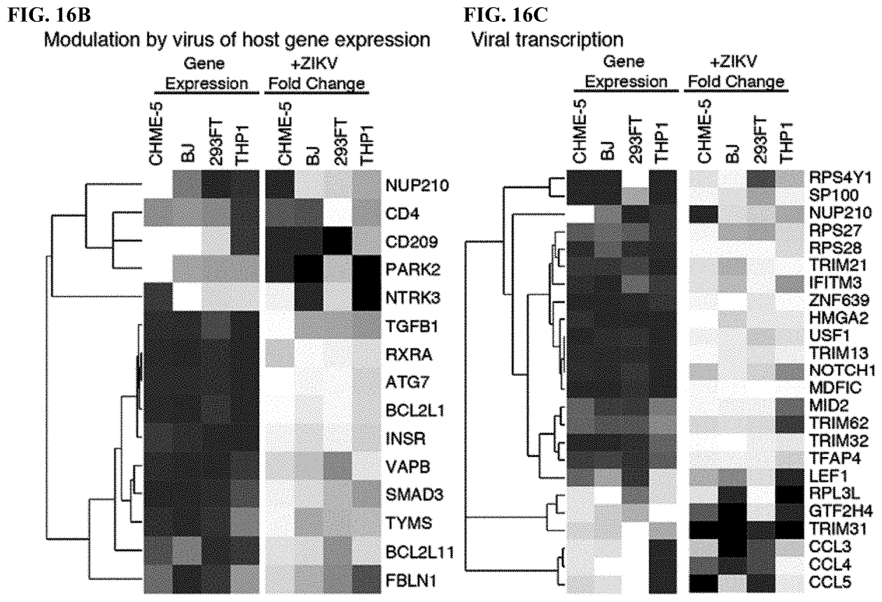

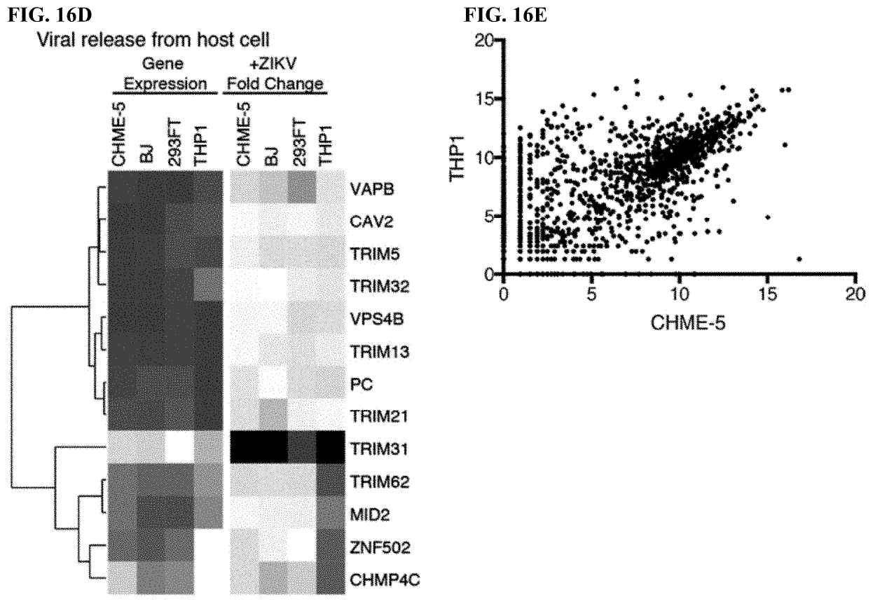

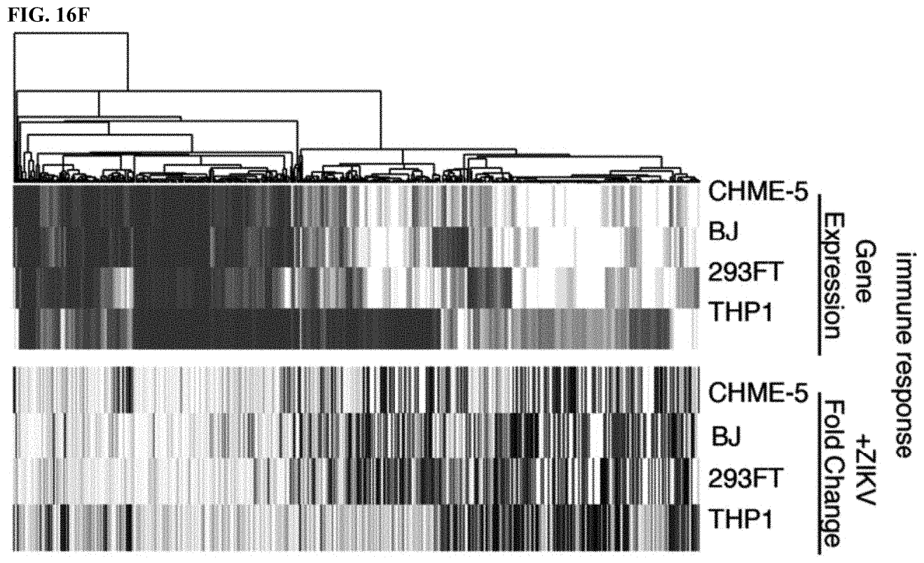

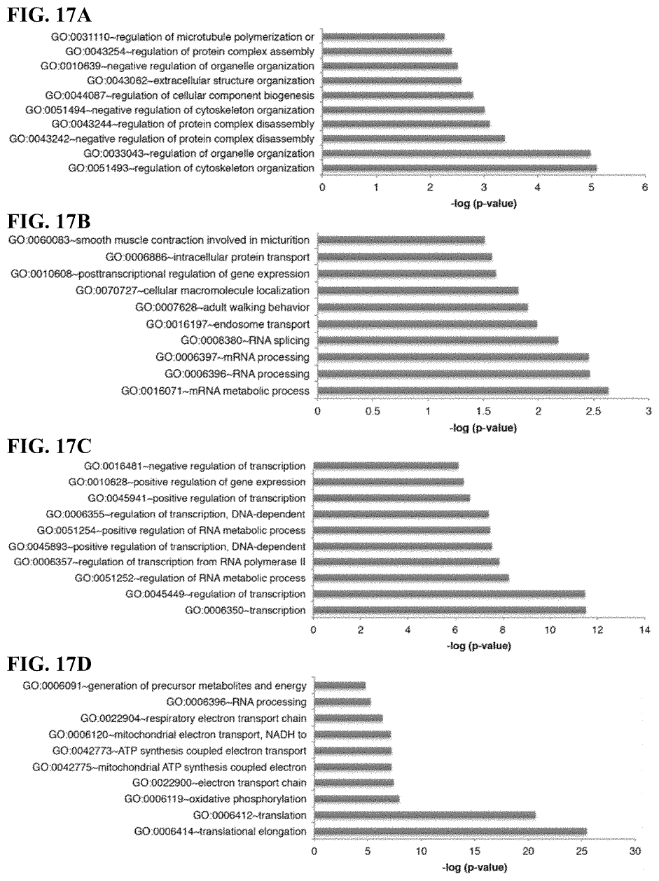

Since metabolic processes are the most dysregulated pathways during ZIKV infection at the transcriptome level, we hypothesized that inhibition of these pathways could be exploited for therapeutic effects. To determine the role of host cell metabolism in ZIKV replication, we utilized nucleoside metabolic inhibitors because nucleoside analogs have been reported to inhibit flaviviruses by interfering with RNA synthesis, methyl transferases, and thymidine synthesis pathway [25-27]. We utilized nucleoside metabolic inhibitors flurouracil and floxuridine in our experiments. Microglia cells were treated with flurouracil or floxuridine and inoculated with ZIKV. The effect of the antimetabolites floxuridine (FIGS. 14A-14B) and flurouracil (FIGS. 14C-14D) on ZIKV replication was assessed 48 hpi at the RNA and protein level by RT-qPCR and immunohistochemistry, respectively. Although both nucleoside analog affected ZIKV, but the treatment of floxuridine inhibited ZIKV more efficiently in a dose dependent manner than flurouracil. These findings show that ZIKV replication is sensitive to nucleoside metabolic pathways revealed by transcriptomic analyses.

Discussion.

We analyzed the transcriptional profiles of human microglia, fibroblast, kidney, and macrophage cell lines to explore the host factors that contribute to susceptibility to ZIKV infection, viral replication, and host symptomology. ZIKV expression was significantly different among the cell lines, with a notably large difference between microglial cells and THP-1 monocyte-derived cells, despite both being macrophage cell lines. A recent study analyzing the cell line susceptibility in across cell types—including placental, genitourinary, neuromuscular, retinal, respiratory and liver—and species further validate our findings. Similar to our results, the study showed that THP-1 derived macrophages are relatively resistant to viral infection when compared to HEK, HeLa and SF268 neurons [28].

By analyzing steady-state gene expression levels in cells before ZIKV infection, we identified several antiviral response genes that may contribute to the significant difference in ZIKV expression between cell types. For example, the Toll-like receptors TLR7 and TLR8 are functionally related genes and are highly expressed in THP-1 derived macrophages compared with microglia, 293FT, and BJ cells. Since TLR7 and TLR8 are activated by ssRNAs, they likely allow THP-1 macrophages to recognize flaviviruses and produce a more robust innate immune response [29-30]. Moreover, THP-1 derived macrophages express TNF-α as well as CD86, a co-stimulatory molecule that has been implicated in the early and late acute phases of dengue infection. However, it is important to note that differences in basal level gene expression between cell types does not necessarily equate to functional significance. Further experiments will be required to determine which of these antiviral response genes regulate ZIKV expression.

Other flaviviruses, such as West Nile (WNV), dengue, yellow fever, and Japanese encephalitis viruses, have shown a remarkable ability to evade the innate and adaptive immune systems [31]. Complement proteins recognize target pathogens and act as opsonins to promote recruitment of phagocytes and lysis of infected cells. Previous studies have shown that the complement system can be compromised by the flavivirus nonstructural protein NS1, which interacts with the complement regulatory glycoprotein factor H [32]. In addition, flaviviruses are able to evade the antibody and cellular immune response by affecting antigen presentation. The error-prone nature of flavivirus RNA polymerases leads to the accumulation of mutations and subsequent alterations in viral proteins that may help them to escape recognition by neutralizing or inhibitory antibodies [32].

We showed that ZIKV affects the adaptive immune response and complement cascade by modulating genes such as IL1B, CD4, IL27RA, and A2M. Flaviviruses downregulate CD4 mRNA through an NS5-dependent mechanism, thereby dysregulating both the innate and adaptive immune systems [33]. Moreover, these findings are corroborated by a recent study analyzing transcriptional changes in ZIKV infected human neural stem cells in which leukocyte activation, cytokine production and defense response pathways were significantly dysregulated [34]. In addition, IL1B has previously been linked to WNV. IL-1β is present at increased levels in the plasma and cortical neurons of WNV patients, and it plays a key role in restricting virus replication [35]. IL-1β acts in concert with type I IFN and the NLRP3 inflammasome to restrict WNV replication. Interestingly, IL1B expression was upregulated by ZIKV in THP-1 derived macrophages cells but downregulated in microglia cells, which is consistent with our finding that viral replication is higher in the microglial-derived than in the monocyte-derived macrophage. These data imply that, while ZIKV is actively targeted by the innate and adaptive immune responses via monocyte-derived macrophages, it is able to effectively evade the microglial immune response once it passes through the blood-brain barrier and reaches the central nervous system.

Endothelial cells are one of the cell types infected by dengue, also a flavivirus, and the breakdown in endothelial barrier function causes the vascular leakage associated with hemorrhagic fever. Dengue virus type 2 suppresses TNF-α-mediated hyperpermeability and angiogenesis by modulating type I IFN [36]. Cases of thrombocytopenia and subcutaneous bleeding have been observed in ZIKV patients [37]. Our data suggest that ZIKV may affect angiogenesis and endothelial cell integrity. These findings may provide insights into the molecular mechanisms by which ZIKV passes through the blood-brain barrier.

We identified a large number of differentially expressed genes associated with cellular metabolic processes following ZIKV infection. Previous studies have drawn attention to the role of viral-mediated reprogramming of host metabolic processes in pathogenesis. In accordance with our findings, transcriptional alterations in human neural stem cells inoculated with MR766 strain ZIKV revealed significant remodeling of nucleic acid metabolic processes and macromolecule biosynthesis [34]. RNA viruses such as influenza and dengue alter fatty acid synthesis and induce glycolysis to promote viral replication, late gene synthesis, and virion assembly [22]. The role of dengue NS3 recruitment of fatty acid synthase to sites of viral replication has been dissected using RNAi and small molecule inhibitors in an effort to identify potential therapeutic targets [38]. Many other viruses redistribute host cell resources to promote viral replication by altering the localization of lipids, as seen with dengue. In addition, viral infection alters the rate of host RNA metabolism to enhance the availability of nucleotides [24].

The molecular mechanisms by which viruses redirect cellular resources and exploit the host metabolic machinery are largely unknown. Recent work showed that the adenovirus gene element E4ORF1 upregulates glucose metabolism by altering the epigenetic landscape. E4ORF1 interacts with MYC to enhance transcription of glycolytic enzymes and nucleotide biosynthesis [39]. Here, we identified several genes associated with metabolic regulation that are differentially expressed between cell types following ZIKV infection, including CREB5, TERT, CNIH1, ADAM12, and USE1. One gene that ZIKV may exploit to regulate the host cell is CREB5. CREB5 (CAMP-responsive element-binding protein 5) is a transcription factor that regulates nucleotide and nucleic acid metabolism, transcription, and signal transduction, and is also highly upregulated in patients with HIV encephalitis and vaccinia virus infections [40,41]. Because cellular metabolism is often a limiting factor in viral replication, nucleoside/nucleotide-based therapeutics have been developed against a variety of viruses, including HIV, HBV, HCV, HSV, and VZV [23]. Indeed, nucleoside analogs including floxuridine, also known to be effective against other flaviviruses such as dengue, displayed dose-dependent inhibition of ZIKV replication [25-27]. Further mechanistic studies will be required to gain a better understanding of how ZIKV hijacks the host cell metabolic machinery and to aid in the development of ZIKV-targeted therapeutics.

Conclusions. ZIKV infection is an emerging disease associated with increased incidence of neurological disorders including congenital microcephaly and Guillain-Barre syndrome. Here we analyzed the transcriptomic changes associated with ZIKV infection across multiple cell types to identify novel therapeutic targets and understand the host-pathogen interaction for sustained ZIKV replication. The response to ZIKV is cell type specific with the greatest replication found in microglia cells. ZIKV is highly expressed in microglia and downregulates immune response genes while high expression of viral response genes in macrophages confers ZIKV resistance. In addition, ZIKV reprograms the host metabolic processes to enhance virus replication through the upregulation of glycolysis and RNA metabolism related genes. Antimetabolites floxuridine abrogated ZIKV replication through inhibition of host nucleoside metabolic pathways. These results reveal that thymidine synthesis pathway can be exploited to develop novel therapeutics to treat ZIKV infections.

Materials and Methods.

Cell Lines and Culture Conditions. Vero, microglia, THP-1, BJ, and 293FT cells were maintained under standard culture conditions at 37° C. in a 5% CO₂ atmosphere. Vero cells, derived from African green monkey kidney cells, were maintained in EMEM supplemented with 10% (vol/vol) fetal bovine serum (FBS) and antibiotics. THP-1 cells, a human leukemia monocytic cell line, were cultured in RPMI 1640 medium supplemented with 10% FBS and 50 μM β-mercaptoethanol (Sigma). THP-1 cells were differentiated into macrophages by treatment with 5 ng/ml phorbol-12-myristate-13-acetate (PMA) overnight. The following day, the medium was replaced with fresh medium without PMA. 293FT human embryonic kidney cells and the human fibroblast cell line BJ were cultured in DMEM (Invitrogen) supplemented with 10% FBS. The human microglial cell line was cultured in DMEM medium with high glucose supplemented with 10% FBS and 1% penicillin/streptomycin.

Zika Virus Propagation and Infection of Cell Lines. ZIKV prototype MR766 was propagated in the low passage Vero cell line. Vero cells were infected with virus at a MOI of 1 in EMEM medium supplemented with 10% FBS. The medium was replaced with fresh medium 24 h after infection and the viral supernatant was collected at 48 h post-infection. Viral titers were assessed using iScript One-Step RT-PCR kit (Bio-Rad) and the viral copy number was calculated from a standard curve of in vitro transcribed viral RNA transcripts. For infection, cell lines were seeded in 6-well culture plates at a density of 1×10⁶ cells per well. ZIKV, diluted to the desired multiplicity of infection (MOI:1), was added to the cells, and the plates were incubated at 37° C. in a 5% CO₂ atmosphere for 6, 12, 24, or 48 h. As controls, cells were incubated with culture supernatants from uninfected Vero cells (mock-infected controls). At the indicated times post-infection, cell supernatants were collected for determination of viral copy number.

Immunofluorescence Microscopy. To assess ZIKV infection, cells were harvested at 24 h following infection and immunostained as described previously [16]. ZIKV- and mock-infected cells were fixed with 4% paraformaldehyde in PBS for 20 min at room temperature. Cells were blocked by incubation in 3% BSA and 0.1% Triton X-100 for 2 h at room temperature and then incubated overnight at 4° C. with ZIKVE/anti-flavivirus group antigen antibody (1:500, mouse, Millipore MAB10216), which is directed against the flavivirus envelope protein. Cells were washed with PBS and incubated for 2 h at room temperature with fluorescein isothiocyanate (FITC)-conjugated anti-mouse IgG. The nuclei were stained with Hoechst 33258. Immunostained cells were imaged using a Leica fluorescence microscope (DMI 3000B).

RNA Extraction, cDNA Synthesis, and qRT-PCR. For cellular mRNA analysis, RNA was extracted from the cell lines using an RNeasy Mini Kit (Qiagen), following the manufacturer's instructions. RNA samples were treated with RNase-free DNase (Qiagen) and cDNA was generated from total RNA (500 ng/sample) using iScript Mastermix (Bio-Rad), according to the manufacturer's instructions. qPCR was performed with SYBR Green PCR Master Mix (Bio-Rad) using a Roche LightCycler 480.

RNA-Seq and Data Analysis. For RNA-seq analysis, RNA was extracted from the cell lines using an RNeasy Mini Kit (Qiagen), following the manufacturer's instructions. RNA was ribo-depleted, and RNA sequencing was performed using an Illumina NextSeq 500 with an average of 20 million reads per sample at The Scripps Research Institute NGS Core facility. The single-end reads that passed Illumina filters were filtered for reads aligning to tRNA, rRNA, adapter sequences, and spike-in controls. The reads were then aligned to UCSC hg19 reference genome using TopHat (v 1.4.1). DUST scores were calculated with PRINSEQ Lite (v 0.20.3), and low-complexity reads (DUST>4) were removed from the BAM files. The alignment results were parsed via the SAMtools to generate SAM files. Read counts to each genomic feature were obtained with the htseq-count program (v 0.6.0) using the “union” option. After removing absent features (zero counts in all samples), the raw counts were imported into R/Bioconductor package DESeq2 to identify differentially expressed genes among the samples. DESeq2 normalizes counts by dividing each column of the count table (samples) by the size factor of this column. The size factor is calculated by dividing the samples by the geometric means of the genes. This brings the count values to a common scale suitable for comparison. P values for differential expression were calculated using binomial test for differences between the base means of two conditions. The p values were adjusted for multiple test correction using the Benjamini-Hochberg algorithm to control the false discovery rate. Cluster analyses, including principal component analysis and hierarchical clustering, were performed using standard algorithms and metrics. Gene ontology analyses on biological processes were performed using The Database for Annotation, Visualization and Integrated Discovery (DAVID) [42]. Grouped functional pathway/gene ontology network analyses were performed using Cytoscape with the ClueGo add-on [43,44]

Drug Treatment. Human microglial cell line was infected with ZIKV virus at MOI of 1 in cell medium containing metabolic inhibitors such as 5-Fluorouracil (Abcam, ab142387) and Floxuridine (Tocris, 4659) in cell medium containing 1 μM and 10 μM of each drug or 1% (vol/vol) DMSO as a control. After 48 h post-infection cellular RNA was extracted using Trizol and cDNA was synthesized by iScript Mastermix (Bio-Rad), as per manufacturer instructions. ZIKV RNA was quantified by using specific primers by SYBR Green PCR Master Mix (Bio-Rad) using a Roche LightCycler 480. Further immunocytochemistry for was also performed in microglial cells for detection of ZIKV infection using flavivirus group antigen specific antibody (Millipore).

Tables (Example 2).

TABLE 1
Gene expression of viral response genes, Related
to FIG. 12A. Values represent log2(RPKM + 1).

| Associated | | | | |
|---|---|---|---|---|
| Gene Name | chme | bj | 293 | thp1 |
| FGF7 | 1.91838623 | 8.84830996 | 1.81557543 | 3.26603689 |
| LMCD1 | 6.15845822 | 8.36181222 | 6.39351917 | 0 |
| COX8A | 10.947615 | 10.229648 | 9.97955381 | 10.9550105 |
| DKKL1 | 2.49313492 | 1.79493566 | 3.35473424 | 1.28688115 |
| MSTN | 0 | 1.79493566 | 1.422233 | 1.95233357 |
| CHAF1B | 9.49215363 | 8.46107011 | 9.55939621 | 4.58436125 |
| ATP1B1 | 10.3670652 | 9.31286027 | 8.45145841 | 8.03606369 |
| HIST1H3D | 11.284211 | 11.5114908 | 5.00225245 | 7.62811741 |
| C1orf210 | 0 | 0 | 1.422233 | 3.03210084 |
| SNTG1 | 0 | 1.79493566 | 0 | 4.07038933 |
| IST1 | 11.6720452 | 10.662793 | 10.3900292 | 11.5676567 |
| FABP1 | #N/A | #N/A | #N/A | #N/A |
| TMEM173 | 7.41438927 | 9.43352285 | 4.79960542 | 8.9561438 |
| MYOM1 | 2.23572706 | 3.59693514 | 3.23112516 | 4.66789213 |
| CCDC36 | 0 | 5.20006486 | 0 | 1.28688115 |
| CRNKL1 | 10.3861751 | 10.094632 | 9.94436068 | 9.58535693 |
| FXR2 | 10.8262068 | 10.6294932 | 10.1533114 | 10.6784857 |
| DNAAF2 | 7.86795822 | 7.9518675 | 8.61787147 | 8.49257426 |
| RFWD3 | 11.3086633 | 10.9240331 | 11.15108 | 9.82815236 |
| CA7 | 1.51096192 | 0 | 2.12432814 | 2.40871186 |
| PDK4 | 3.0721058 | 1.79493566 | 5.24488706 | 3.26603689 |
| ALPK1 | 6.46123386 | 7.93740325 | 6.89857116 | 6.90989308 |
| STOM | 9.37623394 | 11.4517778 | 8.6209529 | 12.2645511 |
| MDH1 | 10.5881524 | 11.4125116 | 11.2513226 | 11.7651866 |
| FCGR3B | #N/A | #N/A | #N/A | #N/A |
| REEP2 | 7.90189263 | 7.62234472 | 9.23713824 | 3.03210084 |
| EIF2AK4 | 10.3569141 | 11.5960919 | 10.0255687 | 9.84403094 |
| HILPDA | 7.66554911 | 7.26575568 | 8.00045077 | 6.66633054 |
| SERPINB10 | 0 | 1.79493566 | 1.422233 | 1.95233357 |
| MRPS10 | 10.4668004 | 10.2147562 | 9.96266469 | 12.2783856 |
| CD163L1 | 0.94860085 | 5.50430258 | 1.422233 | 4.39985467 |
| CREB3 | 10.3218267 | 10.6539915 | 7.93769749 | 10.6568271 |
| DDX58 | 8.49785184 | 8.61871585 | 7.05245937 | 8.41578401 |
| LILRB1 | 0 | 0 | 0 | 5.08915913 |
| PHYHIP | 3.36036428 | 3.73660488 | 1.422233 | 4.18903382 |
| MB21D1 | 8.92389197 | 8.76081973 | 2.37851162 | 8.90517638 |
| KCNQ1 | 0.94860085 | 0 | 2.592158 | 5.79701298 |
| YIPF1 | 8.87347487 | 8.13386145 | 8.08316017 | 9.63314055 |
| UBA52 | 12.4517881 | 12.1573375 | 12.2104765 | 12.7400485 |
| SMURF1 | 9.49956689 | 9.78558325 | 8.50910199 | 10.0958585 |
| CPA3 | 5.62029315 | 0 | 0 | 1.28688115 |
| ASB2 | 0 | 0 | 1.81557543 | 3.46727948 |
| CLDN7 | 2.49313492 | 2.56803211 | 3.46858332 | 6.92706681 |
| PNPO | 7.40658769 | 8.43562859 | 8.51573926 | 10.053573 |
| MAP1A | 9.7927415 | 12.2052098 | 9.60829179 | 4.82273015 |
| MBD5 | 7.00999653 | 7.59507095 | 6.78240857 | 7.2465981 |
| ZNF681 | 0 | 5.15055968 | 5.39643353 | 0 |
| SLC35B3 | 9.34777637 | 9.63041275 | 7.60607203 | 8.52109001 |
| HCAR1 | 1.51096192 | 0 | 0.87970577 | 0 |
| MAVS | 9.78517453 | 10.0617222 | 9.2806084 | 8.62972089 |
| KIAA1549L | 7.11019618 | 9.65266575 | 6.03584382 | 4.29865832 |
| PTPN22 | 5.95814667 | 3.59693514 | 1.81557543 | 6.64573048 |
| ITPKC | 7.21286112 | 7.94462422 | 6.67990229 | 7.91247024 |
| SLC25A19 | 5.28540222 | 5.88141991 | 7.65492239 | 7.50803243 |
| FGF14 | 0 | 7.25417829 | 0 | 0 |
| PYDC1 | 0 | 0 | 1.422233 | 0 |
| MRPS2 | 8.6520562 | 8.15896306 | 9.26886945 | 8.04395687 |
| HIST1H3F | 12.025977 | 11.984625 | 10.4141884 | 6.78384949 |
| IL12B | 0.94860085 | 0 | 0.87970577 | 1.28688115 |
| C8orf59 | 6.64616266 | 6.92872562 | 6.2890967 | 7.20506011 |
| FAM13B | 9.62338771 | 10.0995056 | 9.40367249 | 10.1442886 |
| ACKR3 | 6.02125762 | 4.09000653 | 0 | 7.85990745 |
| LSM4 | 11.3165474 | 10.7208599 | 11.1590451 | 11.0312739 |
| PPY | 0 | 0 | 0 | 0 |
| EIF2S1 | 11.6551461 | 11.8982537 | 11.688753 | 11.675516 |
| HIST1H3H | 11.6241375 | 11.4788303 | 12.0773131 | 7.2465981 |
| ANXA5 | 13.4499882 | 13.8543191 | 11.323477 | 14.4576502 |
| GABRA5 | 0.94860085 | 1.15704371 | 0.87970577 | 0 |
| HPR | 0.94860085 | 0 | 0 | 0 |
| ACIN1 | 11.1046119 | 11.164643 | 11.2730088 | 10.7512428 |
| CHST3 | 11.3399502 | 11.3600185 | 8.68706069 | 6.68664059 |
| SLC1A3 | 8.26129574 | 1.15704371 | 9.60518317 | 12.6957459 |
| SIN3A | 9.49215363 | 9.52085436 | 10.3320477 | 8.94356975 |
| SCN1A | 0.94860085 | 1.15704371 | 0 | 0 |
| CD93 | 0.94860085 | 1.15704371 | 1.422233 | 2.40871186 |
| LENG9 | 5.21490191 | 5.61735726 | 4.45811948 | 5.59723319 |
| TEAD4 | 8.39835892 | 7.98732087 | 9.01881219 | 5.6395216 |
| HIST1H3A | 9.86046626 | 10.4478478 | 11.1516128 | 5.26115467 |
| TREM1 | 0.94860085 | 2.23266076 | 0 | 6.32228872 |
| RBM18 | 9.54502237 | 9.61687962 | 9.04884103 | 9.65644274 |
| IL23R | #N/A | #N/A | #N/A | #N/A |
| PEX3 | 8.87347487 | 8.67200096 | 8.47889093 | 7.54179359 |
| AIM2 | 0.94860085 | 1.15704371 | 0 | 6.00495076 |
| STAT2 | 10.0875557 | 10.5174529 | 8.69289443 | 9.9836492 |
| PEX13 | 9.29980329 | 10.0263436 | 8.86635166 | 10.6309132 |
| HIST1H3I | 8.67514551 | 9.77140693 | 9.32280697 | 5.68060567 |
| ALKBH5 | 11.1269045 | 11.0136577 | 11.8788211 | 10.4953354 |
| HIST1H3J | 10.017045 | 10.8860014 | 9.73443867 | 5.26115467 |
| CSPG5 | 2.49313492 | 3.4409522 | 7.30295873 | 4.29865832 |
| DPF3 | 4.21800615 | 7.33351318 | 5.58466191 | 8.22804905 |
| MYLK | 12.3154808 | 13.9034375 | 7.62461271 | 8.09032418 |
| TMEM39B | 7.7548875 | 7.20672136 | 7.19140473 | 6.97727992 |
| SUPT3H | 6.68565988 | 5.9112122 | 7.33333428 | 6.83920379 |
| PML | 7.87369018 | 8.58202905 | 7.55550943 | 7.27360913 |
| MTSS1 | 4.41886458 | 5.8509994 | 7.07948478 | 13.0445615 |
| HIST1H3G | 11.0092338 | 11.4248591 | 0 | 5.36562256 |
| SNRPD1 | 10.5449451 | 10.1315741 | 10.9788391 | 9.58805872 |
| IL4 | 0.94860085 | 0 | 0 | 0 |
| NUP93 | 11.3685447 | 10.5999965 | 11.0762478 | 9.70691038 |
| FAM131B | 4.84799691 | 5.29351763 | 5.0395771 | 5.97223312 |
| HIST1H3C | 11.4677074 | 11.5582485 | 0.87970577 | 5.90472493 |
| TRAF3IP2 | 8.76085299 | 8.63669712 | 6.55673595 | 8.52109001 |
| KRT6A | 0 | 0 | 0 | 0 |
| ZNF189 | 8.95921954 | 8.80522796 | 8.99052948 | 9.05688153 |
| SNRPB2 | 10.1358888 | 10.1814505 | 10.2299003 | 9.37046946 |
| HIST1H3E | 10.3039519 | 11.3313089 | 6.70334992 | 2.7548875 |
| NTHL1 | 7.72996056 | 7.66682759 | 8.93966736 | 6.70653055 |
| FANCL | 8.05555359 | 8.21989453 | 8.69289443 | 7.75828985 |
| SELK | 8.92389197 | 9.06638524 | 8.89965903 | 9.83724947 |
| HIST1H3B | 12.8031463 | 12.435876 | 12.8302478 | 6.09866378 |
| SNRPB | 11.6342711 | 11.2334879 | 12.1488992 | 10.863412 |
| IL12RB1 | 0 | 0 | 1.422233 | 5.75915583 |
| GMIP | 6.19554446 | 7.15775114 | 6.67990229 | 9.95261022 |
| ZCCHC17 | 9.18735207 | 8.64562241 | 8.87408995 | 8.33463084 |
| IL23A | 2.71369582 | 1.15704371 | 2.94673086 | 3.80012335 |
| SLC35C1 | 8.58747751 | 8.84058912 | 6.54380518 | 6.80258099 |
| FGFBP1 | 1.51096192 | 0 | 2.12432814 | 0 |
| TMEM39A | 9.82553125 | 9.59172803 | 8.60547952 | 9.66666784 |
| TNIK | 10.1167468 | 9.30724636 | 9.04884103 | 8.12789118 |
| TRIM6 | 7.2917693 | 6.54643149 | 6.70334992 | 1.95233357 |
| TMEM203 | 9.40609869 | 9.12174071 | 8.81618368 | 9.55537521 |
| ACTRT1 | #N/A | #N/A | #N/A | #N/A |
| APOBEC3G | 1.91838623 | 4.81403765 | 3.93168306 | 8.09032418 |
| APOBEC3F | 3.89724043 | 5.46434153 | 5.36737107 | 5.14893411 |
| REP15 | 2.49313492 | 3.07038933 | 2.12432814 | 4.18903382 |
| SLC37A4 | 6.6055536 | 7.34447346 | 9.15881163 | 7.13298804 |
| TLR3 | 3.22342255 | 2.56803211 | 5.75114233 | 2.7548875 |
| SERINC5 | 6.73674016 | 6.94333532 | 6.79337571 | 7.14771372 |
| SERINC3 | 11.7797768 | 12.2324509 | 10.3737026 | 11.9367852 |
| IFIH1 | 6.10118782 | 4.68144927 | 6.2890967 | 8.77402928 |
| MX1 | 5.86937792 | 5.38162947 | 2.94673086 | 2.40871186 |
| CCL4 | 0.94860085 | 0 | 0 | 11.6060674 |
| MX2 | 5.93640238 | 3.73660488 | 1.422233 | 5.55397481 |
| OAS2 | 2.49313492 | 3.73660488 | 1.81557543 | 8.02020215 |
| LAMTOR5 | 9.41591074 | 9.4514172 | 8.81082866 | 10.8101539 |
| HYAL1 | 0 | 1.79493566 | 2.12432814 | 2.7548875 |
| BATF3 | 5.38474059 | 4.74899785 | 5.83844759 | 4.18903382 |
| CXCL12 | 9.75764002 | 10.6726375 | 5.0395771 | 2.7548875 |
| OASL | 2.49313492 | 2.56803211 | 0.87970577 | 6.58225491 |
| FGR | 2.23572706 | 0 | 0 | 11.7326677 |
| CHUK | 10.3640252 | 10.2676645 | 10.3936371 | 10.298727 |
| RSAD2 | 0 | 1.15704371 | 0 | 6.58225491 |
| HYAL3 | 5.17871464 | 5.88141991 | 5.65849714 | 6.90989308 |
| TRIM22 | 6.65949648 | 9.88676351 | 0.87970577 | 6.26959425 |
| IRF7 | 7.00999653 | 5.42357817 | 6.33467553 | 10.1696996 |
| TBK1 | 9.82257083 | 9.62815387 | 9.10268437 | 10.9339783 |
| TNF | 1.91838623 | 1.15704371 | 0 | 10.4852457 |
| AP1S1 | 10.8690077 | 10.6964112 | 9.22908316 | 11.2865809 |
| XPR1 | 10.077697 | 10.1140286 | 9.66902677 | 10.6973325 |
| FOSL1 | 9.22804905 | 9.71748794 | 1.422233 | 7.21897486 |
| OAS1 | 2.23572706 | 0 | 0 | 7.84197312 |
| BTBD17 | 0 | 0 | 0.87970577 | 0 |
| SRC | 8.6218321 | 9.06638524 | 8.17397689 | 10.5830734 |
| PIM2 | 8.03529399 | 7.31124879 | 8.23950306 | 7.02580399 |
| CCDC130 | 8.57353359 | 8.18363538 | 7.7977912 | 9.28000515 |
| CXCL10 | 0 | 0 | 1.422233 | 4.58436125 |
| CFL1 | 13.9592802 | 13.6097536 | 11.9441776 | 13.4709181 |
| IFNGR2 | 9.79122802 | 9.64158215 | 8.57723998 | 12.7119014 |
| IL12A | 4.06608919 | 4.68144927 | 3.46858332 | 3.26603689 |
| ABCE1 | 11.258248 | 11.8019064 | 12.815041 | 11.4074908 |
| IFNB1 | 0 | 0 | 0 | 1.95233357 |
| HMGA2 | 8.47549026 | 11.4403034 | 9.72159482 | 10.8711814 |
| ENO1 | 15.4215356 | 14.5200962 | 14.9424746 | 14.3163622 |
| EIF2AK2 | 9.78821261 | 10.8087712 | 10.7221757 | 9.90151557 |
| OAS3 | 7.35032044 | 7.33351318 | 7.90267659 | 9.60679383 |
| IFNAR2 | 6.78607355 | 6.15542543 | 5.77346893 | 8.27565895 |
| CCL11 | #N/A | #N/A | #N/A | #N/A |
| NPC2 | 9.49215363 | 10.0399198 | 8.26336311 | 11.8685795 |
| DUOX2 | 0 | 1.15704371 | 0 | 3.26603689 |
| CYP1A1 | 0.94860085 | 1.15704371 | 3.76447355 | 2.7548875 |
| DDX1 | 10.6207788 | 11.4943306 | 11.5147535 | 10.2904682 |
| CCL22 | 0 | 0 | 1.422233 | 2.40871186 |
| CCL19 | 0 | 0 | 0 | 0 |
| XCL1 | 0.94860085 | 0 | 3.35473424 | 0 |
| DCLK1 | 8.25252352 | 6.86913112 | 6.30451104 | 1.95233357 |
| SPACA3 | #N/A | #N/A | #N/A | #N/A |
| MST1R | 2.90496572 | 1.79493566 | 4.28243981 | 1.95233357 |
| IFNGR1 | 10.3961338 | 10.0550519 | 9.16093027 | 11.5267975 |
| APOB | 0 | 1.15704371 | 0 | 1.95233357 |
| URI1 | 10.6814668 | 10.3571781 | 10.1156159 | 10.0280027 |
| CXCR4 | 2.23572706 | 0 | 8.25941374 | 8.49833066 |
| CCL8 | 0 | 0 | 0 | 3.03210084 |
| DDX60 | 6.94637997 | 7.74355536 | 7.03408363 | 9.36732738 |
| IFITM1 | 2.23572706 | 7.24241196 | 3.76447355 | 3.03210084 |
| BCL3 | 6.35102755 | 5.04701482 | 3.09592442 | 7.47346224 |
| CCT5 | 13.1552831 | 12.8278726 | 13.2009691 | 13.7841052 |
| IFITM2 | 1.91838623 | 8.35098337 | 6.24184018 | 7.48502423 |
| EEF1G | 0 | 0 | 0.87970577 | 3.03210084 |
| HSPB1 | 10.4060243 | 11.1975853 | 7.42071818 | 9.81440622 |
| TNFSF4 | 3.7059779 | 6.63865312 | 3.35473424 | 5.14893411 |
| HNRNPUL1 | 12.1079232 | 11.517571 | 11.5213551 | 11.0391727 |
| BCL2L1 | 9.4526325 | 10.627242 | 8.19888699 | 10.1277107 |
| BANF1 | 9.31036284 | 8.77712378 | 8.49908847 | 8.49257426 |
| CCL5 | 1.51096192 | 1.15704371 | 0 | 9.60145511 |
| IFIT3 | 9.00419237 | 8.9050861 | 3.67242534 | 9.4257614 |
| BST2 | 0 | 1.79493566 | 2.592158 | 10.5279852 |
| IFIT1 | 7.04078287 | 9.19942697 | 6.97750891 | 8.02818235 |
| CLU | 6.74926608 | 7.79246496 | 9.73159057 | 3.03210084 |
| IFIT2 | 7.04078287 | 8.10181813 | 2.7803101 | 7.00977264 |
| ACTA2 | 9.30193054 | 10.2132622 | 3.46858332 | 0 |
| CREBZF | 9.51244419 | 9.92934719 | 10.0622756 | 9.34837408 |
| DHX58 | 3.89724043 | 5.9692426 | 3.57410151 | 6.96069704 |
| IFNK | 0 | 0 | 2.12432814 | 1.28688115 |
| HMGA1 | 9.49401552 | 12.4400438 | 11.3073261 | 13.6423293 |
| ISG20 | 3.0721058 | 3.98185265 | 3.67242534 | 7.64896869 |
| RPS15A | 10.9209632 | 10.658399 | 10.8666144 | 10.7306569 |
| CHRM2 | 0 | 9.35149141 | 0 | 2.7548875 |
| IVNS1ABP | 11.6956155 | 11.3667703 | 10.0092268 | 12.5226821 |
| PSMA2 | 9.02231232 | 8.37794796 | 8.84805952 | 8.59797804 |
| ADAR | 12.0362011 | 12.0329687 | 12.4456486 | 13.1433976 |
| DDX3X | 13.0981851 | 13.2568603 | 13.2517457 | 13.2024223 |
| FOXP3 | 0 | 1.15704371 | 2.37851162 | 0 |
| CDK6 | 12.4787418 | 12.8081658 | 11.9039948 | 13.620772 |
| IKBKG | 7.63894267 | 7.44997399 | 5.96000193 | 7.97973957 |
| HYAL2 | 8.46797229 | 9.06971828 | 8.04701482 | 8.90517638 |
| IFI44 | 6.54951516 | 6.52732061 | 0.87970577 | 7.07274895 |
| IFITM3 | 8.6013251 | 9.72171443 | 5.27649667 | 7.58563861 |
| ZC3HAV1 | 10.644361 | 10.9539345 | 10.6380738 | 10.4707097 |
| MEF2C | 3.22342255 | 6.86913112 | 8.35124846 | 10.6296116 |
| ODC1 | 11.7513599 | 12.1094324 | 12.6198073 | 12.3178931 |
| TPT1 | 12.6331996 | 13.4574155 | 12.0984467 | 14.34214 |
| DDX21 | 13.0513874 | 12.7507363 | 13.4345175 | 12.656798 |
| PRKRA | 9.30827076 | 9.37577797 | 9.2806084 | 8.96448527 |
| TLR8 | 0 | 0 | 0 | 2.7548875 |
| IFNAR1 | 10.5864082 | 10.5403315 | 9.47002838 | 11.8746664 |
| TBX21 | 0.94860085 | 0 | 2.94673086 | 6.21470767 |
| DHX36 | 10.0286106 | 10.2266891 | 9.91956355 | 9.75087444 |
| IRAK3 | 5.10265813 | 8.20789285 | 3.23112516 | 8.65051327 |
| IFNW1 | #N/A | #N/A | #N/A | #N/A |
| STMN1 | 13.5339952 | 12.7048482 | 11.8100253 | 9.00135189 |
| IFNG | #N/A | #N/A | #N/A | #N/A |
| IKBKB | 9.50694225 | 9.61006514 | 8.83753333 | 10.2330684 |
| IFNA17 | #N/A | #N/A | #N/A | #N/A |
| IFNA4 | #N/A | #N/A | #N/A | #N/A |
| IFNA7 | #N/A | #N/A | #N/A | #N/A |
| MYD88 | 7.21286112 | 8.38331549 | 5.14567746 | 8.42181238 |
| GTF2F1 | 11.1785777 | 10.5486678 | 10.5846807 | 10.9736902 |
| GATA3 | 1.51096192 | 2.83995959 | 6.78240857 | 2.7548875 |
| LSM14A | 10.6190828 | 10.4038855 | 10.5688302 | 10.6164661 |
| IRF3 | 9.48284828 | 8.96327181 | 8.63622583 | 10.1015293 |
| TSPAN6 | 8.47175649 | 8.80125568 | 11.0587629 | 6.99356115 |
| C1QBP | 11.8386406 | 11.5053848 | 12.9290161 | 11.5456402 |
| TKFC | #N/A | #N/A | #N/A | #N/A |
| BIRC3 | 7.71094416 | 6.79103262 | 4.84247606 | 10.1146794 |
| BIRC2 | 11.2761477 | 12.3209838 | 9.54486786 | 11.0558247 |
| NLRX1 | 7.11019618 | 7.8242587 | 8.33632803 | 8.34739991 |
| ITCH | 11.5565013 | 11.4949456 | 10.9094131 | 11.7876414 |
| IL2RA | #N/A | #N/A | #N/A | #N/A |
| PCBP2 | 9.58560105 | 9.79762911 | 10.7721743 | 8.84784036 |
| TRIM38 | 6.6727082 | 8.63223186 | 7.06156021 | 9.55261147 |
| HTRA1 | 13.2552086 | 10.7355391 | 6.71493235 | 2.40871186 |
| PPM1B | 8.91835642 | 9.37042587 | 9.26101359 | 9.31621364 |
| TRAF3IP1 | 8.53799488 | 8.44584255 | 8.72471848 | 7.70963528 |
| ELMOD2 | 9.2457666 | 10.1092038 | 10.5672336 | 8.45762712 |
| TRAF3 | 10.0311774 | 9.79962161 | 9.70283067 | 10.746422 |
| SPN | 0.94860085 | 0 | 0 | 9.86418615 |
| HERC5 | 4.84799691 | 3.59693514 | 9.2451486 | 5.90472493 |
| TSPAN32 | 1.51096192 | 3.07038933 | 1.422233 | 2.7548875 |
| IL27 | 0 | 1.15704371 | 0 | 2.40871186 |
| MICB | 8.41019692 | 9.07301685 | 7.6368421 | 8.75495429 |
| TARBP2 | 8.69783636 | 8.72342204 | 9.09384008 | 8.97275009 |
| MUL1 | 9.57161993 | 9.40230862 | 9.13962809 | 9.01343434 |
| AP1B1 | 10.9651061 | 10.8008109 | 10.7907476 | 10.6746866 |
| B2M | 14.1453511 | 13.7308809 | 11.1758863 | 15.6891367 |
| HLA-A | 11.5759032 | 10.9130016 | 8.93224429 | 13.0366888 |
| AP2A2 | 10.3039519 | 10.1220256 | 10.0486231 | 10.0398649 |
| AP2S1 | 11.6245625 | 11.2835862 | 10.4817793 | 12.3767603 |
| ATP6V1H | 9.97780939 | 10.5698841 | 9.32658678 | 12.4618887 |
| ELMO1 | 0.94860085 | 0 | 5.24488706 | 6.72628629 |
| CD4 | 3.80426012 | 3.59693514 | 4.00898878 | 7.98788928 |
| HCK | 0.94860085 | 1.15704371 | 0.87970577 | 8.66590436 |
| AP1G1 | 12.0285415 | 11.3388812 | 11.3749797 | 11.4143417 |
| PACS1 | 9.42177031 | 9.81944466 | 7.71376454 | 7.83289001 |
| AP1S3 | 6.19554446 | 7.06781078 | 7.50969584 | 6.78384949 |
| CD247 | 1.91838623 | 0 | 0 | 0 |
| AP2A1 | 10.6105543 | 11.003293 | 9.65708617 | 10.1822454 |
| PAK2 | 11.6732737 | 10.9102305 | 10.1437148 | 11.3766409 |
| CD8B | 0 | 0 | 0 | 1.28688115 |
| DOCK2 | 8.04039932 | 7.52911816 | 0 | 11.6252203 |
| ARF1 | 13.1377373 | 12.6516572 | 12.0314945 | 13.3603574 |
| LCK | 0 | 0 | 0 | 0 |
| AP2B1 | 10.2718715 | 11.4875286 | 11.6417944 | 10.3583545 |
| CD28 | 1.91838623 | 1.15704371 | 0 | 0 |
| AP1S2 | 10.4010822 | 10.0074618 | 9.44594598 | 13.4173607 |
| RAC1 | 11.475242 | 11.417726 | 10.433575 | 10.2671144 |
| AP1M2 | 1.91838623 | 1.15704371 | 4.34340782 | 0 |
| AP2M1 | 13.8235722 | 13.1254829 | 11.2958148 | 12.4415385 |
| AP1M1 | 10.6976626 | 9.91466916 | 9.70137578 | 9.70194752 |
| FYN | 11.0463737 | 9.22937162 | 9.92331263 | 9.48480276 |
| TNFAIP3 | 9.14072754 | 8.30108459 | 6.30451104 | 10.5349934 |
| RNF216 | 10.6992688 | 10.3380678 | 10.0474784 | 9.81440622 |
| IFNLR1 | 2.23572706 | 0 | 7.65492239 | 1.95233357 |
| IL15 | 5.84624257 | 6.07852445 | 3.35473424 | 2.7548875 |
| PM20D1 | 1.91838623 | 3.07038933 | 3.46858332 | 1.28688115 |
| USP17L2 | 0.94860085 | 0 | 1.81557543 | 0 |
| IFNA13 | #N/A | #N/A | #N/A | #N/A |
| NCBP3 | #N/A | #N/A | #N/A | #N/A |
| ITGAX | 1.91838623 | 0 | 2.12432814 | 12.0109791 |
| ISG15 | 7.11977116 | 8.56807012 | 5.30742853 | 9.49920809 |
| SPON2 | 2.23572706 | 8.06242397 | 3.93168306 | 3.03210084 |
| ZNF175 | 7.54187101 | 7.81634371 | 7.14944229 | 7.41388175 |
| RELA | 9.42177031 | 9.20847824 | 8.87408995 | 9.18705509 |
| ILF3 | 11.9717519 | 11.9811106 | 12.6029772 | 11.2341947 |
| CD207 | 0 | 0 | 3.57410151 | 0 |
| PYCARD | 0 | 6.48816149 | 0 | 8.50406418 |
| IL33 | 0 | 1.79493566 | 0 | 3.46727948 |
| FADD | 6.98913901 | 7.9227925 | 7.05245937 | 8.06732679 |
| TRIM5 | 9.86046626 | 9.39705422 | 7.45557381 | 8.07505123 |
| POLR3F | 8.68814538 | 9.06638524 | 8.72471848 | 7.80535695 |
| POLR3E | 9.72956976 | 9.40494994 | 10.2055 | 9.47901224 |
| IFIT5 | 9.48844271 | 10.1410598 | 8.86114896 | 7.2465981 |
| IFNA2 | #N/A | #N/A | #N/A | #N/A |
| CD40 | 0 | 5.46434153 | 3.35473424 | 7.08799315 |
| CXCL9 | 0 | 0 | 0 | 0 |
| FCN3 | 1.51096192 | 1.15704371 | 0.87970577 | 0 |
| IFNA8 | #N/A | #N/A | #N/A | #N/A |
| IFNA1 | 0 | 1.79493566 | 0 | 0 |
| IFNL3 | #N/A | #N/A | #N/A | #N/A |
| IFNL4 | #N/A | #N/A | #N/A | #N/A |
| TRIM11 | 8.79135827 | 8.68502907 | 8.9173124 | 8.96448527 |
| ABCC9 | 3.80426012 | 8.6411486 | 3.76447355 | 0 |
| APOBEC3A | #N/A | #N/A | #N/A | #N/A |
| APOBEC3H | #N/A | #N/A | #N/A | #N/A |
| UNC13D | 2.90496572 | 1.79493566 | 3.09592442 | 7.13298804 |
| IFIT1B | #N/A | #N/A | #N/A | #N/A |
| IRF9 | 3.80426012 | 2.56803211 | 3.09592442 | 4.7473874 |
| KCNJ8 | 0 | 8.35640795 | 8.41290852 | 1.95233357 |
| IFNE | 2.49313492 | 5.24754781 | 0 | 0 |
| IFNL2 | #N/A | #N/A | #N/A | #N/A |
| PRF1 | 1.51096192 | 0 | 1.422233 | 3.26603689 |
| IFNL1 | 0.94860085 | 0 | 0 | 1.95233357 |
| NLRP3 | 1.91838623 | 1.15704371 | 1.422233 | 11.0042837 |
| LYST | 9.96181141 | 10.1981995 | 8.7753469 | 13.2504332 |
| IL6 | 9.68317008 | 5.88141991 | 0.87970577 | 4.07038933 |
| TRIM25 | 8.64205169 | 9.73011345 | 9.43548275 | 8.2349611 |
| STAT1 | 10.5323657 | 11.6290378 | 9.89179889 | 10.8822074 |
| TICAM1 | 6.50604967 | 6.29792505 | 5.14567746 | 7.05723364 |
| ADARB1 | 7.52747701 | 8.00129559 | 8.32880954 | 11.6020725 |
| CARD9 | 5.14118753 | 7.95901662 | 5.60969587 | 9.41970742 |
| SLFN11 | 0 | 9.77952239 | 11.6034038 | 7.64896869 |
| CRCP | 9.8431045 | 9.64380209 | 8.78907713 | 9.83497662 |
| MICA | 9.94969542 | 10.0716769 | 7.48968743 | 8.13524755 |
| FAM111A | 9.71692232 | 11.4118605 | 5.17951105 | 9.03729985 |
| APOBEC3C | 8.5052558 | 9.04636009 | 8.08316017 | 7.10318288 |
| TLR7 | 0 | 0 | 0 | 2.40871186 |
| BNIP3 | 8.52352275 | 9.58942644 | 9.12887076 | 9.28331991 |
| DNAJC3 | 11.2267915 | 11.9305119 | 10.2726065 | 12.6606444 |
| NLRC5 | 8.91004304 | 8.71073757 | 5.45252955 | 9.89716478 |
| RNASEL | 7.02036885 | 7.61331064 | 7.04330075 | 9.09148828 |
| DEFA1 | #N/A | #N/A | #N/A | #N/A |
| APOBEC3D | 0 | 1.15704371 | 5.17951105 | 2.40871186 |
| POLR3G | 7.02036885 | 7.67553369 | 9.33784487 | 6.42240122 |
| GBP3 | 9.24797497 | 10.3693356 | 1.422233 | 7.8509994 |
| DEFA1B | #N/A | #N/A | #N/A | #N/A |
| CXADR | 8.76394266 | 3.86393845 | 10.2618011 | 8.50973542 |
| APOBEC3B | 5.64731451 | 6.69125498 | 6.05419729 | 3.03210084 |
| DDX41 | 9.29980329 | 9.48904513 | 9.31901723 | 10.4277214 |
| DEFA3 | 0 | 0 | 0 | 2.40871186 |
| IFNA5 | #N/A | #N/A | #N/A | #N/A |
| POLR3H | 9.5733821 | 9.62590971 | 9.89689242 | 9.1103528 |
| POLR3D | 10.2445779 | 9.76324557 | 9.66008506 | 10.4216862 |
| DDIT4 | 8.06058786 | 5.9692426 | 8.54194843 | 9.19768359 |
| IRF5 | 3.22342255 | 1.79493566 | 2.94673086 | 9.69446236 |
| AZU1 | 0 | 0 | 0.87970577 | 9.92088874 |
| IL10RB | 8.7822775 | 8.30108459 | 6.84699469 | 10.8678579 |
| F2RL1 | 8.15269046 | 4.68144927 | 8.92976142 | 2.7548875 |
| ABCF3 | 10.2837135 | 9.85446325 | 8.63926835 | 9.38603491 |
| POLR3C | 9.29342567 | 9.05305702 | 9.04884103 | 10.6503876 |
| POLR3B | 8.87061131 | 9.23232498 | 9.75139342 | 7.51932174 |
| PLSCR1 | 8.98056804 | 8.49105138 | 8.32503556 | 9.00537192 |
| CD86 | 0 | 0 | 0 | 3.03210084 |
| UNC93B1 | 8.27858884 | 7.73518367 | 6.97750891 | 10.2535875 |
| POLR3A | 9.46593366 | 9.92018907 | 10.7749435 | 9.47609811 |
| TRIM56 | 9.11444511 | 8.95968323 | 7.41362793 | 9.19414124 |
| BCL2 | 7.951343 | 6.10454634 | 7.7977912 | 10.1893675 |
| ATG7 | 9.17582397 | 9.22937162 | 8.64835758 | 11.3929184 |
| SAMHD1 | 9.32086823 | 9.28745844 | 10.3386027 | 11.3193955 |
| IFI44L | 3.36036428 | 4.68144927 | 1.422233 | 3.80012335 |
| DMBT1 | 2.49313492 | 0 | 0.87970577 | 1.28688115 |
| BNIP3L | 10.3851829 | 11.0767822 | 9.83858941 | 11.4782185 |
| EXOSC4 | 7.26594316 | 8.06910063 | 8.70165301 | 9.91230556 |
| GPAM | 9.79122802 | 9.13126536 | 10.1501154 | 8.42181238 |
| GBP1 | 10.7498359 | 9.71748794 | 1.422233 | 6.74577515 |
| CD8A | 0 | 0 | 0.87970577 | 3.26603689 |
| IFNA6 | #N/A | #N/A | #N/A | #N/A |
| APOBEC1 | 0 | 0 | 0 | 0 |
| IFI16 | 10.2555597 | 10.2117667 | 3.46858332 | 10.7184044 |
| BECN1 | 9.71532713 | 9.61233407 | 9.13532452 | 10.0882582 |
| IFNA21 | #N/A | #N/A | #N/A | #N/A |
| IFNA16 | 0 | 0 | 0 | 0 |
| POLR3K | 8.07553263 | 8.27262979 | 9.07151599 | 7.71945728 |
| IFNA10 | 0 | 0 | 0 | 1.28688115 |
| IFNA14 | 0 | 0 | 0 | 1.95233357 |
| AIMP1 | 9.88612332 | 10.1860399 | 9.74857864 | 9.68943895 |
| OPRK1 | 0 | 1.15704371 | 4.56437817 | 0 |
| TRIM34 | 2.23572706 | 2.83995959 | 0 | 1.95233357 |
| IRF1 | 9.12884499 | 6.9140861 | 6.71493235 | 9.41970742 |
| PTPRC | 0.94860085 | 3.4409522 | 0.87970577 | 11.4614129 |
| EXOSC5 | 8.78528898 | 7.24241196 | 8.77257851 | 8.08273452 |
| PMAIP1 | 10.3239104 | 9.67459836 | 10.3534887 | 9.15104192 |
| BCL2L11 | 6.52073653 | 4.53543092 | 9.14391886 | 7.90388185 |
| BAD | 8.51625158 | 8.31233844 | 7.61845892 | 8.24863919 |
| MAPK14 | 10.6551819 | 10.7552298 | 10.3235727 | 11.0163417 |
| PENK | 0 | 4.37364821 | 2.12432814 | 1.95233357 |
| IKBKE | 7.49817107 | 7.49008872 | 4.96393645 | 10.103419 |
| MAPK11 | 5.82273015 | 7.48018432 | 5.65849714 | 6.99356115 |
| GLI2 | 6.71149491 | 8.28406063 | 7.42071818 | 2.7548875 |
| LGALS9 | 0.94860085 | 2.23266076 | 2.94673086 | 9.77945673 |
| PSMB9 | 6.31777437 | 4.53543092 | 4.66277517 | 8.69620252 |
| ANKRD17 | 10.669204 | 11.5978151 | 11.7991966 | 11.0183393 |
| PUM1 | 10.9664406 | 10.7187469 | 11.0678108 | 10.4924541 |
| TRIM15 | 0 | 0 | 1.422233 | 1.95233357 |
| PUM2 | 9.37623394 | 10.8166797 | 10.5767772 | 10.219229 |

TABLE 2
Gene expression of cell surface genes, Related
to FIG. 12C. Values represent log2(RPKM + 1).

| Associated | Control: | Control: | Control: | Control: |
|---|---|---|---|---|
| Gene Name | CHME_C2 | BJ_C3 | 293_C2 | THP_C2 |
| SERPINA5 | 0 | 0 | 4.2 | 0 |
| GSR | 816.46 | 1060.37 | 2044.87 | 3828.89 |
| IGHG1 | 0 | 2.47 | 0.84 | 0 |
| ITGAX | 2.78 | 0 | 3.36 | 4126.29 |
| FCER2 | 0 | 0 | 0 | 40.23 |
| CD80 | 0.93 | 0 | 2.52 | 2.87 |
| GPC4 | 3.71 | 56.72 | 225.72 | 2104.81 |
| CXCL12 | 864.65 | 1631.24 | 31.89 | 5.75 |
| THY1 | 1889.63 | 5680.35 | 5.03 | 0 |
| EPOR | 48.19 | 51.79 | 55.38 | 67.53 |
| FCGR3A | 0 | 0 | 0 | 14.37 |
| LAMP1 | 4984.96 | 6167.38 | 2294.92 | 5380.56 |
| P4HB | 6100.76 | 8571.7 | 2824.39 | 7545.71 |
| CD36 | 15.75 | 56.72 | 5.87 | 17426.1 |
| IGHA1 | 1.85 | 0 | 0 | 4.31 |
| MCAM | 3140.74 | 337.84 | 564.71 | 31.61 |
| HFE | 236.32 | 265.09 | 1.68 | 2.87 |
| TNF | 2.78 | 1.23 | 0 | 1432.42 |
| IGHD | #N/A | #N/A | #N/A | #N/A |
| TNFRSF13B | 0 | 0 | 0 | 0 |
| ITGA3 | 7291.62 | 5418.96 | 125.86 | 474.12 |
| IL13 | 0 | 0 | 0 | 2.87 |
| CTSV | 34.29 | 9.86 | 543.73 | 87.64 |
| SELL | 0.93 | 0 | 3.36 | 0 |
| HEG1 | 5699.48 | 4219.27 | 437.17 | 1077.55 |
| ALCAM | 13497.1 | 8786.24 | 1165.5 | 20703.29 |
| CD5 | 0 | 1.23 | 1.68 | 4.31 |
| CD40 | 0 | 43.15 | 9.23 | 135.05 |
| CCR7 | 0.93 | 4.93 | 0 | 1.44 |
| ECE1 | 3806.14 | 3458.52 | 854.2 | 715.49 |
| CXCL10 | 0 | 0 | 1.68 | 22.99 |
| PDPN | 35.22 | 13.56 | 573.1 | 231.31 |
| CXCL9 | 0 | 0 | 0 | 0 |
| IL12RB2 | 0 | 7.4 | 18.46 | 45.98 |
| ANPEP | 529.17 | 8394.15 | 2.52 | 13962.15 |
| SCUBE1 | 6.49 | 0 | 33.56 | 28.73 |
| CDH13 | 2046.25 | 578.27 | 1.68 | 0 |
| KCNJ5 | 0 | 0 | 0 | 94.82 |
| ITGA2B | 1.85 | 2.47 | 75.52 | 2.87 |
| SPN | 0.93 | 0 | 0 | 931 |
| ITGAE | 316.95 | 422.91 | 301.23 | 360.62 |
| CHRNB2 | 12.05 | 7.4 | 20.98 | 44.54 |
| IGHE | 0 | 0 | 0 | 1.44 |
| LILRB1 | 0 | 0 | 0 | 33.04 |
| GYPA | #N/A | #N/A | #N/A | #N/A |
| IGHG4 | 0 | 0 | 0 | 1.44 |
| CLCN3 | 919.33 | 1679.32 | 3204.5 | 1193.92 |
| MUC17 | 0 | 0 | 1.68 | 14.37 |
| SLAMF1 | 0 | 0 | 0 | 1.44 |
| SCNN1G | 0 | 0 | 0 | 0 |
| NGFR | 0.93 | 0 | 21.82 | 14.37 |
| CD74 | 6.49 | 3.7 | 10.91 | 502.86 |
| IGHV3-23 | #N/A | #N/A | #N/A | #N/A |
| B4GALT1 | 2853.45 | 2168.82 | 321.37 | 1647.93 |
| ANTXR2 | 830.36 | 2404.32 | 301.23 | 3245.57 |
| ENPEP | 2.78 | 17.26 | 104.05 | 0 |
| RTN4RL1 | 21.32 | 6.16 | 21.82 | 15.8 |
| IL2RG | 0 | 1.23 | 0.84 | 1.44 |
| CCR1 | 0 | 0 | 0 | 530.15 |
| BCAM | 45.41 | 73.98 | 75.52 | 24.42 |
| CD83 | 155.69 | 59.18 | 151.04 | 4025.72 |
| TRDC | 0 | 0 | 0 | 35.92 |
| CALR | 14371.95 | 13169.49 | 4740.88 | 15456.35 |
| CD27 | 0 | 1.23 | 0 | 0 |
| P2RY12 | 0.93 | 0 | 4.2 | 2.87 |
| EPHA5 | 1241.84 | 38.22 | 65.45 | 1.44 |
| TAS2R16 | 0 | 0 | 0 | 2.87 |
| CTLA4 | 0 | 0 | 0 | 1.44 |
| HLA-DRB1 | 0 | 0 | 63.77 | 327.57 |
| SLC22A11 | 0.93 | 0 | 1.68 | 5.75 |
| FASLG | #N/A | #N/A | #N/A | #N/A |
| ASTN1 | 0 | 0 | 5.03 | 1.44 |
| KCNJ3 | 0 | 51.79 | 17.62 | 0 |
| CD4 | 12.97 | 11.1 | 15.1 | 252.86 |
| CLPTM1 | 1431.82 | 1474.65 | 916.29 | 1261.45 |
| FGA | #N/A | #N/A | #N/A | #N/A |
| TLR4 | 163.11 | 876.65 | 0 | 1084.73 |
| LDLR | 2811.74 | 1258.88 | 432.97 | 867.79 |
| ITGA1 | 632.97 | 976.52 | 229.07 | 218.38 |
| ACE | 1.85 | 12.33 | 19.3 | 31.61 |
| IL6 | 821.1 | 57.95 | 0.84 | 15.8 |
| TNFRSF18 | 9.27 | 0 | 1.68 | 0 |
| ICOS | #N/A | #N/A | #N/A | #N/A |
| TNFRSF14 | 1.85 | 66.58 | 0.84 | 294.53 |
| IL6ST | 10855.88 | 14455.49 | 1390.38 | 1790.17 |
| KLRK1 | 0 | 0 | 6.71 | 0 |
| CD14 | 146.43 | 4.93 | 2.52 | 1107.72 |
| CD69 | 0 | 0 | 0 | 7.18 |
| ANXA5 | 11189.51 | 14809.36 | 2561.75 | 22499.2 |
| S1PR1 | 66.73 | 1740.97 | 52.02 | 4.31 |
| MS4A2 | 0 | 0 | 0 | 1.44 |
| KLRD1 | 0 | 3.7 | 0.84 | 0 |
| CXCR3 | 0 | 0 | 2.52 | 4.31 |
| TGFBR3 | 50.97 | 573.34 | 719.1 | 61.78 |
| SPA17 | 88.97 | 108.5 | 84.75 | 117.81 |
| TFR2 | 9.27 | 9.86 | 5.03 | 11.49 |
| CD40LG | 0 | 0 | 0.84 | 0 |
| RTN4RL2 | 3.71 | 0 | 22.66 | 56.03 |
| PECAM1 | #N/A | #N/A | #N/A | #N/A |
| CDH5 | 1.85 | 0 | 4.2 | 2.87 |
| CXCR5 | 0 | 0 | 0 | 35.92 |
| IGHV3OR16-9 | #N/A | #N/A | #N/A | #N/A |
| AMOT | 172.37 | 45.62 | 6269.7 | 33.04 |
| TNFRSF11A | 309.53 | 3.7 | 142.65 | 337.63 |
| DNAI2 | 0 | 0 | 0 | 0 |
| CD3E | 1.85 | 2.47 | 0.84 | 1.44 |
| ABCG1 | 0 | 0 | 40.28 | 5.75 |
| IL2RA | 0 | 0 | 0.84 | 0 |
| CD226 | 6.49 | 19.73 | 2.52 | 498.55 |
| CD9 | 764.56 | 1099.82 | 422.06 | 5101.83 |
| AQP4 | 0.93 | 0 | 21.82 | 10.06 |
| TNFRSF9 | 357.72 | 1.23 | 9.23 | 57.47 |
| CD79A | 0 | 1.23 | 0 | 0 |
| IGHG2 | #N/A | #N/A | #N/A | #N/A |
| ATP6AP2 | 2344.66 | 2721.19 | 1200.74 | 8345.97 |
| CD48 | 0 | 0 | 0 | 10.06 |
| GFRA3 | 9.27 | 0 | 3.36 | 1.44 |
| ENG | 6.49 | 876.65 | 10.91 | 640.78 |
| MFGE8 | 663.55 | 4594.09 | 380.11 | 127.87 |
| SCNN1B | 0.93 | 0 | 0.84 | 4.31 |
| ADA | 30.58 | 77.68 | 168.66 | 100.57 |
| CHRNA4 | 0 | 3.7 | 5.87 | 11.49 |
| VCAM1 | 1071.32 | 0 | 0 | 10.06 |
| GPIHBP1 | 0 | 2.47 | 0 | 8.62 |
| ICAM1 | 3634.69 | 184.95 | 7.55 | 4853.28 |
| NLGN1 | 65.8 | 242.9 | 117.47 | 7.18 |
| CD8B | 0 | 0 | 0 | 1.44 |
| FCER1A | 0 | 0 | 0 | 0 |
| CCR5 | 0.93 | 0 | 0 | 5.75 |
| SEMA7A | 812.75 | 2812.43 | 44.47 | 4060.2 |
| IL2RB | 3.71 | 3.7 | 2.52 | 0 |
| IL1RL1 | 1.85 | 0 | 0 | 2.87 |
| SLC2A4 | 1.85 | 1.23 | 46.15 | 5.75 |
| CD79B | 0 | 2.47 | 3.36 | 8.62 |
| ADGRE1 | #N/A | #N/A | #N/A | #N/A |
| ITGB1 | 27850.52 | 46725.12 | 2770.69 | 17013.76 |
| TFRC | 19994.51 | 11254.67 | 4138.41 | 10202.22 |
| TNFRSF13C | 3.71 | 0 | 5.03 | 1.44 |
| THBS1 | 65649.63 | 171900.02 | 12.59 | 2146.48 |
| CD28 | 2.78 | 1.23 | 0 | 0 |
| CD24 | #N/A | #N/A | #N/A | #N/A |
| CD86 | 0 | 0 | 0 | 7.18 |
| ENOX2 | 500.44 | 304.55 | 357.45 | 373.55 |
| IGLL5 | #N/A | #N/A | #N/A | #N/A |
| TMEM123 | 3243.6 | 4732.19 | 2818.51 | 4554.44 |
| CD276 | 1343.78 | 1525.2 | 1057.26 | 5818.76 |
| PLET1 | #N/A | #N/A | #N/A | #N/A |
| TGFBR2 | 716.37 | 2530.08 | 124.19 | 755.72 |
| SELP | 0 | 1.23 | 0 | 0 |
| ITGA5 | 10868.86 | 8666.64 | 369.2 | 16439.07 |
| IGLL1 | 0 | 0 | 0 | 0 |
| NRCAM | 7.41 | 14.8 | 221.52 | 373.55 |
| IL31RA | 8.34 | 1.23 | 0 | 10.06 |
| TRPV1 | 2.78 | 3.7 | 5.03 | 2.87 |
| FAS | 96.38 | 1336.55 | 23.49 | 8.62 |
| IL4 | 0.93 | 0 | 0 | 0 |
| FCRL6 | 0.93 | 0 | 0 | 133.62 |
| ITGA6 | 1820.13 | 9981 | 654.49 | 5346.07 |
| CD19 | 0 | 0 | 19.3 | 10.06 |
| CTSB | 5729.13 | 19545.25 | 794.62 | 64134.21 |
| NRG1 | 818.32 | 1087.49 | 14.26 | 3090.41 |
| CD274 | 265.98 | 1018.44 | 5.03 | 41.67 |
| GLRA1 | 2.78 | 1.23 | 5.03 | 2.87 |
| C10orf54 | 349.38 | 288.52 | 23.49 | 360.62 |
| CD244 | 0.93 | 1.23 | 0 | 277.29 |
| FGB | 0 | 0 | 0.84 | 0 |
| LAG3 | 7.41 | 1.23 | 5.03 | 0 |
| CD8A | 0 | 0 | 0.84 | 8.62 |
| ITGB6 | 2.78 | 4.93 | 0.84 | 0 |
| FGG | 0.93 | 0 | 0 | 0 |
| IL17A | 0.93 | 0 | 0.84 | 1.44 |
| FGF8 | 0.93 | 0 | 0 | 2.87 |
| CD2 | 0 | 0 | 0 | 0 |
| IFNG | #N/A | #N/A | #N/A | #N/A |
| B2M | 18119.7 | 13594.87 | 2312.54 | 52831.45 |
| P2RX7 | 31.51 | 6.16 | 3.36 | 310.33 |
| CD33 | 1.85 | 0 | 1.68 | 402.28 |
| IL12RB1 | 0 | 0 | 1.68 | 53.16 |
| FCER1G | 21.32 | 3.7 | 0.84 | 6139.15 |
| TRPM8 | 9.27 | 2.47 | 0.84 | 162.35 |
| NCAM1 | 1.85 | 28.36 | 417.87 | 3583.21 |
| ABCA1 | 993.47 | 1371.08 | 20.14 | 1673.79 |
| ITGA2 | 312.31 | 8622.25 | 213.97 | 1741.32 |
| CCR4 | #N/A | #N/A | #N/A | #N/A |
| ADGRA3 | #N/A | #N/A | #N/A | #N/A |
| PLPPR4 | #N/A | #N/A | #N/A | #N/A |
| TMC1 | 0 | 6.16 | 0 | 25.86 |
| CD34 | 2.78 | 8.63 | 1.68 | 1.44 |
| MS4A1 | 0 | 0 | 0 | 5.75 |
| ITGAV | 8557.56 | 7863.97 | 1924.88 | 7252.62 |
| UMODL1 | 2.78 | 1.23 | 0.84 | 1.44 |
| PDCD1 | 0 | 1.23 | 1.68 | 0 |
| BMPR1A | 1376.22 | 1195.99 | 811.4 | 313.21 |
| VTCN1 | 0 | 0 | 2.52 | 0 |
| STAB2 | 3.71 | 2.47 | 2.52 | 2.87 |
| KIT | 15.75 | 156.59 | 259.28 | 8.62 |
| PTPRC | 0.93 | 9.86 | 0.84 | 2818.87 |
| IL7R | 684.86 | 2346.37 | 0.84 | 1284.44 |
| SDC1 | 2092.59 | 2477.06 | 166.14 | 96.26 |
| DEFB125 | #N/A | #N/A | #N/A | #N/A |
| INTU | 343.82 | 263.86 | 658.69 | 71.84 |
| DEFB126 | #N/A | #N/A | #N/A | #N/A |
| DEFB127 | 0 | 0 | 0 | 4.31 |
| HLA-E | 2625.47 | 2870.38 | 560.51 | 17693.34 |
| TMX3 | 4077.67 | 4949.19 | 933.91 | 2418.02 |
| HLA-DPB1 | 0.93 | 27.13 | 77.2 | 1.44 |
| GP6 | 0 | 0 | 0.84 | 5.75 |
| CLSTN1 | 2759.84 | 2068.95 | 1345.07 | 649.4 |
| ADAMTS15 | 126.04 | 103.57 | 80.55 | 433.89 |
| DEFB128 | 0 | 0 | 0 | 2.87 |
| DEFB129 | #N/A | #N/A | #N/A | #N/A |
| TREML1 | 0 | 0 | 0 | 60.34 |
| PVR | 2796.91 | 3094.79 | 539.54 | 1030.14 |
| PLAU | 2690.34 | 1626.3 | 18.46 | 10226.65 |
| PSEN1 | 1368.8 | 1232.98 | 963.28 | 2409.4 |
| DEFB110 | 0 | 0 | 0 | 0 |
| FAP | 499.52 | 3030.67 | 0 | 166.66 |
| HLA-B | 8261.92 | 2716.26 | 1006.91 | 11506.77 |
| DEFB112 | #N/A | #N/A | #N/A | #N/A |
| PVRL2 | 1138.97 | 1057.9 | 453.95 | 1657.99 |
| ENPP1 | 1018.49 | 1452.45 | 882.73 | 10.06 |
| HNRNPM | 3212.1 | 2785.31 | 3958 | 1596.21 |
| TMEM102 | 21.32 | 36.99 | 64.61 | 186.77 |
| ITGA8 | 0 | 110.97 | 198.03 | 2.87 |
| PLA2R1 | 110.28 | 400.72 | 15.94 | 1.44 |
| TIMP2 | 1309.49 | 6331.37 | 232.43 | 372.11 |
| LRFN2 | 0 | 0 | 1.68 | 22.99 |
| EPPIN | 0 | 0 | 0 | 1.44 |
| DEFB132 | #N/A | #N/A | #N/A | #N/A |
| ITGA7 | 215 | 157.82 | 162.78 | 295.97 |
| PLXNB2 | 1633.85 | 3714.98 | 542.89 | 4109.05 |
| BMPR2 | 1161.21 | 3140.41 | 1145.36 | 768.65 |
| CD1B | 0 | 1.23 | 0 | 2.87 |
| EPHA4 | 485.61 | 1341.49 | 667.92 | 0 |
| K1SS1R | 1.85 | 0 | 4.2 | 7.18 |
| CPM | 86.19 | 110.97 | 67.13 | 366.37 |
| HPN | 2.78 | 0 | 0.84 | 0 |
| ADAM8 | 25.02 | 38.22 | 28.53 | 110.63 |
| SHH | 0.93 | 0 | 5.03 | 10.06 |
| SLC46A2 | 1.85 | 0 | 1.68 | 1.44 |
| EPPIN- | #N/A | #N/A | #N/A | #N/A |
| WFDC6 | | | | |
| RSPO2 | 0 | 2.47 | 6.71 | 5.75 |
| HFE2 | 0 | 0 | 1.68 | 0 |
| NT5E | 5570.66 | 10477.89 | 10.91 | 11357.35 |
| SLC6A3 | 0 | 0 | 1.68 | 10.06 |
| AOC3 | 6.49 | 1.23 | 26.01 | 0 |
| EPHB6 | 2.78 | 17.26 | 57.9 | 10.06 |
| FZD1 | 227.05 | 632.52 | 235.79 | 476.99 |
| BACE2 | 3244.53 | 634.99 | 338.99 | 10.06 |
| PLAT | 587.56 | 2657.08 | 380.11 | 12.93 |
| VAMP7 | #N/A | #N/A | #N/A | #N/A |
| ADAMTS9 | 56.53 | 22.19 | 166.14 | 4.31 |
| AAMP | 1613.46 | 1400.67 | 1829.22 | 1719.77 |
| SRPX2 | 67.65 | 415.52 | 6.71 | 311.77 |
| NOTCH4 | 23.17 | 16.03 | 19.3 | 43.1 |
| AREG | 0 | 1.23 | 0.84 | 234.19 |
| IL1R1 | 1002.74 | 1033.24 | 27.69 | 2274.34 |
| TNFRSF10B | 1149.16 | 2384.59 | 961.6 | 1678.1 |
| MMP7 | 0 | 2.47 | 0 | 66.09 |
| HLA-C | 1488.35 | 3525.1 | 2200.1 | 1759.99 |
| AQP11 | 10.19 | 29.59 | 39.44 | 33.04 |
| IL1RAPL1 | 0 | 7.4 | 1.68 | 0 |
| ITGB3 | 252.07 | 182.48 | 2.52 | 1395.07 |
| DCBLD2 | 18375.48 | 12247.22 | 1267.03 | 1182.43 |
| TSPAN14 | 582.92 | 1001.18 | 314.66 | 1145.07 |
| TLN1 | 13822.39 | 15083.08 | 2081.79 | 24480.45 |
| BACE1 | 944.35 | 1437.66 | 319.69 | 370.68 |
| PAM | 3327.94 | 10582.69 | 1517.08 | 6860.39 |
| TGFB1 | 420.74 | 522.78 | 105.73 | 1495.64 |
| CNTN2 | 6.49 | 3.7 | 5.87 | 0 |
| LRFN5 | 0 | 1.23 | 63.77 | 38.79 |
| HYAL4 | 0 | 0 | 0 | 0 |
| NEU1 | 1231.64 | 757.05 | 871.82 | 6440.86 |
| ANXA1 | 10183.07 | 19419.48 | 353.26 | 1718.33 |
| NOD2 | 0 | 1.23 | 16.78 | 1.44 |
| TEK | 2.78 | 165.22 | 5.87 | 0 |
| ADAM15 | 1040.73 | 1115.85 | 587.37 | 1188.18 |
| SULF1 | 1404.94 | 14641.67 | 57.06 | 1.44 |
| ROS1 | 40.78 | 16.03 | 0 | 1.44 |
| ADGRG2 | #N/A | #N/A | #N/A | #N/A |
| C1QBP | 3661.57 | 2906.14 | 7797.69 | 2988.4 |
| DEFB116 | #N/A | #N/A | #N/A | #N/A |
| FGFR3 | 70.43 | 32.06 | 360.81 | 11.49 |
| SORT1 | 442.06 | 1918.52 | 1380.31 | 403.72 |
| PRLR | 12.97 | 102.34 | 2.52 | 126.43 |
| FZD4 | 25.95 | 655.95 | 287.81 | 45.98 |
| SDC3 | 113.99 | 638.69 | 176.21 | 360.62 |
| CCR6 | #N/A | #N/A | #N/A | #N/A |
| TSPAN15 | 186.28 | 11.1 | 238.3 | 127.87 |
| RER1 | 2869.2 | 2903.67 | 1359.33 | 2476.92 |
| HLA-DMA | 12.05 | 18.49 | 15.94 | 12.93 |
| CTSG | 0.93 | 0 | 0 | 260.05 |
| DEFB115 | 0 | 0 | 0 | 0 |
| ISLR2 | 6.49 | 1.23 | 2.52 | 5.75 |
| VAMP4 | 283.58 | 307.01 | 196.35 | 283.04 |
| GRIN2A | 124.18 | 0 | 2.52 | 0 |
| VLDLR | 438.35 | 340.3 | 93.14 | 553.14 |
| TRPV2 | 0.93 | 387.16 | 27.69 | 3130.64 |
| HLA-A | 3051.77 | 1927.15 | 487.51 | 8402 |
| KCNK2 | 151.06 | 940.77 | 8.39 | 0 |
| FZD6 | 963.81 | 1036.94 | 445.56 | 905.14 |
| ADGRA2 | #N/A | #N/A | #N/A | #N/A |
| ENTPD2 | 0.93 | 0 | 0.84 | 1.44 |
| CAV1 | 7135.93 | 14644.14 | 137.61 | 1567.47 |
| ACVR2A | 243.73 | 194.81 | 224.88 | 1089.04 |
| EPCAM | 0 | 9.86 | 29.37 | 2.87 |
| PROM2 | 6.49 | 1.23 | 9.23 | 7.18 |
| DPP6 | 0.93 | 1.23 | 0 | 10.06 |
| TGFA | 2.78 | 429.08 | 6.71 | 30.17 |
| RAMP3 | #N/A | #N/A | #N/A | #N/A |
| HILPDA | 202.03 | 152.89 | 255.08 | 100.57 |
| ANGPTL3 | 3.71 | 7.4 | 20.14 | 4.31 |
| FZD5 | 84.33 | 44.39 | 378.43 | 191.09 |
| ITGAM | 5.56 | 0 | 1.68 | 2543.01 |
| AGER | 25.02 | 4.93 | 11.75 | 30.17 |
| SFRP4 | 3.71 | 14.8 | 5.87 | 0 |
| FZD9 | 60.24 | 16.03 | 94.82 | 110.63 |
| AXL | 8602.97 | 12761.37 | 11.75 | 77.58 |
| TNFRSF4 | 0.93 | 1.23 | 0 | 15.8 |
| APOH | #N/A | #N/A | #N/A | #N/A |
| PDGFB | 42.63 | 1.23 | 4.2 | 102.01 |
| SCUBE3 | 26.88 | 636.22 | 88.1 | 30.17 |
| RAMP1 | 174.23 | 23.43 | 0 | 459.75 |
| DEFB131 | 0 | 0 | 1.68 | 0 |
| ATPIF1 | 1312.27 | 890.21 | 1329.96 | 476.99 |
| HLA-F | 119.55 | 48.09 | 94.82 | 0 |
| USP14 | 463.37 | 1421.63 | 1088.3 | 364.93 |
| VEGFA | 494.88 | 389.62 | 264.31 | 2316.01 |
| IGSF5 | 0 | 0 | 0.84 | 0 |
| BGN | 0.93 | 960.49 | 0 | 0 |
| NDP | 4.63 | 1.23 | 2.52 | 0 |
| HSP90AA1 | 19879.59 | 19462.64 | 25695.55 | 17723.51 |
| PLVAP | 0 | 0 | 0 | 50.29 |
| PDGFA | 152.91 | 202.21 | 37.76 | 8097.41 |
| PDGFC | 1682.04 | 2290.88 | 476.6 | 459.75 |
| PKD1 | 1806.22 | 1681.79 | 453.95 | 1114.9 |
| ABCA7 | 28.73 | 41.92 | 160.27 | 58.91 |
| CASR | 0 | 0 | 0.84 | 8.62 |
| CA4 | 0 | 0 | 0.84 | 2.87 |
| WNT6 | 0.93 | 0 | 5.03 | 12.93 |
| LAYN | 896.16 | 2523.92 | 208.93 | 11.49 |
| DSCAML1 | 0 | 0 | 0.84 | 11.49 |
| AMELX | 0.93 | 0 | 0 | 1.44 |
| RTP1 | #N/A | #N/A | #N/A | #N/A |
| SFRP1 | 57.46 | 5580.48 | 860.07 | 11.49 |
| CAPN5 | 125.11 | 204.68 | 69.64 | 8.62 |
| ITGB4 | 9.27 | 3.7 | 12.59 | 7.18 |
| BMP2 | 2.78 | 16.03 | 372.56 | 304.59 |
| PHB2 | 2374.32 | 2266.22 | 4521.03 | 1254.27 |
| RC3H2 | 3561.48 | 2580.63 | 2155.63 | 1518.62 |
| TFPI | 1318.76 | 1229.28 | 125.86 | 117.81 |
| LILRB2 | 0 | 1.23 | 1.68 | 5.75 |
| EGFR | 1550.44 | 4208.17 | 265.99 | 24.42 |
| IL17RC | 226.13 | 297.15 | 82.23 | 209.76 |
| TPBG | 1430.89 | 3515.23 | 312.14 | 195.4 |
| IGF2R | 5385.31 | 6237.66 | 2798.38 | 10809.96 |
| CAV3 | 2.78 | 6.16 | 3.36 | 1.44 |
| PPFIA4 | 14.83 | 22.19 | 86.43 | 155.17 |
| P2RY1 | 0 | 1.23 | 197.19 | 25.86 |
| NOTCH2 | 7605.79 | 7362.14 | 5396.21 | 4916.49 |
| CLSTN2 | 11.12 | 112.2 | 29.37 | 583.31 |
| MICB | 339.19 | 537.58 | 198.03 | 431.02 |
| F3 | 9729.89 | 5384.44 | 41.12 | 349.13 |
| NRDC | #N/A | #N/A | #N/A | #N/A |
| KCNN2 | 0 | 91.24 | 8.39 | 91.95 |
| HLA-DRA | 0 | 0 | 5.03 | 2.87 |
| HLA-DPA1 | 2.78 | 36.99 | 73 | 11.49 |
| LPAR2 | 11.12 | 0 | 35.24 | 102.01 |
| RTP5 | #N/A | #N/A | #N/A | #N/A |
| MPL | 1.85 | 0 | 0.84 | 21.55 |
| VASN | 1258.52 | 1649.73 | 43.63 | 33.04 |
| CD109 | 569.95 | 3114.51 | 352.42 | 11847.28 |
| TNFRSF10A | 44.48 | 88.77 | 99.01 | 443.95 |
| WNT4 | 22.24 | 7.4 | 14.26 | 8.62 |
| ADAM10 | 4097.14 | 3844.44 | 1270.39 | 4950.97 |
| ACHE | 1.85 | 4.93 | 13.43 | 11.49 |
| STX4 | 392.94 | 484.56 | 281.94 | 1517.19 |
| FOLR2 | 0 | 0 | 0 | 4.31 |
| VAMP1 | 50.97 | 29.59 | 88.1 | 28.73 |
| CST8 | 0 | 0 | 0.84 | 0 |
| GOT2 | 2876.61 | 2511.59 | 3760.82 | 685.32 |
| NLGN4X | 0 | 20.96 | 0.84 | 1.44 |
| ROBO1 | 2049.96 | 1352.58 | 3197.78 | 268.67 |
| NLGN2 | 240.03 | 224.4 | 261.8 | 22.99 |
| TSPAN32 | 1.85 | 7.4 | 1.68 | 5.75 |
| FLT3LG | 0.93 | 0 | 1.68 | 2.87 |
| RALA | 1279.83 | 2124.43 | 521.92 | 543.08 |
| DEFB118 | 0 | 0 | 0 | 0 |
| NOTCH1 | 428.16 | 660.88 | 272.71 | 3455.34 |
| TSPEAR | 0 | 0 | 0.84 | 1.44 |
| CLEC2D | 19.46 | 34.52 | 62.09 | 97.7 |
| ACVR1B | 236.32 | 341.54 | 470.73 | 403.72 |
| PRNP | 3003.58 | 8878.71 | 387.66 | 435.33 |
| PCSK6 | 0.93 | 6.16 | 573.1 | 2370.61 |
| MET | 4298.24 | 9022.97 | 1319.89 | 5883.41 |
| TNS1 | 242.81 | 1433.96 | 68.81 | 685.32 |
| CD38 | 0.93 | 0 | 0 | 21.55 |
| GPRC5B | 336.41 | 9.86 | 213.13 | 0 |
| CLEC9A | 0 | 0 | 0 | 0 |
| PROM1 | 0 | 0 | 2.52 | 1.44 |
| APP | 7446.39 | 12740.41 | 3200.3 | 2630.65 |
| EPHA2 | 1506.89 | 2158.95 | 250.89 | 64.65 |
| IL17RB | 38 | 59.18 | 133.42 | 18.68 |
| DEFB104B | #N/A | #N/A | #N/A | #N/A |
| CX3CL1 | 6.49 | 1.23 | 0 | 1.44 |
| TNFRSF1A | 1099.12 | 1051.73 | 253.41 | 929.56 |
| KCNE1 | 3.71 | 7.4 | 1.68 | 27.3 |
| NLGN4Y | 1.85 | 340.3 | 0 | 18.68 |
| F2R | 2846.96 | 670.74 | 422.9 | 130.74 |
| MST1R | 6.49 | 2.47 | 18.46 | 2.87 |
| FGFR2 | 1019.42 | 0 | 612.54 | 0 |
| SLITRK6 | 0 | 34.52 | 5.87 | 0 |
| LRPAP1 | 925.82 | 2080.04 | 2327.64 | 2285.84 |
| CNTNAP2 | 15.75 | 0 | 391.02 | 7.18 |
| ANO6 | 2874.76 | 3986.23 | 1102.57 | 5188.03 |
| KCNH1 | 29.66 | 64.12 | 0.84 | 0 |
| CXCR4 | 3.71 | 0 | 305.43 | 360.62 |
| TNFSF18 | 0 | 17.26 | 0 | 211.2 |
| CD59 | 11243.26 | 17745.09 | 1050.54 | 4234.04 |
| WNT1 | 0 | 0 | 0 | 1.44 |
| SCARB1 | 99.16 | 397.02 | 535.34 | 479.87 |
| TGFB3 | 84.33 | 32.06 | 73 | 905.14 |
| RTP2 | 0 | 0 | 0 | 0 |
| APOA1 | 0 | 0 | 0 | 5.75 |
| 42615 | 9398.11 | 11122.74 | 5421.38 | 4913.62 |
| DEFB108B | 0 | 0 | 0.84 | 0 |
| SLC7A11 | 1925.77 | 3377.14 | 1023.69 | 10483.82 |
| ACKR3 | 63.95 | 16.03 | 0 | 231.31 |
| CCR2 | 0 | 0 | 0 | 0 |
| SULF2 | 128.82 | 207.14 | 307.11 | 241.37 |
| ELANE | 0.93 | 0 | 0.84 | 318.95 |
| NFAM1 | 1.85 | 0 | 2.52 | 91.95 |
| ERP44 | 926.74 | 893.91 | 568.07 | 1061.74 |
| GJD3 | 0 | 2.47 | 0 | 1.44 |
| DMD | 360.5 | 103.57 | 394.37 | 5.75 |
| HSP90AB1 | 17579.41 | 17217.37 | 26755.32 | 31876.74 |
| TNFSF4 | 12.05 | 98.64 | 9.23 | 34.48 |
| MRC1 | 0 | 0 | 0.84 | 2.87 |
| ROBO2 | 0 | 25.89 | 273.54 | 0 |
| DEFB123 | #N/A | #N/A | #N/A | #N/A |
| IL15 | 56.53 | 66.58 | 9.23 | 5.75 |
| GRIN2B | 1.85 | 0 | 10.91 | 1.44 |
| LPAR1 | 316.95 | 3804.99 | 110.76 | 214.07 |
| KCNQ3 | 1.85 | 27.13 | 0.84 | 97.7 |
| MOG | 0 | 0 | 0 | 1.44 |
| ADCYAP1R1 | 0 | 4.93 | 129.22 | 0 |
| ULBP2 | 187.2 | 187.41 | 40.28 | 451.13 |
| ANXA9 | 26.88 | 23.43 | 52.02 | 63.22 |
| LRFN1 | 1.85 | 1.23 | 117.47 | 14.37 |
| GRIA1 | 9.27 | 32.06 | 0.84 | 1.44 |
| TIGIT | 3.71 | 2.47 | 0.84 | 15.8 |
| HAVCR2 | 7.41 | 1.23 | 1.68 | 6175.07 |
| BMP10 | 0 | 0 | 0 | 15.8 |
| WNT7A | 0 | 0 | 0.84 | 2.87 |
| ITGB8 | 366.06 | 25.89 | 53.7 | 1303.11 |
| SERPINF2 | 0 | 0 | 0 | 2.87 |
| MICA | 987.91 | 1075.16 | 178.73 | 280.16 |
| C5AR1 | 9.27 | 3.7 | 7.55 | 6800.05 |
| SLC9A1 | 239.1 | 447.57 | 177.89 | 297.4 |
| SLC1A4 | 781.25 | 660.88 | 204.74 | 2344.74 |
| BST2 | 0 | 2.47 | 5.03 | 1475.52 |
| DPP4 | 56.53 | 1257.64 | 45.31 | 37.35 |
| DEFB124 | 0 | 0 | 0 | 0 |
| AMBP | 0 | 1.23 | 1.68 | 0 |
| TACR1 | 3.71 | 8.63 | 11.75 | 10.06 |
| MBL2 | 0 | 1.23 | 0 | 0 |
| SLC1A2 | 19.46 | 122.07 | 17.62 | 7.18 |
| CLU | 106.58 | 220.7 | 849.16 | 7.18 |
| KCNA1 | 0 | 2.47 | 37.76 | 0 |
| NRP1 | 5861.66 | 7359.68 | 1056.42 | 9143.35 |
| ADGRF5 | #N/A | #N/A | #N/A | #N/A |
| SLC34A1 | 1.85 | 2.47 | 0 | 1.44 |
| KCNH2 | 0.93 | 2.47 | 83.07 | 21.55 |
| SLIT2 | 506.93 | 4476.96 | 5873.65 | 11.49 |
| HSPA2 | 351.24 | 1006.11 | 277.74 | 11.49 |
| ANXA2 | 25181.49 | 34633.26 | 1884.6 | 11707.92 |
| CSF1R | 28.73 | 9.86 | 2.52 | 6659.25 |
| ATP5B | 9864.27 | 8677.73 | 9815.71 | 9950.8 |
| LGALS1 | 15675.88 | 18436.79 | 36.92 | 14558.39 |
| SDC4 | 2178.78 | 3317.96 | 232.43 | 1274.38 |
| TREML2 | 0 | 0 | 0.84 | 2.87 |
| SLC1A3 | 305.83 | 1.23 | 777.84 | 6633.38 |
| LRP4 | 85.26 | 161.52 | 340.67 | 5.75 |
| HMMR | 2825.64 | 1721.24 | 1414.71 | 41.67 |
| TPO | 0 | 0 | 2.52 | 0 |
| FUT4 | 74.14 | 139.33 | 158.59 | 412.34 |
| NTRK1 | 0 | 1.23 | 4.2 | 30.17 |
| HCST | 2.78 | 6.16 | 1.68 | 228.44 |
| PPFIA3 | 49.12 | 33.29 | 271.87 | 364.93 |
| ENTPD6 | 557.9 | 865.55 | 631.84 | 1581.84 |
| ITGA4 | 840.56 | 2779.14 | 101.53 | 1390.76 |
| ABCB1 | 1.85 | 6.16 | 335.64 | 767.21 |
| SLC46A1 | 8.34 | 127 | 163.62 | 10.06 |
| CORIN | 8.34 | 67.81 | 47.83 | 15.8 |
| PTN | 227.98 | 356.33 | 0.84 | 0 |
| LBP | 0 | 3.7 | 0.84 | 8.62 |
| VAMP5 | 31.51 | 170.15 | 2.52 | 5.75 |
| KCNH5 | 0 | 0 | 0 | 1.44 |
| RARA | 66.73 | 196.04 | 164.46 | 294.53 |
| GHR | 4.63 | 53.02 | 125.02 | 17.24 |
| WNT5A | 3125.91 | 9417.52 | 673.79 | 11.49 |
| KRT4 | 0 | 0 | 2.52 | 1.44 |
| CD93 | 0.93 | 1.23 | 1.68 | 4.31 |
| PLA2G1B | 0 | 0 | 0 | 0 |
| MIF | 3.71 | 0 | 3.36 | 2.87 |
| LPL | 16.68 | 4.93 | 57.9 | 8696.53 |
| RGMA | 0.93 | 6.16 | 135.09 | 35.92 |
| FURIN | 1091.7 | 1736.04 | 648.62 | 3894.98 |
| WNT3A | 0.93 | 0 | 5.03 | 5.75 |
| NRXN1 | 0 | 3.7 | 39.44 | 2.87 |
| CXADR | 433.72 | 13.56 | 1226.75 | 363.49 |
| OTOA | 0 | 3.7 | 0 | 35.92 |
| CCR10 | 1.85 | 7.4 | 8.39 | 0 |
| FOLR1 | 0 | 7.4 | 5.03 | 4.31 |
| HNRNPU | 13535.1 | 10796 | 18000.22 | 7328.76 |
| TRPV4 | 11.12 | 73.98 | 43.63 | 0 |
| DSG2 | 2194.53 | 3.7 | 2941.86 | 8.62 |
| HMGB1 | 3941.44 | 3274.8 | 3229.67 | 1441.04 |
| DEFB105A | #N/A | #N/A | #N/A | #N/A |
| TNN | 0 | 0 | 0 | 21.55 |
| CEP41 | 640.38 | 356.33 | 229.91 | 168.1 |
| DEFB106A | #N/A | #N/A | #N/A | #N/A |
| SLC39A6 | 3077.72 | 3096.02 | 2691.81 | 1745.63 |
| DEFB119 | #N/A | #N/A | #N/A | #N/A |
| CYP2W1 | 12.05 | 0 | 1.68 | 12.93 |
| ITGAD | 0 | 0 | 0 | 11.49 |
| ERP29 | 1138.04 | 1210.79 | 1036.28 | 1169.5 |
| NCL | 6730.94 | 9053.79 | 12308.66 | 4459.61 |
| MSLNL | 2.78 | 0 | 0 | 11.49 |
| CHRNA1 | 0 | 0 | 0 | 0 |
| HM13 | 925.82 | 1623.84 | 844.13 | 2709.67 |
| GPRC6A | 0 | 0 | 2.52 | 1.44 |
| FSHR | 0 | 0 | 0 | 1.44 |
| CFTR | 0 | 0 | 0 | 0 |
| SCARA5 | 3.71 | 0 | 12.59 | 2.87 |
| HTR3B | 0 | 0 | 1.68 | 0 |
| PDIA4 | 7894.01 | 4655.74 | 4646.06 | 5713.88 |
| DEFB121 | #N/A | #N/A | #N/A | #N/A |
| IDE | 1798.81 | 1387.11 | 2008.79 | 999.96 |
| CD55 | 1032.39 | 578.27 | 435.49 | 601.99 |
| SLC11A2 | 668.18 | 774.31 | 789.59 | 1249.96 |
| ART1 | 0 | 0 | 1.68 | 5.75 |
| CD63 | 4658.74 | 12189.27 | 1426.46 | 27445.86 |
| LTF | 0.93 | 1.23 | 0 | 0 |
| SDC2 | 2325.2 | 1292.17 | 849.16 | 25055.14 |
| DEFB135 | 0 | 0 | 0.84 | 0 |
| CLDND1 | 1820.13 | 674.44 | 601.63 | 1120.65 |
| HYAL2 | 353.09 | 536.35 | 263.48 | 478.43 |
| PLG | #N/A | #N/A | #N/A | #N/A |
| ABCC2 | 12.05 | 7.4 | 31.89 | 4.31 |
| GPA33 | 0 | 0 | 0.84 | 0 |
| FERMT2 | 3622.64 | 4988.65 | 1468.41 | 2136.42 |
| CDON | 109.36 | 51.79 | 612.54 | 178.15 |
| DEFB104A | #N/A | #N/A | #N/A | #N/A |
| NTSR1 | 1.85 | 1.23 | 2.52 | 446.82 |
| SLC3A2 | 4065.63 | 2416.65 | 2212.69 | 4867.64 |
| DEFB134 | 0 | 0 | 4.2 | 0 |
| KCNE2 | 0 | 2.47 | 3.36 | 0 |
| PTPRK | 2879.39 | 3060.26 | 839.93 | 854.85 |
| CR1 | 0 | 0 | 0.84 | 0 |
| TMEM206 | 345.68 | 208.37 | 260.12 | 636.47 |
| CLMP | 1010.15 | 4830.83 | 44.47 | 701.12 |
| BSPH1 | 0 | 1.23 | 0 | 0 |
| CCR9 | 0 | 2.47 | 3.36 | 2.87 |
| DIP2A | 598.68 | 622.66 | 620.09 | 619.23 |
| SLC12A1 | 0.93 | 0 | 0 | 1.44 |
| ALPP | 2.78 | 0 | 0 | 1.44 |
| TRPC4 | 226.13 | 482.1 | 4.2 | 0 |
| CLSTN3 | 141.79 | 125.76 | 407.8 | 43.1 |
| NLGN3 | 0 | 0 | 10.91 | 10.06 |
| SLC9A3 | 38 | 14.8 | 18.46 | 109.19 |
| PKD1L3 | 9.27 | 1.23 | 6.71 | 5.75 |
| TLR2 | 67.65 | 1.23 | 0 | 4869.08 |
| LMAN2 | 2015.67 | 1701.52 | 1616.93 | 3905.03 |
| CXCR2 | 0 | 2.47 | 0 | 1.44 |
| UNC5D | 0.93 | 0 | 5.03 | 4.31 |
| ARSB | 506.93 | 1330.39 | 443.04 | 1126.4 |
| LY9 | 0 | 1.23 | 0 | 7.18 |
| ADAMTS7 | 66.73 | 101.1 | 20.98 | 68.96 |
| ADGRV1 | #N/A | #N/A | #N/A | #N/A |
| CD58 | 386.45 | 106.04 | 78.04 | 992.78 |
| RAMP2 | 0.93 | 4.93 | 8.39 | 0 |
| ITGB7 | 17.61 | 11.1 | 22.66 | 7410.66 |
| CD209 | 0 | 0 | 1.68 | 254.3 |
| TF | 3.71 | 4.93 | 1.68 | 4.31 |
| CSPG4 | 1107.46 | 4628.62 | 184.6 | 5452.39 |
| MPZL1 | 2875.69 | 4459.7 | 1355.14 | 425.27 |
| BSN | 3.71 | 8.63 | 172.01 | 54.6 |
| DEFB106B | #N/A | #N/A | #N/A | #N/A |
| PCSK9 | 0.93 | 4.93 | 0.84 | 0 |
| ITGB5 | 2682.92 | 4758.08 | 330.6 | 11916.24 |
| SYNJ2BP | 385.53 | 495.66 | 432.97 | 228.44 |
| STRC | 0 | 1.23 | 5.03 | 2.87 |
| HSPA5 | 9589.95 | 11079.58 | 3310.22 | 8166.38 |
| SLC6A1 | 0 | 0 | 2.52 | 8.62 |
| LRP6 | 1013.86 | 1811.25 | 1961.8 | 334.76 |
| IGSF3 | 635.75 | 13.56 | 647.78 | 22.99 |
| CLEC5A | 0 | 0 | 2.52 | 173.84 |
| PPFIA2 | 1.85 | 2.47 | 20.98 | 70.4 |
| ADAM2 | 0 | 0 | 0 | 2.87 |
| ADIPOQ | 0 | 0 | 0 | 1.44 |
| ANXA4 | 507.86 | 940.77 | 157.75 | 2067.46 |
| VAMP3 | 3101.81 | 3133.01 | 1464.22 | 968.36 |
| PTPRT | 0 | 2.47 | 0 | 2.87 |
| MAS1 | 0 | 0 | 0 | 0 |
| PROCR | 124.18 | 237.97 | 192.99 | 183.9 |
| RPS6KB1 | 733.98 | 880.35 | 1190.67 | 800.26 |
| SRPX | 345.68 | 438.94 | 44.47 | 30.17 |
| FOLH1 | 0 | 0 | 7.55 | 0 |
| APOA4 | 0 | 0 | 0 | 0 |
| SPARC | 57829.76 | 44322.04 | 1261.16 | 4.31 |
| PTPRJ | 1197.35 | 432.78 | 202.22 | 2231.24 |
| LIPC | 0 | 0 | 0 | 1.44 |
| TNR | 0 | 1.23 | 0.84 | 7.18 |
| LY6D | 0 | 1.23 | 0 | 7.18 |
| ACE2 | 0 | 0 | 38.6 | 0 |
| CRYAB | 4.63 | 404.42 | 0 | 0 |
| PDIA3 | 5239.81 | 5045.37 | 2471.97 | 5277.11 |
| ITGAL | 0.93 | 0 | 5.03 | 337.63 |
| TSPAN33 | 6.49 | 1.23 | 260.12 | 137.93 |
| HBEGF | 655.21 | 360.03 | 115.79 | 6509.83 |
| TDGF1 | 0 | 0 | 0 | 0 |
| FAM89B | 88.97 | 80.14 | 34.4 | 79.02 |
| MFI2 | 1454.06 | 48.09 | 63.77 | 928.13 |
| CLEC17A | 0.93 | 0 | 0 | 8.62 |
| DEFB133 | #N/A | #N/A | #N/A | #N/A |
| DEFB105B | #N/A | #N/A | #N/A | #N/A |
| APMAP | 2555.03 | 2361.16 | 1298.92 | 1343.34 |
| CD44 | 8078.43 | 15258.16 | 31.05 | 5098.96 |
| SLC26A9 | 0 | 0 | 0 | 11.49 |
| ADAMTS13 | 28.73 | 24.66 | 72.16 | 12.93 |
| SPTB | 17.61 | 69.05 | 255.92 | 12.93 |
| ANTXR1 | 1074.1 | 2781.61 | 429.62 | 66.09 |
| WNT5B | 1189.01 | 4162.55 | 90.62 | 43.1 |
| GP1BA | 4.63 | 2.47 | 10.07 | 1.44 |
| CLIC4 | 21061.19 | 23371.19 | 2396.45 | 7327.33 |
| TNFRSF12A | 4668.01 | 3546.06 | 57.9 | 1764.31 |
| SCN5A | 8.34 | 48.09 | 125.02 | 7.18 |
| ACVRL1 | 0.93 | 125.76 | 3.36 | 202.58 |
| CD46 | 3627.28 | 2697.77 | 885.24 | 1976.94 |
| PHB | 1395.68 | 1446.29 | 2807.61 | 1543.05 |
| LRFN3 | 80.63 | 82.61 | 57.9 | 132.18 |
| HHIP | 63.95 | 30.82 | 21.82 | 4.31 |
| EPO | 0.93 | 1.23 | 4.2 | 0 |
| MXRA8 | 1561.56 | 3856.77 | 109.08 | 37.35 |
| EMP2 | 602.38 | 1049.27 | 454.79 | 54.6 |
| TYROBP | 3.71 | 4.93 | 0 | 4159.33 |
| LIPG | 103.8 | 48.09 | 40.28 | 37.35 |
| CD1D | 0 | 0 | 12.59 | 281.6 |
| PTGFRN | 2385.44 | 271.26 | 1443.24 | 68.96 |
| RTN4R | 21.32 | 13.56 | 41.95 | 57.47 |
| GREM1 | 22408.67 | 27412.91 | 5.03 | 0 |
| HSPD1 | 7264.75 | 9067.36 | 16483.14 | 6369.03 |
| FGF22 | 0.93 | 3.7 | 4.2 | 2.87 |
| FZD10 | 0 | 0 | 48.67 | 4.31 |
| CDH17 | 0 | 0 | 0 | 5.75 |
| CD53 | 4.63 | 0 | 5.03 | 10462.27 |
| GHRHR | 0 | 0 | 1.68 | 0 |
| FAM234A | #N/A | #N/A | #N/A | #N/A |
| DCSTAMP | 0 | 1.23 | 0 | 2160.84 |
| NTRK2 | 0 | 28.36 | 29.37 | 0 |
| GRIN1 | 3.71 | 3.7 | 4.2 | 15.8 |
| MMP16 | 1448.5 | 2273.62 | 422.9 | 10.06 |
| FGFBP1 | 1.85 | 0 | 3.36 | 0 |
| PTGER3 | 2.78 | 60.42 | 0.84 | 91.95 |
| FZD3 | 3.71 | 54.25 | 1356.81 | 329.01 |
| ADAM9 | 6785.62 | 11556.75 | 1445.76 | 23152.91 |
| ADAM17 | 854.46 | 641.15 | 644.42 | 7614.67 |
| AIMP1 | 945.28 | 1163.94 | 859.23 | 824.68 |
| NPTN | 3350.18 | 3957.88 | 1011.95 | 2890.7 |
| THBD | 13.9 | 12.33 | 20.98 | 12960.75 |
| GHSR | 0 | 1.23 | 0.84 | 0 |
| PKD2L1 | 0 | 0 | 3.36 | 4.31 |
| CIITA | 12.97 | 3.7 | 9.23 | 5.75 |
| IL6R | 46.34 | 104.8 | 103.21 | 10480.95 |
| LRFN4 | 518.05 | 425.38 | 137.61 | 522.97 |
| L1CAM | 315.09 | 2055.38 | 9.23 | 86.2 |
| IQGAP2 | 15.75 | 0 | 873.5 | 1747.06 |
| LRRC8A | 1452.21 | 1965.37 | 495.9 | 1748.5 |
| KCNC1 | 2.78 | 3.7 | 88.1 | 21.55 |
| MSLN | 12.05 | 3.7 | 1.68 | 12.93 |
| TMEM8B | 45.41 | 51.79 | 53.7 | 10.06 |
| EGFLAM | 2.78 | 43.15 | 28.53 | 4.31 |
| FGF10 | 0 | 1.23 | 1.68 | 1.44 |
| ANK3 | 22.24 | 0 | 510.17 | 20.11 |
| ASIC1 | 34.29 | 34.52 | 173.69 | 7.18 |
| ITGB2 | 84.33 | 1.23 | 9.23 | 60433.2 |
| FCN1 | 0 | 0 | 0 | 2.87 |
| SERPINE2 | 1060.2 | 15343.24 | 185.44 | 5369.06 |
| ARSA | 203.88 | 229.33 | 80.55 | 903.7 |
| CUBN | 16.68 | 6.16 | 15.1 | 37.35 |
| F10 | 1.85 | 203.44 | 6.71 | 2.87 |
| GGTLC3 | 0 | 0 | 0.84 | 0 |
| PKHD1 | 0 | 0 | 4.2 | 5.75 |
| GGT6 | 0 | 0 | 0.84 | 4.31 |
| GGTLC1 | 0 | 0 | 0 | 1.44 |
| GGTLC2 | #N/A | #N/A | #N/A | #N/A |
| GGT2 | 0.93 | 0 | 0.84 | 0 |
| GGT1 | 5.56 | 11.1 | 12.59 | 99.13 |
| GGT7 | 83.41 | 178.78 | 138.45 | 53.16 |
| GGT5 | 0.93 | 41.92 | 0 | 10.06 |
| EFNA5 | 373.48 | 9.86 | 853.36 | 22.99 |
| SPIRE2 | 42.63 | 27.13 | 135.93 | 58.91 |
| SVIL | 430.94 | 797.74 | 400.25 | 965.48 |
| STAMBP | 372.55 | 461.14 | 933.91 | 808.88 |
| MEN1 | 435.57 | 329.21 | 787.07 | 352 |
| SSH1 | 696.91 | 1824.81 | 440.52 | 1402.25 |
| RHOB | 261.34 | 2446.24 | 1559.87 | 1538.74 |
| ECT2 | 7855.08 | 3380.84 | 1538.06 | 307.46 |
| NDE1 | 683.94 | 409.35 | 336.48 | 593.37 |
| RDX | 2478.11 | 3427.69 | 2886.48 | 1874.93 |
| WDR73 | 398.5 | 356.33 | 735.05 | 603.43 |
| RAB11A | 2263.11 | 2643.52 | 2510.57 | 1991.31 |
| MYH10 | 2296.47 | 3358.65 | 4572.22 | 22.99 |
| OR2A4 | #N/A | #N/A | #N/A | #N/A |
| RAB21 | 1267.79 | 1980.17 | 1142.84 | 1695.34 |
| PLCD3 | 198.32 | 410.58 | 365.84 | 168.1 |
| SPIRE1 | 877.63 | 981.45 | 401.09 | 1051.69 |
| RAB11FIP3 | 620.92 | 815 | 603.31 | 376.42 |
| PLEKHG6 | 12.05 | 12.33 | 2.52 | 5.75 |
| NF2 | 4767.17 | 5857.9 | 1955.93 | 1782.98 |
| PLK4 | 707.11 | 870.49 | 724.14 | 40.23 |
| MYH9 | 25389.08 | 38543.05 | 4544.53 | 18536.7 |
| RHOC | 2790.43 | 2981.35 | 412.83 | 8746.82 |
| ARF6 | 1402.16 | 1914.82 | 688.06 | 2025.79 |
| PDXP | 57.46 | 32.06 | 62.93 | 31.61 |
| PKN1 | 1402.16 | 1950.58 | 1598.47 | 2216.88 |
| RAB11FIP4 | 12.97 | 6.16 | 819.79 | 1313.17 |
| PKN2 | 1518.01 | 1840.84 | 2245.41 | 456.88 |
| FSD1 | 274.32 | 149.19 | 430.45 | 1.44 |
| 42619 | 382.75 | 392.09 | 848.32 | 170.97 |
| MYLK | 5096.17 | 15322.28 | 196.35 | 271.54 |
| MASTL | 1404.94 | 872.95 | 995.16 | 281.6 |
| PSTPIP1 | 4.63 | 3.7 | 0.84 | 155.17 |
| LIMA1 | 4424.28 | 8031.65 | 672.11 | 1073.24 |
| RACGAP1 | 3694.93 | 3748.27 | 1490.23 | 216.95 |
| DCTN3 | 970.3 | 744.72 | 292 | 928.13 |
| 42625 | 0 | 0 | 4.2 | 1.44 |
| 42620 | 5790.3 | 5566.92 | 1887.96 | 3558.78 |
| ZFYVE19 | 187.2 | 304.55 | 234.95 | 308.9 |
| PITPNM1 | 877.63 | 360.03 | 224.04 | 935.31 |
| CEP55 | 3899.74 | 2687.9 | 1152.91 | 66.09 |
| PPP1CC | 2543.91 | 3158.9 | 4695.56 | 2711.11 |
| RHOA | 7032.13 | 11043.83 | 4682.14 | 11876.01 |
| CEACAM5 | 0 | 1.23 | 0 | 0 |

TABLE 3
Gene expression of immune response genes, Related
to FIG. S2F. Values represent log2(RPKM + 1)

| | Control: | Control: | Control: | Control: |
|---|---|---|---|---|
| gene name | CHME_C2 | BJ_C3 | 293_C2 | THP_C2 |
| C3 | 9.1572206 | 6.75848974 | 2.94673086 | 10.4426947 |
| FCER1G | 4.48026512 | 2.23266076 | 0.87970577 | 12.5840582 |
| EPHB6 | 1.91838623 | 4.19061486 | 5.88019573 | 3.46727948 |
| GATA3 | 1.51096192 | 2.83995959 | 6.78240857 | 2.7548875 |
| IL20RB | 4.59514557 | 3.59693514 | 4.40190347 | 4.7473874 |
| SPN | 0.94860085 | 0 | 0 | 9.86418615 |
| ZP3 | 2.49313492 | 2.23266076 | 3.76447355 | 0 |
| FCER1A | 0 | 0 | 0 | 0 |
| FCN2 | 0.94860085 | 0 | 0.87970577 | 0 |
| FCN1 | 0 | 0 | 0 | 1.95233357 |
| FCN3 | 1.51096192 | 1.15704371 | 0.87970577 | 0 |
| MASP1 | 0 | 10.3061073 | 2.37851162 | 0 |
| MBL2 | 0 | 1.15704371 | 0 | 0 |
| KRT1 | 1.91838623 | 0 | 0.87970577 | 0 |
| MASP2 | 4.06608919 | 3.98185265 | 5.70542504 | 3.64270157 |
| A2M | 5.14118753 | 9.20244221 | 3.35473424 | 15.3594658 |
| SERPING1 | 0 | 7.34447346 | 3.76447355 | 0 |
| CTSC | 8.74224206 | 11.7732258 | 9.34717842 | 13.1142401 |
| MICB | 8.41019692 | 9.07301685 | 7.6368421 | 8.75495429 |
| RAET1E | 0 | 2.56803211 | 0.87970577 | 1.95233357 |
| MICA | 9.94969542 | 10.0716769 | 7.48968743 | 8.13524755 |
| CTSH | 4.35473424 | 6.32030415 | 8.17816686 | 12.0024177 |
| EMP2 | 9.23692307 | 10.0365445 | 8.83222546 | 5.79701298 |
| RIPK3 | 0.94860085 | 4.28466219 | 0 | 7.08799315 |
| LILRB1 | 0 | 0 | 0 | 5.08915913 |
| PPP3CB | 9.64859127 | 9.58248074 | 9.13962809 | 9.42273759 |
| PTPRC | 0.94860085 | 3.4409522 | 0.87970577 | 11.4614129 |
| IL7R | 9.42177031 | 11.1968296 | 0.87970577 | 10.3280466 |
| HLA-E | 11.3589094 | 11.4875286 | 9.13316791 | 14.1110003 |
| FADD | 6.98913901 | 7.9227925 | 7.05245937 | 8.06732679 |
| HLA-A | 11.5759032 | 10.9130016 | 8.93224429 | 13.0366888 |
| IL12A | 4.06608919 | 4.68144927 | 3.46858332 | 3.26603689 |
| XCL1 | 0.94860085 | 0 | 3.35473424 | 0 |
| IL12B | 0.94860085 | 0 | 0.87970577 | 1.28688115 |
| STX7 | 10.7152671 | 10.2905719 | 9.93572493 | 10.1840199 |
| IL23R | 0 | 0 | 0.87970577 | 0 |
| HLA-B | 13.012436 | 11.4079369 | 9.97715111 | 13.4903207 |
| B2M | 14.1453511 | 13.7308809 | 11.1758863 | 15.6891367 |
| P2RX7 | 5.02281165 | 2.83995959 | 2.12432814 | 8.28230079 |
| IL12RB1 | 0 | 0 | 1.422233 | 5.75915583 |
| IL23A | 2.71369582 | 1.15704371 | 2.94673086 | 3.80012335 |
| C6 | 0 | 1.79493566 | 0 | 3.03210084 |
| CD59 | 13.4569011 | 14.1152136 | 10.038288 | 12.0481599 |
| MSH2 | 10.3405066 | 10.7060297 | 11.8397234 | 9.15830673 |
| MLH1 | 10.3801688 | 10.2340869 | 7.88233736 | 9.78886512 |
| PYCARD | 0 | 6.48816149 | 0 | 8.50406418 |
| TRIM5 | 9.86046626 | 9.39705422 | 7.45557381 | 8.07505123 |
| TBK1 | 9.82257083 | 9.62815387 | 9.10268437 | 10.9339783 |
| TMEM173 | 7.41438927 | 9.43352285 | 4.79960542 | 8.9561438 |
| MB21D1 | 8.92389197 | 8.76081973 | 2.37851162 | 8.90517638 |
| MAVS | 9.78517453 | 10.0617222 | 9.2806084 | 8.62972089 |
| NLRC4 | 2.49313492 | 2.56803211 | 4.00898878 | 7.35182265 |
| PYDC1 | 0 | 0 | 1.422233 | 0 |
| TLR4 | 7.35851934 | 9.77750191 | 0 | 10.0844497 |
| SIN3A | 9.49215363 | 9.52085436 | 10.3320477 | 8.94356975 |
| HMGB1 | 11.9448731 | 11.6776316 | 11.6576177 | 10.4938955 |
| AIM2 | 0.94860085 | 1.15704371 | 0 | 6.00495076 |
| IFI16 | 10.2555597 | 10.2117667 | 3.46858332 | 10.7184044 |
| CD36 | 4.06608919 | 5.8509994 | 2.7803101 | 14.0890449 |
| PGLYRP3 | 0 | 0 | 0 | 0 |
| PGLYRP4 | 0 | 0 | 0 | 2.7548875 |
| PGLYRP2 | 0 | 1.15704371 | 0 | 1.95233357 |
| MARCO | 0 | 0 | 0.87970577 | 6.37312616 |
| PGLYRP1 | 1.91838623 | 1.15704371 | 1.81557543 | 2.7548875 |
| DMBT1 | 2.49313492 | 0 | 0.87970577 | 1.28688115 |
| TRAF6 | 8.64871709 | 8.3400281 | 8.35124846 | 9.37046946 |
| PSMB9 | 6.31777437 | 4.53543092 | 4.66277517 | 8.69620252 |
| PSME3 | 10.6096959 | 10.9744146 | 11.5052062 | 10.8689691 |
| CARD11 | 0.94860085 | 1.15704371 | 0 | 1.28688115 |
| ITPR3 | 9.35801377 | 10.8245451 | 7.50311018 | 6.39797405 |
| FBXW11 | 11.0875093 | 10.986738 | 10.59646 | 11.8201032 |
| PSMC6 | 10.6968545 | 10.6704527 | 10.2608137 | 10.8193568 |
| PPP3R1 | 8.26129574 | 9.5684882 | 8.94213326 | 8.82975444 |
| UBE2M | 10.1022251 | 9.85252951 | 10.0947375 | 10.4292184 |
| CHUK | 10.3640252 | 10.2676645 | 10.3936371 | 10.298727 |
| RELA | 9.42177031 | 9.20847824 | 8.87408995 | 9.18705509 |
| TAB2 | 10.4304526 | 10.0109057 | 10.1522468 | 11.5744705 |
| NFATC2 | 5.02281165 | 4.37364821 | 5.4799419 | 3.64270157 |
| PSMB4 | 12.1831639 | 11.3914799 | 11.6341985 | 13.4676285 |
| PSMA7 | 11.9091656 | 11.864782 | 11.4981062 | 12.0437485 |
| PSME4 | 9.96849401 | 11.4099 | 11.3813483 | 10.3134613 |
| PSMB8 | 8.70106383 | 7.99429686 | 0 | 11.5239833 |
| MAP3K14 | 7.08087081 | 6.56544486 | 6.33467553 | 8.04395687 |
| UBE2V1 | 5.38474059 | 5.24754781 | 5.58466191 | 5.86962469 |
| PSMD12 | 10.8239881 | 11.1085506 | 11.6368602 | 10.5830734 |
| PSMC2 | 11.8610947 | 11.2013643 | 10.670373 | 11.4948306 |
| SRC | 8.6218321 | 9.06638524 | 8.17397689 | 10.5830734 |
| PSMB11 | 0 | 0 | 0 | 1.95233357 |
| LYN | 7.71094416 | 9.30444259 | 8.25148241 | 10.9633441 |
| PSMD1 | 12.0960595 | 12.2104735 | 12.0019887 | 12.0324143 |
| PSMC5 | 9.68804045 | 10.1489087 | 9.65257612 | 9.75328362 |
| UBE2D2 | 10.9469355 | 10.7324049 | 10.8311726 | 10.6581133 |
| PSMD10 | 10.2190112 | 10.1644922 | 10.6509261 | 9.92301544 |
| UBA52 | 12.4517881 | 12.1573375 | 12.2104765 | 12.7400485 |
| UBC | 13.4396764 | 13.4964194 | 12.1089001 | 13.7277727 |
| PSMB1 | 11.9810428 | 11.080078 | 11.2876085 | 11.8394636 |
| PRKACB | 9.88045595 | 10.0683752 | 8.49908847 | 8.31524015 |
| PSMB3 | 10.3650429 | 10.638445 | 11.2227345 | 11.5197542 |
| PSMD5 | 9.88612332 | 10.5319467 | 2.12432814 | 7.73903798 |
| RPS27A | 11.6338489 | 12.3918719 | 12.0447322 | 12.2767147 |
| PLCG2 | 2.71369582 | 3.2675358 | 7.21606678 | 8.61393677 |
| PSMC4 | 11.1611697 | 10.7176936 | 10.4783854 | 11.3120775 |
| UBE2D1 | 8.70746267 | 8.23174919 | 8.89965903 | 9.0333404 |
| ITPR2 | 9.41396635 | 10.2863036 | 9.64805193 | 10.3662348 |
| PSMA5 | 11.4869356 | 11.1592091 | 10.999091 | 12.5279559 |
| CALM1 | 12.4099 | 12.7602542 | 11.8655825 | 12.9502843 |
| CLEC7A | 0 | 0 | 0.87970577 | 5.20633065 |
| NFKB1 | 10.0602636 | 9.95106611 | 9.21897486 | 11.0558247 |
| PSMD13 | 11.5640304 | 10.986738 | 10.4673101 | 11.624567 |
| KRAS | 9.58560105 | 9.90356556 | 10.2449465 | 8.9891674 |
| CASP8 | 7.951343 | 8.28974227 | 8.10527998 | 9.26322225 |
| PPP3CA | 8.96993328 | 10.8953708 | 9.51042793 | 9.45557381 |
| PSMD9 | 8.03529399 | 7.87817356 | 7.34082928 | 7.60703435 |
| PDPK1 | 9.62338771 | 9.82338316 | 9.26692706 | 9.65644274 |
| NRAS | 12.0997193 | 11.176223 | 11.5332519 | 13.2913247 |
| SKP1 | 11.314453 | 11.1823571 | 11.0655508 | 11.4105415 |
| ITPR1 | 6.76155123 | 7.30003218 | 9.25507579 | 11.6525224 |
| ICAM2 | 0 | 3.07038933 | 1.81557543 | 2.7548875 |
| EP300 | 9.47348253 | 9.74262944 | 10.7420989 | 10.1442886 |
| CARD9 | 5.14118753 | 7.95901662 | 5.60969587 | 9.41970742 |
| PSMD7 | 10.7428315 | 10.2370426 | 10.0531792 | 10.2704705 |
| PSMC3 | 12.3383798 | 11.622848 | 11.3263115 | 12.1474466 |
| PSMD3 | 10.9870081 | 11.3278558 | 11.7714152 | 11.5225766 |
| KLRK1 | 0 | 0 | 2.94673086 | 0 |
| PSMB6 | 11.0298463 | 10.3103174 | 10.4664538 | 11.2444828 |
| TAB3 | 9.12644637 | 8.77307325 | 8.99765936 | 9.2766595 |
| PSMB10 | 6.69863522 | 5.65306002 | 3.23112516 | 8.71111629 |
| RPS6KA5 | 5.84624257 | 5.24754781 | 7.45557381 | 7.33904828 |
| PSMD6 | 10.9850307 | 10.9744146 | 11.0879733 | 10.6230493 |
| PRKACA | 8.85626989 | 8.85218616 | 8.57088032 | 7.78672704 |
| CLEC4D | 0 | 0 | 0.87970577 | 0 |
| PSMA2 | 9.02231232 | 8.37794796 | 8.84805952 | 8.59797804 |
| ICAM3 | 7.20388631 | 6.8992965 | 6.9582627 | 8.12044526 |
| PAK2 | 11.6732737 | 10.9102305 | 10.1437148 | 11.3766409 |
| MALT1 | 10.6944363 | 10.8008109 | 9.27865853 | 10.8353241 |
| PSMD4 | 11.4625074 | 10.6682645 | 11.2914758 | 12.9257213 |
| SYK | 0.94860085 | 1.79493566 | 8.44799238 | 11.0183393 |
| CALM2 | 12.868074 | 14.3045118 | 11.9820773 | 13.5509884 |
| PSMB2 | 11.0948628 | 10.440184 | 10.9540363 | 9.9400052 |
| IKBKG | 7.63894267 | 7.44997399 | 5.96000193 | 7.97973957 |
| TAB1 | 8.18522234 | 8.38331549 | 8.97859607 | 8.60332962 |
| PRKCD | 3.59931779 | 7.519479 | 6.03584382 | 8.00416428 |
| RAF1 | 11.2970194 | 11.30905 | 10.5870461 | 11.5839572 |
| CLEC4E | 0 | 0 | 0 | 1.95233357 |
| PSMD2 | 14.0123496 | 13.5118823 | 11.5047892 | 12.0496274 |
| HRAS | 10.0924665 | 9.47161425 | 9.45975947 | 10.0100245 |
| PSMA8 | #N/A | #N/A | #N/A | #N/A |
| CLEC6A | 0 | 0 | 0 | 0 |
| PSMD8 | 11.918867 | 11.6452351 | 11.1420049 | 12.2230967 |
| UBB | 11.599792 | 11.0899006 | 11.7163565 | 11.682468 |
| CALM3 | 11.6879355 | 11.9176965 | 11.4964344 | 11.8091733 |
| CD209 | 0 | 0 | 1.422233 | 7.99604973 |
| UBA3 | 10.5059405 | 10.1629451 | 9.58640823 | 10.4589807 |
| NFKBIA | 10.649364 | 8.85602083 | 8.13576058 | 12.218757 |
| NFKB2 | 9.77454 | 8.32346015 | 8.0606419 | 10.2954246 |
| BTRC | 8.92662216 | 9.13126536 | 9.86738686 | 8.36005695 |
| PAK1 | 7.77333705 | 9.66586883 | 8.91480368 | 8.37885838 |
| PSMF1 | 9.01714246 | 9.66368262 | 10.0056947 | 8.24179253 |
| PSME2 | 9.97249163 | 9.4840777 | 8.76980479 | 10.63744 |
| PAK3 | 6.93545975 | 3.07038933 | 5.88019573 | 2.7548875 |
| MAP3K7 | 10.7790463 | 10.5873369 | 10.7271802 | 10.797394 |
| PSME1 | 10.0362698 | 9.18727783 | 9.20261362 | 11.1445627 |
| UBE2N | 10.9962051 | 11.4486016 | 11.0996042 | 11.0431093 |
| CLEC4C | 0 | 0 | 0 | 2.7548875 |
| IL1B | 7.57727775 | 5.5428771 | 0.87970577 | 16.4900765 |
| FYN | 11.0463737 | 9.22937162 | 9.92331263 | 9.48480276 |
| NFATC1 | 4.59514557 | 5.65306002 | 7.33333428 | 8.06732679 |
| PSMD11 | 10.4920835 | 10.9185279 | 11.7260393 | 10.8055262 |
| PSMD14 | 11.6597016 | 11.4543199 | 10.6866231 | 11.5676567 |
| PSMB7 | 11.9192134 | 11.3511932 | 11.1680139 | 11.2072706 |
| IKBKB | 9.50694225 | 9.61006514 | 8.83753333 | 10.2330684 |
| PRKACG | 0 | 0 | 0 | 0 |
| NFATC3 | 9.1454482 | 8.27262979 | 8.88950396 | 9.2766595 |
| PSMA6 | 6.46123386 | 5.38162947 | 6.19298317 | 7.97148608 |
| PSMC1 | 9.80922149 | 9.52569698 | 9.21693982 | 9.86641348 |
| PSMA1 | 9.96716861 | 9.38646609 | 9.32469811 | 10.0613577 |
| CUL1 | 10.9777593 | 10.8420989 | 11.0863021 | 9.94210392 |
| CLEC4A | 0 | 1.79493566 | 2.12432814 | 2.7548875 |
| PSMA4 | 11.4206866 | 11.2562146 | 12.3125256 | 11.7031293 |
| BCL10 | 9.56985561 | 9.42836017 | 8.35495458 | 9.25986093 |
| PSMB5 | 10.9346934 | 10.645127 | 10.6243294 | 10.8124581 |
| RELB | 9.31247459 | 5.9112122 | 5.24488706 | 10.103419 |
| CREBBP | 9.66020987 | 9.70689312 | 10.3764293 | 9.21521749 |
| PSMA3 | 10.8350161 | 10.5581433 | 11.0587629 | 11.2849166 |
| TLR5 | 3.48413813 | 0 | 3.93168306 | 2.40871186 |
| FOS | 3.59931779 | 3.4409522 | 4.84247606 | 10.0901654 |
| TICAM2 | 0.94860085 | 0 | 0.87970577 | 0 |
| MAPK10 | 3.0721058 | 2.56803211 | 4.15218342 | 4.66789213 |
| LY96 | 1.91838623 | 7.40871186 | 0 | 7.70963528 |
| ATF1 | 8.68814538 | 8.77307325 | 8.71035875 | 8.40968793 |
| MAPK14 | 10.6551819 | 10.7552298 | 10.3235727 | 11.0163417 |
| NOD1 | 7.17642251 | 6.52732061 | 6.98709344 | 5.59723319 |
| CTSL | 10.6131172 | 11.610951 | 8.9616812 | 15.3276109 |
| IRF7 | 7.00999653 | 5.42357817 | 6.33467553 | 10.1696996 |
| MAPK3 | 8.21703679 | 9.02261749 | 9.2451486 | 8.94778303 |
| DUSP6 | 6.91348784 | 10.2326128 | 5.00225245 | 14.2847244 |
| UBE2D3 | 11.5480416 | 12.3529566 | 12.1621679 | 12.9455627 |
| NOD2 | 0 | 1.15704371 | 4.15218342 | 1.28688115 |
| TRAF3 | 10.0311774 | 9.79962161 | 9.70283067 | 10.746422 |
| CREB1 | 10.4030653 | 9.92751132 | 9.8504369 | 10.4604559 |
| RPS6KA1 | 8.2569163 | 6.40667272 | 9.59269996 | 13.3234264 |
| BIRC3 | 7.71094416 | 6.79103262 | 4.84247606 | 10.1146794 |
| DUSP3 | 10.094909 | 10.1075963 | 10.4587347 | 11.336328 |
| IKBKE | 7.49817107 | 7.49008872 | 4.96393645 | 10.103419 |
| ITGAM | 2.71369582 | 0 | 1.422233 | 11.3128886 |
| CNPY3 | 9.29556233 | 8.82495874 | 8.57723998 | 10.6772263 |
| ELK1 | 9.87048802 | 9.4075652 | 9.45112867 | 8.24179253 |
| MAPK8 | 10.067757 | 10.3679825 | 9.66902677 | 9.33880322 |
| MAPK7 | 7.74872957 | 7.23064495 | 7.65492239 | 6.96069704 |
| MAP2K7 | 8.73910551 | 8.38331549 | 8.86375265 | 9.09529152 |
| MAP2K4 | 10.0400842 | 9.84095097 | 9.74716918 | 9.65128494 |
| MAPK1 | 11.8661198 | 11.9722834 | 11.261713 | 11.9525593 |
| MAPKAPK2 | 10.9387857 | 10.7219109 | 9.10708627 | 11.132609 |
| PELI1 | 6.49137212 | 6.98595576 | 8.28290308 | 8.69118517 |
| MAP2K6 | 4.06608919 | 4.37364821 | 8.25941374 | 0 |
| LGMN | 9.73271014 | 9.81944466 | 9.45458719 | 6.15785217 |
| IRAK2 | 8.15269046 | 6.48816149 | 6.51759066 | 11.1482223 |
| CTSS | 2.90496572 | 5.65306002 | 2.37851162 | 13.7168838 |
| MAPK11 | 5.82273015 | 7.48018432 | 5.65849714 | 6.99356115 |
| TLR3 | 3.22342255 | 2.56803211 | 5.75114233 | 2.7548875 |
| TICAM1 | 6.50604967 | 6.29792505 | 5.14567746 | 7.05723364 |
| IRAK4 | 9.01457847 | 8.94532678 | 7.58097456 | 9.25306744 |
| PIK3R4 | 10.1299398 | 9.70049174 | 10.4168397 | 9.04122108 |
| MEF2A | 8.77922688 | 9.26160133 | 9.69554151 | 11.2521509 |
| CD14 | 7.20388631 | 2.56803211 | 1.81557543 | 10.1146794 |
| TLR7 | 0 | 0 | 0 | 2.40871186 |
| RP11-330H6.5 | #N/A | #N/A | #N/A | #N/A |
| TNIP2 | 8.7106687 | 9.19031855 | 8.70456077 | 10.1332578 |
| TLR9 | 0 | 0 | 0 | 0 |
| RIPK1 | 10.1745762 | 9.64603662 | 9.03965935 | 9.83043598 |
| LBP | 0 | 2.23266076 | 0.87970577 | 3.26603689 |
| JUN | 10.6798407 | 11.3987437 | 11.3487613 | 10.4186643 |
| SARM1 | 5.21490191 | 5.72219276 | 5.68229237 | 3.94204526 |
| BTK | 0.94860085 | 0 | 2.7803101 | 10.4095501 |
| IRAK1 | 11.0387681 | 11.4383812 | 11.5940353 | 12.7979533 |
| RIPK2 | 8.21703679 | 9.23529626 | 7.97183092 | 9.07620099 |
| TIRAP | 4.35473424 | 6.05202456 | 5.75114233 | 6.74577515 |
| ATF2 | 10.3405066 | 10.0749844 | 9.79428589 | 10.4619194 |
| DUSP7 | 7.7548875 | 9.1842059 | 8.07868455 | 10.9716226 |
| MEF2C | 3.22342255 | 6.86913112 | 8.35124846 | 10.6296116 |
| CD86 | 0 | 0 | 0 | 3.03210084 |
| DUSP4 | 6.33449677 | 9.67895219 | 6.78240857 | 12.4681225 |
| UNC93B1 | 8.27858884 | 7.73518367 | 6.97750891 | 10.2535875 |
| MAP3K8 | 7.09064175 | 4.74899785 | 6.40786257 | 8.97687912 |
| MAPK9 | 9.13121391 | 9.50380587 | 9.69700229 | 9.62528876 |
| PPP2R5D | 10.4265688 | 9.46658634 | 10.3497788 | 10.2105131 |
| CTSK | 5.14118753 | 11.2963714 | 3.23112516 | 5.90472493 |
| TLR2 | 6.10118782 | 1.15704371 | 0 | 12.2497298 |
| MAPKAPK3 | 7.82145512 | 9.29879576 | 9.7258945 | 11.8326606 |
| TLR8 | 0 | 0 | 0 | 2.7548875 |
| CTSB | 12.4843522 | 14.2546042 | 9.63593573 | 15.968829 |
| BIRC2 | 11.2761477 | 12.3209838 | 9.54486786 | 11.0558247 |
| HSP90B1 | 14.0374543 | 14.0106776 | 12.9110549 | 13.665327 |
| TLR6 | 5.21490191 | 5.68790052 | 5.75114233 | 9.2766595 |
| TLR1 | 0 | 2.56803211 | 3.09592442 | 9.71433139 |
| PIK3C3 | 10.6830999 | 9.85252951 | 8.73897045 | 9.99384416 |
| NFKBIB | 8.84758984 | 8.46609686 | 8.25545352 | 8.67606285 |
| TLR10 | 0 | 0 | 0.87970577 | 2.40871186 |
| TANK | 9.51060595 | 9.53292081 | 8.04242547 | 11.1518664 |
| MAP3K1 | 9.3782515 | 8.96327181 | 9.56098238 | 9.3983803 |
| RPS6KA2 | 9.12166302 | 8.74015178 | 7.36299581 | 11.8206776 |
| PELI2 | 5.21490191 | 6.50795317 | 9.18192248 | 5.79701298 |
| MAP2K1 | 10.2412682 | 10.2177518 | 9.91707366 | 10.7691684 |
| MYD88 | 7.21286112 | 8.38331549 | 5.14567746 | 8.42181238 |
| MAP2K3 | 8.27858884 | 9.94024018 | 7.75922243 | 9.08387817 |
| IRF3 | 9.48284828 | 8.96327181 | 8.63622583 | 10.1015293 |
| RPS6KA3 | 10.3487282 | 10.5307477 | 10.59646 | 12.7590122 |
| ITGB2 | 6.41498114 | 1.15704371 | 3.35473424 | 15.8830776 |
| HIST1H2BJ | 12.1617303 | 11.7199064 | 12.4860909 | 7.55282277 |
| HIST1H2BB | 5.59275601 | 10.7552298 | 2.37851162 | 5.6395216 |
| HIST1H2BC | 12.2753185 | 11.7204324 | 12.0589895 | 10.6018729 |
| HIST1H2BF | 10.3609787 | 10.4592574 | 9.89562072 | 6.56056212 |
| HIST1H2BE | 7.08087081 | 10.4793357 | 3.93168306 | 3.94204526 |
| HIST1H2BI | 9.18735207 | 11.3714772 | 0.87970577 | 4.96208625 |
| HIST1H2BK | 11.3776227 | 11.158433 | 11.6508588 | 10.4794468 |
| DEFB1 | 0 | 0 | 0 | 2.7548875 |
| HIST2H2BE | 10.8945222 | 10.4452943 | 11.4317379 | 9.28331991 |
| NOS2 | 0 | 0 | 5.85947267 | 4.89384756 |
| DEFA1 | #N/A | #N/A | #N/A | #N/A |
| PLA2G1B | 0 | 0 | 0 | 0 |
| DEFA1B | #N/A | #N/A | #N/A | #N/A |
| DEFA3 | 0 | 0 | 0 | 2.40871186 |
| RPL39 | 10.4573193 | 10.2072584 | 10.7293233 | 10.892057 |
| LTF | 0.94860085 | 1.15704371 | 0 | 0 |
| H2BFS | 2.23572706 | 1.15704371 | 2.12432814 | 2.40871186 |
| BPIFB1 | 0 | 0 | 0 | 1.28688115 |
| IL4 | 0.94860085 | 0 | 0 | 0 |
| HIST1H2BG | 12.0711104 | 11.7645565 | 10.1243152 | 8.49257426 |
| APOA4 | 0 | 0 | 0 | 0 |
| RNASE3 | 0 | 0 | 0 | 4.18903382 |
| FAU | 11.9971583 | 11.4319572 | 11.6546181 | 12.3388506 |
| KLRD1 | 0 | 2.23266076 | 0.87970577 | 0 |
| KLRC2 | 1.91838623 | 0 | 1.422233 | 0 |
| CH507-9B2.1 | #N/A | #N/A | #N/A | #N/A |
| IFNA13 | #N/A | #N/A | #N/A | #N/A |
| CD1A | 0 | 1.79493566 | 0 | 5.68060567 |
| CD1C | 0 | 0 | 0 | 5.46270675 |
| JAK3 | 0.94860085 | 6.22843392 | 4.79960542 | 0 |
| TNFRSF17 | 0.94860085 | 0 | 0 | 0 |
| LAT2 | 3.89724043 | 4.45680615 | 4.51222689 | 12.1151054 |
| CD1B | 0 | 1.15704371 | 0 | 1.95233357 |
| LILRA2 | 0 | 1.15704371 | 0 | 5.31433399 |
| TEC | 4.98093927 | 5.04701482 | 6.0902183 | 6.70653055 |
| LILRB4 | 1.51096192 | 0 | 1.422233 | 8.96032056 |
| OTUB1 | 10.2922756 | 9.5661492 | 9.72446269 | 10.2671144 |
| LILRA3 | 0 | 0 | 0 | 0 |
| TRPM4 | 7.23083705 | 8.06910063 | 6.55673595 | 8.49833066 |
| TAP2 | 8.39835892 | 7.79246496 | 8.07868455 | 9.17634771 |
| TNFRSF13B | 0 | 0 | 0 | 0 |
| NEDD4 | 10.4677482 | 11.6368783 | 8.85068693 | 9.2291072 |
| LILRA6 | 0 | 0 | 3.67242534 | 3.26603689 |
| ANXA1 | 13.3140266 | 14.2452912 | 8.46866477 | 10.7476308 |
| DBNL | 11.2312512 | 11.2771479 | 10.3216801 | 11.9117178 |
| TAP1 | 10.0640152 | 9.0191737 | 8.87924628 | 11.5973962 |
| IFNA2 | #N/A | #N/A | #N/A | #N/A |
| TNFRSF21 | 9.36411276 | 9.12809746 | 8.97378345 | 10.6426294 |
| IFNA8 | 0.94860085 | 0 | 0 | 0 |
| IFNA1 | 0 | 1.79493566 | 0 | 0 |
| IL2 | #N/A | #N/A | #N/A | #N/A |
| CD6 | 0 | 0 | 2.12432814 | 4.29865832 |
| IFNB1 | 0 | 0 | 0 | 1.95233357 |
| LAIR1 | 0.94860085 | 0 | 2.37851162 | 9.51345127 |
| JAK2 | 8.50155857 | 8.55393642 | 7.16641418 | 9.17275252 |
| PTK2B | 6.04154965 | 5.65306002 | 5.07553263 | 11.4399348 |
| ERAP2 | 9.91133206 | 8.17137698 | 8.46180683 | 6.90989308 |
| LILRA4 | 0 | 0 | 0 | 0 |
| CD1E | 0 | 0 | 1.422233 | 3.46727948 |
| LILRB2 | 0 | 1.15704371 | 1.422233 | 2.7548875 |
| ERAP1 | 9.24355484 | 8.56807012 | 8.46523987 | 10.229624 |
| 42437 | 9.17582397 | 8.51554216 | 8.92976142 | 8.86127305 |
| PIK3CD | 10.1251551 | 8.90878289 | 6.77135741 | 6.94392133 |
| CTLA4 | 0 | 0 | 0 | 1.28688115 |
| HMHB1 | 0 | 0 | 0 | 1.95233357 |
| IFNE | 2.49313492 | 5.24754781 | 0 | 0 |
| RAG1 | 3.80426012 | 5.33842442 | 4.75542174 | 4.89384756 |
| THEMIS | 0 | 0 | 0.87970577 | 0 |
| CD4 | 3.80426012 | 3.59693514 | 4.00898878 | 7.98788928 |
| LILRA1 | 0 | 0 | 0 | 0 |
| FGA | #N/A | #N/A | #N/A | #N/A |
| TNFSF18 | 0 | 4.19061486 | 0 | 7.72928085 |
| DCLRE1C | 9.10231694 | 8.44584255 | 8.64230446 | 8.21417339 |
| FCAMR | 0.94860085 | 0 | 0 | 4.07038933 |
| ORAI1 | 7.14842574 | 7.9518675 | 6.78240857 | 6.70653055 |
| JCHAIN | #N/A | #N/A | #N/A | #N/A |
| TRAT1 | 0 | 1.79493566 | 0 | 0 |
| LILRB3 | 2.23572706 | 3.07038933 | 2.37851162 | 3.46727948 |
| JAM3 | 7.66554911 | 10.3200897 | 10.2716264 | 3.94204526 |
| FCGR1B | 0 | 0 | 0 | 4.18903382 |
| SKAP1 | 1.51096192 | 0 | 2.12432814 | 0 |
| CLEC4M | 0 | 1.15704371 | 0 | 1.28688115 |
| TSC1 | 9.95509771 | 10.204241 | 9.81594361 | 10.0748773 |
| CAMK4 | 8.9963605 | 6.72519582 | 8.00045077 | 6.82094479 |
| LILRB5 | 0 | 0 | 1.422233 | 1.28688115 |
| CLEC10A | 0 | 0 | 0 | 1.95233357 |
| TNFRSF11A | 8.27858884 | 2.23266076 | 7.16641418 | 8.40356598 |
| CSK | 8.59443658 | 9.41017572 | 10.1941782 | 10.3069275 |
| LIME1 | 5.25058267 | 3.98185265 | 4.40190347 | 3.80012335 |
| IFNK | 0 | 0 | 2.12432814 | 1.28688115 |
| CD79A | 0 | 1.15704371 | 0 | 0 |
| GPR183 | 3.59931779 | 3.07038933 | 3.8509994 | 11.7520252 |
| RNF125 | 4.21800615 | 3.07038933 | 8.69581987 | 4.82273015 |
| PRKD2 | 7.97813842 | 7.85530455 | 7.65492239 | 6.80258099 |
| ITK | 0.94860085 | 0 | 0 | 2.40871186 |
| IFNA5 | #N/A | #N/A | #N/A | #N/A |
| CD8B | 0 | 0 | 0 | 1.28688115 |
| EOMES | 0 | 1.15704371 | 5.79545527 | 8.20716078 |
| PIK3CG | 0.94860085 | 0 | 0 | 10.0220348 |
| CD7 | 0 | 0 | 0 | 3.46727948 |
| PAG1 | 6.50604967 | 8.4148966 | 9.55618031 | 11.8658067 |
| CD79B | 0 | 1.79493566 | 2.12432814 | 3.26603689 |
| ICOSLG | 4.98093927 | 5.9112122 | 5.5068431 | 9.18705509 |
| RNF19B | 8.10496561 | 7.519479 | 7.77036657 | 9.16914834 |
| ADGRE1 | #N/A | #N/A | #N/A | #N/A |
| TNFRSF13C | 2.23572706 | 0 | 2.592158 | 1.28688115 |
| LAX1 | 1.51096192 | 0 | 0.87970577 | 1.28688115 |
| PRKCB | 2.49313492 | 0 | 9.02580399 | 10.3942269 |
| TFEB | 5.14118753 | 6.07852445 | 5.68229237 | 9.90800225 |
| LAT | 2.90496572 | 0 | 2.12432814 | 3.64270157 |
| CRACR2A | #N/A | #N/A | #N/A | #N/A |
| PIK3CA | 10.7598965 | 10.4478478 | 8.25941374 | 9.86418615 |
| NLRP10 | 4.75221337 | 7.44997399 | 0 | 0 |
| TXK | 1.91838623 | 2.56803211 | 3.35473424 | 1.95233357 |
| BTLA | 0 | 0 | 1.81557543 | 2.40871186 |
| FGB | 0 | 0 | 0.87970577 | 0 |
| IFNW1 | #N/A | #N/A | #N/A | #N/A |
| CD46 | 11.8250701 | 11.3980863 | 9.79155363 | 10.949783 |
| IFNG | #N/A | #N/A | #N/A | #N/A |
| IFNA6 | #N/A | #N/A | #N/A | #N/A |
| PIK3CB | 8.30843005 | 8.03556893 | 8.6239912 | 9.46734062 |
| LAMP3 | 0.94860085 | 1.79493566 | 4.88410945 | 2.40871186 |
| TFE3 | 9.26978124 | 9.88111396 | 8.18651046 | 9.98977765 |
| BTNL8 | 0 | 0 | 0.87970577 | 0 |
| IFNA21 | #N/A | #N/A | #N/A | #N/A |
| IFNA16 | 0 | 0 | 0 | 0 |
| IFNA17 | #N/A | #N/A | #N/A | #N/A |
| IFNA4 | #N/A | #N/A | #N/A | #N/A |
| IFNA10 | 0 | 0 | 0 | 1.28688115 |
| IFNA14 | 0 | 0 | 0 | 1.95233357 |
| IFNA7 | #N/A | #N/A | #N/A | #N/A |
| BTN3A1 | 8.94301291 | 6.95768249 | 7.41362793 | 8.17881422 |
| ZAP70 | 1.91838623 | 0 | 1.81557543 | 3.46727948 |
| SIT1 | 0 | 0 | 0 | 3.64270157 |
| VTCN1 | 0 | 0 | 1.81557543 | 0 |
| FCRL4 | 0 | 0 | 0.87970577 | 0 |
| GBF1 | 11.0868926 | 11.005028 | 10.8364765 | 9.98159582 |
| C8orf4 | 1.51096192 | 8.65888982 | 2.94673086 | 0 |
| SLAMF1 | 0 | 0 | 0 | 1.28688115 |
| DOCK2 | 8.04039932 | 7.52911816 | 0 | 11.6252203 |
| PRKCE | 6.56376828 | 8.31233844 | 7.67877617 | 5.02724254 |
| SBNO2 | 10.4658521 | 9.71115071 | 8.30606169 | 10.6784857 |
| HAVCR2 | 3.0721058 | 1.15704371 | 1.422233 | 12.5924734 |
| TYROBP | 2.23572706 | 2.56803211 | 0 | 12.0224823 |
| CX3CR1 | 0.94860085 | 0 | 0.87970577 | 3.26603689 |
| PSEN1 | 10.4197496 | 10.2691033 | 9.91330832 | 11.2350569 |
| EIF2AK4 | 10.3569141 | 11.5960919 | 10.0255687 | 9.84403094 |
| LCP1 | 7.61191007 | 3.73660488 | 4.28243981 | 15.4003945 |
| F2RL1 | 8.15269046 | 4.68144927 | 8.92976142 | 2.7548875 |
| APBB1IP | 6.47638169 | 7.48018432 | 0 | 9.26322225 |
| ICAM1 | 11.8280135 | 7.53877094 | 3.09592442 | 12.2450416 |
| ITGAL | 0.94860085 | 0 | 2.592158 | 8.40356598 |
| SEMA4A | 2.49313492 | 3.59693514 | 4.00898878 | 4.96208625 |
| ZFPM1 | 5.10265813 | 5.8509994 | 5.53325185 | 6.29609026 |
| STAT6 | 8.82416321 | 10.5522272 | 7.34082928 | 12.2441854 |
| RPS6 | 14.3659813 | 14.1478202 | 13.657563 | 15.2185992 |
| SLC11A1 | 3.0721058 | 3.07038933 | 2.12432814 | 9.80980028 |
| ADA | 4.98093927 | 6.29792505 | 7.40650266 | 6.66633054 |
| ITFG2 | 7.76725753 | 7.50000529 | 7.82476785 | 6.39797405 |
| CDH17 | 0 | 0 | 0 | 2.7548875 |
| LFNG | 9.34777637 | 6.22843392 | 4.924575 | 8.09792677 |
| NOTCH2 | 12.8930721 | 12.8461054 | 12.3979981 | 12.2637064 |
| BCL3 | 6.35102755 | 5.04701482 | 3.09592442 | 7.47346224 |
| DLL1 | 4.64961546 | 2.56803211 | 6.05419729 | 6.97727992 |
| MFNG | 2.90496572 | 0 | 3.35473424 | 0 |
| NKX2-3 | 0 | 0 | 4.88410945 | 1.28688115 |
| LGALS1 | 13.9363508 | 14.1703781 | 5.24488706 | 13.8296623 |
| GAPT | 0 | 1.15704371 | 0 | 0 |
| ABL1 | 11.0911907 | 10.8925656 | 10.0746767 | 9.49633451 |
| CD180 | 0 | 0 | 0 | 6.5158575 |
| PLCL2 | 7.90189263 | 7.40871186 | 8.6085875 | 8.08273452 |
| CD244 | 0.94860085 | 1.15704371 | 0 | 8.12044526 |
| SH2D1B | 0 | 1.15704371 | 0 | 0 |
| MR1 | 7.30879407 | 8.79324564 | 6.12536181 | 8.96032056 |
| HSPD1 | 12.826896 | 13.146626 | 14.008791 | 12.6370845 |
| DLG1 | 12.6482744 | 11.254757 | 9.74998672 | 9.31295101 |
| KIT | 4.06608919 | 7.30003218 | 8.02392065 | 3.26603689 |
| TREM1 | 0.94860085 | 2.23266076 | 0 | 6.32228872 |
| POU2F2 | 4.21800615 | 6.83832152 | 3.35473424 | 8.9561438 |
| HLA-DRB1 | 0 | 0 | 6.01725384 | 8.36005695 |
| HLA-DQB1 | 0 | 0 | 6.71493235 | 3.03210084 |
| IL6 | 9.68317008 | 5.88141991 | 0.87970577 | 4.07038933 |
| IL6R | 5.5649878 | 6.72519582 | 6.70334992 | 13.3556195 |
| IFNL2 | #N/A | #N/A | #N/A | #N/A |
| IFNLR1 | 2.23572706 | 0 | 7.65492239 | 1.95233357 |
| FFAR2 | 0 | 0 | 0.87970577 | 6.56056212 |
| OTUD7B | 10.456375 | 10.2458617 | 9.75978834 | 10.1929092 |
| FFAR3 | 0 | 0 | 0 | 2.40871186 |
| PIGR | 0 | 0 | 2.12432814 | 9.19768359 |
| RAB17 | 0 | 2.23266076 | 0 | 5.97223312 |
| PRF1 | 1.51096192 | 0 | 1.422233 | 3.26603689 |
| GCNT3 | 1.51096192 | 0 | 1.422233 | 0 |
| C3AR1 | 2.49313492 | 0 | 1.422233 | 7.92106733 |
| C5AR1 | 3.36036428 | 2.23266076 | 3.09592442 | 12.7315418 |
| CR1 | 0 | 0 | 0.87970577 | 0 |
| GPLD1 | 1.91838623 | 2.56803211 | 6.69167374 | 3.64270157 |
| RASGRP1 | 4.80115866 | 6.50795317 | 7.13226837 | 4.89384756 |
| PNMA1 | 9.77605463 | 9.63264993 | 9.45803743 | 9.30970371 |
| IL1F10 | #N/A | #N/A | #N/A | #N/A |
| IL25 | 0 | 1.15704371 | 0 | 0 |
| IL36RN | 1.51096192 | 0 | 0 | 6.82094479 |
| NOTCH1 | 8.74537181 | 9.37042587 | 8.09650433 | 11.7550294 |
| IL37 | 0 | 0 | 0 | 0 |
| CCR2 | 0 | 0 | 0 | 0 |
| IL1A | 3.48413813 | 3.73660488 | 1.422233 | 8.76927586 |
| RBPJ | 9.902978 | 11.255483 | 11.15108 | 11.6376347 |
| IL2RA | 0 | 0 | 0.87970577 | 0 |
| IL1RN | 2.71369582 | 1.79493566 | 0.87970577 | 12.5667391 |
| IL36G | 0 | 0 | 0 | 3.26603689 |
| HMGB2 | 11.2593372 | 11.0248003 | 11.8668771 | 8.10548952 |
| IL36A | #N/A | #N/A | #N/A | #N/A |
| KDM6B | 5.21490191 | 5.42357817 | 6.53075746 | 8.20011392 |
| IL36B | 0 | 0 | 0 | 5.08915913 |
| IL5RA | 0.94860085 | 1.15704371 | 0 | 0 |
| OPRM1 | 0 | 0 | 1.81557543 | 2.40871186 |
| ELANE | 0.94860085 | 0 | 0.87970577 | 8.32170266 |
| BDKRB2 | 6.52073653 | 7.93014595 | 2.37851162 | 2.40871186 |
| NPFF | #N/A | #N/A | #N/A | #N/A |
| IL31RA | 3.22342255 | 1.15704371 | 0 | 3.46727948 |
| TNF | 1.91838623 | 1.15704371 | 0 | 10.4852457 |
| AHCY | 10.8768546 | 10.7428231 | 11.4500256 | 10.8477543 |
| EXO1 | 9.55216954 | 9.77952239 | 10.807049 | 7.04154965 |
| BTN3A2 | 9.0325417 | 7.9518675 | 8.16977471 | 9.26659917 |
| GNL1 | 10.0875557 | 9.81352469 | 10.0679183 | 10.8101539 |
| BTN3A3 | 7.97280751 | 6.32030415 | 5.53325185 | 6.44625623 |
| MYO1G | 2.23572706 | 1.15704371 | 2.12432814 | 7.10318288 |
| FOXP3 | 0 | 1.15704371 | 2.37851162 | 0 |
| JAG1 | 12.2133199 | 7.41911748 | 8.09650433 | 11.8326606 |
| CD8A | 0 | 0 | 0.87970577 | 3.26603689 |
| KDM5D | 9.49401552 | 9.29314974 | 0 | 9.38603491 |
| RFTN1 | 9.37221211 | 10.242924 | 7.64291813 | 11.3120775 |
| TGFB1 | 8.72021005 | 9.03281716 | 6.73782194 | 10.5475115 |
| UNC13D | 2.90496572 | 1.79493566 | 3.09592442 | 7.13298804 |
| BCL6 | 8.18522234 | 7.90809234 | 5.55918587 | 10.5265873 |
| CXCL13 | 0 | 0 | 0 | 1.28688115 |
| KLHL6 | 1.91838623 | 1.15704371 | 0 | 10.1202768 |
| ADAM17 | 9.74055659 | 9.32676653 | 9.33409447 | 12.8947553 |
| HCK | 0.94860085 | 1.15704371 | 0.87970577 | 8.66590436 |
| FUT7 | 0 | 0 | 0 | 4.82273015 |
| SNAP23 | 10.2621066 | 9.70049174 | 9.24114898 | 10.6309132 |
| PI4K2A | 9.30403183 | 9.45394963 | 8.51900715 | 11.4158579 |
| WAS | 1.91838623 | 0 | 0 | 7.94649694 |
| HFE | 7.89068988 | 8.05577048 | 1.422233 | 1.95233357 |
| RC3H1 | 9.99756057 | 10.2487815 | 9.95903112 | 10.3248106 |
| TNFAIP3 | 9.14072754 | 8.30108459 | 6.30451104 | 10.5349934 |
| PKN1 | 10.4544638 | 10.9304269 | 10.6433782 | 11.1149656 |
| FOXJ1 | 1.51096192 | 0 | 0 | 3.46727948 |
| CLEC4G | 0 | 1.79493566 | 0 | 0 |
| DUSP22 | 7.93504715 | 7.58578881 | 9.38930867 | 6.42240122 |
| ZBTB1 | 10.4226745 | 9.85252951 | 8.99528664 | 10.9029253 |
| TRIL | #N/A | #N/A | #N/A | #N/A |
| CLC | 0 | 0 | 0 | 0 |
| C10orf54 | 8.45277662 | 8.1775192 | 4.61412087 | 8.49833066 |
| SASH3 | 0 | 0 | 1.81557543 | 5.83364913 |
| FZD5 | 6.41498114 | 5.50430258 | 8.56768994 | 7.58563861 |
| TNFSF4 | 3.7059779 | 6.63865312 | 3.35473424 | 5.14893411 |
| TRAF2 | 9.12166302 | 8.42012866 | 8.44799238 | 8.60332962 |
| BST2 | 0 | 1.79493566 | 2.592158 | 10.5279852 |
| ATG5 | 10.8165997 | 10.01607 | 10.0185897 | 10.9392633 |
| IL10 | 0 | 0 | 0 | 5.75915583 |
| APOA1 | 0 | 0 | 0 | 2.7548875 |
| ANGPT1 | 4.14404637 | 11.2850091 | 2.37851162 | 1.28688115 |
| APOA2 | 0 | 0 | 0 | 2.40871186 |
| TNFRSF14 | 1.51096192 | 6.07852445 | 0.87970577 | 8.20716078 |
| WNT5A | 11.610522 | 13.2012847 | 9.39829478 | 3.64270157 |
| TRIM6 | 7.2917693 | 6.54643149 | 6.70334992 | 1.95233357 |
| REG3G | 0 | 0 | 0 | 2.7548875 |
| IRAK3 | 5.10265813 | 8.20789285 | 3.23112516 | 8.65051327 |
| TNIP1 | 10.8276762 | 9.9220194 | 9.17147707 | 11.0332509 |
| TNIP3 | 2.49313492 | 3.59693514 | 0.87970577 | 6.06802584 |
| SKAP2 | 3.36036428 | 8.60962201 | 9.20057988 | 10.1804562 |
| PPP2R3C | 8.54874486 | 8.74432931 | 8.51573926 | 10.1071778 |
| IRG1 | 0 | 0 | 0 | 4.58436125 |
| HLA-G | 0 | 0 | 0 | 0 |
| FGR | 2.23572706 | 0 | 0 | 11.7326677 |
| CD40 | 0 | 5.46434153 | 3.35473424 | 7.08799315 |
| KIR2DL1 | #N/A | #N/A | #N/A | #N/A |
| KLK3 | 0 | 0 | 0.87970577 | 2.40871186 |
| SPINK5 | 0 | 1.15704371 | 0 | 1.28688115 |
| PGC | 0 | 1.79493566 | 0 | 1.95233357 |
| KLK5 | 0 | 0 | 0 | 0 |
| KLK7 | 0 | 0 | 0 | 0 |
| DUSP10 | 8.53075746 | 8.37260392 | 6.12536181 | 10.9115568 |
| IRF1 | 9.12884499 | 6.9140861 | 6.71493235 | 9.41970742 |
| SAMSN1 | 0 | 0 | 0 | 4.07038933 |
| TRIM27 | 10.4799722 | 10.1363247 | 10.6961417 | 10.3895991 |
| ALOX15 | 0 | 0 | 2.7803101 | 2.7548875 |
| SIRT1 | 9.93610787 | 9.25287827 | 10.268694 | 9.39221008 |
| IL6ST | 13.406322 | 13.8194297 | 10.4423008 | 10.8066866 |
| IL33 | 0 | 1.79493566 | 0 | 3.46727948 |
| IL1RL1 | 1.51096192 | 0 | 0 | 1.95233357 |
| IL27RA | 8.28720446 | 7.43978934 | 7.51633039 | 8.40356598 |
| ECM1 | 9.22134544 | 9.43095427 | 2.94673086 | 10.194683 |
| NDFIP1 | 10.6418531 | 10.6769884 | 10.3357923 | 11.1158955 |
| IFNL1 | 0.94860085 | 0 | 0 | 1.95233357 |
| CD74 | 2.90496572 | 2.23266076 | 3.57410151 | 8.97687912 |
| NLRP3 | 1.91838623 | 1.15704371 | 1.422233 | 11.0042837 |
| IDO1 | 0 | 0 | 0 | 0 |
| HRG | 0 | 1.15704371 | 0 | 3.26603689 |
| CD24 | #N/A | #N/A | #N/A | #N/A |
| PVR | 11.4501338 | 11.5960919 | 9.07825757 | 10.0100245 |
| PVRL2 | 10.1547801 | 10.0483506 | 8.82956419 | 10.6960895 |
| NCR3 | 0 | 0 | 0.87970577 | 3.26603689 |
| CD226 | 2.90496572 | 4.37364821 | 1.81557543 | 8.96448527 |
| CRTAM | 0 | 0 | 0 | 3.46727948 |
| NLRP6 | 0 | 1.15704371 | 0 | 3.64270157 |
| GPX1 | 10.1323198 | 10.361242 | 9.8943479 | 13.6319171 |
| CD28 | 1.91838623 | 1.15704371 | 0 | 0 |
| ADCYAP1 | 0 | 0 | 0 | 0 |
| VIMP | 9.86619709 | 9.94386274 | 9.12670447 | 10.2704705 |
| CNR1 | 5.25058267 | 0 | 2.7803101 | 8.90950311 |
| LTA | 0 | 0 | 0.87970577 | 5.14893411 |
| CCR7 | 0.94860085 | 2.56803211 | 0 | 1.28688115 |
| SPNS2 | 3.22342255 | 3.98185265 | 5.55918587 | 9.19768359 |
| ZP4 | #N/A | #N/A | #N/A | #N/A |
| PTPN6 | 4.06608919 | 1.79493566 | 4.66277517 | 11.601404 |
| HPX | 2.49313492 | 3.2675358 | 1.81557543 | 0 |
| FCER2 | 0 | 0 | 0 | 5.36562256 |
| HLA-DOB | 0.94860085 | 0 | 2.592158 | 3.03210084 |
| CCL14 | 0 | 0 | 0 | 1.95233357 |
| CCL3 | 0.94860085 | 1.15704371 | 0 | 12.5426232 |
| HLA-DPB1 | 0.94860085 | 4.81403765 | 6.2890967 | 1.28688115 |
| PKHD1L1 | 0 | 0 | 1.422233 | 1.95233357 |
| ACKR2 | 1.91838623 | 0 | 2.12432814 | 0 |
| HLA-DOA | 0 | 0 | 7.62461271 | 0 |
| CCL15 | 0 | 2.23266076 | 0 | 3.03210084 |
| CCL4 | 0.94860085 | 0 | 0 | 11.6060674 |
| TNFRSF10C | 1.91838623 | 5.81966818 | 4.15218342 | 3.94204526 |
| SP2 | 5.53636379 | 5.5428771 | 6.01725384 | 5.6395216 |
| LTB | 0.94860085 | 1.15704371 | 2.37851162 | 5.36562256 |
| TNFSF12- | #N/A | #N/A | #N/A | #N/A |
| TNFSF13 | | | | |
| HLA-DRA | 0 | 0 | 2.592158 | 1.95233357 |
| CRHR1 | 0.94860085 | 0 | 1.81557543 | 3.03210084 |
| SUSD2 | 3.48413813 | 4.09000653 | 3.09592442 | 3.26603689 |
| OAS2 | 2.49313492 | 3.73660488 | 1.81557543 | 8.02020215 |
| C1R | 8.24374524 | 8.89390822 | 5.79545527 | 1.28688115 |
| ENPP1 | 9.99363191 | 10.5052657 | 9.78746185 | 3.46727948 |
| EXOSC9 | 10.2837135 | 10.0897418 | 9.86868368 | 8.45762712 |
| GPSM3 | 5.02281165 | 6.18010807 | 4.56437817 | 10.4307243 |
| TNFRSF11B | 6.59170933 | 13.3870965 | 1.81557543 | 4.18903382 |
| HLA-DQB2 | 0 | 0 | 0 | 1.28688115 |
| CXCL12 | 9.75764002 | 10.6726375 | 5.0395771 | 2.7548875 |
| PTGDR2 | 1.91838623 | 2.56803211 | 3.09592442 | 3.26603689 |
| CRIP1 | 2.49313492 | 1.79493566 | 1.81557543 | 3.26603689 |
| TNFSF12 | 5.14118753 | 6.25190841 | 3.09592442 | 2.40871186 |
| TNFRSF1B | 2.71369582 | 3.73660488 | 0.87970577 | 12.6803551 |
| OASL | 2.49313492 | 2.56803211 | 0.87970577 | 6.58225491 |
| KIR2DS2 | #N/A | #N/A | #N/A | #N/A |
| KIR2DL3 | 0 | 1.15704371 | 0 | 0 |
| FCGR3A | 0 | 0 | 0 | 3.94204526 |
| HLA-DMB | 0.94860085 | 1.15704371 | 6.03584382 | 1.95233357 |
| SMAD6 | 8.2569163 | 8.06910063 | 9.1114487 | 6.76487159 |
| TNFSF10 | 5.53636379 | 1.79493566 | 0 | 2.40871186 |
| TNFSF14 | 0 | 0 | 0 | 8.05961486 |
| FCGR2B | 0 | 0 | 0 | 5.79701298 |
| CNIH1 | 10.0464283 | 10.7028307 | 9.90574801 | 10.350442 |
| IRF8 | 0 | 2.23266076 | 6.83642913 | 9.20823436 |
| RFX1 | 6.74926608 | 6.48816149 | 7.03408363 | 6.03672311 |
| CCL3L3 | 0.94860085 | 0 | 0 | 10.7110044 |
| IL5 | 0 | 0 | 0 | 0 |
| PRG2 | #N/A | #N/A | #N/A | #N/A |
| IL1R1 | 9.9711699 | 10.0143553 | 4.84247606 | 11.1518664 |
| TNFRSF10B | 10.1676189 | 11.2201304 | 9.91079261 | 10.7134724 |
| HLA-C | 10.5404671 | 11.7838577 | 11.104009 | 10.782171 |
| IGHA1 | 1.51096192 | 0 | 0 | 2.40871186 |
| FCAR | 0 | 0 | 0 | 10.0319631 |
| CCL7 | 0 | 2.23266076 | 0 | 3.64270157 |
| ARHGDIB | 4.28762004 | 7.52911816 | 1.422233 | 12.4604201 |
| KIR3DL1 | 0 | 0 | 0 | 1.28688115 |
| TRIM22 | 6.65949648 | 9.88676351 | 0.87970577 | 6.26959425 |
| C8B | 0 | 1.79493566 | 0 | 1.95233357 |
| TINAGL1 | 5.25058267 | 7.75181182 | 0 | 3.03210084 |
| ATP6V0A2 | 9.8431045 | 9.35149141 | 9.76676078 | 9.97953952 |
| ACKR4 | 1.91838623 | 8.8288981 | 2.592158 | 2.40871186 |
| HLA-DQA1 | 0.94860085 | 0 | 2.7803101 | 2.40871186 |
| CD164 | 12.7930099 | 12.8111378 | 11.9817778 | 13.7601035 |
| CCR8 | 0.94860085 | 0 | 0.87970577 | 0 |
| TNFSF13 | 3.36036428 | 3.07038933 | 4.61412087 | 3.64270157 |
| IL13 | 0 | 0 | 0 | 1.95233357 |
| PRELID1 | 8.87061131 | 8.71073757 | 9.104887 | 8.6244665 |
| PRG4 | 3.48413813 | 0 | 2.592158 | 2.7548875 |
| CCL3L1 | 0 | 0 | 0 | 10.8985863 |
| OAS1 | 2.23572706 | 0 | 0 | 7.84197312 |
| C1QBP | 11.8386406 | 11.5053848 | 12.9290161 | 11.5456402 |
| COLEC12 | 0 | 8.40968793 | 8.51900715 | 3.03210084 |
| ETS1 | 10.9543636 | 11.2445303 | 7.81941273 | 12.1592754 |
| IL18RAP | 0 | 0 | 2.12432814 | 2.40871186 |
| RGS1 | 3.36036428 | 0 | 0 | 14.408743 |
| CCR6 | #N/A | #N/A | #N/A | #N/A |
| CCL23 | 0 | 0 | 0 | 3.94204526 |
| RELT | 7.52747701 | 7.18249368 | 7.33333428 | 9.34837408 |
| HLA-DMA | 3.7059779 | 4.28466219 | 4.08236197 | 3.80012335 |
| XXbac- | #N/A | #N/A | #N/A | #N/A |
| BPG181M17.5 | | | | |
| CTSG | 0.94860085 | 0 | 0 | 8.02818235 |
| AIRE | 0.94860085 | 2.23266076 | 0 | 2.40871186 |
| CXCL10 | 0 | 0 | 1.422233 | 4.58436125 |
| GZMH | 0 | 0 | 0 | 0 |
| CXCL8 | #N/A | #N/A | #N/A | #N/A |
| VPREB3 | 0 | 0 | 1.81557543 | 0 |
| CXCL9 | 0 | 0 | 0 | 0 |
| CD70 | 9.45073701 | 1.79493566 | 3.35473424 | 7.14771372 |
| PTAFR | 3.0721058 | 2.23266076 | 3.46858332 | 3.64270157 |
| DPP8 | 10.1652965 | 10.5162417 | 10.1134296 | 10.1497725 |
| LST1 | 0 | 0 | 0.87970577 | 10.4866943 |
| AL928654.7 | #N/A | #N/A | #N/A | #N/A |
| FCGR3B | 0 | 0 | 0 | 0 |
| 42430 | 8.07055033 | 5.94063661 | 7.348197 | 5.93875626 |
| CXCL11 | 0 | 0 | 0 | 3.26603689 |
| CEBPG | 11.0118283 | 9.71326621 | 9.97955381 | 10.2347935 |
| CCL18 | 0 | 0 | 0 | 4.29865832 |
| HRH2 | 0 | 0 | 1.81557543 | 4.07038933 |
| TNFRSF4 | 0.94860085 | 1.15704371 | 0 | 4.07038933 |
| VIPR1 | 0.94860085 | 1.15704371 | 4.79960542 | 4.29865832 |
| HAMP | 0 | 2.56803211 | 0 | 3.64270157 |
| OSM | 0 | 0 | 0.87970577 | 6.5158575 |
| IGHE | 0 | 0 | 0 | 1.28688115 |
| IFI6 | 6.93545975 | 8.93445753 | 7.46931621 | 11.7799984 |
| OAS3 | 7.35032044 | 7.33351318 | 7.90267659 | 9.60679383 |
| VAV1 | 2.23572706 | 1.15704371 | 4.88410945 | 10.3101924 |
| DEFB4B | #N/A | #N/A | #N/A | #N/A |
| HLA-F | 6.91348784 | 5.61735726 | 6.58225491 | 0 |
| CSF3 | 1.91838623 | 2.56803211 | 2.592158 | 5.59723319 |
| XXbac- | #N/A | #N/A | #N/A | #N/A |
| BPG246D15.9 | | | | |
| TAPBP | 11.5164437 | 10.4337628 | 8.67228389 | 11.9341405 |
| HLA-DQA2 | 0 | 0 | 0 | 0 |
| CCL11 | #N/A | #N/A | #N/A | #N/A |
| NGFR | 0.94860085 | 0 | 4.51222689 | 3.94204526 |
| ADGRE5 | #N/A | #N/A | #N/A | #N/A |
| KIR3DS1 | #N/A | #N/A | #N/A | #N/A |
| IGHV3-23 | #N/A | #N/A | #N/A | #N/A |
| CCL16 | 0 | 0 | 0.87970577 | 0 |
| TNFSF15 | 0.94860085 | 1.15704371 | 1.81557543 | 9.13637598 |
| IL2RG | 0 | 1.15704371 | 0.87970577 | 1.28688115 |
| HLA-DRB3 | #N/A | #N/A | #N/A | #N/A |
| TNFRSF10D | 9.67336208 | 11.3139487 | 7.86164526 | 10.8008837 |
| CCR1 | 0 | 0 | 0 | 9.05297553 |
| ENPP3 | 0 | 2.23266076 | 2.94673086 | 2.40871186 |
| PTGER4 | 6.59170933 | 4.19061486 | 4.00898878 | 11.8428532 |
| CD96 | 1.51096192 | 1.15704371 | 1.81557543 | 0 |
| CNR2 | 0 | 0 | 1.422233 | 1.28688115 |
| CCL4L2 | 0 | 0 | 0 | 6.21470767 |
| TNFSF9 | 8.42193858 | 3.98185265 | 6.15946773 | 7.67962084 |
| P2RY14 | 0.94860085 | 0 | 4.08236197 | 0 |
| HLA-DPA1 | 1.91838623 | 5.24754781 | 6.20945337 | 3.64270157 |
| HLA-DRB4 | #N/A | #N/A | #N/A | #N/A |
| CXCL2 | 5.53636379 | 3.59693514 | 0 | 9.49059019 |
| CD27 | 0 | 1.15704371 | 0 | 0 |
| TNFRSF10A | 5.50716035 | 6.48816149 | 6.64400045 | 8.79749942 |
| BMP6 | 4.06608919 | 4.45680615 | 6.53075746 | 7.85990745 |
| TNFRSF8 | 2.49313492 | 0 | 2.592158 | 3.94204526 |
| XCL2 | 0 | 0 | 0 | 0 |
| MADCAM1 | 3.0721058 | 0 | 2.12432814 | 2.40871186 |
| CCL22 | 0 | 0 | 1.422233 | 2.40871186 |
| TNFSF8 | 0 | 1.15704371 | 0 | 1.95233357 |
| CCL19 | 0 | 0 | 0 | 0 |
| TNFSF13B | 0.94860085 | 1.79493566 | 1.422233 | 4.96208625 |
| RAET1G | 3.36036428 | 3.2675358 | 3.09592442 | 2.7548875 |
| FASLG | #N/A | #N/A | #N/A | #N/A |
| CCL27 | #N/A | #N/A | #N/A | #N/A |
| CHST4 | 0.94860085 | 0 | 4.21878117 | 0 |
| IL19 | 0 | 0 | 1.81557543 | 1.28688115 |
| TMIGD2 | 0 | 0 | 0.87970577 | 2.40871186 |
| SEMA4D | 7.07092595 | 5.88141991 | 7.83010318 | 6.70653055 |
| IL20 | 0 | 0 | 0 | 0 |
| COL4A3BP | 9.02231232 | 9.44122201 | 8.93966736 | 9.47027248 |
| CXCL6 | 7.65892551 | 4.53543092 | 2.37851162 | 7.41388175 |
| OTUD7A | 1.91838623 | 0 | 5.79545527 | 3.80012335 |
| IL24 | 3.0721058 | 2.23266076 | 0 | 11.5947165 |
| CX3CL1 | 2.90496572 | 1.15704371 | 0 | 1.28688115 |
| TNFRSF1A | 10.1034452 | 10.0399198 | 7.99101157 | 9.86195536 |
| CHIA | 0 | 0 | 0.87970577 | 4.18903382 |
| PVRL1 | 6.15845822 | 7.17022553 | 8.02392065 | 6.60377469 |
| PF4V1 | #N/A | #N/A | #N/A | #N/A |
| CCL17 | 0 | 0 | 0 | 3.03210084 |
| LTB4R | 5.75007049 | 5.50430258 | 6.85748343 | 5.72027847 |
| CCL8 | 0 | 0 | 0 | 3.03210084 |
| MAP4K2 | 6.96786027 | 7.85530455 | 7.070604 | 7.68964861 |
| IL3 | #N/A | #N/A | #N/A | #N/A |
| FTH1 | 8.20349484 | 8.87128923 | 6.65606727 | 11.5683409 |
| IL9 | 0 | 0 | 0 | 0 |
| CCL15-CCL14 | #N/A | #N/A | #N/A | #N/A |
| IFITM2 | 1.91838623 | 8.35098337 | 6.24184018 | 7.48502423 |
| CCL28 | 0.94860085 | 5.9112122 | 0 | 6.60377469 |
| ULBP1 | 5.53636379 | 5.50430258 | 6.55673595 | 4.58436125 |
| GPR65 | 0 | 0 | 0 | 7.92961349 |
| PRG3 | 0 | 0 | 0 | 1.28688115 |
| HLA-DRB5 | 0 | 0 | 1.81557543 | 3.26603689 |
| TNFRSF18 | 3.36036428 | 0 | 1.422233 | 0 |
| S1PR4 | 1.51096192 | 0 | 0.87970577 | 3.94204526 |
| ICOS | #N/A | #N/A | #N/A | #N/A |
| IL15 | 5.84624257 | 6.07852445 | 3.35473424 | 2.7548875 |
| ULBP3 | 6.00045077 | 5.68790052 | 6.79337571 | 4.39985467 |
| ULBP2 | 7.55612282 | 7.55773173 | 5.36737107 | 8.82059384 |
| GZMA | #N/A | #N/A | #N/A | #N/A |
| CCL13 | 0 | 1.79493566 | 0 | 0 |
| LCP2 | 0.94860085 | 1.15704371 | 1.422233 | 10.6018729 |
| CCL25 | 0.94860085 | 0 | 0 | 0 |
| NRROS | 3.48413813 | 1.15704371 | 2.37851162 | 9.65386615 |
| GTPBP1 | 9.69773213 | 9.3731044 | 9.81995552 | 9.57180952 |
| FCGR1A | 0 | 0 | 0 | 7.21897486 |
| MS4A2 | 0 | 0 | 0 | 1.28688115 |
| CCL5 | 1.51096192 | 1.15704371 | 0 | 9.60145511 |
| FYB | 0 | 0 | 0 | 6.03672311 |
| IK | 10.9536945 | 10.3652945 | 10.5077847 | 10.4247017 |
| LY75 | 0 | 0 | 0 | 4.18903382 |
| TGFBR3 | 5.69960715 | 9.16576123 | 9.49205346 | 5.97223312 |
| CD40LG | 0 | 0 | 0.87970577 | 0 |
| TNFSF11 | 0 | 0 | 0 | 2.7548875 |
| CXCR5 | 0 | 0 | 0 | 5.20633065 |
| AZGP1 | #N/A | #N/A | #N/A | #N/A |
| GBP6 | 0.94860085 | 0 | 0.87970577 | 3.03210084 |
| SBSPON | 2.71369582 | 7.50000529 | 4.96393645 | 2.40871186 |
| IGKV1-5 | #N/A | #N/A | #N/A | #N/A |
| IGKV4-1 | #N/A | #N/A | #N/A | #N/A |
| IL18 | 0.94860085 | 6.13031315 | 2.37851162 | 7.61765112 |
| CHIT1 | 0 | 0 | 0.87970577 | 6.72628629 |
| SLPI | 0 | 1.15704371 | 0 | 6.83920379 |
| GEM | 7.96214411 | 8.24939793 | 5.24488706 | 12.5796624 |
| IGKV3-11 | #N/A | #N/A | #N/A | #N/A |
| IGKV5-2 | #N/A | #N/A | #N/A | #N/A |
| TNFRSF9 | 8.48671437 | 1.15704371 | 3.35473424 | 5.86962469 |
| CSF2 | 1.51096192 | 0 | 0 | 3.94204526 |
| CMA1 | 0.94860085 | 0 | 0.87970577 | 1.28688115 |
| MYLPF | 2.23572706 | 2.56803211 | 2.94673086 | 4.29865832 |
| SECTM1 | 2.23572706 | 5.88141991 | 0.87970577 | 3.64270157 |
| XBP1 | 11.808019 | 10.8963021 | 10.0232554 | 11.1851852 |
| SEMA3C | 12.1494201 | 12.2398252 | 8.66035251 | 8.88340699 |
| CCR10 | 1.51096192 | 3.07038933 | 3.23112516 | 0 |
| CST7 | 1.51096192 | 2.23266076 | 1.81557543 | 0 |
| OPRD1 | 0.94860085 | 1.15704371 | 3.09592442 | 0 |
| IL1R2 | 0.94860085 | 2.23266076 | 2.12432814 | 5.50906239 |
| SERPINB9 | 8.46797229 | 7.68425771 | 1.422233 | 3.03210084 |
| ILF2 | 12.7860225 | 11.9445693 | 12.920297 | 13.2302982 |
| CCL1 | 0 | 0 | 0 | 5.20633065 |
| CCR5 | 0.94860085 | 0 | 0 | 2.7548875 |
| ITGAD | 0 | 0 | 0 | 3.64270157 |
| C8A | #N/A | #N/A | #N/A | #N/A |
| PXDN | 12.2761273 | 13.2497549 | 9.57535015 | 3.64270157 |
| SEMA7A | 9.66844183 | 11.4581144 | 5.5068431 | 11.9876904 |
| EDA | 0 | 1.79493566 | 6.55673595 | 2.40871186 |
| IL10RB | 8.7822775 | 8.30108459 | 6.84699469 | 10.8678579 |
| RP11-872D17.8 | 0 | 2.23266076 | 0 | 0 |
| PDCD1LG2 | 9.33335664 | 10.8284697 | 0 | 4.82273015 |
| IL21 | 0 | 0 | 0 | 1.28688115 |
| CXCL14 | 2.71369582 | 3.4409522 | 3.67242534 | 3.26603689 |
| IGKV1D-16 | #N/A | #N/A | #N/A | #N/A |
| IL16 | 1.51096192 | 0 | 3.35473424 | 3.03210084 |
| CR2 | 0 | 1.15704371 | 5.21256934 | 0 |
| IFITM3 | 8.6013251 | 9.72171443 | 5.27649667 | 7.58563861 |
| SH2D6 | 0 | 0 | 2.7803101 | 0 |
| THBS1 | 16.0025212 | 17.3912186 | 3.76447355 | 11.068429 |
| IKBKAP | 10.4892961 | 10.6953414 | 11.3111409 | 10.0240177 |
| CTSW | 3.98458935 | 1.79493566 | 1.422233 | 5.20633065 |
| IGSF6 | 0 | 1.15704371 | 2.592158 | 7.68964861 |
| CCR9 | 0 | 1.79493566 | 2.12432814 | 1.95233357 |
| CXCL1 | 7.46825747 | 6.448075 | 0.87970577 | 9.21171798 |
| TENM1 | 0 | 1.15704371 | 3.8509994 | 0 |
| CCL26 | 3.98458935 | 2.56803211 | 0 | 1.95233357 |
| PNP | 9.40215937 | 9.24996673 | 9.71440008 | 11.955174 |
| GBP2 | 9.4314976 | 5.78790256 | 1.81557543 | 5.36562256 |
| CD276 | 10.3931545 | 10.5757283 | 10.0474784 | 12.5067439 |
| APLN | 3.48413813 | 2.23266076 | 2.7803101 | 1.28688115 |
| CCL2 | 9.46403515 | 9.54726087 | 2.37851162 | 5.02724254 |
| AQP9 | 0 | 0 | 0 | 4.39985467 |
| LTBR | 0 | 10.4929748 | 3.8509994 | 9.87967514 |
| CCL24 | 0 | 0 | 0 | 6.68664059 |
| CCL20 | 2.49313492 | 0 | 0 | 13.1916068 |
| CCL21 | 0 | 1.15704371 | 1.422233 | 3.46727948 |
| NCF4 | 0 | 0 | 0 | 9.24284065 |
| TNFRSF25 | 5.35226417 | 5.29351763 | 4.66277517 | 4.39985467 |
| IGLL1 | 0 | 0 | 0 | 0 |
| C1QC | 0 | 0 | 0 | 7.31324585 |
| LIF | 9.62170392 | 5.78790256 | 1.81557543 | 7.31324585 |
| FAS | 6.6055536 | 10.3853771 | 4.61412087 | 3.26603689 |
| RAET1L | 0 | 0 | 0 | 0 |
| PRKRA | 9.30827076 | 9.37577797 | 9.2806084 | 8.96448527 |
| ADAMDEC1 | 1.51096192 | 0 | 0 | 11.9399538 |
| IL1RAP | 9.29980329 | 7.24241196 | 6.90869284 | 8.90517638 |
| TNFRSF6B | #N/A | #N/A | #N/A | #N/A |
| IL18R1 | 2.49313492 | 1.79493566 | 6.50430258 | 5.93875626 |
| NFIL3 | 8.60477559 | 8.22583404 | 7.27974985 | 10.4910213 |
| ENPP2 | 7.53465306 | 10.4843094 | 5.36737107 | 1.95233357 |
| PROCR | 6.96786027 | 7.90068571 | 7.59983848 | 7.53060142 |
| ENDOU | 0.94860085 | 1.15704371 | 3.09592442 | 0 |
| DEFB4A | #N/A | #N/A | #N/A | #N/A |
| VTN | 1.51096192 | 3.2675358 | 2.7803101 | 0 |
| SAMHD1 | 9.32086823 | 9.28745844 | 10.3386027 | 11.3193955 |
| PF4 | 0 | 0 | 0.87970577 | 1.28688115 |
| CD274 | 8.06058786 | 9.99356115 | 2.592158 | 5.41515021 |
| PPBP | 0 | 0 | 0 | 7.05723364 |
| CXCL5 | 7.2917693 | 4.09000653 | 0.87970577 | 6.37312616 |
| KAAG1 | 0 | 0 | 1.422233 | 0 |
| CFP | 3.0721058 | 1.79493566 | 1.81557543 | 4.82273015 |
| IRAK1BP1 | 6.08172309 | 5.8509994 | 7.07948478 | 5.20633065 |
| IGF1R | 9.87762157 | 10.8991001 | 11.5052062 | 5.14893411 |
| BPI | 0 | 2.83995959 | 0 | 2.40871186 |
| SMAD3 | 11.1257041 | 9.53772316 | 9.02812708 | 6.62497818 |
| CXCL3 | 4.53853816 | 3.2675358 | 2.12432814 | 10.5377814 |
| CLNK | #N/A | #N/A | #N/A | #N/A |
| IL17A | 0.94860085 | 0 | 0.87970577 | 1.28688115 |
| IL26 | #N/A | #N/A | #N/A | #N/A |
| CYP11B1 | 0 | 0 | 0.87970577 | 2.40871186 |
| CMKLR1 | 0 | 0 | 0 | 6.42240122 |
| IL17B | 0 | 0 | 0.87970577 | 3.64270157 |
| TCF12 | 10.6560047 | 11.2569399 | 9.82129566 | 12.0510968 |
| FCGRT | 6.1771205 | 9.32952728 | 7.64889681 | 12.8887547 |
| VPREB1 | 0 | 0 | 0 | 0 |
| CCR4 | #N/A | #N/A | #N/A | #N/A |
| IL4R | 9.72325137 | 9.29879576 | 7.37033868 | 10.6098528 |
| TNFAIP1 | 9.49030939 | 10.1204323 | 9.39289696 | 9.18705509 |
| ZEB1 | 10.1358888 | 11.1615413 | 9.80245174 | 10.9560567 |
| IL32 | 8.82416321 | 3.98185265 | 1.81557543 | 3.46727948 |
| TCF7 | 6.23150921 | 5.8509994 | 7.85118684 | 5.41515021 |
| CEBPB | 9.8269933 | 9.25287827 | 7.41362793 | 11.5697085 |
| MBP | 7.49817107 | 4.37364821 | 6.25776497 | 12.2737869 |
| CYSLTR2 | 0 | 0 | 0 | 3.80012335 |
| OPRK1 | 0 | 1.15704371 | 4.56437817 | 0 |
| BMPR1A | 10.4275433 | 10.2251954 | 9.66604643 | 8.29558529 |
| CEACAM8 | 0 | 1.15704371 | 0 | 0 |
| CIITA | 3.80426012 | 2.23266076 | 3.35473424 | 2.7548875 |
| IL7 | 4.59514557 | 4.68144927 | 2.592158 | 1.95233357 |
| TINAG | 0 | 0 | 0 | 0 |
| IL22 | 0 | 0 | 0 | 2.40871186 |
| C1QB | 0 | 0 | 0 | 3.26603689 |
| CFD | 3.0721058 | 1.79493566 | 6.01725384 | 10.889869 |
| IGHG1 | 0 | 1.79493566 | 0.87970577 | 0 |
| CFB | 4.06608919 | 3.2675358 | 4.21878117 | 3.46727948 |
| C2 | 1.51096192 | 1.15704371 | 2.37851162 | 4.39985467 |
| C1QA | 0 | 0 | 0 | 9.13267965 |
| RGCC | 2.23572706 | 4.74899785 | 4.96393645 | 13.901179 |
| C7 | 0 | 0 | 0 | 0 |
| IGHG4 | 0 | 0 | 0 | 1.28688115 |
| C4B | 0.94860085 | 1.79493566 | 0 | 3.64270157 |
| C4A | 2.49313492 | 0 | 2.592158 | 1.95233357 |
| CLU | 6.74926608 | 7.79246496 | 9.73159057 | 3.03210084 |
| CFH | 5.14118753 | 9.93116594 | 0.87970577 | 1.95233357 |
| C5 | 7.56323441 | 7.38758657 | 3.23112516 | 5.46270675 |
| IGHG2 | #N/A | #N/A | #N/A | #N/A |
| C4B_2 | #N/A | #N/A | #N/A | #N/A |
| CFHR1 | 0 | 1.15704371 | 1.81557543 | 1.28688115 |
| C1S | 8.85046816 | 10.8459683 | 4.66277517 | 3.26603689 |
| C9 | 0.94860085 | 1.15704371 | 0 | 3.46727948 |
| C8G | 2.90496572 | 2.23266076 | 3.09592442 | 4.39985467 |
| CFHR5 | #N/A | #N/A | #N/A | #N/A |
| VSIG4 | 0 | 0 | 0 | 4.58436125 |
| SUSD4 | 5.17871464 | 4.37364821 | 6.22573765 | 1.28688115 |
| C4BPB | 0 | 0 | 0 | 2.40871186 |
| IGHV3OR16-9 | #N/A | #N/A | #N/A | #N/A |
| CFI | 4.89384756 | 3.07038933 | 0.87970577 | 2.40871186 |
| C4BPA | 0 | 0 | 0 | 1.28688115 |
| CD55 | 10.0131691 | 9.17809214 | 8.76980479 | 9.23599027 |
| IGLL5 | #N/A | #N/A | #N/A | #N/A |
| C1RL | 7.04078287 | 7.83983365 | 5.11103131 | 4.49505553 |
| ST6GAL1 | 6.68565988 | 5.04701482 | 7.40650266 | 10.0633007 |
| SH2D1A | #N/A | #N/A | #N/A | #N/A |
| PDCD1 | 0 | 1.15704371 | 1.422233 | 0 |
| LY86 | 0 | 0 | 0 | 7.80535695 |
| YTHDF2 | 10.8364765 | 10.607996 | 10.8331668 | 10.7048028 |
| CD83 | 7.2917693 | 5.9112122 | 7.24830712 | 11.9753894 |
| BLNK | 0 | 0 | 0.87970577 | 3.03210084 |
| TREM2 | 2.23572706 | 1.15704371 | 0 | 12.3081172 |
| GPI | 11.7466991 | 11.3132459 | 11.913125 | 11.3750829 |
| MNX1 | 0.94860085 | 0 | 7.86684618 | 3.26603689 |
| BST1 | 8.95384723 | 7.80844941 | 2.37851162 | 7.96318509 |
| POU2AF1 | 0 | 0 | 0 | 3.46727948 |
| BCL2 | 7.951343 | 6.10454634 | 7.7977912 | 10.1893675 |
| TRAF3IP2 | 8.76085299 | 8.63669712 | 6.55673595 | 8.52109001 |
| PAX5 | 0 | 0 | 2.94673086 | 3.94204526 |
| MS4A1 | 0 | 0 | 0 | 2.7548875 |
| EBI3 | 4.21800615 | 2.83995959 | 1.422233 | 8.91808803 |
| DEFB103B | #N/A | #N/A | #N/A | #N/A |
| DEFB103A | #N/A | #N/A | #N/A | #N/A |
| GAS6 | 11.0157077 | 13.0965307 | 5.97933945 | 6.58225491 |
| TGFB3 | 6.41498114 | 5.04701482 | 6.20945337 | 9.82359016 |
| TGFB2 | 10.7994435 | 8.08884123 | 3.93168306 | 5.68060567 |
| CUEDC2 | 9.05283972 | 9.17809214 | 8.6270596 | 9.35155766 |
| IGHD | #N/A | #N/A | #N/A | #N/A |
| IL13RA2 | 0 | 7.54828248 | 0 | 6.21470767 |
| INPP5D | 1.91838623 | 3.4409522 | 1.422233 | 10.5571669 |
| SLA2 | 0.94860085 | 0 | 0.87970577 | 3.26603689 |
| DEFA4 | #N/A | #N/A | #N/A | #N/A |
| CST9 | 0 | 0 | 0 | 0 |
| AZU1 | 0 | 0 | 0.87970577 | 9.92088874 |
| VIP | 0 | 0 | 0 | 0 |
| ADM | 9.6301579 | 11.6819719 | 6.55673595 | 8.75495429 |
| SEMG2 | 0 | 0 | 0 | 0 |
| ANG | 4.14404637 | 4.45680615 | 3.35473424 | 2.40871186 |
| DEFA6 | #N/A | #N/A | #N/A | #N/A |
| APP | 12.8625192 | 13.6372373 | 11.6444422 | 11.3617519 |
| TAC1 | 0 | 1.15704371 | 2.592158 | 1.28688115 |
| NPY | 0 | 0 | 0 | 4.07038933 |
| RNASE7 | 0.94860085 | 0 | 0 | 0 |
| DEFA5 | #N/A | #N/A | #N/A | #N/A |
| PLA2G6 | 5.28540222 | 5.38162947 | 5.83844759 | 6.03672311 |
| SEMG1 | 0 | 0 | 0 | 0 |
| CALCA | 0.94860085 | 0 | 0 | 3.03210084 |
| BPIFA1 | #N/A | #N/A | #N/A | #N/A |
| PR0S1 | 9.32294213 | 7.79246496 | 8.64835758 | 9.22216698 |
| CHGA | 0 | 1.79493566 | 3.35473424 | 4.39985467 |
| HMOX1 | 8.2699682 | 9.69620252 | 10.2459448 | 12.5622901 |
| NR4A3 | 2.71369582 | 5.099716 | 5.27649667 | 7.33904828 |
| LGALS9 | 0.94860085 | 2.23266076 | 2.94673086 | 9.77945673 |
| CD300A | 0 | 0 | 0.87970577 | 8.83428142 |
| FER | 9.93884446 | 10.5319467 | 9.55618031 | 9.05297553 |
| GFI1 | 4.64961546 | 2.23266076 | 6.63183185 | 9.00537192 |
| NFKBIL1 | 7.25729355 | 7.04111154 | 7.27192984 | 8.75495429 |
| TYRO3 | 8.90728127 | 9.29029538 | 9.14391886 | 7.33904828 |
| ARRB2 | 6.91348784 | 6.448075 | 9.4886837 | 11.0519226 |
| LGR4 | 10.821774 | 9.83899902 | 10.9601251 | 6.34801991 |
| CACTIN | 8.98848588 | 8.6676438 | 8.32129678 | 8.74533819 |
| IRF4 | 0.94860085 | 2.23266076 | 3.09592442 | 3.64270157 |
| NLRP2P | #N/A | #N/A | #N/A | #N/A |
| PIK3AP1 | 1.51096192 | 2.83995959 | 7.24031433 | 11.0856982 |
| CYBA | 9.53964351 | 10.0766286 | 8.5929802 | 12.8387017 |
| UBQLN1 | 12.0285415 | 12.0046769 | 10.7659325 | 11.577188 |
| FLOT1 | 10.2368394 | 10.7386665 | 9.47173617 | 10.6835386 |
| WDFY1 | 10.734227 | 11.3313089 | 9.88028758 | 11.0253195 |
| CAV1 | 12.8010879 | 13.8381344 | 7.11488753 | 10.6151422 |
| PTPN22 | 5.95814667 | 3.59693514 | 1.81557543 | 6.64573048 |
| S100A14 | 0 | 1.15704371 | 0 | 2.7548875 |
| NR1D1 | 5.06350294 | 6.29792505 | 4.66277517 | 3.46727948 |
| DAB2IP | 7.20388631 | 9.24410215 | 8.7361313 | 4.58436125 |
| RAB7B | #N/A | #N/A | #N/A | #N/A |
| NR1H3 | 5.69960715 | 5.68790052 | 6.58225491 | 7.3262497 |
| RSAD2 | 0 | 1.15704371 | 0 | 6.58225491 |
| MX1 | 5.86937792 | 5.38162947 | 2.94673086 | 2.40871186 |
| ISG15 | 7.11977116 | 8.56807012 | 5.30742853 | 9.49920809 |
| TRIM56 | 9.11444511 | 8.95968323 | 7.41362793 | 9.19414124 |
| SP100 | 9.69449719 | 10.3018848 | 2.94673086 | 10.1750879 |
| MEFV | 1.51096192 | 0 | 0 | 7.33904828 |
| TRIM21 | 7.71726515 | 7.98031098 | 7.070604 | 9.51629099 |
| GCH1 | 7.78555055 | 4.87578006 | 8.37695017 | 8.73562372 |
| UBD | #N/A | #N/A | #N/A | #N/A |
| CXCL16 | 3.0721058 | 3.4409522 | 6.65606727 | 7.00977264 |
| SNCA | 4.53853816 | 2.83995959 | 7.73667252 | 0 |
| CALCOCO2 | 9.88612332 | 10.5089337 | 9.95415268 | 10.5488893 |
| CYP27B1 | 3.89724043 | 4.37364821 | 4.84247606 | 6.12845838 |
| IFITM1 | 2.23572706 | 7.24241196 | 3.76447355 | 3.03210084 |
| KYNU | 0 | 2.56803211 | 2.37851162 | 10.9539709 |
| CITED1 | 3.0721058 | 1.15704371 | 5.58466191 | 0 |
| SEC61A1 | 13.0285277 | 13.3817728 | 11.7865719 | 12.7032266 |
| SLC30A8 | 0 | 1.15704371 | 0.87970577 | 1.28688115 |
| ADAMTS13 | 4.89384756 | 4.68144927 | 6.19298317 | 3.80012335 |
| DNAJA3 | 10.516025 | 10.3004784 | 10.4783854 | 10.6256815 |
| ADAM8 | 4.70154906 | 5.29351763 | 4.88410945 | 6.80258099 |
| FGF7 | 1.91838623 | 8.84830996 | 1.81557543 | 3.26603689 |
| FGF17 | 0 | 0 | 1.81557543 | 3.03210084 |
| FGF20 | 0 | 1.15704371 | 1.81557543 | 0 |
| PEBP1 | 11.7032547 | 11.8879669 | 11.8073267 | 10.7880494 |
| RICTOR | 9.62846377 | 10.3773841 | 9.83991236 | 11.3990749 |
| GSK3B | 10.5636253 | 11.0162372 | 10.2095996 | 10.280504 |
| FGF16 | #N/A | #N/A | #N/A | #N/A |
| CD80 | 0.94860085 | 0 | 1.81557543 | 1.95233357 |
| FRS2 | 9.28701972 | 9.59633886 | 9.25507579 | 8.52109001 |
| RASAL3 | 0 | 1.15704371 | 0.87970577 | 5.46270675 |
| SHC2 | 1.51096192 | 5.099716 | 5.07553263 | 6.70653055 |
| NRG2 | 3.36036428 | 1.79493566 | 5.14567746 | 3.03210084 |
| FRS3 | 3.80426012 | 4.9349882 | 5.30742853 | 5.36562256 |
| CUL3 | 10.390169 | 10.736588 | 10.7135498 | 10.4262123 |
| VAV2 | 8.49785184 | 9.00539999 | 7.33333428 | 6.42240122 |
| RPS6KB2 | 8.90728127 | 8.91984656 | 8.68411741 | 9.46734062 |
| FGF5 | 5.5649878 | 11.3484681 | 3.76447355 | 0 |
| LAMTOR2 | 8.92389197 | 8.30108459 | 8.6453342 | 10.5010609 |
| FOXO4 | 7.61867915 | 6.65635335 | 8.50243404 | 7.47346224 |
| GRB2 | 10.0615197 | 10.4195494 | 10.0656585 | 11.6774158 |
| TEK | 1.91838623 | 7.37695017 | 2.7803101 | 0 |
| MAP2K2 | 11.0285623 | 10.6136882 | 10.3395383 | 10.6005355 |
| FGFR3 | 6.15845822 | 5.04701482 | 8.49908847 | 3.64270157 |
| MTOR | 11.6654736 | 11.0718377 | 11.9370942 | 10.6098528 |
| PHLPP2 | 10.2522751 | 9.67678573 | 9.15668987 | 7.90388185 |
| CDKN1B | 10.1346958 | 9.81747937 | 9.60984359 | 8.14261766 |
| RASGRF1 | 2.23572706 | 3.2675358 | 1.81557543 | 5.72027847 |
| FOXO1 | 4.89384756 | 7.05452292 | 9.098269 | 6.39797405 |
| GRIN2A | 6.96786027 | 0 | 1.81557543 | 0 |
| RASA1 | 11.004691 | 11.0510254 | 9.17357721 | 12.131828 |
| SPTBN5 | 5.67355642 | 3.59693514 | 3.8509994 | 7.75828985 |
| NEFL | 0 | 4.9349882 | 10.6181836 | 1.95233357 |
| GAB2 | 5.77478706 | 8.97036478 | 6.63183185 | 9.73150572 |
| AKT1S1 | 9.32712595 | 9.06307145 | 8.80542145 | 8.98507343 |
| PHLPP1 | 9.12884499 | 8.25526467 | 8.80271022 | 8.84784036 |
| SPTBN2 | 7.69167374 | 5.5428771 | 10.1733648 | 5.59723319 |
| GAB1 | 7.02036885 | 7.68425771 | 9.38750041 | 5.31433399 |
| PDGFB | 5.44724857 | 1.15704371 | 2.37851162 | 6.68664059 |
| RASA4 | 6.12060077 | 5.81966818 | 5.21256934 | 7.59633886 |
| PDGFA | 7.26594316 | 7.66682759 | 5.27649667 | 12.983423 |
| RAPGEF2 | 9.08273452 | 10.333938 | 10.4630339 | 9.05688153 |
| TNRC6B | 10.2009476 | 11.0618842 | 10.8503197 | 9.85302879 |
| KSR1 | 4.59514557 | 6.60243906 | 7.57470705 | 6.12845838 |
| RASA3 | 8.99112498 | 10.6406788 | 7.40650266 | 12.5166825 |
| GSK3A | 8.53438071 | 7.83206719 | 7.80322704 | 8.58725246 |
| SYNGAP1 | 6.46123386 | 6.82273015 | 6.58225491 | 6.42240122 |
| FGFR4 | 1.91838623 | 2.56803211 | 7.57470705 | 5.08915913 |
| EGFR | 10.5993922 | 12.0393201 | 8.0606419 | 4.66789213 |
| TRIB3 | 8.19893609 | 8.25526467 | 7.62461271 | 9.85525783 |
| AGO4 | 8.51258246 | 8.14644139 | 8.78907713 | 9.13637598 |
| LAMTOR3 | 8.58402294 | 9.43866712 | 9.32469811 | 10.1479426 |
| PAQR3 | 9.2805852 | 9.49890903 | 10.1079624 | 8.78816366 |
| RASGRF2 | 3.89724043 | 8.28974227 | 7.13226837 | 5.02724254 |
| PTPRA | 9.53243385 | 9.95644876 | 9.77231457 | 9.61474665 |
| MDM2 | 10.3640252 | 12.0899768 | 11.3445789 | 10.4953354 |
| BRAF | 8.75157753 | 9.43866712 | 9.74716918 | 9.45852965 |
| RASA2 | 9.94156119 | 10.229648 | 8.64230446 | 9.92088874 |
| IL17RD | 6.56376828 | 4.81403765 | 7.9572762 | 4.82273015 |
| PIK3R1 | 8.2125207 | 10.6148203 | 8.55485741 | 10.194683 |
| ACTN2 | 0 | 0 | 5.30742853 | 3.26603689 |
| FGF1 | 7.64565843 | 9.63711389 | 0 | 3.26603689 |
| FGFR2 | 9.99494737 | 0 | 9.26101359 | 0 |
| AGO1 | 10.4148649 | 10.1268077 | 11.1842369 | 9.93153533 |
| PTK2 | 10.7660568 | 10.8206178 | 10.8542451 | 9.54430764 |
| CAMK2D | 8.82416321 | 11.7989132 | 9.57216966 | 11.0577684 |
| AGO3 | 9.56985561 | 9.4840777 | 9.73159057 | 9.42273759 |
| GRAP2 | 2.23572706 | 0 | 0.87970577 | 3.26603689 |
| FGF19 | 0 | 0 | 0 | 1.28688115 |
| EREG | 2.71369582 | 9.78357124 | 1.422233 | 1.28688115 |
| GRIN2C | 2.90496572 | 3.59693514 | 4.00898878 | 3.64270157 |
| GRIN2B | 1.51096192 | 0 | 3.57410151 | 1.28688115 |
| FGF23 | 0 | 0 | 2.37851162 | 0 |
| ERBB3 | 2.71369582 | 4.74899785 | 7.39934202 | 3.64270157 |
| CASP9 | 6.98913901 | 6.58406053 | 7.43471162 | 7.78672704 |
| NF1 | 9.82553125 | 11.5078293 | 11.87399 | 11.1527792 |
| BRAP | 9.21235047 | 9.24117282 | 9.19231748 | 9.48769937 |
| JAK1 | 12.3980569 | 12.1456074 | 10.6792602 | 10.8689691 |
| FGF6 | #N/A | #N/A | #N/A | #N/A |
| BTC | 0 | 0 | 0 | 3.46727948 |
| CDKN1A | 8.98584194 | 12.6152664 | 6.22573765 | 15.0672528 |
| CSF2RA | #N/A | #N/A | #N/A | #N/A |
| KBTBD7 | 7.26594316 | 7.21878117 | 8.44107674 | 6.76487159 |
| MAPKAP1 | 11.5250901 | 11.2334879 | 10.3064605 | 11.0999329 |
| RASAL1 | 0 | 0 | 3.46858332 | 3.26603689 |
| YWHAB | 12.0896325 | 12.5520999 | 11.915323 | 12.5457464 |
| PDGFRB | 9.06037169 | 11.0392412 | 1.422233 | 5.31433399 |
| EGF | 5.67355642 | 6.67383906 | 4.924575 | 0 |
| FGFR1 | 10.734227 | 11.7283202 | 9.20057988 | 9.918744 |
| CAMK2B | 3.22342255 | 3.73660488 | 4.88410945 | 6.00495076 |
| FGF9 | 2.23572706 | 2.23266076 | 5.33771109 | 4.7473874 |
| AKAP9 | 11.3170734 | 11.3354462 | 10.7749435 | 10.1929092 |
| IL3RA | #N/A | #N/A | #N/A | #N/A |
| NRG4 | 3.98458935 | 3.73660488 | 6.24184018 | 3.46727948 |
| RASGEF1A | 2.23572706 | 2.23266076 | 7.39934202 | 4.18903382 |
| PDGFRA | 3.89724043 | 10.7542027 | 5.55918587 | 6.80258099 |
| PIK3R2 | 0 | 0 | 0 | 2.7548875 |
| AGO2 | 8.48300958 | 9.85637884 | 10.265744 | 12.7927049 |
| SPTA1 | 0 | 0 | 0 | 1.95233357 |
| MARK3 | 10.6326227 | 10.6091418 | 10.3636311 | 11.230747 |
| DUSP5 | 8.23491321 | 10.1582815 | 7.08841725 | 12.6657578 |
| DLG4 | 8.01992426 | 8.13386145 | 5.96000193 | 8.60332962 |
| AKT1 | 11.0285623 | 11.3980863 | 9.78608989 | 10.5681366 |
| IRS2 | 8.65538761 | 9.26451297 | 8.04242547 | 11.2948505 |
| THEM4 | 7.72996056 | 7.44997399 | 8.34753279 | 8.65563805 |
| LCK | 0 | 0 | 0 | 0 |
| IRS1 | 8.9565504 | 11.0915346 | 7.17472599 | 5.93875626 |
| SPTBN1 | 12.9891106 | 13.4034408 | 12.5959544 | 10.9623032 |
| FGF4 | 0 | 0 | 0 | 0 |
| IL2RB | 2.23572706 | 2.23266076 | 1.81557543 | 0 |
| FGF3 | 0 | 0 | 0 | 0 |
| DUSP9 | 1.91838623 | 2.56803211 | 7.78135971 | 5.55397481 |
| SPTBN4 | 3.7059779 | 4.45680615 | 6.37903173 | 3.46727948 |
| PLCG1 | 9.71849863 | 10.1981995 | 10.0645272 | 7.41388175 |
| RBX1 | 9.24797497 | 8.63669712 | 8.93966736 | 9.02541645 |
| MOV10 | 9.10231694 | 8.87887858 | 9.12887076 | 9.60412694 |
| FGF2 | 10.7896559 | 13.7888386 | 8.13576058 | 7.92106733 |
| ERBB2 | 9.18962692 | 9.92386227 | 9.44594598 | 3.46727948 |
| RAC1 | 11.475242 | 11.417726 | 10.433575 | 10.2671144 |
| WDR83 | 5.5649878 | 5.33842442 | 6.12536181 | 5.26115467 |
| BAD | 8.51625158 | 8.31233844 | 7.61845892 | 8.24863919 |
| MLST8 | 8.56292926 | 8.08884123 | 9.50211575 | 8.66590436 |
| RASAL2 | 10.8553591 | 10.4273233 | 9.28255564 | 10.9612688 |
| CAMK2G | 9.09745278 | 9.36503196 | 9.34717842 | 7.97148608 |
| SPRED1 | 9.82257083 | 11.2349671 | 8.49908847 | 10.8533641 |
| CD19 | 0 | 0 | 4.34340782 | 3.46727948 |
| SHC3 | 2.23572706 | 6.46825747 | 3.09592442 | 5.50906239 |
| NRG1 | 9.67828322 | 10.0881124 | 3.93168306 | 11.5940493 |
| CSF2RB | 0 | 2.56803211 | 1.422233 | 8.69620252 |
| PTPN11 | 12.452267 | 12.7138784 | 12.2660955 | 11.8246843 |
| KLB | 3.0721058 | 4.61058196 | 5.55918587 | 3.94204526 |
| TNRC6A | 10.5233071 | 10.6910456 | 10.3414076 | 8.92662216 |
| RASGRP4 | 1.91838623 | 1.15704371 | 1.81557543 | 2.7548875 |
| SPTAN1 | 13.0493381 | 13.1976928 | 12.0691678 | 11.7574234 |
| FOXO3 | 9.61829373 | 10.0930081 | 10.2881855 | 10.8843995 |
| HBEGF | 9.35801377 | 8.49597491 | 6.86777294 | 12.6686258 |
| SOS1 | 8.66530038 | 9.71748794 | 9.79292041 | 8.75975506 |
| PPP5C | 10.3374101 | 10.0195073 | 10.4247962 | 9.6383454 |
| SPTB | 4.21800615 | 6.13031315 | 8.00517539 | 3.80012335 |
| FGF8 | 0.94860085 | 0 | 0 | 1.95233357 |
| PTEN | 11.1352476 | 11.237915 | 10.5511891 | 9.86418615 |
| VAV3 | 3.59931779 | 1.15704371 | 7.37764436 | 13.433562 |
| ARAF | 9.27195318 | 8.94892201 | 9.06926178 | 8.87458182 |
| NCAM1 | 1.51096192 | 4.87578006 | 8.71035875 | 11.8074395 |
| FGF22 | 0.94860085 | 2.23266076 | 2.37851162 | 1.95233357 |
| ERBB4 | 0.94860085 | 0 | 6.668034 | 5.02724254 |
| TSC2 | 10.1477264 | 9.88487261 | 10.4265478 | 9.74121416 |
| PEA15 | 11.8415565 | 12.568006 | 9.1114487 | 12.5650283 |
| GRIN1 | 2.23572706 | 2.23266076 | 2.37851162 | 4.07038933 |
| RASGRP3 | 2.23572706 | 4.74899785 | 4.21878117 | 10.8600705 |
| PRKCQ | 1.91838623 | 1.15704371 | 7.54256761 | 4.49505553 |
| SHC1 | 11.3044426 | 11.8657371 | 8.53869335 | 10.8556082 |
| GRIN2D | 2.49313492 | 5.15055968 | 5.11103131 | 7.87768291 |
| KL | 3.0721058 | 0 | 0 | 3.64270157 |
| TNRC6C | 6.87982827 | 8.85602083 | 8.49908847 | 6.76487159 |
| CAMK2A | 4.48026512 | 3.59693514 | 0.87970577 | 6.34801991 |
| NR4A1 | 3.36036428 | 6.20457114 | 6.80426012 | 3.26603689 |
| FGF10 | 0 | 1.15704371 | 1.422233 | 1.28688115 |
| FGF18 | 3.36036428 | 1.15704371 | 2.592158 | 3.46727948 |
| KITLG | 11.3816349 | 12.200304 | 9.21693982 | 10.0748773 |
| BRK1 | 10.8632803 | 10.6395668 | 9.96751448 | 11.7241131 |
| FCGR2A | 0.94860085 | 0 | 0.87970577 | 7.26011977 |
| ARPC1A | 11.4094281 | 11.1380542 | 10.3718746 | 10.1858046 |
| MYO1C | 12.3274965 | 12.7106988 | 9.70863559 | 10.6683355 |
| MYO10 | 10.0714892 | 12.7450482 | 10.5308745 | 12.2883643 |
| NCK1 | 8.94573644 | 9.06971828 | 8.43758564 | 9.83951875 |
| PLPP4 | #N/A | #N/A | #N/A | #N/A |
| NCKIPSD | 7.77333705 | 8.03556893 | 8.68116812 | 9.53595573 |
| ARPC1B | 10.7529409 | 10.6910456 | 7.52293456 | 12.0559704 |
| CFL1 | 13.9592802 | 13.6097536 | 11.9441776 | 13.4709181 |
| ELMO2 | 8.90728127 | 9.37042587 | 8.89714965 | 11.6830999 |
| HSP90AA1 | 14.279073 | 14.2484939 | 14.6492871 | 14.1134581 |
| CYFIP2 | 3.48413813 | 8.03556893 | 9.97835296 | 4.39985467 |
| CRK | 11.7028523 | 11.8931195 | 10.8555537 | 11.6140242 |
| CDC42 | 12.6557521 | 12.5990155 | 11.8944881 | 13.0138147 |
| ACTR3 | 13.3344912 | 13.701405 | 11.7109528 | 14.4603189 |
| ARPC2 | 12.6139207 | 12.9335136 | 10.6350288 | 13.7968326 |
| ELMO1 | 0.94860085 | 0 | 5.24488706 | 6.72628629 |
| CD3G | 0 | 0 | 0 | 0 |
| ABI1 | 10.2642784 | 9.96892594 | 9.18816846 | 11.4941156 |
| ARPC5 | 13.3230562 | 12.9068944 | 10.4944156 | 13.2129401 |
| WASF2 | 10.5287471 | 10.2132622 | 9.85698569 | 10.412591 |
| HSP90AB1 | 14.1016811 | 14.071661 | 14.7075921 | 14.9602617 |
| DOCK1 | 10.8016922 | 11.043458 | 10.0825083 | 0 |
| ARPC4 | 10.7056929 | 10.9185279 | 8.63926835 | 12.5619466 |
| BAIAP2 | 8.85337194 | 8.03556893 | 7.80322704 | 9.88626053 |
| CD247 | 1.91838623 | 0 | 0 | 0 |
| ACTR2 | 11.8909748 | 13.0225707 | 11.6519845 | 13.1631921 |
| WASL | 9.61829373 | 9.70689312 | 8.98575657 | 8.26893961 |
| WIPF3 | 2.49313492 | 2.83995959 | 5.42458623 | 3.03210084 |
| WIPF1 | 6.41498114 | 8.92718536 | 7.05245937 | 9.22216698 |
| ACTG1 | 15.8482085 | 16.4399607 | 14.5365478 | 15.7377419 |
| WIPF2 | 9.28701972 | 9.47660446 | 9.113638 | 9.50491828 |
| PLD2 | 7.37469175 | 7.31124879 | 7.62461271 | 7.88642821 |
| ARPC3 | 11.1883539 | 11.0417755 | 10.319785 | 12.2514321 |
| LIMK1 | 9.49215363 | 10.4554608 | 7.91778976 | 10.9286738 |
| MYH2 | 0 | 1.15704371 | 0 | 0 |
| YES1 | 11.2467406 | 10.9083851 | 10.2147077 | 9.40143421 |
| ACTB | 16.207816 | 16.5133911 | 13.9956603 | 15.7744085 |
| NCKAP1 | 12.5558186 | 12.934651 | 11.9367852 | 9.07620099 |
| CYFIP1 | 11.2262376 | 11.6770898 | 10.7477398 | 13.3855618 |
| NCKAP1L | 2.49313492 | 1.15704371 | 0.87970577 | 12.8173235 |
| DDX58 | 8.49785184 | 8.61871585 | 7.05245937 | 8.41578401 |
| IFIH1 | 6.10118782 | 4.68144927 | 6.2890967 | 8.77402928 |
| LSM14A | 10.6190828 | 10.4038855 | 10.5688302 | 10.6164661 |
| TSPAN6 | 8.47175649 | 8.80125568 | 11.0587629 | 6.99356115 |
| TKFC | #N/A | #N/A | #N/A | #N/A |
| DHX58 | 3.89724043 | 5.9692426 | 3.57410151 | 6.96069704 |
| NLRX1 | 7.11019618 | 7.8242587 | 8.33632803 | 8.34739991 |
| IL18BP | 5.28540222 | 5.46434153 | 5.39643353 | 5.50906239 |
| RORC | 0 | 0 | 0.87970577 | 2.40871186 |
| ATP7A | 8.65867564 | 9.23232498 | 9.30569698 | 9.54708732 |
| TUBB | 14.292661 | 14.2343504 | 13.861718 | 12.7097387 |
| GZMB | 0 | 0 | 0 | 5.6395216 |
| CLEC2A | #N/A | #N/A | #N/A | #N/A |
| LYST | 9.96181141 | 10.1981995 | 8.7753469 | 13.2504332 |
| SLAMF7 | 0.94860085 | 0 | 0 | 10.082535 |
| PRDX1 | 13.0130836 | 12.705114 | 13.3457919 | 12.8387017 |
| TUBB4B | 12.5507035 | 12.8332439 | 11.8522174 | 11.7460062 |
| NCR1 | 0 | 0 | 0 | 3.80012335 |
| PIK3R6 | 0 | 0 | 0 | 3.94204526 |
| SERPINB4 | 2.23572706 | 1.79493566 | 0 | 3.80012335 |
| CADM1 | 6.02125762 | 8.92350577 | 11.1462823 | 9.32916846 |
| SPON2 | 2.23572706 | 8.06242397 | 3.93168306 | 3.03210084 |
| S100A13 | 8.99112498 | 9.49151242 | 7.7868577 | 6.60377469 |
| MILR1 | 0 | 1.79493566 | 0 | 8.73562372 |
| CPLX2 | 1.51096192 | 0 | 2.94673086 | 2.40871186 |
| PLA2G3 | 2.71369582 | 1.15704371 | 1.81557543 | 0 |
| MRGPRX2 | 0 | 0 | 0 | 0 |
| GATA2 | 8.09032418 | 10.3598923 | 8.96895473 | 4.7473874 |
| FES | 2.71369582 | 6.80696843 | 4.34340782 | 6.8750428 |
| STXBP2 | 3.0721058 | 5.65306002 | 5.99842119 | 9.09905845 |
| C12orf4 | 9.93747681 | 9.87164347 | 9.74716918 | 9.24968236 |
| RAC2 | 4.41886458 | 4.99186193 | 0.87970577 | 10.9350398 |
| CD84 | 0 | 1.15704371 | 0 | 8.10548952 |
| RABGEF1 | 5.5649878 | 4.28466219 | 3.8509994 | 5.46270675 |
| FOXF1 | 0 | 8.9050861 | 4.56437817 | 5.55397481 |
| ADORA2B | 8.5198721 | 5.99729241 | 8.6085875 | 8.86573327 |
| VAMP7 | #N/A | #N/A | #N/A | #N/A |
| VAMP2 | 5.53636379 | 5.24754781 | 6.43612852 | 5.83364913 |
| STX4 | 8.6218321 | 8.92350577 | 8.14435234 | 10.5681366 |
| STXBP3 | 10.1155509 | 10.1379005 | 9.74998672 | 11.5539364 |
| ANXA3 | 9.54859075 | 7.02746372 | 6.92872562 | 1.28688115 |
| PRAM1 | 2.23572706 | 1.79493566 | 1.422233 | 3.26603689 |
| ABR | 11.0832001 | 10.3800497 | 9.54810905 | 12.4011062 |
| BCR | 9.3681572 | 10.0600609 | 10.603478 | 9.91445988 |
| RAB27A | 8.2569163 | 7.60421962 | 6.0902183 | 7.11821435 |
| CORO1A | 3.59931779 | 5.46434153 | 5.58466191 | 12.2222304 |
| KLRF2 | 0 | 0 | 0.87970577 | 0 |
| LAMP1 | 12.2836556 | 12.5906759 | 11.1648567 | 12.3938087 |
| AP1G1 | 12.0285415 | 11.3388812 | 11.3749797 | 11.4143417 |
| LEXM | #N/A | #N/A | #N/A | #N/A |
| LEF1 | 5.38474059 | 3.07038933 | 8.05609576 | 1.28688115 |
| BATF | 0 | 0 | 0 | 7.13298804 |
| DEFB125 | #N/A | #N/A | #N/A | #N/A |
| KIR2DS4 | 0 | 0 | 0 | 0 |
| DEFB126 | #N/A | #N/A | #N/A | #N/A |
| DEFB127 | 0 | 0 | 0 | 2.40871186 |
| NLRP2 | 0 | 0 | 0.87970577 | 0 |
| DEFB128 | 0 | 0 | 0 | 1.95233357 |
| DEFB129 | #N/A | #N/A | #N/A | #N/A |
| TNK1 | 4.64961546 | 3.86393845 | 2.7803101 | 4.89384756 |
| TREML1 | 0 | 0 | 0 | 5.93875626 |
| CRISP3 | #N/A | #N/A | #N/A | #N/A |
| CD300E | 0 | 0 | 0 | 1.28688115 |
| DEFB110 | 0 | 0 | 0 | 0 |
| TBKBP1 | 5.82273015 | 7.32237887 | 8.47549026 | 8.54345722 |
| AGER | 4.70154906 | 2.56803211 | 3.67242534 | 4.96208625 |
| DEFB112 | #N/A | #N/A | #N/A | #N/A |
| PDE1A | 2.23572706 | 4.68144927 | 2.12432814 | 0 |
| LCN2 | 0 | 0 | 0 | 1.95233357 |
| PRSS2 | #N/A | #N/A | #N/A | #N/A |
| DEFB132 | #N/A | #N/A | #N/A | #N/A |
| PRKCA | 10.3951378 | 10.8255313 | 9.25507579 | 9.8949996 |
| TRIM15 | 0 | 0 | 1.422233 | 1.95233357 |
| NLRP4 | 0 | 0 | 0 | 0 |
| XRCC5 | 13.4627157 | 13.415376 | 13.299115 | 13.1233293 |
| UBA7 | 6.08172309 | 6.94333532 | 2.12432814 | 8.45174687 |
| IPO7 | 12.278281 | 12.9464165 | 12.4805933 | 12.4407939 |
| ADCY7 | 6.52073653 | 7.90809234 | 6.0902183 | 8.20011392 |
| TRIM26 | 9.5768811 | 9.33505533 | 9.21693982 | 9.04908617 |
| DDOST | 12.5480247 | 11.977584 | 11.5494959 | 12.5071009 |
| CAPZA2 | 12.0926977 | 12.0287176 | 10.7314548 | 12.7724053 |
| PDE1B | 0 | 0 | 5.4799419 | 9.23943146 |
| PRKD1 | 7.69807954 | 9.3542053 | 7.60607203 | 0 |
| ANKHD1 | 5.31903982 | 5.099716 | 6.19298317 | 5.36562256 |
| SIRT2 | 9.34777637 | 9.07961811 | 8.66334462 | 10.7035662 |
| PYDC2 | #N/A | #N/A | #N/A | #N/A |
| POLR3F | 8.68814538 | 9.06638524 | 8.72471848 | 7.80535695 |
| ARHGEF2 | 11.2155937 | 10.4414607 | 9.44941693 | 13.4721958 |
| MID2 | 5.47767733 | 7.44997399 | 7.70804925 | 4.58436125 |
| RNF135 | 6.64616266 | 7.50000529 | 7.80322704 | 9.20823436 |
| S100A7 | 0 | 0 | 0 | 0 |
| ADAM15 | 10.0247657 | 10.1252197 | 9.20057988 | 10.2157514 |
| POLR3E | 9.72956976 | 9.40494994 | 10.2055 | 9.47901224 |
| LILRA5 | 0 | 0 | 0 | 1.28688115 |
| POLR2K | 9.92102268 | 9.39175913 | 9.26692706 | 10.199979 |
| DEFB116 | #N/A | #N/A | #N/A | #N/A |
| IFIT5 | 9.48844271 | 10.1410598 | 8.86114896 | 7.2465981 |
| DEFB115 | 0 | 0 | 0 | 0 |
| IFNL3 | #N/A | #N/A | #N/A | #N/A |
| UBE2L6 | 8.17137698 | 7.87054967 | 6.59484709 | 8.12789118 |
| OTULIN | #N/A | #N/A | #N/A | #N/A |
| CYBB | 0.94860085 | 0 | 3.8509994 | 11.1140286 |
| DTX4 | 0.94860085 | 5.65306002 | 5.63430284 | 8.89214813 |
| IRGM | 1.91838623 | 2.56803211 | 0 | 2.40871186 |
| HERC5 | 4.84799691 | 3.59693514 | 9.2451486 | 5.90472493 |
| TRIM11 | 8.79135827 | 8.68502907 | 8.9173124 | 8.96448527 |
| AXL | 13.0707868 | 13.6396087 | 3.67242534 | 6.29609026 |
| APOL1 | 2.71369582 | 3.07038933 | 0 | 6.39797405 |
| S100A9 | 0.94860085 | 1.15704371 | 0 | 10.8800044 |
| EIF2AK2 | 9.78821261 | 10.8087712 | 10.7221757 | 9.90151557 |
| S100A12 | 0 | 0 | 0 | 5.20633065 |
| ZBP1 | 0 | 0 | 1.422233 | 0 |
| APOBEC3A | 0 | 0 | 0.87970577 | 0 |
| APOBEC3H | 0 | 0 | 0 | 0 |
| DEFB131 | 0 | 0 | 1.422233 | 0 |
| XRCC6 | 13.6841591 | 12.9627749 | 13.7641625 | 13.0925722 |
| HMGB3 | 10.2077465 | 9.75503778 | 10.1079624 | 8.59260653 |
| SIGLEC14 | 0 | 0 | 0 | 0 |
| TRIM8 | 10.1107182 | 10.1906025 | 8.63926835 | 11.0173443 |
| POLR2L | 10.1203545 | 10.7695651 | 9.65408104 | 10.1750879 |
| CASP10 | 2.23572706 | 4.53543092 | 7.27192984 | 8.12044526 |
| ADCY8 | 1.51096192 | 0 | 0 | 10.2105131 |
| BPIFB3 | 0 | 0 | 0.87970577 | 2.40871186 |
| TRIM28 | 12.836798 | 12.3199402 | 12.6263319 | 11.4955503 |
| TRIM35 | 8.69463647 | 8.97036478 | 8.4093485 | 8.99729241 |
| NCF2 | 3.98458935 | 5.46434153 | 2.12432814 | 9.03729985 |
| POLR2E | 10.7467326 | 10.3933368 | 10.6976018 | 10.5279852 |
| ADCY3 | 7.70452619 | 9.86785014 | 8.83222546 | 10.2518966 |
| TOLLIP | 8.9565504 | 9.23529626 | 7.94263179 | 10.5082008 |
| CD300LB | 0.94860085 | 0 | 0 | 7.27360913 |
| PADI4 | 1.51096192 | 1.15704371 | 0 | 0 |
| LRRFIP1 | 10.0752117 | 10.7480083 | 9.33035672 | 10.7584398 |
| TRIM31 | 1.51096192 | 1.79493566 | 0 | 2.7548875 |
| TRIM32 | 9.50509698 | 9.60093501 | 8.5961152 | 5.41515021 |
| DEFB118 | 0 | 0 | 0 | 0 |
| RNF216 | 10.6992688 | 10.3380678 | 10.0474784 | 9.81440622 |
| NAIP | 4.89384756 | 4.28466219 | 4.08236197 | 4.7473874 |
| ATG12 | 10.3435966 | 10.3747026 | 9.45631334 | 9.99791335 |
| PIN1 | 10.6190828 | 10.1331551 | 10.3827969 | 10.4692552 |
| PRKCSH | 12.3009817 | 11.5845539 | 10.7825232 | 11.258937 |
| BLK | 0.94860085 | 2.83995959 | 1.81557543 | 2.7548875 |
| ITCH | 11.5565013 | 11.4949456 | 10.9094131 | 11.7876414 |
| DEFB104B | #N/A | #N/A | #N/A | #N/A |
| ADCY1 | 4.59514557 | 3.07038933 | 9.50211575 | 8.82518141 |
| MST1R | 2.90496572 | 1.79493566 | 4.28243981 | 1.95233357 |
| GZMM | 0 | 0 | 2.37851162 | 2.7548875 |
| PPARG | 4.53853816 | 6.63865312 | 8.04242547 | 10.4837956 |
| SIRPB1 | 0 | 1.15704371 | 0.87970577 | 2.7548875 |
| TNK2 | 8.46417814 | 8.9050861 | 7.99101157 | 7.71945728 |
| DDX60 | 6.94637997 | 7.74355536 | 7.03408363 | 9.36732738 |
| NCF1 | 0 | 0 | 0 | 9.86862195 |
| BMX | 0 | 0 | 0 | 0 |
| DEFB108B | 0 | 0 | 0.87970577 | 0 |
| TRIM62 | 5.47767733 | 6.22843392 | 6.17632277 | 3.94204526 |
| IL1RL2 | 2.90496572 | 3.07038933 | 0.87970577 | 2.40871186 |
| LGALS3 | 9.83140226 | 9.82338316 | 7.02480723 | 12.3864715 |
| TRIM25 | 8.64205169 | 9.73011345 | 9.43548275 | 8.2349611 |
| PRKAR1A | 12.1900469 | 12.8103969 | 11.7609902 | 12.9310385 |
| SRPK2 | 10.1441866 | 9.84481559 | 9.62371692 | 8.50973542 |
| SDHAF4 | #N/A | #N/A | #N/A | #N/A |
| DEFB123 | #N/A | #N/A | #N/A | #N/A |
| ADCY5 | 3.59931779 | 3.98185265 | 4.45811948 | 1.95233357 |
| ADARB1 | 7.52747701 | 8.00129559 | 8.32880954 | 11.6020725 |
| PTX3 | 9.11444511 | 10.7978155 | 4.00898878 | 7.14771372 |
| BCL2L1 | 9.4526325 | 10.627242 | 8.19888699 | 10.1277107 |
| S100B | 0 | 0 | 0.87970577 | 4.07038933 |
| CRCP | 9.8431045 | 9.64380209 | 8.78907713 | 9.83497662 |
| NLRP1 | 6.26659917 | 8.92350577 | 7.42071818 | 3.26603689 |
| TXN | 12.2693722 | 12.0257348 | 11.737429 | 12.0224823 |
| PRKAR1B | 7.77945673 | 8.08225551 | 8.13576058 | 7.38939472 |
| PTK6 | 0.94860085 | 2.23266076 | 1.422233 | 5.75915583 |
| IL34 | 1.51096192 | 1.79493566 | 0.87970577 | 2.7548875 |
| POLR2F | 10.045159 | 9.21747308 | 10.0290525 | 9.95887167 |
| TREX1 | 7.61867915 | 6.40667272 | 4.79960542 | 6.70653055 |
| APOBEC3C | 8.5052558 | 9.04636009 | 8.08316017 | 7.10318288 |
| F12 | 4.35473424 | 3.73660488 | 5.79545527 | 1.28688115 |
| FRK | 3.0721058 | 3.73660488 | 6.98709344 | 2.40871186 |
| PANX1 | 9.39621944 | 9.71748794 | 8.9616812 | 11.3913779 |
| DEFB124 | 0 | 0 | 0 | 0 |
| TNFAIP8L2 | 1.51096192 | 1.15704371 | 2.7803101 | 9.87967514 |
| PRKDC | 13.2417449 | 13.679744 | 14.2825542 | 12.8527811 |
| SUGT1 | 10.8754268 | 11.4033476 | 11.0885299 | 10.6708775 |
| PRKCG | 1.51096192 | 1.15704371 | 3.8509994 | 3.94204526 |
| NLRC5 | 8.91004304 | 8.71073757 | 5.45252955 | 9.89716478 |
| CSF1R | 4.89384756 | 3.4409522 | 1.81557543 | 12.7013606 |
| APOBEC3D | 0 | 1.15704371 | 5.17951105 | 2.40871186 |
| SERINC5 | 6.73674016 | 6.94333532 | 6.79337571 | 7.14771372 |
| ATRIP | 7.61191007 | 8.31233844 | 8.10527998 | 6.66633054 |
| SIGLEC15 | 2.71369582 | 3.07038933 | 1.422233 | 5.6395216 |
| POLR3G | 7.02036885 | 7.67553369 | 9.33784487 | 6.42240122 |
| IL27 | 0 | 1.15704371 | 0 | 2.40871186 |
| MAP3K5 | 6.36719631 | 9.18115226 | 8.43408607 | 10.3036552 |
| MIF | 2.23572706 | 0 | 2.12432814 | 1.95233357 |
| OTUD5 | 9.74990294 | 9.21147465 | 9.50710087 | 9.29987196 |
| TRIM14 | 6.41498114 | 8.04237074 | 8.21529031 | 7.07274895 |
| ADAR | 12.0362011 | 12.0329687 | 12.4456486 | 13.1433976 |
| PCBP2 | 9.58560105 | 9.79762911 | 10.7721743 | 8.84784036 |
| APOBEC3B | 5.64731451 | 6.69125498 | 6.05419729 | 3.03210084 |
| DDX3X | 13.0981851 | 13.2568603 | 13.2517457 | 13.2024223 |
| DDX41 | 9.29980329 | 9.48904513 | 9.31901723 | 10.4277214 |
| PRSS3 | 0.94860085 | 6.42760617 | 0 | 3.03210084 |
| SRMS | 0 | 2.23266076 | 0 | 3.80012335 |
| APCS | 0 | 0 | 0 | 0 |
| ADCY2 | 0 | 0 | 1.422233 | 4.07038933 |
| POLR3H | 9.5733821 | 9.62590971 | 9.89689242 | 9.1103528 |
| DEFB105A | #N/A | #N/A | #N/A | #N/A |
| DEFB106A | #N/A | #N/A | #N/A | #N/A |
| POLR3D | 10.2445779 | 9.76324557 | 9.66008506 | 10.4216862 |
| DEFB119 | #N/A | #N/A | #N/A | #N/A |
| CYLD | 10.9737763 | 10.9876122 | 9.4751457 | 10.4441763 |
| DEFB121 | #N/A | #N/A | #N/A | #N/A |
| MRE11A | 9.16188768 | 9.25580756 | 9.95781305 | 8.20011392 |
| TRIM13 | 9.27628728 | 9.45649816 | 8.87408995 | 11.5155126 |
| PRKAR2A | 10.7120799 | 11.2119065 | 11.3281925 | 11.4444041 |
| TYK2 | 10.2088806 | 10.0600609 | 9.53675231 | 11.3226324 |
| ART1 | 0 | 0 | 1.422233 | 2.7548875 |
| AKIRIN2 | 8.15739747 | 8.24355484 | 8.5992434 | 9.51910546 |
| GPER1 | 7.19475685 | 7.32237887 | 5.33771109 | 5.75915583 |
| CSF1 | 9.12166302 | 8.65004648 | 6.15946773 | 10.2157514 |
| DEFB135 | 0 | 0 | 0.87970577 | 0 |
| REL | 6.91348784 | 6.70832521 | 7.88746435 | 8.46352437 |
| DEFB104A | #N/A | #N/A | #N/A | #N/A |
| POLR3C | 9.29342567 | 9.05305702 | 9.04884103 | 10.6503876 |
| DEFB134 | 0 | 0 | 2.37851162 | 0 |
| ZC3HAV1 | 10.644361 | 10.9539345 | 10.6380738 | 10.4707097 |
| SSC5D | 6.13975598 | 6.50795317 | 0.87970577 | 3.94204526 |
| POLR3B | 8.87061131 | 9.23232498 | 9.75139342 | 7.51932174 |
| POLR1C | 8.93484081 | 8.34549396 | 9.40008958 | 9.92728908 |
| CAPZA1 | 12.8553201 | 12.8985315 | 12.0395668 | 14.2505716 |
| PRKAR2B | 7.69807954 | 9.05975004 | 8.67525139 | 5.83364913 |
| TRIM10 | 0 | 1.79493566 | 0.87970577 | 0 |
| PML | 7.87369018 | 8.58202905 | 7.55550943 | 7.27360913 |
| SFTPD | 0 | 0 | 1.81557543 | 3.94204526 |
| CASP1 | 4.21800615 | 6.62073302 | 0 | 7.71945728 |
| SERINC3 | 11.7797768 | 12.2324509 | 10.3737026 | 11.9367852 |
| POLR3A | 9.46593366 | 9.92018907 | 10.7749435 | 9.47609811 |
| VNN1 | 0.94860085 | 0 | 0 | 0 |
| STYK1 | 3.98458935 | 6.56544486 | 3.35473424 | 0 |
| PDE1C | 10.8732903 | 10.7857794 | 5.17951105 | 3.46727948 |
| DEFB106B | #N/A | #N/A | #N/A | #N/A |
| KLRG1 | 0 | 1.15704371 | 5.45252955 | 3.03210084 |
| PSTPIP1 | 2.49313492 | 2.23266076 | 0.87970577 | 7.28697353 |
| CLEC5A | 0 | 0 | 1.81557543 | 7.44989147 |
| CTNNB1 | 13.2883297 | 13.1792093 | 13.1474069 | 14.4612383 |
| ADCY4 | 0.94860085 | 5.8509994 | 2.12432814 | 4.58436125 |
| POLR2H | 10.0961353 | 9.21147465 | 9.66902677 | 9.60145511 |
| S100A8 | 0 | 0 | 0 | 7.62811741 |
| POLR3GL | 7.54187101 | 7.63139535 | 7.48968743 | 9.1141067 |
| SIKE1 | 10.2066115 | 9.84481559 | 11.2140944 | 10.9222796 |
| CHID1 | 9.65026194 | 9.63711389 | 9.21897486 | 9.54154194 |
| ADRBK1 | 10.1311239 | 9.79762911 | 9.93943229 | 11.158231 |
| ABL2 | 11.7318366 | 12.3580495 | 10.0991637 | 11.6020725 |
| KRT16 | 0.94860085 | 3.2675358 | 1.422233 | 3.03210084 |
| DHX36 | 10.0286106 | 10.2266891 | 9.91956355 | 9.75087444 |
| SAA1 | 0 | 2.23266076 | 0 | 0 |
| TXNIP | 7.43771047 | 9.4590217 | 8.01925712 | 11.7502213 |
| DEFB133 | #N/A | #N/A | #N/A | #N/A |
| DEFB105B | #N/A | #N/A | #N/A | #N/A |
| MATK | 0 | 2.23266076 | 6.67990229 | 12.036366 |
| TRIM4 | 8.5415613 | 9.3731044 | 8.47208155 | 8.90517638 |
| MSRB1 | 7.76725753 | 7.44997399 | 7.55550943 | 7.27360913 |
| NCR2 | 0 | 0 | 0 | 0 |
| CD1D | 0 | 0 | 3.76447355 | 8.14261766 |
| POLR1D | 9.23914501 | 9.52569698 | 9.90196803 | 9.95887167 |
| FBXO9 | 9.7775841 | 9.62590971 | 9.80650129 | 10.1911333 |
| ADCY6 | 9.23914501 | 9.80162753 | 8.75027152 | 9.29987196 |
| POLR3K | 8.07553263 | 8.27262979 | 9.07151599 | 7.71945728 |
| SRPK1 | 11.209959 | 11.1356836 | 12.1984205 | 10.7762768 |
| ECSIT | 9.13121391 | 7.80051168 | 9.11800665 | 8.61393677 |
| ADCY9 | 8.84467438 | 10.0897418 | 9.20261362 | 9.07620099 |
| DHX9 | 12.8248693 | 12.5476489 | 13.1524687 | 12.1506422 |
| ANKRD17 | 10.669204 | 11.5978151 | 11.7991966 | 11.0183393 |
| TAX1BP1 | 11.613085 | 11.8537696 | 10.5894077 | 11.8126346 |
| APOBEC3G | 1.91838623 | 4.81403765 | 3.93168306 | 8.09032418 |
| APOBEC3F | 3.89724043 | 5.46434153 | 5.36737107 | 5.14893411 |
| XIAP | 10.7072901 | 10.9130016 | 10.0825083 | 11.4533015 |
| MMP2 | 10.4187697 | 13.3077444 | 5.60969587 | 7.96318509 |
| COCH | 4.53853816 | 2.23266076 | 9.55134298 | 2.40871186 |
| GBP5 | 3.98458935 | 0 | 0 | 1.28688115 |
| PLSCR1 | 8.98056804 | 8.49105138 | 8.32503556 | 9.00537192 |
| AICDA | 0 | 0 | 0 | 0 |
| EXOSC6 | 10.152437 | 9.58016433 | 9.26886945 | 8.86127305 |
| EXOSC3 | 9.19421513 | 8.81711157 | 8.5992434 | 9.53595573 |
| RNF168 | 10.5565348 | 9.7652861 | 9.29029538 | 8.75975506 |
| RNF8 | 8.89890365 | 8.89017306 | 9.66753737 | 9.56086777 |
| MSH6 | 11.0782642 | 11.3755011 | 12.0848582 | 9.22564127 |
| NBN | 10.6384993 | 11.0476693 | 10.9680043 | 11.3411296 |
| ERCC1 | 9.99756057 | 9.6811154 | 8.65438542 | 11.5967255 |
| LIG4 | 8.83003978 | 9.15643707 | 8.53543092 | 10.2501918 |
| SWAP70 | 10.6096959 | 10.6148203 | 9.07376669 | 10.3199543 |
| APLF | 7.72996056 | 6.74200621 | 6.33467553 | 7.04154965 |
| SUPT6H | 10.0438885 | 11.2364388 | 11.3876889 | 10.9434818 |
| MYB | 4.84799691 | 5.04701482 | 5.45252955 | 5.36562256 |
| SOCS5 | 8.82416321 | 12.1102353 | 8.30222765 | 8.48103248 |
| HLX | 5.82273015 | 7.54828248 | 6.81506302 | 10.2416615 |
| PRKCZ | 1.91838623 | 4.19061486 | 7.63074036 | 5.14893411 |
| RARA | 6.08172309 | 7.62234472 | 7.37033868 | 8.20716078 |
| DRD2 | 1.51096192 | 0 | 2.12432814 | 3.26603689 |
| NMI | 7.35851934 | 6.22843392 | 5.14567746 | 9.17990909 |
| MUL1 | 9.57161993 | 9.40230862 | 9.13962809 | 9.01343434 |
| TRAFD1 | 9.62170392 | 9.15017885 | 9.66604643 | 9.61474665 |
| UNG | 10.1711017 | 9.84095097 | 10.9558606 | 8.34100726 |
| PAXIP1 | 8.23931211 | 7.58578881 | 9.31331389 | 7.21897486 |
| PHB | 10.4477858 | 10.4991383 | 11.4556406 | 10.5925038 |
| IGF2 | 0.94860085 | 7.15775114 | 0 | 3.80012335 |
| SLAMF6 | 0 | 0 | 0 | 2.40871186 |
| STAT5B | 8.96993328 | 9.49890903 | 9.26495859 | 9.9692426 |
| LAG3 | 3.0721058 | 1.15704371 | 2.592158 | 0 |
| SLC25A6 | #N/A | #N/A | #N/A | #N/A |
| CLCF1 | 8.14801892 | 7.96613049 | 1.422233 | 6.66633054 |
| THOC1 | 9.17814195 | 8.92350577 | 9.40724652 | 8.40356598 |
| WHSC1 | 11.2788385 | 12.080974 | 11.9172005 | 9.36104455 |
| TBX21 | 0.94860085 | 0 | 2.94673086 | 6.21470767 |
| KIR3DL2 | 0 | 0 | 0 | 1.28688115 |
| OSCAR | 1.51096192 | 1.79493566 | 1.422233 | 5.83364913 |
| KLRB1 | 1.51096192 | 0 | 3.23112516 | 1.95233357 |
| CD300LG | 1.51096192 | 0 | 1.422233 | 0 |
| CD300LF | 0 | 0 | 0 | 4.66789213 |
| KIR2DL2 | #N/A | #N/A | #N/A | #N/A |
| SH2B2 | 4.35473424 | 3.2675358 | 5.14567746 | 6.49313492 |
| SELL | 0.94860085 | 0 | 2.12432814 | 0 |
| CDH1 | 1.91838623 | 1.15704371 | 5.85947267 | 0 |
| KIR2DL4 | 0 | 0 | 0 | 3.26603689 |
| TREML4 | 0 | 0 | 0 | 0 |
| CLEC2B | 0 | 5.58044702 | 1.422233 | 1.28688115 |
| COL2A1 | 1.91838623 | 1.15704371 | 10.3226267 | 3.03210084 |
| FKBP1A | 9.71054817 | 11.0718377 | 10.1123352 | 9.90800225 |
| LAIR2 | 0 | 0 | 0.87970577 | 0 |
| CLEC2D | 4.35473424 | 5.15055968 | 5.97933945 | 6.62497818 |
| CD81 | 10.9077995 | 11.648007 | 10.2299003 | 12.0348849 |
| CD200 | 0.94860085 | 0 | 0 | 0 |
| SIGLEC9 | 0 | 0 | 0.87970577 | 7.79610452 |
| CD300C | 0 | 0 | 0 | 8.73562372 |
| CD3E | 1.51096192 | 1.79493566 | 0.87970577 | 1.28688115 |
| TREML2 | 0 | 0 | 0.87970577 | 1.95233357 |
| HCST | 1.91838623 | 2.83995959 | 1.422233 | 7.84197312 |
| ITGA4 | 9.71692232 | 11.4409418 | 6.67990229 | 10.4426947 |
| CD200R1 | 0.94860085 | 1.15704371 | 1.81557543 | 1.28688115 |
| CXADR | 8.76394266 | 3.86393845 | 10.2618011 | 8.50973542 |
| VCAM1 | 10.0665198 | 0 | 0 | 3.46727948 |
| KLRF1 | 0 | 0 | 0 | 1.95233357 |
| ITGB1 | 14.7654684 | 15.5119416 | 11.4365502 | 14.0544992 |
| CD300LD | #N/A | #N/A | #N/A | #N/A |
| KLRC1 | 0 | 0 | 0 | 1.28688115 |
| CD3D | 0 | 0 | 0 | 0 |
| SPPL2B | 9.07780377 | 8.59589149 | 7.61227877 | 8.40356598 |
| ITGB7 | 4.21800615 | 3.59693514 | 4.56437817 | 12.855581 |
| NCR3LG1 | 7.23083705 | 8.12747852 | 9.69991942 | 6.92706681 |
| SPPL2A | 10.4649133 | 10.4208445 | 8.81886973 | 11.4324532 |
| ICAM4 | #N/A | #N/A | #N/A | #N/A |
| JAML | #N/A | #N/A | #N/A | #N/A |
| SIGLEC7 | 0 | 0 | 0 | 4.58436125 |
| CD160 | 3.98458935 | 4.09000653 | 3.93168306 | 4.58436125 |
| COL3A1 | 5.41616417 | 14.6703055 | 6.94848404 | 2.40871186 |
| CD33 | 1.51096192 | 0 | 1.422233 | 8.65563805 |
| COL1A2 | 16.8202651 | 16.8390953 | 0 | 1.28688115 |
| COL1A1 | 15.0282778 | 15.4184486 | 7.31822644 | 4.89384756 |
| CD34 | 1.91838623 | 3.2675358 | 1.422233 | 1.28688115 |
| COL17A1 | 4.48026512 | 4.28466219 | 2.592158 | 1.95233357 |
| FCRLB | 4.84799691 | 6.46825747 | 3.8509994 | 5.31433399 |
| AMBP | 0 | 1.15704371 | 1.422233 | 0 |
| IFNL4 | 0 | 1.15704371 | 0 | 0 |
| SPG21 | 10.004094 | 9.73638502 | 9.26692706 | 11.6395849 |
| BCAR1 | 10.9223465 | 11.1623158 | 9.32091334 | 9.56635853 |
| RFTN2 | 4.84799691 | 3.86393845 | 4.51222689 | 1.28688115 |
| THY1 | 10.8846513 | 12.4720181 | 2.592158 | 0 |
| RLTPR | 5.72519582 | 3.73660488 | 7.02480723 | 4.7473874 |
| PDE4D | 8.18982456 | 8.42012866 | 8.44799238 | 7.65921102 |
| RP4-583P15.14 | #N/A | #N/A | #N/A | #N/A |
| THEMIS2 | 5.50716035 | 4.19061486 | 6.55673595 | 9.73393049 |
| CACNB4 | 3.98458935 | 2.83995959 | 7.74799153 | 9.39529849 |
| STK11 | 10.7303003 | 10.0583977 | 9.92456016 | 10.1461104 |
| VASP | 11.59418 | 11.5493756 | 8.50243404 | 13.6845032 |
| RBCK1 | 9.10959563 | 9.64380209 | 8.18651046 | 9.44966454 |
| STOML2 | 11.1340605 | 10.4880309 | 10.8205061 | 10.5749435 |
| RNF31 | 7.71094416 | 7.99429686 | 7.19140473 | 8.19308177 |
| RP4-583P15.15 | 0 | 1.79493566 | 1.422233 | 0 |
| PDE4B | 7.87369018 | 9.1842059 | 5.14567746 | 6.56056212 |
| PTPRJ | 10.2268336 | 8.76081973 | 7.66689858 | 11.1242764 |
| CACNB3 | 8.51625158 | 8.43562859 | 6.49089098 | 5.20633065 |
| SPPL3 | 7.95675366 | 8.24939793 | 8.54194843 | 7.7772882 |
| ENAH | 12.7694577 | 12.2478919 | 12.7593868 | 8.06732679 |
| CD38 | 0.94860085 | 0 | 0 | 4.49505553 |
| MNDA | 0 | 0 | 0 | 6.12845838 |
| NFAM1 | 1.51096192 | 0 | 1.81557543 | 6.53838296 |
| PLEKHA1 | 9.73271014 | 9.73430317 | 9.73301532 | 3.03210084 |
| GCSAML | 0 | 0 | 0 | 0 |
| GCSAM | 4.98093927 | 4.99186193 | 5.00225245 | 0 |
| CACNA1F | 0 | 0 | 0.87970577 | 2.7548875 |
| LPXN | 7.16711703 | 9.70901487 | 4.70984202 | 11.3226324 |
| FCRL5 | 0 | 0 | 1.81557543 | 2.7548875 |
| PAWR | 11.5556149 | 11.0980387 | 10.9878111 | 4.66789213 |
| PTPN2 | 9.00938077 | 8.63669712 | 8.6453342 | 9.44373096 |
| UBASH3A | 0 | 0 | 0 | 1.95233357 |
| PRNP | 11.5529476 | 13.1162969 | 8.60236483 | 8.76927586 |
| ELF1 | 8.82119998 | 9.16576123 | 8.86114896 | 10.8008837 |
| PHPT1 | 9.5432252 | 9.28745844 | 8.27122956 | 10.0863618 |
| EZR | 12.4785067 | 12.2500852 | 11.0439295 | 10.7691684 |
| GBP1 | 10.7498359 | 9.71748794 | 1.422233 | 6.74577515 |
| BTNL2 | 0 | 1.15704371 | 0 | 0 |
| STAP1 | 0 | 0 | 0.87970577 | 2.40871186 |
| SLC39A10 | 10.0438885 | 10.6910456 | 10.1361965 | 11.068429 |
| CMTM3 | 8.55228484 | 8.28406063 | 8.36233838 | 10.3344744 |
| PRKCH | 2.90496572 | 3.4409522 | 7.93274625 | 7.98788928 |
| TESPA1 | 0.94860085 | 0 | 0 | 1.28688115 |
| KCNN4 | 7.23964626 | 2.83995959 | 0 | 12.6819346 |
| RPS19 | 13.0512293 | 13.2091959 | 12.9715364 | 12.9115324 |
| INS | 0 | 1.79493566 | 0 | 0 |
| PARP9 | 7.54187101 | 7.43978934 | 7.57470705 | 9.18348651 |
| SOCS3 | 8.91004304 | 9.91282947 | 5.17951105 | 9.13637598 |
| IFNGR2 | 9.79122802 | 9.64158215 | 8.57723998 | 12.7119014 |
| PTPN1 | 10.5583155 | 10.3827213 | 9.99861864 | 10.9455828 |
| IRF9 | 3.80426012 | 2.56803211 | 3.09592442 | 4.7473874 |
| IRF6 | 1.91838623 | 2.56803211 | 4.66277517 | 1.95233357 |
| IFNGR1 | 10.3961338 | 10.0550519 | 9.16093027 | 11.5267975 |
| STAT1 | 10.5323657 | 11.6290378 | 9.89179889 | 10.8822074 |
| IFI30 | 0.94860085 | 0 | 0.87970577 | 6.64573048 |
| TRIM38 | 6.6727082 | 8.63223186 | 7.06156021 | 9.55261147 |
| IRF2 | 7.32553033 | 7.70141044 | 7.05245937 | 9.7676217 |
| MID1 | 9.79574747 | 9.62366205 | 11.8307766 | 0 |
| MT2A | 11.234584 | 12.3181982 | 7.08841725 | 6.68664059 |
| IRF5 | 3.22342255 | 1.79493566 | 2.94673086 | 9.69446236 |
| SUMO1 | 11.1316769 | 10.6693634 | 10.9024731 | 10.7997268 |
| TRIM68 | 7.74247787 | 7.78443855 | 7.21606678 | 5.55397481 |
| CD44 | 12.9800378 | 13.8973879 | 5.00225245 | 12.3162702 |
| TRIM34 | 2.23572706 | 2.83995959 | 0 | 1.95233357 |
| SOCS1 | 5.25058267 | 4.19061486 | 2.592158 | 4.7473874 |
| PIAS1 | 8.45277662 | 8.6676438 | 8.63012148 | 8.77873424 |
| CDC37 | 12.2248187 | 12.0072726 | 11.5466678 | 12.7272674 |
| MED1 | 9.58560105 | 10.3089192 | 10.9943534 | 10.1015293 |
| NR1H2 | 10.4089772 | 9.94205993 | 7.41362793 | 10.2771711 |
| MX2 | 5.93640238 | 3.73660488 | 1.422233 | 5.55397481 |
| XAF1 | 0.94860085 | 6.79103262 | 0 | 7.78672704 |
| IFNAR2 | 6.78607355 | 6.15542543 | 5.77346893 | 8.27565895 |
| IP6K2 | 9.83140226 | 9.16884759 | 9.04196025 | 9.83271599 |
| USP18 | 3.98458935 | 6.56544486 | 8.41645977 | 7.27360913 |
| IFIT3 | 9.00419237 | 8.9050861 | 3.67242534 | 9.4257614 |
| IFIT1 | 7.04078287 | 9.19942697 | 6.97750891 | 8.02818235 |
| IFIT2 | 7.04078287 | 8.10181813 | 2.7803101 | 7.00977264 |
| RNASEL | 7.02036885 | 7.61331064 | 7.04330075 | 9.09148828 |
| ISG20 | 3.0721058 | 3.98185265 | 3.67242534 | 7.64896869 |
| EGR1 | 5.79908731 | 6.18010807 | 2.94673086 | 8.83428142 |
| STAT2 | 10.0875557 | 10.5174529 | 8.69289443 | 9.9836492 |
| IFI35 | 6.41498114 | 5.88141991 | 5.36737107 | 9.59342847 |
| IFNAR1 | 10.5864082 | 10.5403315 | 9.47002838 | 11.8746664 |
| UBE2K | 9.33127527 | 10.3693356 | 11.393546 | 9.66922169 |
| GPRC5B | 8.39835892 | 3.4409522 | 7.74234312 | 0 |
| FOXP1 | 10.1641276 | 10.1220256 | 9.77783063 | 8.20716078 |
| RC3H2 | 11.7986662 | 11.3340665 | 11.074563 | 10.5694949 |
| ERBB2IP | 12.9129717 | 12.8598104 | 11.0924136 | 12.7821485 |
| HSPA1B | 9.78971295 | 8.31233844 | 13.2523653 | 9.5961152 |
| HSPA1A | 9.67664471 | 8.70649602 | 13.5537733 | 7.41388175 |
| AIF1 | 1.51096192 | 0 | 0.87970577 | 8.67097485 |
| ASS1 | 10.7904137 | 9.50870595 | 7.31061278 | 10.7950656 |
| GAPDH | 14.4926563 | 14.71874 | 13.8579723 | 14.3363399 |
| EPRS | 13.1818573 | 12.664045 | 12.5053749 | 12.6885432 |
| DAPK3 | 10.7820891 | 10.645127 | 9.54324453 | 10.0787112 |
| MRC1 | 0 | 0 | 0.87970577 | 1.95233357 |
| SYNCRIP | 12.0707985 | 12.3017334 | 12.2904653 | 11.5055684 |
| AQP4 | 0.94860085 | 0 | 4.51222689 | 3.46727948 |
| SLC26A6 | 8.51258246 | 8.56334883 | 8.32129678 | 10.4692552 |
| RPL13A | 13.3363029 | 13.4659298 | 12.6257568 | 13.446432 |
| EDN1 | 7.1004521 | 9.45649816 | 4.84247606 | 5.02724254 |
| STAR | 0.94860085 | 2.23266076 | 1.81557543 | 1.95233357 |
| DAPK1 | 10.3911684 | 7.10706011 | 9.53184929 | 10.3488499 |
| CD58 | 8.59786633 | 6.74200621 | 6.30451104 | 9.9567827 |
| TDGF1 | 0 | 0 | 0 | 0 |
| RORA | 1.51096192 | 7.42944859 | 7.9227925 | 1.28688115 |
| LY9 | 0 | 1.15704371 | 0 | 3.03210084 |
| USP17L2 | 0.94860085 | 0 | 1.81557543 | 0 |
| PUM1 | 10.9664406 | 10.7187469 | 11.0678108 | 10.4924541 |
| PUM2 | 9.37623394 | 10.8166797 | 10.5767772 | 10.219229 |
| VAMP8 | 1.91838623 | 2.83995959 | 6.86777294 | 11.04801 |
| SNX4 | 9.97780939 | 9.2788908 | 9.66008506 | 10.3134613 |
| BAX | 9.5663395 | 9.99007559 | 9.19024446 | 9.7192519 |

TABLE 4
Differentially expressed genes in mock vs
ZIKV treated cells, Related to FIG. 13A.

| gene_name | CHME | BJ | 293FT | THP1 |
|---|---|---|---|---|
| PRKDC | 0.55856801 | #N/A | −0.5171828 | 0.5678045 |
| ATP5J2 | 0.03250525 | #N/A | 0.12618779 | −0.7223692 |
| CUX1 | 0.33250463 | #N/A | −0.7744567 | 0.59583179 |
| KIAA0754 | 0.69031731 | #N/A | −0.4817685 | 0.70147337 |
| SHANK3 | 0.1325234 | #N/A | −0.1932178 | 0.67542576 |
| ZBED6 | 0.23744485 | #N/A | −0.1458937 | 0.65822503 |
| PEG10 | 0.38703256 | #N/A | 0.05514638 | 3.34969884 |
| RPL36A | −0.5163688 | #N/A | 0.71993133 | 0.33299626 |
| SMIM20 | 0.11472651 | #N/A | −0.0788868 | −0.6510827 |
| ITGB3 | −0.4062701 | #N/A | 1.07866076 | 0.35009084 |
| TMEM141 | −0.2728154 | #N/A | −0.0280894 | −0.6319133 |
| ETV5 | −0.4302263 | #N/A | 0.68421692 | 0.50675169 |
| PCDHGA11 | −0.7805866 | #N/A | −0.4143672 | 0.70705975 |
| HIST1H3F | −0.1981391 | #N/A | 0.01109358 | −0.6276276 |
| OAS1 | −1.1541368 | 9.58142668 | 5.3096096 | 0.90513148 |
| NPY | 5.9728042 | 3.48888859 | 3.73845785 | −8.6155662 |
| PRDM16 | −2.1052731 | 0.02462381 | −0.3462215 | −8.6155662 |
| LPAR2 | −0.3416272 | 7.92084439 | −0.0173286 | 0.9173359 |
| KRT80 | 0.5004813 | −0.0203586 | 5.30202839 | −6.7410313 |
| MXRA5 | 2.84875627 | −0.6585835 | −1.8433505 | −6.7410313 |
| PSMB8 | −0.0257905 | 0.43896737 | 6.72832517 | −0.4983962 |
| GIPC3 | 3.59074958 | 6.44630765 | −0.4725947 | 0.86615677 |
| MATK | 6.40679881 | −0.9016165 | −0.1026186 | −0.2989202 |
| LYZ | 6.3945123 | 3.4332996 | −0.0464664 | −0.5886975 |
| OAS2 | 0.63106751 | 3.81151293 | −6.385417 | 1.12260925 |
| LAPTM5 | −0.4429111 | 0.1635659 | −6.385417 | 0.41452932 |
| MMP1 | −0.1052119 | −0.1527386 | −6.385417 | −0.4904597 |
| DKK1 | −0.2418678 | 0.15045916 | 0.28262609 | 6.37288581 |
| WAS | 1.10196892 | 6.06626917 | 6.30963839 | 0.69193795 |
| NUP210 | 6.22554827 | 0.25025596 | −0.2636351 | 0.56760781 |
| SSTR1 | 0.63948628 | −0.4193241 | −1.0261776 | −6.1560313 |
| COL4A5 | 0.42751738 | −0.1978899 | −0.045773 | −6.1560313 |
| IL1RN | 0.66167517 | −6.067605 | −4.8004512 | 0.50176743 |
| DOCK2 | 0.27488063 | −0.0610358 | 6.04365971 | 0.45958114 |
| SPI1 | −5.9015357 | 3.4332996 | −5.8004174 | 0.38688844 |
| CD52 | −1.1724122 | 3.4332996 | −5.8004174 | −0.7118497 |
| MX2 | 0.58615835 | 3.668701 | −5.8004174 | 1.60389444 |
| ITGAM | −2.169796 | 3.48888859 | −5.8004174 | 0.37023318 |
| KRT7 | −0.1314643 | 0.21904008 | −5.8004174 | 5.38519517 |
| C5AR2 | −0.9400716 | 5.78928385 | 3.73845785 | −0.2420397 |
| UNC5C | −0.5846658 | 5.4613723 | −0.5331489 | 1.46334881 |
| CACNA1C | −0.0046296 | −0.5821937 | −0.8378071 | 5.36040653 |
| CD163 | −2.1687748 | 4.43069973 | 5.32430546 | 0.58211974 |
| VCAM1 | −0.6958874 | 3.48888859 | 5.30202839 | −2.1887125 |
| PRTN3 | −1.1021635 | 3.48888859 | 5.30202839 | 0.57108024 |
| CLEC11A | 5.22547271 | 0.35352936 | 0.16020668 | −0.2368824 |
| ALDH7A1 | 0.33541349 | −0.6986587 | −0.4720292 | −5.1560457 |
| LUM | −0.321423 | 0.00161738 | 0.18579467 | −5.1560457 |
| HRH3 | −4.9015622 | −1.3299475 | 0.28799462 | −5.1021092 |
| ADCY8 | −0.5636555 | 5.07649491 | 3.71793484 | 0.52645495 |
| MERTK | −2.7511542 | 5.07649491 | −0.1266408 | 0.43068354 |
| PIK3R5 | −0.7306224 | −5.0676274 | −0.1116798 | 0.38149518 |
| LCP2 | −4.9015622 | −5.0676274 | −1.035987 | 0.47272564 |
| NOVA1 | 0.22588338 | −5.0676274 | −1.1821583 | −3.4084252 |
| RBM20 | 4.58739662 | −5.0676274 | −0.6143299 | −2.7134571 |
| HCN4 | −0.3044137 | −5.0676274 | −0.1952518 | −4.0920678 |
| ELMO1 | −4.9015622 | 5.05623944 | 0.19035631 | 0.89102265 |
| LINGO1 | −1.1367624 | 5.05623944 | −0.8259569 | 0.26520487 |
| PPAP2B | 0.50024667 | −0.2495042 | 0.10833637 | 4.96601755 |
| LRRN1 | 0.50223481 | −1.8622136 | 4.73927048 | 4.94952467 |
| ID4 | −0.0410974 | 0.07143908 | 0.2720259 | 4.94952467 |
| MIDI | 0.31719771 | 0.13354129 | −0.1175922 | 4.93228693 |
| RUNX1T1 | −4.9015622 | −0.7329959 | −1.1621057 | −2.3915117 |
| PCDH19 | −4.9015622 | 0.10850427 | −0.8945431 | 4.37278862 |
| ASB18 | −4.9015622 | −0.2890079 | −2.8460059 | 0.19350137 |
| MMP19 | −0.203705 | −0.0882791 | −4.8004512 | 0.62125278 |
| TNFRSF1B | −0.4218794 | 0.27427814 | −4.8004512 | 0.29266972 |
| IL7R | 0.39151831 | −0.0430196 | −4.8004512 | 0.15542917 |
| SLC43A3 | −0.1352096 | −0.0764032 | −4.8004512 | 0.34169585 |
| SEMA6D | 4.58739662 | 0.07908043 | −0.0674083 | 0.92417597 |
| LST1 | 4.58739662 | 4.43069973 | 0.55958861 | −0.8234998 |
| CNR1 | −0.5056187 | 4.49134188 | 0.40180746 | 0.59870044 |
| SLCO5A1 | −0.3259786 | −0.4013902 | −0.3957229 | −4.4049425 |
| ACTA2 | −0.6319022 | 0.23195277 | −1.4478652 | 4.3976275 |
| GFRA1 | 0.43716748 | 0.29025356 | −0.2804988 | 4.3976275 |
| THY1 | −0.2593801 | 0.19149247 | −1.6163056 | 4.37278862 |
| ARL10 | −0.0806352 | −0.1264517 | −1.3374969 | 4.37278862 |
| EMX1 | 3.66367464 | 0.29143604 | 0.83321132 | −4.2848041 |
| ZFHX4 | 0.31941866 | −0.281322 | −1.1811921 | −4.2126614 |
| MX1 | 1.13932495 | 4.20656455 | 0.35482213 | 1.10676095 |
| CREB5 | 0.15233725 | −0.0121701 | −0.6610423 | −4.106626 |
| SYNGR3 | 0.04624613 | 1.09678061 | 0.27369681 | −4.0920678 |
| SDK2 | −0.515328 | −0.5422419 | −0.7294643 | −3.9619542 |
| CHI3L1 | −1.022842 | −0.589369 | 3.73845785 | 0.40325958 |
| IL24 | −0.0427296 | −1.3117922 | 3.73845785 | −0.4118264 |
| SEMA5A | 0.46474002 | −0.2663807 | 3.73845785 | 0.94484013 |
| NCF4 | 3.59074958 | 3.4332996 | 3.73845785 | −0.503469 |
| RAMP1 | −0.1123147 | 0.60674164 | 3.73845785 | −0.5679737 |
| COL1A2 | 0.20670315 | 0.00303122 | 3.71793484 | 0.1691995 |
| PIK3CG | −0.1367247 | 3.4332996 | 3.71793484 | 0.38469389 |
| SH3BP4 | 0.23393648 | −0.0600997 | −0.3223726 | −3.697865 |
| PROX1 | 3.66367464 | −0.9511435 | −0.7272002 | 0.60853134 |
| MLXIPL | 3.59074958 | 1.00141192 | −0.1497451 | 0.82336116 |
| CRIPAK | 3.57571573 | −1.890997 | −1.1982972 | 0.18550482 |
| HOPX | −1.2135108 | 3.48888859 | −0.4928824 | −1.0797892 |
| BTK | 1.20778658 | 3.4332996 | −0.1421229 | −0.4641776 |
| MSR1 | −0.3675987 | 3.4332996 | 0.96923679 | −0.3189994 |
| MAMDC2 | −0.09718 | 0.63440714 | −1.6840843 | 3.39560669 |
| CNN1 | 0.14887662 | 0.328907 | 0.62863108 | 3.39560669 |
| XIRP1 | 0.88192383 | −1.7266461 | −3.350767 | 0.95095787 |
| LMOD1 | −0.5227811 | 0.4424635 | −0.4400164 | 3.34969884 |
| KIF5C | −0.2096428 | 0.61973636 | −0.0581768 | −3.327976 |
| CYP46A1 | 1.53634708 | 1.68969137 | 0.37109241 | −3.176782 |
| KIF21B | 0.03336338 | 3.15919917 | 0.06643995 | 0.51426548 |
| LPHN3 | 0.66870543 | −2.865779 | −0.10518 | 0.24998614 |
| IL8 | −0.195687 | −0.5106556 | −2.8433506 | −0.7594216 |
| AUTS2 | 0.2345527 | −1.0777958 | −1.0442454 | −2.7870897 |
| CTD- | −0.1799742 | −1.7355251 | −1.3830569 | −2.7838653 |
| 3088G3.8 | | | | |
| DIP2C | 0.34259199 | −0.033156 | −0.0165371 | −2.69401 |
| MT-ND6 | 0.10501022 | 0.78976666 | 2.66993642 | −1.6975222 |
| FLNC | 0.68010938 | −0.1862298 | −0.019039 | −2.393736 |
| IGSF9B | −0.6255443 | −0.6109856 | −0.8907965 | −2.3705094 |
| ACAP1 | −0.3346675 | −0.2731026 | 0.46329324 | −2.3439557 |
| SLC1A3 | 0.02634705 | 2.29186556 | −0.0390104 | 0.27651271 |
| CCDC88C | 1.64303969 | −2.2909711 | −0.1159312 | 0.42054278 |
| NCKAP1L | −1.8845369 | −0.3029683 | 2.28806291 | 0.31893842 |
| STU | 0.05887474 | −2.2787969 | −0.5491355 | 0.33799939 |
| IFIH1 | 0.08893951 | 2.25581554 | −0.1415286 | 0.49912749 |
| TERT | 0.37596158 | −0.3317167 | 0.06170395 | −2.252293 |
| GYPC | −2.1687748 | 0.18068601 | 0.05783795 | −0.3746608 |
| MMP9 | −2.1272702 | 1.26233647 | −0.7120835 | 0.28539908 |
| TUBB4A | −0.5477682 | −0.3317167 | −1.6115098 | −2.1217145 |
| THBD | −2.0855064 | −0.7273101 | −0.2533527 | 0.46792244 |
| MT-CYB | −0.1903529 | 0.48389271 | 2.0707826 | −1.0098223 |
| IFIT1 | 0.73373208 | 2.05464698 | −0.0606624 | 0.73885966 |
| GPRC5A | 0.38137344 | −0.0572162 | −0.6374925 | −2.0465405 |
| EVI2A | 0.54548126 | 0.43280834 | −2.0404567 | −0.6103066 |
| EPPK1 | 2.02470531 | −1.1137453 | −0.6468634 | −1.2439358 |
| EGR2 | 2.02043905 | 0.02466681 | −0.1891361 | 0.42731675 |
| MT-ND5 | 0.08356319 | 0.56786435 | 2.01919912 | −1.162643 |
| PRKCD | 1.2211035 | −1.991135 | −0.8944593 | 0.41397933 |
| MDK | −0.1001546 | −0.0524536 | −0.109649 | 1.98957322 |
| COMP | −0.1367109 | −1.2619042 | −1.0392533 | −1.9764558 |
| MT-ATP8 | 0.0818921 | 0.71675793 | 1.96964005 | −1.837835 |
| ILDR2 | 0.58175194 | 0.68246955 | −0.2755375 | −1.9660671 |
| FOSL1 | 0.10405813 | −0.0220373 | 1.96399611 | 0.76615599 |
| GAS7 | −1.9441854 | −0.044558 | −0.1643115 | 0.30183955 |
| POLR2A | 0.16205426 | −0.2040257 | −1.8721099 | 0.40085782 |
| MT-ND1 | −0.1745864 | 0.77002376 | 1.8135832 | −1.2644244 |
| COL5A1 | 0.20319389 | −0.0099103 | −0.1728984 | −1.799472 |
| KMT2D | 0.21193923 | −0.5173759 | −1.791409 | 0.37477863 |
| CASP4 | −0.582906 | 0.02620174 | 1.77288201 | −0.4271332 |
| GPRIN3 | −1.7557724 | 0.39971002 | −0.461149 | 0.73153932 |
| VAMP8 | −1.7557724 | −0.2859506 | −0.1387797 | −0.6752804 |
| ZNF385A | 0.13253733 | 1.57153385 | −1.7531018 | 0.55200081 |
| WNK2 | −1.7217701 | 0.28519585 | −0.7243857 | −0.5807958 |
| GLI3 | 0.61838097 | −0.2275776 | −1.2886567 | 1.69744465 |
| REXO1L1 | 0.30180717 | 0.43750793 | −1.6925744 | −1.6966166 |
| PLEKHH1 | −0.6089734 | −0.648367 | −0.1653834 | 1.68163149 |
| MT-ND4 | −0.1118824 | 0.42706104 | 1.66095625 | −0.8697342 |
| RMI1 | −0.0847928 | −1.0028894 | −1.6287174 | −0.687812 |
| SLIT2 | −0.2163053 | −0.3665019 | −0.5662179 | 1.61161863 |
| PARP9 | 0.464091 | 1.60496377 | −0.1841645 | 0.56527382 |
| RAPH1 | −0.021561 | −0.0524204 | −1.5888207 | −0.2970959 |
| FUS | 0.33108673 | −1.5808059 | −0.3294648 | −0.0545556 |
| A2M | −1.5784032 | −0.1397512 | −1.3287134 | 0.32472385 |
| MT-CO2 | −0.3970466 | 0.27714226 | 1.57779709 | −0.4191846 |
| OLFML2B | −0.2362135 | −0.1238046 | 0.3111769 | 1.56808491 |
| SMG7 | 0.45614341 | −1.0275234 | −1.5534647 | 0.29014321 |
| TNFSF15 | −1.1719671 | 1.53428229 | −0.6259549 | 0.44739545 |
| ALPK3 | −0.118396 | 1.52534352 | −1.1010232 | 0.60929055 |
| IL4I1 | −0.3675987 | −0.2936225 | −1.5195513 | 0.35301686 |
| FRAS1 | 0.2818312 | −0.4792134 | −0.7574238 | −1.5157248 |
| PCLO | 1.40523564 | −0.225452 | −1.4921478 | 0.87746628 |
| SDHC | 0.12989616 | −1.4812605 | −0.5428579 | −0.1694983 |
| SLCO3A1 | 0.25499804 | −1.4713173 | −0.63527 | −0.2624378 |
| C3 | −0.0454665 | −0.3404185 | −1.4574456 | 0.45247309 |
| PLBD2 | −0.1682737 | 0.22232424 | −1.4517393 | 0.24813893 |
| SMARCD3 | −0.0965059 | 0.19877868 | 0.21943459 | −1.4414646 |
| MT-ATP6 | −0.2520609 | 0.2168809 | 1.43728376 | −0.6207972 |
| SRCAP | 0.17784799 | −0.3278129 | −1.430524 | 0.41888201 |
| SORL1 | −1.4237604 | −0.0662354 | −0.4453764 | 0.21578459 |
| MT-ND4L | −0.0744306 | 0.72292561 | 1.42143397 | −1.0814801 |
| DSP | 0.00362152 | −0.1222561 | −0.3700848 | 1.41934258 |
| C1orf226 | −0.8219199 | 0.16983364 | −1.4177899 | 0.0694358 |
| ONECUT2 | 0.84433511 | −0.3151689 | −0.9857611 | 1.39786017 |
| EP400 | 0.50858744 | −0.8116141 | −1.3965709 | 0.80082717 |
| CFD | −0.5700285 | 1.39524113 | 0.27054919 | −0.6135203 |
| RERE | −0.0374192 | −0.398216 | −1.3942566 | −0.1021657 |
| CYFIP2 | −0.5871374 | 0.04913455 | −0.4701895 | −1.3914874 |
| QDPR | 0.23043737 | −1.3900388 | −0.640424 | −0.5224256 |
| ADAM12 | 0.27546743 | −0.0226244 | −0.1796894 | −1.3842479 |
| IGF2BP1 | −0.1407351 | −0.2648655 | −0.5942332 | −1.3842479 |
| MT-ND2 | −0.2423961 | 0.56063061 | 1.3806633 | −1.3224996 |
| NOVA2 | 0.0804315 | −1.3299475 | −1.1529025 | −1.3692276 |
| RHPN1 | 1.36635457 | −0.8293821 | 0.71566425 | 0.10830908 |
| PLCG2 | −1.3644571 | −0.6584898 | −0.4526192 | 0.56848258 |
| STX16 | 0.11789007 | −1.2712963 | −1.345218 | −0.0586111 |
| ATN1 | 0.13059953 | −0.3258559 | −1.1490467 | 1.33363436 |
| COL12A1 | 0.5439358 | −0.2276631 | −0.5238261 | 1.32635874 |
| MBD6 | 0.12842861 | −0.305501 | −1.1072236 | 1.31939386 |
| FILIP1L | −0.0115303 | 0.02802335 | −1.3052451 | 0.70583022 |
| CSF1R | 0.11756287 | −1.3029882 | 0.69886113 | 0.53882988 |
| ABCC3 | −0.2206392 | −0.1372932 | −1.3009373 | 0.42875484 |
| KDM6B | 0.31010062 | −0.5881058 | −1.2971445 | 0.40586579 |
| ZFHX3 | 0.13343599 | −0.4458125 | −1.2888444 | 0.09676354 |
| RFX7 | 0.34253599 | −0.2184657 | −1.286543 | 0.70147993 |
| ATF7 | −0.1561397 | −0.531292 | −1.2850829 | 0.90567832 |
| CTSH | −1.2793388 | 0.36311133 | −0.2898567 | −0.3039583 |
| VAV3 | −1.1671817 | 1.27225733 | 0.01844571 | 0.25766211 |
| PHF16 | 0.03366559 | −0.4136876 | −0.4699743 | 1.26745174 |
| KIAA1199 | −0.4592599 | −0.3373217 | −1.262705 | 0.43347718 |
| CHERP | 0.34595099 | −0.5713322 | −0.7094687 | 1.26085851 |
| USP14 | 0.3756032 | −0.7925867 | −1.245602 | 0.0790873 |
| LGALS9 | −0.1742321 | 0.82372476 | −1.2327962 | 0.44460882 |
| KIF20A | 0.16385481 | 0.2421316 | 0.06779917 | 1.23205536 |
| TOP1 | 0.31373711 | −0.5496323 | −1.2301152 | −0.0696299 |
| POM121C | 0.38114235 | −0.3506261 | −1.2284191 | 0.43954652 |
| ARMCX4 | 0.30956809 | −1.227344 | −0.7015578 | 0.70017075 |
| NFIC | 0.37044793 | −0.4279061 | −1.2252027 | −0.0411676 |
| NEIL3 | 0.12741199 | 0.04325957 | 0.07042456 | −1.2233172 |
| UCP2 | −0.0663055 | −0.0269073 | −0.8780106 | 1.21644916 |
| MT-CO1 | −0.3729258 | 0.40780064 | 1.21150156 | 0.5683562 |
| FCER1G | −1.2008899 | −0.5715409 | −0.0464664 | 0.3191262 |
| TMEM56 | 0.39382906 | 0.5776071 | −0.1787177 | −1.1932676 |
| 42616 | 1.1928605 | −0.1680268 | −0.5904962 | −0.1724611 |
| CIT | 0.29451654 | −0.1200916 | −0.2221024 | 1.19055104 |
| SPEN | 0.32643477 | −0.3938108 | −1.1884296 | 0.75326 |
| EIF4E3 | 1.18017908 | −0.3720995 | −0.7396968 | 0.04360486 |
| EVI2B | 1.1798008 | 0.05193235 | −0.8178706 | 0.8387684 |
| TCF7L2 | 0.31021404 | 0.06709651 | −1.1764611 | 0.01715123 |
| SRRM2 | 0.34610083 | −0.7069288 | −1.173218 | 0.75927743 |
| WDFY4 | −1.1730145 | −0.708919 | −1.0459601 | 0.57420167 |
| PIK3CB | 0.12421695 | −0.4666868 | −1.1727642 | 0.0693577 |
| DOCK8 | −1.1727419 | −0.881099 | −0.4861799 | 0.61320715 |
| ARID1A | 0.44436448 | −0.382666 | −1.1676271 | 0.77124323 |
| MT-CO3 | −0.4835282 | 0.33258417 | 1.16501721 | 0.29632562 |
| CD14 | −0.2255508 | 1.1618252 | −0.0394213 | 0.39682377 |
| CCNK | −0.0803625 | −0.2435617 | −1.1615384 | −0.0180263 |
| SON | 0.2930706 | −0.723822 | −1.1568301 | 0.47072176 |
| FAM65A | 0.02114799 | 0.10674684 | −1.153156 | 0.57175245 |
| RUNX2 | 0.26753735 | −0.0673458 | −1.1509865 | 0.62001825 |
| NFAT5 | 0.43966001 | −0.3438441 | −1.1497174 | 0.487456 |
| EMR2 | −1.1482367 | −0.3646666 | −0.5344985 | 0.46457246 |
| LOX | −0.0097495 | −0.7443344 | −1.1457178 | −0.0312525 |
| ANP32B | 0.14811145 | −1.1378669 | −0.7758419 | −0.1525176 |
| DACH1 | −1.1367624 | 0.29143604 | −0.734726 | 0.87622688 |
| SAMD9L | 0.31020835 | 0.60597837 | 1.13509854 | 0.81730475 |
| LRP1 | 0.61268665 | −1.1348502 | −0.9422073 | 0.48383424 |
| MXRA8 | 0.32984102 | 0.15374021 | 0.37564714 | −1.1340805 |
| MAP1B | 0.5115714 | −0.2087157 | −0.8254015 | 1.13346819 |
| GBP1 | 0.03477046 | 0.44000044 | 1.1303804 | 0.32261185 |
| CCDC88B | 1.12953438 | 0.91736448 | −0.0559382 | 0.49998204 |
| PGF | −0.5222585 | 0.11458045 | 0.20421268 | −1.1259466 |
| WASF1 | 0.05036229 | −0.2231193 | −0.13366 | 1.11911897 |
| ABCA1 | 0.42721694 | 0.08687954 | −0.4123377 | 1.11647413 |
| DSG2 | 0.20236697 | 1.11575342 | −0.6391983 | 0.66454434 |
| SRGAP1 | −0.0349331 | −0.3728424 | −1.1070133 | 0.64166275 |
| TRANK1 | 0.99444151 | −0.3991849 | −1.1068927 | 0.69698881 |
| HIC2 | 0.11527956 | −0.2538066 | −0.0461373 | 1.10425129 |
| KIRREL | 0.25838526 | −0.1205885 | −0.1702649 | −1.104014 |
| MKI67 | 0.4867459 | −0.3081071 | −1.1037905 | 0.70931992 |
| HIST1H4K | −0.2156987 | −0.2934607 | 0.94457425 | −1.1018549 |
| EYS | 0.37334583 | −0.5396395 | −1.1018225 | 0.31710378 |
| NDUFB1 | −0.1938241 | 0.24759739 | 0.30761455 | −1.098758 |
| ZFP36 | 0.48479605 | 0.45087036 | −0.8839947 | 1.09444208 |
| RGCC | 1.08461982 | 0.1789841 | 0.15342221 | −0.4831311 |
| TICRR | 0.2108576 | −0.4013499 | −0.3351883 | 1.0796965 |
| BCORL1 | 0.29674532 | −0.7578045 | −1.0621236 | 1.07842476 |
| QSER1 | 0.21001806 | −0.3012598 | −1.0745008 | 0.28312994 |
| ZNF804A | −0.1552824 | −0.1641029 | −0.1476532 | 1.07130476 |
| CCDC107 | −0.3016173 | 0.79366283 | 0.67974971 | −1.0712081 |
| TRIM14 | 0.30554472 | 0.49553909 | −0.3121917 | 1.06671873 |
| SEPHS2 | 0.07188116 | −1.0654829 | −0.2648831 | −0.2372006 |
| OAS3 | −0.1419907 | 1.06430862 | −0.5552335 | 0.96237942 |
| HIVEP3 | 1.06120211 | −0.3847046 | −0.9928498 | 0.55795123 |
| DAP | 0.03699283 | −0.7187563 | −1.0611614 | 0.14920605 |
| EP300 | 0.22775632 | −0.2058586 | −1.0605785 | 0.63766128 |
| HIPK2 | 0.30973459 | −0.625363 | −1.0593831 | 0.77960613 |
| CRYL1 | −0.4342605 | 0.1986127 | −0.1967358 | −1.0572807 |
| ADNP | 0.09764623 | −0.3730778 | −1.0556177 | 0.36032707 |
| UBL5 | −0.0398895 | 0.2158329 | 0.1225837 | −1.0554302 |
| LONRF2 | 0.19257195 | −1.0522365 | −0.4813123 | 0.11330967 |
| ZMIZ1 | 0.39051171 | −0.3416421 | −1.0463175 | 0.71269777 |
| PEX7 | −0.2555182 | 0.74870317 | 0.2318023 | −1.0461369 |
| TRIM22 | −0.1291734 | 0.43486453 | −1.0458303 | 0.66443171 |
| HIST1H3C | −0.236828 | 0.09898507 | −1.0458303 | −0.2756738 |
| DUSP23 | −0.5736764 | 0.07823363 | −1.042597 | −0.6506835 |
| PML | 0.17509999 | −0.1606207 | −0.1263692 | 1.0409416 |
| PTGS1 | −0.644067 | −0.1423909 | −1.0392153 | 0.24579125 |
| PLCD4 | 0.58829374 | −0.3698972 | −1.0366483 | −0.6118727 |
| MRPL43 | 0.02434269 | 0.30720908 | 0.0673465 | −1.0358881 |
| LMLN | 0.0584856 | −0.2993258 | −1.0356128 | 0.43055772 |
| TPP1 | 0.25787154 | −0.0224647 | −1.0352202 | 0.80260583 |
| HIST1H4D | −0.1523439 | 0.02678756 | 0.07260453 | −1.0338073 |
| BHLHE22 | 0.84447004 | −0.3029683 | 1.0333753 | 0.31425891 |
| RAC2 | 0.35808516 | 0.50528824 | −1.0261776 | 0.33668025 |
| CDCP1 | −0.3434544 | −0.6335388 | −1.0260552 | 0.50099988 |
| KMT2A | 0.49947188 | −0.3113809 | −1.0244917 | 0.9623194 |
| TCIRG1 | −0.0568849 | 0.0708318 | 1.01924123 | 0.3686595 |
| FYCO1 | 0.61438847 | −0.997829 | −1.018591 | 0.48093687 |
| NAV1 | 0.38924411 | −0.4341749 | −1.0147577 | 0.16921684 |
| CENPF | 0.42862904 | 0.03319866 | −0.1285281 | 1.01437395 |
| PTPN12 | 0.13059914 | −0.7024465 | −1.008294 | 0.09625941 |
| MAMDC4 | −0.0337778 | −0.2717828 | 1.00766345 | 0.07979122 |
| PEAK1 | 0.42636878 | −0.6044548 | −1.0027593 | 0.06128411 |
| AHNAK2 | 1.00267349 | −0.5150554 | −0.7209908 | 0.80047227 |
| NCOA6 | 0.59679242 | −0.4330229 | −1.0003214 | 0.72866641 |
| PTRHD1 | −0.2496686 | 0.26284598 | 0.03301546 | −0.9999594 |
| IGFBP3 | −0.0083693 | 0.23051347 | 0.9997056 | 0.05070075 |
| GSE1 | 0.3012102 | −0.5855585 | −0.4458471 | 0.99851194 |
| HPRT1 | −0.0138153 | −0.9976442 | −0.8006981 | −0.2256058 |
| INCENP | 0.33677122 | −0.0505206 | 0.02128117 | 0.99726922 |
| RPRD2 | 0.21639054 | −0.0227392 | −0.6406683 | 0.99383304 |
| PLEKHG2 | 0.14959398 | −0.1803355 | −0.3843273 | 0.99164809 |
| MTUS1 | 0.9891786 | −0.7554818 | −0.3079831 | 0.45861058 |
| ROMO1 | −0.075124 | 0.25388424 | 0.11375394 | −0.9849351 |
| SCAF4 | 0.23897134 | −0.5273573 | −0.9839867 | 0.73519729 |
| ZMYM3 | 0.1437086 | 0.11495333 | −0.3044171 | 0.98066388 |
| ZNF32 | 0.12872572 | 0.29144493 | 0.05536028 | −0.9779282 |
| HTRA1 | −0.2280955 | 0.14265745 | 0.11840367 | −0.9764346 |
| PRRC2C | 0.35071405 | −0.1652916 | −0.9756104 | 0.90514103 |
| SGCB | 0.12121462 | −0.3350771 | −0.4887875 | 0.9748984 |
| PRR12 | 0.31763133 | −0.6850117 | −0.9695994 | 0.69110584 |
| NIPBL | 0.33050465 | −0.7355763 | −0.9680475 | 0.24550172 |
| HIST1H4C | −0.1672742 | 0.10211209 | 0.02360819 | −0.9672796 |
| PMVK | 0.15279751 | −0.9665166 | 0.09814498 | −0.308098 |
| MAP2K3 | 0.08241877 | −0.6944746 | −0.9601106 | 0.13790536 |
| LAMTOR4 | −0.1491644 | 0.18264958 | 0.00561578 | −0.9597033 |
| EPHB4 | 0.30139358 | 0.06608928 | 0.05046969 | −0.9592366 |
| C21orf67 | 0.00976938 | 0.95765745 | −0.2309025 | 0.32537538 |
| ZMIZ2 | −0.1680968 | 0.09659525 | −0.289224 | 0.95741421 |
| RPS18 | −0.1046989 | 0.18103916 | 0.07115236 | −0.9563046 |
| PHACTR4 | 0.14878318 | −0.1290776 | −0.4388188 | 0.95574589 |
| S100A6 | −0.0449636 | 0.17273931 | 0.9549477 | −0.6335333 |
| RUNX3 | −0.1285263 | −0.9547057 | 0.05886197 | 0.39110508 |
| PTP4A1 | 0.12562581 | −0.7162147 | −0.9501469 | −0.0399866 |
| NSRP1 | −0.0840151 | 0.29114616 | 0.07523641 | 0.94934661 |
| CHAMP1 | 0.2641776 | −0.061881 | −0.9452495 | 0.92827972 |
| SEC22A | −0.1763303 | 0.04969652 | 0.01218869 | −0.9443834 |
| RPS10 | 0.0521097 | 0.32133216 | 0.187609 | −0.943421 |
| EFNA5 | 0.09591948 | 0.28201694 | −0.4482544 | −0.9404129 |
| ZFP36L1 | 0.09255463 | −0.03629 | −0.9375689 | 0.5600723 |
| TBC1D7 | −0.0627878 | 0.05883048 | −0.1853933 | −0.9365536 |
| FOS | −0.0068944 | 0.93566798 | −0.1515304 | 0.37993188 |
| ADAMTS5 | 0.17378947 | −0.597037 | −0.5808157 | −0.9341167 |
| C15orf52 | −0.2118316 | −0.7227749 | 0.13276681 | −0.9341167 |
| NUDT2 | −0.1455226 | 0.25967918 | 0.30780043 | −0.9323624 |
| CREB3L2 | 0.10023243 | −0.2410613 | −0.9314225 | 0.86276453 |
| SEC63 | 0.14934785 | −0.5354326 | −0.9308545 | 0.13340559 |
| PCBP2 | 0.04987286 | −0.0394607 | −0.9306552 | −0.0776712 |
| EPB41L1 | 0.56541984 | 0.06961527 | −0.6064192 | 0.9253706 |
| RPS3A | 0.07163863 | 0.05682431 | 0.18214075 | −0.9252828 |
| SMNDC1 | −0.0692168 | −0.5590946 | −0.9236545 | 0.21189466 |
| IFI27L2 | −0.0866673 | 0.05998045 | 0.28017713 | −0.922902 |
| VDR | 0.06976031 | 0.13290143 | −0.2664268 | 0.92196745 |
| PTPN14 | 0.48205476 | −0.4965729 | −0.9213774 | 0.78114055 |
| RPA1 | 0.30164496 | −0.9213504 | −0.3120327 | −0.0028605 |
| GPC4 | −0.1090106 | −0.9187263 | −0.607676 | 0.29335569 |
| NAF1 | 0.30257622 | −0.5312605 | −0.9157635 | 0.15843862 |
| GATAD2B | 0.43585142 | −0.3364681 | −0.9139008 | 0.68869668 |
| C10orf12 | 0.40408291 | −0.2807151 | −0.9119921 | 0.82210224 |
| POLE4 | 0.04612104 | 0.14400089 | −0.034875 | −0.9113613 |
| PROSER1 | 0.0222151 | −0.1515912 | −0.9101879 | 0.58193352 |
| R3HDM1 | 0.08283854 | 0.04721923 | −0.9094027 | 0.45148306 |
| STXBP1 | 0.35663077 | −0.4703101 | −0.9092918 | 0.54331046 |
| PIK3CD | 0.22892846 | −0.0045877 | 0.1728646 | 0.908914 |
| KMT2C | 0.26336954 | −0.3605869 | −0.9088592 | 0.6005487 |
| DISP1 | 0.01691058 | −0.2025554 | −0.1646181 | 0.90633633 |
| MED30 | 0.08628878 | −0.0616118 | 0.11439065 | −0.9018805 |
| CNOT6 | 0.40970014 | −0.6256697 | −0.9014494 | 0.21629706 |
| POP7 | −0.1532846 | 0.08626463 | 0.11540445 | −0.9012932 |
| UQCR11 | −0.0467395 | 0.03333818 | 0.00254441 | −0.8998578 |
| MRPL13 | −0.0403306 | 0.13461203 | 0.1482989 | −0.8977186 |
| AFF1 | 0.42366746 | −0.1274649 | −0.6364188 | 0.89763005 |
| USE1 | 0.14787306 | 0.09290884 | −0.116306 | −0.8972467 |
| MED25 | 0.17391447 | −0.013439 | −0.1177897 | 0.89670523 |
| KCNMA1 | 0.19664352 | −0.5673327 | −0.8957927 | 0.00351517 |
| RNF213 | 0.59732716 | −0.1003189 | −0.5463444 | 0.8936837 |
| MRPL54 | −0.0499691 | 0.43212516 | −0.0739844 | −0.8929659 |
| NXT1 | −0.1251647 | 0.12862057 | 0.01323725 | −0.8911934 |
| ASAP1 | 0.25644723 | −0.1445016 | −0.8900354 | 0.6159554 |
| MYRF | 0.31031317 | 0.88970737 | −0.0754236 | 0.61639206 |
| SF3A2 | 0.22604264 | −0.0850332 | −0.2709879 | 0.88689089 |
| BRPF3 | 0.0173829 | −0.4066345 | −0.6844504 | 0.88530968 |
| NDUFA13 | −0.2701047 | 0.07319257 | 0.03117576 | −0.8827915 |
| TMEM18 | −0.2813936 | 0.19101209 | −0.004335 | −0.8783266 |
| MKL2 | 0.52903668 | −0.530769 | −0.8780809 | 0.64455523 |
| NAV2 | 0.21650514 | −0.4126277 | −0.7283668 | 0.87763418 |
| ARHGAP30 | −0.5108134 | −0.8775704 | −0.6567143 | 0.30119336 |
| EEF1B2 | −0.1682227 | 0.16702852 | 0.16339712 | −0.8773841 |
| PGAP1 | 0.50218346 | −0.6667299 | −0.6605524 | 0.87634831 |
| EHD2 | 0.13385363 | 0.00422457 | −0.875667 | 0.70565097 |
| TACC3 | 0.15016803 | −0.4554933 | −0.2779966 | 0.87520113 |
| FBRSL1 | 0.04290985 | −0.2413952 | −0.2078766 | 0.87424255 |
| ZNF704 | −0.8737058 | −0.4653351 | −0.4152791 | 0.03395607 |
| IGFBP4 | 0.24317303 | −0.8733671 | −0.5665169 | −0.0502305 |
| IQSEC1 | 0.39284499 | −0.0026487 | −0.8714528 | 0.45861417 |
| SUZ12 | 0.02440281 | −0.7807132 | −0.8710514 | 0.21634094 |
| NPM3 | −0.163286 | 0.3019828 | 0.12827919 | −0.8701307 |
| HSBP1 | −0.0627247 | 0.12124873 | 0.12726812 | −0.869425 |
| CD4 | −0.8461443 | −0.8693469 | 0.04531473 | 0.68015175 |
| BCL3 | 0.86876902 | 0.11926206 | −0.2072062 | 0.44149448 |
| FBLN1 | 0.29137437 | −0.4219101 | −0.6170268 | −0.8677751 |
| FAM122B | 0.23907077 | −0.3407692 | −0.8673955 | 0.16584691 |
| AFF3 | −0.5720862 | 0.16400536 | −0.7618979 | −0.8669188 |
| SIVA1 | −0.1561811 | 0.3087107 | −0.0712679 | −0.8656761 |
| PHF5A | −0.1701548 | 0.11244088 | 0.13886836 | −0.864815 |
| ABL2 | 0.36216146 | −0.2451381 | −0.8638198 | 0.45179241 |
| RSL24D1 | −0.2389157 | −0.0389964 | 0.30423202 | −0.8637014 |
| PABPC1 | 0.26519502 | −0.6777439 | −0.8630539 | 0.39365829 |
| SKI | 0.38623209 | −0.324062 | −0.8616356 | 0.46164583 |
| CYP51A1 | −0.4211367 | −0.3515389 | 0.16786758 | −0.8610433 |
| KDELR3 | −0.2800236 | 0.16643045 | −0.0794664 | −0.8576793 |
| COX7B | −0.0728593 | 0.02595875 | 0.12631499 | −0.8515755 |
| CREBBP | 0.21570396 | −0.2994133 | −0.8498336 | 0.50680887 |
| PARP10 | −0.0871189 | 0.84909812 | −0.087318 | 0.39780405 |
| FAM98B | 0.19335649 | −0.6535028 | −0.8463523 | 0.15103079 |
| PPTC7 | 0.31887066 | −0.648637 | −0.8459845 | 0.12440082 |
| FUOM | 0.31517029 | −0.1921098 | 0.18463805 | −0.8435553 |
| NCOR2 | 0.08793661 | −0.7958127 | −0.8432483 | 0.27145866 |
| PRADC1 | 0.02138664 | 0.32502702 | 0.15063122 | −0.8404689 |
| PDE4A | 0.41208157 | −0.7789678 | −0.8388351 | 0.71554887 |
| RAB32 | −0.0783444 | 0.1962406 | 0.18568248 | −0.8383766 |
| INO80D | 0.61924075 | −0.2778023 | −0.8366363 | 0.54465976 |
| SNRPG | −0.2083257 | 0.11393086 | 0.15297169 | −0.8362501 |
| GRIN2D | 0.23321949 | −0.5385676 | −0.8356754 | 0.64094294 |
| ANPEP | 0.04920187 | −0.1823364 | 0.83558492 | 0.61663112 |
| POU2F1 | 0.52119873 | −0.3031778 | −0.8354182 | 0.58917193 |
| TNC | 0.23920999 | −0.1158839 | −0.1121215 | 0.83383536 |
| CBFA2T2 | 0.33570197 | −0.8326576 | −0.3465489 | 0.04924959 |
| AAK1 | 0.1856245 | −0.2223762 | −0.831877 | 0.62966734 |
| CLSTN2 | −0.8316578 | −0.0616129 | −0.3307678 | 0.49855509 |
| GTPBP8 | 0.05263293 | 0.08803591 | 0.12022169 | −0.8311549 |
| 42435 | 0.28832563 | −0.477238 | −0.8308778 | 0.19230068 |
| HNRNPD | 0.18338378 | −0.6804024 | −0.8305738 | 0.16050471 |
| MBTPS2 | 0.0157453 | −0.6124772 | −0.8302701 | 0.38606525 |
| RPL39 | −0.0756471 | 0.15926055 | 0.01084592 | −0.829471 |
| SMG1 | 0.44622043 | −0.369879 | −0.8288789 | 0.47281465 |
| PCBP1 | 0.01433314 | −0.8284412 | −0.7390134 | −0.1771649 |
| PRRC2A | 0.31915189 | −0.2012566 | −0.5119764 | 0.82592776 |
| TNS1 | 0.31101782 | −0.4718121 | −0.8250795 | 0.52408358 |
| TIMM8B | −0.2764193 | 0.31007369 | 0.18058668 | −0.8246337 |
| PLAGL2 | 0.14293625 | −0.1158242 | −0.8221324 | 0.37347501 |
| TENM3 | 0.41047309 | −0.091705 | −0.4755345 | 0.82147404 |
| MLLT1 | 0.20208066 | −0.2058262 | −0.0349525 | 0.82104198 |
| LAT2 | 0.82097457 | −0.1832742 | 0.07354103 | −0.279438 |
| SETD1B | 0.40483959 | −0.2945494 | −0.8206969 | 0.48389261 |
| KIAA1958 | 0.59930088 | −0.1756622 | −0.8198741 | 0.07151379 |
| LSM1 | −0.3020394 | 0.34480664 | 0.33368229 | −0.8191136 |
| ZNF592 | 0.00247617 | −0.1202697 | −0.4118149 | 0.8180076 |
| ADAMTSL1 | 0.25156762 | −0.0303982 | −0.8178706 | −0.4151771 |
| ZNF92 | −0.1575241 | 0.12210341 | 0.01481409 | 0.81672964 |
| KIAA1522 | 0.16347146 | −0.4251744 | −0.445729 | 0.81644284 |
| SYNE1 | 0.57991828 | −0.181169 | −0.2037623 | 0.81586155 |
| HCFC1 | 0.35373061 | −0.2882014 | −0.5591182 | 0.8145383 |
| NME1 | −0.1639874 | 0.13818927 | 0.10511808 | −0.8143385 |
| HELZ2 | 0.63364926 | 0.81426182 | −0.1169531 | 0.78278495 |
| CISD3 | −0.0228505 | 0.07775769 | 0.20708068 | −0.813324 |
| ZNF124 | −0.031357 | 0.07475476 | 0.18144363 | −0.8111291 |
| FAM189B | 0.39203737 | −0.8097164 | −0.7890085 | 0.39535966 |
| TPT1 | −0.1378906 | −0.1575933 | 0.02870474 | −0.8093746 |
| RPS7 | −0.103865 | 0.14510085 | 0.16029106 | −0.8092512 |
| COX5B | −0.1190987 | 0.31259579 | 0.11797873 | −0.8091611 |
| RPS14 | −0.1048589 | 0.20532268 | 0.09554905 | −0.8077213 |
| TANC2 | 0.39889691 | −0.2159074 | −0.6530988 | 0.80712455 |
| TMEM14B | 0.0661944 | 0.09143667 | 0.05982025 | −0.8064157 |
| FIS1 | −0.1076721 | 0.17633828 | 0.00063911 | −0.8060077 |
| IGF1R | 0.41095629 | −0.2472216 | −0.4883125 | 0.80494545 |
| PSKH1 | 0.14922312 | −0.8032981 | −0.6000047 | 0.10837456 |
| PPARGC1B | 0.53861154 | −0.8030216 | −0.6181232 | 0.42281767 |
| ZMAT3 | 0.39405547 | −0.316103 | −0.8028064 | 0.26457182 |
| LRRC45 | 0.06369296 | −0.0251713 | 0.39008585 | 0.80272564 |
| AHNAK | 0.80252471 | −0.3304762 | −0.7140354 | 0.64920927 |
| DTX3L | 0.41134465 | 0.80231874 | −0.2993922 | 0.48085892 |
| TGFBI | −0.2570389 | 0.20078175 | 0.80007934 | 0.3704835 |
| ATHL1 | −0.4820053 | 0.05610773 | 0.37925368 | 0.79979154 |
| RPS16 | −0.0793584 | −0.1040892 | 0.27233511 | −0.799492 |
| RPL35 | −0.0450226 | 0.22842584 | 0.08062743 | −0.7976596 |
| SRP68 | 0.30247772 | −0.7973084 | −0.6918842 | 0.11623962 |
| CENPE | 0.23065233 | −0.0413186 | 0.07030087 | 0.79692695 |
| MRPL14 | −0.0778284 | 0.19039514 | 0.11679956 | −0.7968487 |
| MDC1 | 0.18231103 | −0.1840901 | −0.7967524 | 0.39226963 |
| TAGLN | −0.1880335 | 0.23825082 | −0.0002553 | 0.79664514 |
| RRAGC | 0.24873646 | −0.2194246 | −0.7960555 | 0.11665213 |
| PRLR | 0.26642442 | −0.1394492 | −0.2929194 | 0.79468347 |
| LSM10 | 0.04944863 | 0.04640834 | 0.13394797 | −0.7933342 |
| SCOC | −0.0486636 | 0.01196196 | 0.11514851 | −0.7927906 |
| CBLL1 | −0.071392 | 0.11923917 | −0.0644541 | 0.7925952 |
| NID1 | 0.18092863 | −0.1820773 | −0.402495 | 0.79196821 |
| CD9 | −0.2391149 | 0.09497988 | 0.11751343 | −0.789786 |
| COX7A2 | −0.1193055 | 0.35903906 | 0.14123531 | −0.7895833 |
| CD37 | 0.49670855 | −0.1425112 | −0.3714225 | 0.78933122 |
| COPRS | −0.1437362 | 0.28611786 | 0.07522103 | −0.7885504 |
| ASPM | 0.33369108 | −0.0784854 | −0.1079878 | 0.78840583 |
| WIZ | 0.15082338 | −0.0705928 | −0.2584074 | 0.78780715 |
| BST1 | −0.209103 | 0.47712543 | 0.73517824 | −0.7874461 |
| PALLD | 0.39694283 | −0.1592997 | −0.4549311 | 0.78671184 |
| YLPM1 | 0.34176731 | −0.4217563 | −0.7859372 | 0.36717171 |
| NOTCH1 | 0.45086414 | −0.1304755 | −0.2410426 | 0.78579311 |
| CIC | 0.25634133 | −0.1177541 | −0.1046079 | 0.78541621 |
| ZYX | 0.07922594 | 0.06511483 | −0.3625228 | 0.78421339 |
| TIMM10 | −0.1540232 | 0.28267365 | 0.226671 | −0.7840437 |
| NIPSNAP3A | 0.00469986 | 0.02211522 | 0.09744615 | −0.7839678 |
| FIGN | 0.22682535 | −0.0084107 | −0.7837551 | 0.27918817 |
| FAT4 | 0.78308508 | −0.1484281 | −0.6125628 | 0.69316549 |
| ROBO1 | 0.14394207 | 0.00630321 | −0.2918826 | 0.78262364 |
| NR2F2 | 0.34044348 | −0.2904713 | −0.6770023 | 0.78192188 |
| COMMD8 | −0.1638725 | 0.08006183 | 0.07059501 | −0.7818436 |
| SERPINE1 | −0.3523706 | 0.09793959 | 0.78112786 | 0.12019996 |
| TMEM256 | −0.0413693 | −0.0573964 | 0.21214912 | −0.7799032 |
| UBR4 | 0.58840114 | −0.3675312 | −0.7789172 | 0.72565492 |
| HECTD4 | 0.37131594 | −0.3080995 | −0.7786593 | 0.40814595 |
| RPS6 | −0.0963535 | 0.15548676 | 0.08034564 | −0.7784045 |
| CTDNEP1 | 0.29334372 | −0.1067906 | −0.7770331 | 0.2130949 |
| LOXL2 | −0.0336902 | −0.3188399 | −0.2365684 | 0.77685804 |
| USP42 | 0.26802881 | 0.13249488 | −0.2979633 | 0.77638395 |
| PTPRJ | 0.18580018 | −0.2979844 | −0.7740726 | 0.35677512 |
| CABIN1 | 0.30310761 | −0.0876353 | −0.2532643 | 0.77387115 |
| SOGA1 | 0.51105214 | −0.2060559 | −0.7736271 | 0.4511261 |
| DST | 0.7729289 | −0.2501636 | −0.5746267 | 0.70327288 |
| GNG11 | −0.0349802 | 0.01810362 | 0.45306584 | −0.7726608 |
| TRRAP | 0.43809068 | −0.4272642 | −0.772268 | 0.45267478 |
| UROS | −0.0284145 | 0.11843949 | 0.00560396 | −0.7716316 |
| ERH | −0.1117409 | 0.19354875 | 0.16762421 | −0.7711759 |
| RPL34 | −0.0692139 | 0.1183294 | 0.24209863 | −0.7711529 |
| RAB40B | 0.31661695 | −0.1574102 | 0.13823004 | −0.7705081 |
| DDIT3 | 0.03874243 | 0.40775201 | 0.56825392 | −0.7698485 |
| NFIA | 0.37702409 | −0.1676769 | −0.7688595 | 0.21744402 |
| MGRN1 | 0.14211351 | −0.2371295 | −0.7688408 | 0.35480674 |
| GPNMB | −0.0055129 | 0.05361348 | 0.76861783 | 0.28883015 |
| EPAS1 | 0.11013651 | −0.0734968 | −0.3277844 | 0.76809628 |
| TRIP4 | 0.1565607 | −0.7680757 | −0.2943887 | −0.1470014 |
| FAM96B | −0.0488291 | 0.19102142 | 0.11482978 | −0.7676815 |
| LSMD1 | −0.0470343 | 0.26338638 | 0.0664862 | −0.7661386 |
| SERPINB6 | −0.0286489 | 0.01085542 | −0.0527733 | −0.7657182 |
| PODXL | 0.04990762 | −0.7656351 | −0.4796134 | 0.38991377 |
| KIAA0895L | −0.3972427 | 0.0302244 | 0.76470905 | 0.22345461 |
| HAS2 | −0.5691384 | −0.6286967 | −0.7626376 | −0.1724125 |
| DGKH | 0.08549837 | −0.3301811 | −0.762435 | 0.46993266 |
| PTCH1 | −0.4050242 | −0.2322929 | −0.5125555 | 0.76188367 |
| FOXRED2 | 0.2911416 | 0.06242902 | −0.4776138 | 0.76133341 |
| CSF1 | 0.18249634 | −0.0037474 | 0.03016121 | 0.76098925 |
| FLYWCH2 | −0.085166 | 0.07676486 | 0.06937554 | −0.7605429 |
| SAP130 | 0.2998304 | 0.1168796 | −0.587572 | 0.75983942 |
| ALKBH7 | 0.04656605 | 0.14162516 | 0.39077591 | −0.7587919 |
| NDUFA1 | 0.05723049 | 0.23994137 | 0.17208478 | −0.7586538 |
| TARDBP | 0.18082544 | −0.758394 | −0.5073501 | 0.22415123 |
| MYL6 | −0.1088926 | 0.19294293 | 0.07178039 | −0.7582488 |
| PACS1 | 0.21995978 | −0.17497 | −0.7581394 | 0.57138478 |
| GYS1 | 0.0617858 | −0.0821535 | −0.2399361 | 0.75732173 |
| GSTO1 | −0.1683602 | 0.07042909 | 0.06744613 | −0.7560615 |
| TNRC6B | 0.47534861 | −0.2716117 | −0.5624099 | 0.75567458 |
| POLB | −0.0697952 | −0.1862425 | −0.0227756 | −0.7556041 |
| ARHGAP1 | 0.04601187 | −0.1672981 | −0.1700233 | 0.75515187 |
| C10orf76 | 0.1826102 | −0.7550102 | −0.1842698 | 0.09295404 |
| FUNDC2 | −0.0673324 | −0.1121169 | 0.00299169 | −0.7549547 |
| CHTF8 | 0.21582465 | −0.7544197 | −0.0754046 | −0.0249082 |
| SH3PXD2B | 0.16600832 | −0.2602101 | −0.2589705 | 0.75413768 |
| POLR2F | −0.0499993 | 0.25042289 | 0.11836952 | −0.7537528 |
| MPC1 | 0.05449976 | −0.0440483 | 0.2042987 | −0.75338 |
| SELT | −0.0544388 | −0.0187135 | 0.06242997 | −0.7533545 |
| PARP14 | 0.393569 | 0.75271806 | −0.0106497 | 0.50233629 |
| CHCHD2 | −0.1179104 | 0.15237015 | −0.0282455 | −0.7526611 |
| CBL | 0.38774294 | −0.1684004 | −0.577353 | 0.75180148 |
| CGNL1 | 0.53108476 | 0.75071271 | −0.1217007 | −0.3856639 |
| WNK1 | 0.34198436 | −0.3607998 | −0.7485527 | 0.72044065 |
| SNRPD2 | −0.0133937 | 0.23330077 | 0.13849688 | −0.7484995 |
| DLGAP4 | 0.13459903 | −0.3127174 | −0.7482435 | 0.52364621 |
| PDCD10 | −0.0898093 | 0.06343052 | 0.26653888 | −0.7480098 |
| MBNL3 | 0.16061597 | −0.3917519 | −0.500039 | 0.74671738 |
| POGZ | 0.00988818 | −0.0448218 | −0.5682575 | 0.7459217 |
| PFDN5 | −0.1625893 | 0.13888799 | 0.19166138 | −0.7453253 |
| NUP214 | 0.15601117 | −0.0046293 | −0.6036456 | 0.74416114 |
| TBCA | 0.055192 | 0.18619778 | 0.25897634 | −0.7440489 |
| EIF5B | 0.1730178 | 0.04756018 | −0.3010001 | 0.7439431 |
| RNF26 | 0.18626802 | −0.5766802 | −0.7436638 | 0.35169347 |
| DAG1 | 0.20057417 | −0.0143688 | −0.2456884 | 0.7431593 |
| C18orf54 | 0.38093522 | −0.5884877 | −0.7412145 | −0.3347837 |
| DOT1L | 0.16584165 | −0.1330813 | −0.2489036 | 0.74043237 |
| WIPF1 | −0.011798 | −0.2568072 | −0.7399232 | 0.56279199 |
| RPS29 | −0.1852548 | 0.1804712 | 0.15456511 | −0.7378582 |
| PGRMC2 | 0.06212032 | −0.426523 | −0.7372997 | 0.08295774 |
| TET2 | 0.15915101 | −0.2542669 | −0.2007536 | 0.73690033 |
| CCNYL1 | 0.25136473 | −0.7368267 | −0.7003753 | −0.0593294 |
| EIF1 | 0.00609732 | −0.7360113 | −0.4955177 | −0.0752403 |
| MYO18A | 0.73567853 | 0.12921304 | 0.03202898 | 0.42485317 |
| MAML2 | 0.226234 | −0.0442136 | −0.7353574 | 0.58903297 |
| PIP5K1C | 0.29006792 | −0.1600166 | −0.4797228 | 0.73498584 |
| AHDC1 | 0.5993769 | −0.7338343 | −0.5498448 | 0.61378138 |
| TFCP2 | 0.02149895 | −0.732314 | −0.1907679 | −0.0433242 |
| PDE6D | −0.2191787 | 0.18466146 | −0.0721636 | −0.7319449 |
| DNPH1 | −0.0009827 | 0.43033483 | 0.05018163 | −0.731735 |
| MRPL11 | −0.2065935 | 0.20420573 | 0.0968399 | −0.7316706 |
| PSME4 | 0.10201225 | −0.3653041 | −0.7315674 | 0.38802209 |
| RPL36 | −0.084052 | 0.34458076 | 0.08880894 | −0.7315631 |
| SERTAD2 | 0.24002861 | −0.1227343 | −0.730537 | 0.67647607 |
| RPL41 | −0.2716021 | 0.13563552 | 0.52903602 | −0.729796 |
| COL7A1 | 0.11995451 | −0.0878979 | 0.72949751 | 0.65470857 |
| AKNA | 0.28230134 | −0.1777801 | −0.7283938 | 0.65683736 |
| SCD | −0.2419056 | −0.1619073 | −0.1931494 | 0.72703107 |
| CKS2 | −0.1410467 | 0.14312957 | 0.28343282 | −0.7267607 |
| ATXN2L | 0.01859455 | −0.1503499 | −0.7265556 | 0.44844491 |
| HTT | 0.49508707 | −0.5529385 | −0.7259824 | 0.53105367 |
| ERC1 | 0.37104825 | −0.3724619 | −0.7258559 | 0.21760705 |
| NF1 | 0.42180479 | −0.2394217 | −0.725719 | 0.513972 |
| MED12 | 0.27650641 | −0.2342678 | −0.3113031 | 0.72569994 |
| TLE4 | 0.12144083 | 0.12978182 | −0.0361115 | 0.72560109 |
| LPP | 0.50431793 | −0.2446502 | −0.7254972 | 0.49327753 |
| SLC9A8 | 0.03875178 | −0.2144372 | 0.00618336 | 0.72538523 |
| EGFR | 0.38312923 | −0.2998069 | −0.3962258 | −0.7249009 |
| DPY30 | 0.0656528 | 0.21761208 | 0.16088905 | −0.7242833 |
| EPHX4 | −0.1245721 | 0.44176576 | 0.03589257 | −0.7240398 |
| PRPF8 | 0.37693059 | −0.4239123 | −0.7235068 | 0.4921559 |
| ZNF618 | 0.41176559 | −0.7229669 | −0.6866991 | 0.38673342 |
| BCL6 | 0.22823065 | 0.04170323 | −0.2332596 | 0.72287798 |
| CRLS1 | −0.1251186 | 0.1250749 | 0.1587039 | −0.7226358 |
| FAM193A | 0.04144894 | −0.2711487 | −0.2929548 | 0.72248874 |
| NDUFAB1 | −0.0775336 | 0.10604635 | 0.20104417 | −0.7220407 |
| CNOT4 | 0.05292941 | 0.12393884 | −0.4111783 | 0.72189865 |
| INPP5D | −0.7216967 | −0.6089731 | −0.0359974 | 0.50315767 |
| SAMD9 | 0.34438331 | 0.43587608 | 0.23156093 | 0.72114206 |
| FMNL1 | −0.0834219 | −0.7211033 | 0.15431922 | 0.60694859 |
| ID3 | −0.0144504 | 0.27775256 | 0.17374693 | −0.7202375 |
| SEMA6A | 0.01900555 | 0.20727855 | −0.7193101 | −0.2190822 |
| KIAA1671 | 0.36509288 | 0.0321638 | −0.5091207 | 0.718559 |
| BMPR2 | 0.25979634 | −0.3695584 | −0.7181442 | 0.45786169 |
| WWTR1 | 0.09179175 | −0.4702416 | −0.7171373 | 0.10892678 |
| SMARCD1 | −0.0197837 | −0.0282116 | −0.1519902 | 0.71673871 |
| HIST2H2AC | −0.2313772 | 0.13777708 | 0.13924985 | −0.7160745 |
| PITPNB | 0.11279214 | −0.7155989 | −0.5015428 | 0.01916005 |
| STRA13 | 0.04236656 | 0.3744062 | 0.1215577 | −0.7151861 |
| RAVER1 | 0.21478404 | 0.1352911 | −0.6702627 | 0.71509435 |
| MZT2B | −0.0149868 | 0.22641403 | 0.00839144 | −0.7146902 |
| NBEA | 0.71446513 | −0.1443396 | −0.6738816 | −0.6327659 |
| RPS21 | −0.0777348 | 0.12780467 | 0.25215885 | −0.714458 |
| GBF1 | 0.19767302 | −0.1077357 | −0.3115526 | 0.71368254 |
| MACF1 | 0.71273782 | −0.2650445 | −0.6033934 | 0.62222288 |
| TGFB1 | 0.04520907 | −0.4679573 | −0.4839856 | 0.71241656 |
| UBA7 | −0.6277551 | 0.10560167 | −0.7120835 | 0.5755638 |
| PURB | 0.3164115 | −0.4147733 | −0.7119658 | 0.29828456 |
| AXL | 0.17297792 | 0.00271035 | 0.54846146 | 0.71101284 |
| PER2 | 0.71004508 | 0.3531763 | −0.1795497 | 0.5916787 |
| KIFC2 | 0.08580007 | −0.0209064 | 0.70865803 | 0.43097591 |
| ARHGAP17 | 0.15876769 | −0.307002 | −0.7081354 | 0.30144804 |
| MRPL41 | −0.096754 | 0.35368377 | −0.0431797 | −0.7080853 |
| ATG2A | −0.0158203 | −0.0843652 | 0.01655914 | 0.70785673 |
| CLEC16A | 0.12376776 | 0.0158437 | −0.4114063 | 0.707486 |
| LSM6 | 0.00839272 | 0.24468743 | 0.11536032 | −0.7073889 |
| GUK1 | −0.045038 | 0.11951347 | 0.01707708 | −0.7071225 |
| HRSP12 | 0.34779115 | 0.23142426 | 0.38655933 | −0.7069435 |
| MKL1 | 0.12364429 | −0.0876691 | −0.1813776 | 0.70680803 |
| HIGD1A | −0.1541454 | 0.06703937 | 0.13931492 | −0.7068047 |
| BAZ2A | 0.27087643 | −0.2024223 | −0.4109497 | 0.70613196 |
| ZXDC | 0.07622292 | −0.2076556 | 0.03666342 | 0.70611099 |
| OSBP | 0.15427342 | −0.4345115 | −0.7060369 | 0.29556177 |
| ZNF689 | −0.1220564 | 0.25835345 | 0.15268599 | 0.70597013 |
| KLF6 | 0.27019634 | −0.7058116 | −0.6207123 | 0.05114733 |
| PHF10 | 0.05655259 | −0.3345838 | −0.7055559 | −0.0003914 |
| GLRX2 | −0.0154692 | 0.25891862 | 0.35878476 | −0.7053188 |
| CCDC174 | 0.11668438 | 0.14181537 | 0.02682206 | 0.70500187 |
| BLVRB | 0.1336541 | 0.31060346 | 0.10027175 | −0.7044515 |
| PTPN23 | 0.01972183 | −0.4015182 | −0.7041025 | 0.43721098 |
| EMG1 | −0.2200704 | 0.18357813 | 0.07894595 | −0.7038584 |
| MYL12B | −0.1642617 | 0.16047911 | 0.06633748 | −0.7033651 |
| ZNF524 | −0.0261445 | 0.07582754 | −0.0248164 | −0.702734 |
| ATP6V0B | −0.2075754 | 0.21741715 | 0.06651797 | −0.7020792 |
| MYEOV2 | −0.1171961 | 0.05216563 | 0.1165658 | −0.701866 |
| CDKN2AIPNL | −0.0156442 | 0.00657345 | 0.0848714 | −0.7016626 |
| ARL16 | −0.3524384 | 0.0614444 | 0.00331368 | −0.7012973 |
| MAFB | 0.52197647 | 0.70068724 | −0.4615867 | 0.31545919 |
| MTF1 | 0.28471497 | −0.5257905 | −0.5880104 | 0.70009838 |
| MYO1F | 0.54607575 | 0.01897796 | 0.69937628 | 0.30594668 |
| ZCCHC24 | 0.42829079 | −0.1630082 | −0.3868945 | 0.69927792 |
| RP11- | 0.34466969 | −0.6876957 | −0.4673936 | 0.69896676 |
| 1055B8.7 | | | | |
| C20orf24 | −0.0464426 | 0.36200074 | −0.070348 | −0.6983517 |
| HIST1H4B | −0.3025428 | 0.12150336 | 0.04573338 | −0.6965536 |
| RRBP1 | 0.14382158 | 0.06177326 | 0.01469708 | 0.69632725 |
| SF3B4 | −0.0122454 | −0.2830901 | −0.5210705 | 0.69507176 |
| RBM47 | 0.69470234 | 0.31734915 | −0.1631805 | 0.38064037 |
| NFYA | 0.08597344 | −0.6945983 | −0.358348 | 0.09846193 |
| PTPRS | 0.25738687 | −0.0894199 | −0.2596879 | 0.69454441 |
| EPS15L1 | 0.2011242 | −0.694443 | −0.43677 | 0.49688921 |
| PEX2 | −0.0654033 | −0.0165174 | 0.05594961 | −0.6943061 |
| IFITM3 | −0.1671234 | 0.48203094 | −0.0679501 | 0.69341979 |
| PHPT1 | −0.0849785 | 0.22920037 | 0.12021693 | −0.6931218 |
| ZFAT | 0.12955857 | −0.105526 | −0.1542274 | 0.6929942 |
| MLXIP | 0.29574583 | −0.4859208 | −0.6927137 | 0.45213406 |
| SNRPB | −0.0522575 | 0.16015699 | 0.11578603 | −0.692679 |
| SEC16A | 0.33121856 | −0.356288 | −0.5086548 | 0.69236329 |
| NEK9 | 0.2099789 | −0.691938 | −0.1993717 | −0.2291059 |
| CAMK1 | −0.2879642 | −0.1073967 | 0.1886332 | −0.6918794 |
| HUWE1 | 0.64680448 | −0.2516549 | −0.6610089 | 0.69130864 |
| UQCC2 | −0.0780903 | 0.28438104 | −0.0039675 | −0.6912245 |
| ZNF703 | 0.02118213 | −0.4464337 | −0.690966 | 0.59041746 |
| TMEM251 | 0.2529558 | −0.4407704 | −0.1814064 | −0.6902947 |
| BOD1L1 | 0.30661434 | −0.1677215 | −0.2478845 | 0.68990842 |
| RPL31 | −0.1042739 | 0.18195522 | 0.24169139 | −0.689805 |
| RPS8 | −0.0722204 | 0.23475319 | 0.09595389 | −0.6895767 |
| FAM214B | 0.22963929 | 0.1963179 | 0.6893649 | 0.46258683 |
| TMSB4X | −0.0491079 | 0.12849384 | 0.16832894 | −0.6885786 |
| RNF7 | −0.1143546 | 0.14505612 | 0.25273506 | −0.6883554 |
| MAP1LC3B | 0.01897982 | −0.6317002 | −0.6881106 | 0.03555737 |
| BICD1 | 0.20758907 | −0.6880817 | −0.6697518 | 0.27824299 |
| TLK1 | 0.03497865 | −0.1769765 | −0.6880664 | 0.3242051 |
| RPL24 | −0.0182891 | 0.21094074 | 0.07940853 | −0.6876611 |
| MGLL | −0.2369213 | −0.0206939 | −0.6875989 | 0.31725033 |
| PDRG1 | −0.1229914 | 0.06867962 | 0.32776139 | −0.6870351 |
| SLX4 | 0.40052456 | −0.1435945 | −0.3020903 | 0.68636662 |
| TMA7 | −0.061059 | 0.06993837 | 0.21389414 | −0.6857448 |
| TMEM126B | −0.1269055 | 0.09187004 | 0.40594915 | −0.6856293 |
| FOXO1 | −0.6846492 | 0.17118209 | −0.4213793 | 0.58409549 |
| MAP3K7CL | −0.438062 | −0.4262716 | −0.301055 | −0.6833629 |
| POLR2G | −0.1469537 | 0.24141416 | −0.0308052 | −0.683307 |
| CLASRP | −0.2218105 | −0.1034132 | 0.37471402 | 0.68321257 |
| LRRC8C | −0.6827572 | −0.1862476 | −0.4784822 | 0.32815071 |
| MRPL1 | −0.1527666 | −0.024246 | 0.23547482 | −0.6827347 |
| SYVN1 | −0.0001284 | 0.25249928 | −0.1135056 | 0.68217215 |
| SIRPA | 0.03009069 | −0.2327874 | −0.0732276 | 0.68155812 |
| CASP2 | 0.1249507 | 0.03734615 | −0.2469114 | 0.68132013 |
| CDK4 | −0.1193635 | 0.20776206 | 0.04619515 | −0.6807022 |
| RECK | −0.0057595 | −0.0407534 | −0.1237097 | 0.68049107 |
| NDUFS4 | −0.0355866 | 0.21776783 | 0.23579147 | −0.6801904 |
| ZNF460 | 0.44379629 | −0.2456255 | −0.5045584 | 0.67939411 |
| FXYD5 | −0.1721804 | 0.25340178 | 0.13590228 | 0.67927502 |
| ANKRD52 | 0.33265339 | −0.2005057 | −0.6788763 | 0.67651402 |
| RPL35A | −0.0805732 | 0.19241707 | 0.21813899 | −0.6788188 |
| BUD31 | −0.1413041 | 0.20222861 | 0.27506481 | −0.6785299 |
| SGK223 | 0.07891851 | −0.0638443 | −0.0779862 | 0.67818531 |
| UQCRQ | −0.1208104 | 0.29858669 | 0.13249748 | −0.6780903 |
| NR2C2 | 0.35099856 | −0.1815148 | −0.3598921 | 0.6779802 |
| WDTC1 | 0.0369491 | −0.0054894 | −0.0613792 | 0.67762392 |
| RPS19 | −0.0657441 | 0.264047 | 0.03617435 | −0.6769226 |
| DYNC1H1 | 0.67666299 | −0.3286523 | −0.5360687 | 0.66019466 |
| ISCA2 | −0.0930466 | 0.36064507 | 0.06845716 | −0.6766068 |
| EFCAB13 | −0.4879249 | −0.5514199 | −0.5085469 | −0.6763095 |
| LPAR6 | 0.00809963 | 0.04013862 | −0.6762345 | −0.3505345 |
| SNRPA | −0.1132638 | 0.2610254 | −0.0197595 | 0.67610147 |
| RPS9 | −0.0622337 | 0.19768013 | 0.02876004 | −0.6735017 |
| PATL1 | −0.1398499 | −0.0147962 | −0.273253 | 0.67326926 |
| ARID1B | 0.27640285 | −0.2431486 | −0.6725589 | 0.19750408 |
| PRDM2 | 0.12754439 | −0.4792706 | −0.6721999 | 0.2550488 |
| KMT2E | 0.14138375 | 0.03302055 | −0.6721465 | 0.38582263 |
| DPM3 | −0.3141782 | 0.22541281 | 0.14423821 | −0.6718572 |
| NBEAL2 | −0.0259705 | 0.0875084 | 0.01440937 | 0.67159457 |
| HK2 | 0.20256083 | −0.6713647 | −0.3900092 | 0.41017983 |
| ATP6V1G1 | −0.1014226 | 0.19680945 | 0.19800597 | −0.6712824 |
| ZMYND8 | 0.14616929 | 0.03795236 | −0.1947255 | 0.67105713 |
| ZNHIT1 | −0.1638636 | 0.24330263 | −0.0038293 | −0.6708862 |
| RNF111 | 0.25614978 | −0.1891329 | −0.6700811 | 0.39697402 |
| ELMSAN1 | 0.2480769 | −0.0454292 | −0.6698957 | 0.59761705 |
| NDUFA2 | −0.1894207 | 0.04456546 | 0.13522255 | −0.6689256 |
| AP2B1 | 0.22070255 | −0.5345326 | −0.6680332 | 0.21354084 |
| NPRL3 | 0.08138371 | −0.3975667 | −0.667216 | 0.44778492 |
| NDUFB7 | −0.0584689 | 0.50413794 | 0.09651173 | −0.6667978 |
| VDAC3 | −0.1016561 | 0.16172422 | 0.082809 | −0.6664728 |
| COX4I1 | −0.1179971 | 0.19833835 | 0.10473529 | −0.665061 |
| LENG8 | −0.2480121 | −0.290675 | 0.02397865 | 0.66447969 |
| POM121 | −0.0373585 | −0.3781447 | −0.6644068 | 0.61685883 |
| TPRG1L | 0.23797832 | −0.6643566 | −0.2855898 | 0.08067928 |
| ZFYVE1 | 0.25813763 | −0.2455746 | −0.0488257 | 0.66425522 |
| DDX58 | 0.22708479 | 0.66409033 | −0.2960837 | 0.53917234 |
| ANAPC11 | −0.0310072 | −0.0675648 | 0.16452069 | −0.6635919 |
| FAM195A | −0.1280468 | 0.5476451 | 0.02063211 | −0.6634027 |
| FBL | 0.20545222 | −0.6632771 | −0.0995856 | −0.2496504 |
| COL4A1 | 0.06097682 | 0.08222265 | 0.05065298 | 0.66257605 |
| SAP18 | −0.0592676 | 0.25766103 | 0.13588158 | −0.6621962 |
| COL8A1 | 0.34542176 | −0.0039299 | −0.661274 | −0.0296311 |
| GPX1 | −0.2114787 | 0.3234247 | −0.1068436 | −0.6612646 |
| C1QBP | −0.1691133 | 0.20664222 | 0.09452385 | −0.6612329 |
| TRIM25 | 0.13370604 | 0.01988975 | −0.0570708 | 0.6610546 |
| BOLA3 | 0.03820847 | 0.15090116 | 0.1162993 | −0.6604122 |
| AKAP13 | 0.46754593 | −0.1767451 | −0.3475739 | 0.66014789 |
| MTERF | −0.1274601 | −0.1146205 | 0.12889352 | −0.6594901 |
| TTC28 | 0.23124418 | −0.0905523 | −0.6594336 | 0.5301488 |
| VBP1 | −0.0939302 | 0.04120119 | 0.19609118 | −0.6591449 |
| PLEKHM1 | 0.10557984 | −0.346892 | −0.3085159 | 0.65913011 |
| MAP1A | 0.65870448 | −0.1268322 | −0.4585143 | −0.0564045 |
| RFX2 | 0.16660717 | 0.30261854 | −0.0918528 | 0.65826559 |
| SPTBN2 | 0.65773897 | 0.07256079 | 0.06462278 | −0.3736431 |
| SLC9A1 | 0.21555496 | −0.1181106 | −0.3775045 | 0.65755719 |
| RNASEH2C | −0.129623 | 0.20559294 | 0.08920342 | −0.6569488 |
| RNASEH2B | −0.1555055 | 0.0057283 | 0.11718796 | −0.656557 |
| ZNF507 | 0.23508962 | −0.430751 | −0.6555491 | 0.25529056 |
| ANKRD11 | 0.25388253 | −0.4235929 | −0.3709929 | 0.6543245 |
| ZC3H7A | 0.22862688 | −0.2936765 | −0.6540382 | 0.30215403 |
| CD63 | −0.0493749 | 0.12690151 | 0.12110333 | −0.6539269 |
| FASN | 0.32764364 | −0.5301338 | −0.3561973 | 0.65373234 |
| VRK1 | −0.100069 | −0.1563579 | 0.17672029 | −0.651943 |
| SIPA1L3 | 0.25763115 | −0.3247721 | −0.3480234 | 0.65173291 |
| TRAPPC2L | −0.1142541 | 0.13288888 | 0.2544896 | −0.6513163 |
| LSM2 | −0.1948055 | 0.00335126 | 0.18112901 | −0.6512747 |
| RPA3-AS1 | 0.12706361 | 0.02499609 | 0.12440744 | −0.6504324 |
| SGSH | 0.18211862 | −0.6499683 | −0.3447606 | 0.59514005 |
| CNTNAP1 | 0.62568463 | −0.3610579 | 0.18420182 | −0.6498898 |
| ASXL2 | 0.45107431 | −0.3562411 | −0.6497617 | 0.61528649 |
| ZC3HAV1 | 0.36946724 | −0.0151081 | −0.3182312 | 0.64967706 |
| HNRNPUL2 | 0.330445 | −0.649644 | −0.2923807 | 0.57883747 |
| UQCRH | 0.02889511 | 0.1274419 | 0.09708469 | −0.6496262 |
| COA5 | −0.1481261 | 0.31191961 | 0.18182268 | −0.6493659 |
| FOXP4 | 0.27457607 | 0.08373616 | −0.6492951 | 0.59499349 |
| CLSTN1 | 0.09314359 | −0.2178548 | −0.416466 | 0.6492879 |
| HIST1H2AC | −0.221942 | 0.10300129 | −0.0643994 | −0.6488332 |
| RABEPK | −0.2423888 | 0.37029466 | 0.20758468 | −0.6487692 |
| GPATCH8 | 0.36065644 | −0.1654673 | −0.4190748 | 0.64843932 |
| ATP11A | 0.20232777 | −0.3616228 | −0.3798197 | 0.64659016 |
| DROSHA | 0.146996 | −0.2206603 | −0.6461854 | 0.42486847 |
| SAFB2 | 0.12624596 | 0.00681115 | 0.03749066 | 0.645737 |
| SOS1 | 0.16531618 | −0.2765128 | −0.4532642 | 0.64557477 |
| SMC1A | 0.23168708 | 0.000014 | −0.0382686 | 0.64520153 |
| SLC37A2 | −0.4601264 | −0.3077019 | 0.64472407 | 0.46548704 |
| UNC50 | −0.0430429 | 0.2375202 | 0.24490606 | −0.6445635 |
| ACTR10 | 0.01336683 | 0.04280885 | 0.11937769 | −0.6444256 |
| IDS | 0.17345858 | −0.1524527 | −0.6055529 | 0.64438574 |
| IDS | 0.17345858 | −0.1524527 | −0.6055529 | 0.64438574 |
| TMSB10 | 0.00428988 | 0.25987954 | 0.03947516 | −0.6441701 |
| BAG3 | 0.01798977 | 0.02901065 | −0.1994011 | 0.6433041 |
| SERF2 | −0.088517 | 0.14343808 | 0.02776844 | −0.6428479 |
| POLR2L | 0.05690065 | 0.33861544 | 0.00508111 | −0.6426752 |
| LGALS3 | −0.2498782 | 0.00758887 | 0.05489277 | −0.6424445 |
| ENG | 0.23001436 | −0.5838006 | 0.07091641 | 0.64236037 |
| ZFAND1 | −0.1112874 | −0.0511364 | 0.25878969 | −0.6423553 |
| TIAL1 | −0.1217041 | −0.6423042 | −0.5904268 | 0.00568302 |
| RPL38 | −0.0669938 | 0.2804294 | 0.17695113 | −0.6421883 |
| HSPG2 | 0.53877794 | −0.3956217 | −0.4659358 | 0.64198319 |
| SF3A1 | 0.08170197 | −0.2643141 | −0.4414966 | 0.64192559 |
| DMXL2 | 0.37273976 | −0.2201187 | −0.1950206 | 0.64184882 |
| ZCCHC14 | 0.48564047 | −0.2233681 | −0.6417158 | 0.58466688 |
| COMMD10 | −0.0465881 | −0.0667263 | 0.19938607 | −0.641105 |
| THBS1 | −0.0500019 | −0.2527757 | 0.64074912 | 0.21497247 |
| SOX11 | 0.30768901 | −0.3239856 | −0.2209573 | −0.6407452 |
| PHF12 | 0.26237749 | −0.0678985 | −0.2444427 | 0.6403214 |
| HERC1 | 0.63991795 | −0.1599073 | −0.1973418 | 0.48571048 |
| THOC2 | 0.11956975 | 0.0040928 | −0.0469516 | 0.63976694 |
| UBN2 | 0.28652923 | −0.3650121 | −0.6394169 | 0.36378419 |
| MSI2 | 0.6392879 | −0.1500156 | −0.0290522 | 0.30120435 |
| LARP1 | 0.21666992 | −0.4125232 | −0.4583164 | 0.63921391 |
| RWDD1 | −0.1211373 | 0.10119799 | 0.11530031 | −0.639069 |
| RBM25 | 0.07266134 | −0.0357925 | 0.13858936 | 0.63895869 |
| BPTF | 0.30462507 | −0.2992659 | −0.6385147 | 0.43841705 |
| TOB2 | 0.13533031 | 0.04276188 | −0.286996 | 0.63808771 |
| RPS5 | −0.0962698 | 0.2682929 | −0.009331 | −0.6378146 |
| PHF15 | 0.63725539 | −0.2936113 | 0.08938201 | 0.30821255 |
| PPIL2 | 0.08179893 | 0.02592571 | −0.0591188 | 0.63706339 |
| EXOSC2 | −0.0891163 | 0.04418046 | 0.03380647 | −0.6368929 |
| MRPL22 | −0.111474 | 0.04377597 | 0.09855778 | −0.6361087 |
| KIF13B | 0.63609306 | −0.4912713 | 0.04309151 | 0.30249692 |
| BACE1 | 0.08248482 | −0.0932679 | −0.6360369 | 0.36763469 |
| RREB1 | 0.36929873 | −0.2721441 | −0.6356217 | 0.59453005 |
| USP13 | 0.10721109 | −0.1650747 | −0.6354058 | 0.18024643 |
| ASH2L | 0.0284543 | −0.6352788 | −0.1526158 | −0.0244145 |
| SZT2 | 0.20336875 | −0.1464573 | 0.08648636 | 0.63501242 |
| PER1 | 0.17396108 | 0.16514707 | −0.3825164 | 0.63496443 |
| ODC1 | −0.0802411 | 0.07120686 | 0.11517868 | −0.6343775 |
| FAM96A | −0.0696096 | 0.059885 | 0.22833084 | −0.6341768 |
| FDX1 | −0.1965326 | −0.0798717 | 0.01876547 | −0.6334012 |
| SAMD4B | 0.12636222 | −0.029846 | −0.3521489 | 0.63249431 |
| S100A4 | −0.3110807 | 0.20360646 | −0.0618069 | −0.6316783 |
| RUNX1 | 0.2277922 | −0.2247395 | −0.6309345 | 0.49990091 |
| CHD6 | 0.3341391 | 0.0443692 | −0.144227 | 0.62964069 |
| GIGYF1 | −0.0621294 | −0.2072601 | 0.28963001 | 0.62956052 |
| SLC29A1 | −0.1630535 | −0.0247497 | −0.088028 | 0.62940686 |
| FBN1 | 0.45360776 | −0.0980688 | −0.0543201 | 0.62938044 |
| GRINA | −0.1498919 | 0.16185766 | −0.2666019 | 0.62936135 |
| AP1S1 | −0.0780121 | 0.24562243 | 0.11483684 | −0.6290629 |
| NDUFB5 | −0.0104284 | 0.09214701 | 0.30355745 | −0.6288479 |
| COL6A3 | 0.10017429 | −0.208172 | −0.6286416 | 0.56806965 |
| KIAA1549 | 0.49430312 | −0.2430173 | −0.6280905 | 0.0273081 |
| TET1 | 0.14602761 | −0.1757865 | −0.6279997 | −0.2903597 |
| HELZ | 0.55744685 | −0.1048788 | −0.6278457 | 0.49445915 |
| ADAR | 0.22871069 | 0.11872656 | −0.2449334 | 0.62776731 |
| HNRNPA3 | 0.05047238 | −0.2185763 | −0.6277281 | 0.33617163 |
| LYRM4 | −0.1453657 | 0.37440031 | 0.00068331 | −0.6272341 |
| KLHL17 | −0.1640517 | −0.3485359 | 0.62678406 | 0.34482479 |
| ZC3H13 | 0.22032524 | 0.06571592 | −0.2938271 | 0.62665868 |
| ATXN2 | −0.030169 | −0.3537733 | −0.6266146 | 0.38922886 |
| PRDM1 | 0.27835792 | −0.0365875 | −0.2959493 | 0.6264806 |
| IPO8 | 0.27967744 | −0.5675011 | −0.6263311 | 0.04071775 |
| THOC7 | −0.1401775 | 0.04886786 | −0.0402314 | −0.6262613 |
| ABCA2 | 0.1830956 | −0.3900777 | −0.0468332 | 0.62577825 |
| TSC22D2 | 0.34222883 | −0.1724656 | −0.5858485 | 0.62544389 |
| NSMCE2 | 0.05260604 | −0.0605055 | 0.12558923 | −0.6252565 |
| ANLN | 0.16392602 | 0.00018935 | 0.04656657 | 0.62480768 |
| UFM1 | −0.1435817 | −0.096424 | 0.21603745 | −0.6241482 |
| BCL9 | 0.56159461 | −0.0470708 | −0.6241088 | 0.37093781 |
| TMEM131 | 0.08083164 | −0.3000232 | −0.6240924 | 0.52870683 |
| SSH2 | 0.24652964 | −0.3771303 | −0.6238067 | 0.55714596 |
| RAB5B | 0.09809168 | 0.10805603 | −0.429187 | 0.62375699 |
| TNRC18 | 0.48784284 | −0.5145162 | −0.360038 | 0.62357041 |
| ATRAID | −0.0562984 | 0.04785495 | 0.00724475 | −0.6230705 |
| MANBAL | 0.10492491 | −0.6230144 | −0.0353837 | −0.5916132 |
| HIF1AN | 0.26065744 | −0.5477942 | −0.6228683 | 0.1757314 |
| MIER2 | −0.032639 | 0.10288943 | −0.0257409 | 0.62276739 |
| KIAA2018 | 0.44732845 | −0.2438708 | −0.6225954 | 0.58429501 |
| KIAA1644 | 0.6224263 | −0.5491356 | −0.0680371 | 0.30529746 |
| MDN1 | 0.56147949 | −0.3797687 | −0.4797704 | 0.62183271 |
| ACBD6 | −0.2437065 | 0.24631733 | 0.11890892 | −0.6213328 |
| TAF11 | −0.1159853 | 0.05337567 | 0.02537324 | −0.6213313 |
| BCL7C | 0.0559302 | 0.22597951 | −0.0512092 | −0.6197418 |
| PRDX4 | −0.1026349 | 0.03493906 | 0.04270317 | −0.619098 |
| SH3PXD2A | −0.541106 | −0.3694972 | −0.373055 | 0.61827145 |
| NDUFA4 | −0.0603049 | 0.1236355 | 0.19939653 | −0.6182119 |
| COX7A2L | −0.1056601 | 0.19660076 | 0.12680079 | −0.617988 |
| COL1A1 | 0.18281814 | −0.617397 | −0.4845399 | 0.28285743 |
| PLGRKT | −0.311108 | 0.11616741 | 0.01102185 | −0.6173045 |
| PHLPP1 | 0.30927483 | −0.2443999 | −0.1989871 | 0.61716064 |
| RPL7 | −0.2782613 | 0.61682342 | 0.32098952 | −0.2178949 |
| NDUFB11 | −0.067291 | 0.10653609 | 0.00501105 | −0.6162535 |
| TOMM22 | −0.0401889 | 0.21298331 | 0.08402675 | −0.616179 |
| KIAA0195 | 0.07427801 | 0.06880772 | −0.0686125 | 0.61599126 |
| FBN2 | 0.55820473 | −0.1420739 | −0.3621148 | 0.61595954 |
| VAPB | 0.28774822 | −0.3554221 | −0.6152536 | −0.1481935 |
| CSNK1D | 0.41494697 | −0.6152464 | −0.0509978 | 0.44910483 |
| TAF15 | 0.33759619 | −0.3356234 | −0.615073 | 0.37148656 |
| RPS3 | −0.0802883 | 0.10389728 | 0.23470506 | −0.6148063 |
| WDFY3 | 0.56172422 | −0.1519171 | −0.421653 | 0.61439379 |
| CHMP5 | −0.1253241 | 0.14793429 | 0.18705985 | −0.6142318 |
| TIMM17A | −0.1276591 | 0.18164992 | 0.1907365 | −0.614216 |
| KREMEN1 | 0.1334742 | −0.4707499 | −0.3061423 | 0.61393689 |
| CAPZB | 0.14808062 | −0.6138492 | −0.2438958 | 0.10140313 |
| MRPL52 | −0.1489676 | 0.1944622 | 0.16738686 | −0.6124055 |
| SEZ6L2 | 0.04907384 | −0.0422598 | −0.3426457 | 0.61207377 |
| NPEPPS | 0.11861628 | −0.3073185 | −0.6118175 | 0.25492211 |
| ZBTB4 | 0.23113678 | −0.0570435 | 0.19002955 | 0.61172573 |
| EMC4 | −0.1228382 | 0.22689068 | 0.13260175 | −0.611678 |
| SUPT6H | 0.23794797 | −0.1421081 | −0.2582735 | 0.61118681 |
| ZC3H4 | 0.16508414 | −0.213659 | −0.0953573 | 0.61101321 |
| ATP5L | −0.0788192 | 0.21774803 | 0.13371676 | −0.6105035 |
| C14orf119 | −0.2407412 | 0.15430162 | 0.2172443 | −0.6098228 |
| PHIP | 0.39174453 | −0.1444776 | −0.3386133 | 0.6098121 |
| CNOT1 | 0.2701859 | −0.3676084 | −0.6097591 | 0.4193538 |
| PAK2 | 0.10707895 | −0.1414927 | −0.6093931 | 0.47588181 |
| CORO2B | −0.2820641 | −0.5596266 | 0.18289952 | 0.60923332 |
| DHRS3 | −0.063908 | 0.16501223 | −0.6090997 | −0.335804 |
| CAD | 0.25501667 | −0.2856717 | −0.2589896 | 0.60869814 |
| MED14 | 0.11252078 | 0.08768649 | −0.4584954 | 0.60834197 |
| ITPR1 | 0.23215426 | −0.2131143 | −0.1774731 | 0.60807991 |
| EAPP | 0.07055882 | 0.03035581 | 0.03935178 | −0.6080488 |
| PLEC | 0.60803851 | −0.4390697 | −0.0739505 | 0.51655082 |
| IRS2 | 0.40684587 | −0.0133624 | −0.3694001 | 0.60775216 |
| EME2 | −0.0603293 | −0.0819053 | 0.60761526 | 0.57194236 |
| RPL13 | −0.1221995 | 0.22944023 | 0.10649636 | −0.6070348 |
| CISD2 | 0.0203557 | 0.0234977 | 0.01659216 | −0.6069196 |
| CIRH1A | 0.00677022 | −0.6067432 | −0.3364061 | −0.0358311 |
| ASNA1 | −0.0318356 | 0.15325642 | 0.13451551 | −0.6066209 |
| COMMD2 | −0.0810984 | −0.0606433 | 0.223466 | −0.6065546 |
| ECM1 | −0.1654676 | −0.1230485 | 0.54896424 | 0.60632336 |
| HNRNPH3 | 0.01309698 | −0.1492037 | −0.0225629 | 0.60622609 |
| PNISR | −0.2427846 | 0.02565595 | 0.389415 | 0.60572638 |
| ERV3-1 | −0.0732115 | 0.12219481 | 0.24200652 | 0.60567946 |
| MYL6B | 0.09074426 | 0.09537939 | 0.02748779 | −0.6055049 |
| RNF24 | 0.28610542 | −0.4172479 | −0.4189513 | 0.60501631 |
| ARF5 | −0.1432807 | 0.05165503 | 0.12897223 | −0.6045449 |
| TMEM255A | −0.1903353 | 0.29143604 | −0.0083233 | 0.60427502 |
| EML4 | 0.18751478 | −0.1238423 | −0.1185083 | 0.60411615 |
| BDP1 | 0.19032831 | −0.1879546 | −0.0369604 | 0.60390581 |
| NUDT22 | −0.1024498 | 0.20503822 | −0.0921023 | −0.6038555 |
| AHSA2 | −0.6037547 | 0.04159724 | 0.48348561 | 0.28401614 |
| ZNF264 | 0.32088889 | −0.0101949 | −0.0735916 | 0.6035366 |
| CPEB2 | 0.08370522 | −0.2392846 | −0.6034892 | 0.54243457 |
| TTC23 | 0.04798991 | 0.17085039 | 0.22409696 | 0.60342208 |
| TACO1 | −0.0895552 | 0.37167266 | 0.15134275 | −0.6033289 |
| MAP1S | −0.0259994 | −0.2147379 | −0.2504758 | 0.60328444 |
| DNMT3A | 0.20588714 | −0.1486271 | −0.0322866 | 0.60328435 |
| FOXK1 | 0.33591078 | −0.2783668 | −0.2289421 | 0.6032517 |
| TNIK | 0.03369989 | −0.2122443 | −0.1543764 | 0.6032397 |
| SIK3 | −0.1062978 | −0.0653288 | −0.286771 | 0.6028454 |
| NT5C | −0.2495352 | 0.10965321 | 0.27200441 | −0.6025438 |
| CHIC2 | −0.0915761 | 0.08842754 | 0.02863301 | −0.6025347 |
| TCEB2 | −0.0783649 | 0.27046909 | 0.0879852 | −0.602011 |
| SERPINI1 | −0.2388108 | 0.60182979 | −0.1640421 | −0.487902 |
| SRGAP2 | 0.30025924 | −0.2221885 | −0.4238051 | 0.60140293 |
| C19orf60 | 0.01221806 | 0.30748602 | −0.1255583 | −0.6012153 |
| CEP170B | 0.23372876 | −0.0547418 | 0.05162296 | 0.60100307 |
| MAP4 | 0.17126421 | −0.1500567 | −0.4624705 | 0.60095398 |
| MAD2L2 | −0.0952123 | 0.18975257 | −0.1213884 | −0.6006432 |
| RAB14 | 0.17800202 | −0.4698866 | −0.6006394 | 0.21569399 |
| USP4 | 0.17387147 | −0.6005815 | −0.3062356 | 0.08135675 |
| ZFAND6 | −0.0120998 | 0.0618661 | 0.07609014 | −0.6000193 |
| PTGS2 | −0.5999766 | −0.0931668 | 0.22761614 | 0.42806155 |
| DVL3 | 0.08436021 | −0.0199839 | −0.154905 | 0.59987875 |
| E2F7 | 0.00964041 | −0.3086901 | −0.4185547 | 0.59986224 |
| WWP1 | 0.08456919 | −0.3622396 | −0.5998278 | 0.06716568 |
| DIP2B | 0.35171953 | −0.1880126 | −0.2502995 | 0.5996772 |
| LCOR | 0.37842293 | −0.2895198 | −0.5993783 | 0.39642696 |
| SEPW1 | −0.1901097 | 0.17462622 | 0.07091534 | −0.5991882 |
| KIAA0430 | 0.27070115 | −0.2732841 | −0.4988436 | 0.59775008 |
| TAPBP | 0.0185036 | −0.0257369 | −0.2317106 | 0.5974676 |
| UBE2E1 | −0.0565594 | 0.00661879 | 0.10511862 | −0.597427 |
| FAM120A | 0.1037268 | −0.1256171 | −0.3178688 | 0.59705973 |
| TMEM70 | −0.0504234 | 0.07724659 | 0.06232425 | −0.59679 |
| PSMG1 | −0.0780157 | 0.0824987 | 0.09446719 | −0.5960468 |
| PHF2 | 0.0881643 | 0.0027877 | −0.0814045 | 0.59601232 |
| PSMA5 | −0.1296535 | 0.20694361 | 0.1437429 | −0.595803 |
| XAB2 | 0.1574293 | −0.5956765 | −0.332247 | 0.48675126 |
| GLRX5 | −0.2668158 | 0.11946854 | 0.08425422 | −0.5956251 |
| PPP3CB | 0.06921434 | −0.1173311 | −0.595449 | 0.04938018 |
| RBX1 | −0.0213316 | 0.16994897 | 0.07484423 | −0.5948079 |
| AKT1S1 | 0.15038872 | 0.02430038 | 0.06395074 | 0.59440977 |
| MYO9B | 0.25433861 | −0.149191 | −0.1010071 | 0.59415302 |
| PPRC1 | 0.03218728 | −0.091823 | −0.2092112 | 0.59406004 |
| ATIC | −0.1389259 | −0.5939784 | −0.3106664 | 0.49888503 |
| GLCCI1 | 0.44525596 | −0.062164 | −0.5938561 | 0.3116879 |
| KIAA0556 | 0.06188358 | −0.1566237 | −0.1413294 | 0.5937682 |
| SMARCA4 | 0.20541582 | −0.1028729 | −0.2404313 | 0.59320434 |
| CCNI | 0.04095984 | −0.2018985 | −0.5931168 | 0.34594281 |
| RAPGEF2 | 0.23467528 | −0.2363952 | −0.063387 | 0.59307649 |
| CRTC2 | 0.00801942 | −0.1122516 | −0.1973212 | 0.59297285 |
| MIDN | 0.42175967 | −0.5926738 | −0.3772564 | 0.42222665 |
| PRRC2B | 0.36038622 | −0.3142708 | −0.2724403 | 0.59253672 |
| REV3L | 0.24725612 | −0.0979766 | −0.3359959 | 0.59181582 |
| KANSL2 | −0.2920008 | 0.25039404 | 0.03807063 | −0.5914465 |
| TMEM104 | 0.18818904 | 0.12319526 | −0.0770083 | 0.59114827 |
| PXN | 0.08665454 | −0.0901224 | −0.0652852 | 0.59094062 |
| MRPS15 | −0.1130872 | 0.1760317 | 0.0356072 | −0.5909397 |
| NUS1 | 0.08006048 | −0.5907841 | −0.5344542 | −0.0102702 |
| ALG5 | 0.00016011 | 0.08647722 | 0.05725181 | −0.5907632 |
| MEF2D | 0.11596095 | −0.0058267 | −0.4906656 | 0.59071354 |
| NOTCH2 | 0.40154242 | −0.1038908 | −0.3358454 | 0.59040599 |
| MAST4 | 0.00135045 | −0.4313955 | −0.2903037 | 0.59035163 |
| GNA12 | 0.06164555 | −0.5901113 | −0.2360209 | 0.2583498 |
| C11orf31 | −0.0204896 | 0.42842653 | 0.08848079 | −0.590111 |
| ITGA7 | 0.52210286 | −0.4203143 | 0.23098028 | 0.58991014 |
| MRPL15 | −0.2647672 | 0.12103246 | 0.08049235 | −0.5897928 |
| C6orf48 | −0.1475908 | 0.20841532 | 0.27033495 | −0.5897076 |
| IL1B | −0.5886053 | 0.11251734 | −0.0360038 | 0.2320674 |
| UBE2K | 0.06171712 | −0.5884734 | −0.5252505 | 0.21226608 |
| NDUFA11 | 0.01812048 | 0.12635441 | −0.0273753 | −0.5884594 |
| ZSWIM8 | 0.10425038 | −0.0013799 | 0.04770621 | 0.58818643 |
| MYL12A | −0.1560408 | 0.15997282 | 0.1133287 | −0.5881379 |
| ARHGAP35 | 0.28229929 | −0.2836288 | −0.2187161 | 0.5880784 |
| TSC2 | 0.19872905 | 0.04537674 | 0.0250935 | 0.58768937 |
| TMEM14A | 0.31666037 | 0.08435895 | 0.18697062 | −0.5873504 |
| RPL28 | 0.20016387 | −0.5581746 | −0.1748918 | 0.58726685 |
| SEC11C | −0.0940956 | 0.21802591 | 0.03942679 | −0.587025 |
| DAGLA | 0.44643849 | 0.13760796 | −0.2180027 | 0.58681975 |
| EIF1AX | −0.0832341 | −0.1496927 | −0.0268283 | −0.5867702 |
| TGM2 | −0.5866502 | −0.4783712 | −0.2921457 | 0.58223914 |
| CLCN5 | 0.05479201 | −0.167218 | −0.293093 | 0.58637326 |
| TRNAU1AP | −0.0505374 | −0.0212581 | 0.30168615 | −0.5861854 |
| SPTAN1 | 0.51018049 | −0.1569346 | −0.4037615 | 0.58608773 |
| ABL1 | 0.24238688 | −0.1384672 | −0.1989861 | 0.58570561 |
| RBAK | 0.11892143 | 0.03128251 | −0.0493609 | 0.58569666 |
| ENAH | 0.18670088 | 0.02747295 | −0.0428755 | 0.58567301 |
| PHLDB1 | 0.3306915 | −0.1218639 | 0.09210687 | 0.58562536 |
| WDR81 | 0.18992452 | −0.1538014 | −0.0062218 | 0.58527922 |
| MIEN1 | −0.0884499 | 0.02081678 | 0.04860133 | −0.5851208 |
| BAG1 | −0.1090229 | −0.0848868 | 0.03340944 | −0.5851056 |
| SCO1 | −0.0593555 | 0.02941735 | 0.16855935 | −0.5839784 |
| NXF1 | −0.1328586 | −0.05777 | 0.25316062 | 0.58352688 |
| C7orf50 | 0.04299535 | 0.23898008 | 0.02444089 | −0.5833087 |
| RPL32 | −0.0934306 | 0.18937789 | 0.06983722 | −0.5826075 |
| BCOR | 0.47848868 | 0.10037275 | −0.4321334 | 0.58255952 |
| ZNF91 | 0.35273014 | 0.08161759 | −0.2919523 | 0.5823168 |
| KIAA1244 | 0.40223825 | −0.0821994 | −0.5820414 | 0.24385582 |
| ZFC3H1 | 0.07459252 | −0.2966751 | −0.5819577 | 0.50165302 |
| ARHGAP32 | 0.42885941 | −0.2481662 | −0.534148 | 0.58194702 |
| MADD | 0.20516328 | −0.1842336 | −0.1384043 | 0.58184949 |
| NUMA1 | 0.35525434 | −0.0665473 | 0.10531847 | 0.58179356 |
| SAFB | 0.18921843 | −0.581786 | −0.1703209 | 0.48735681 |
| FRY | 0.26128189 | −0.2114183 | 0.23259285 | 0.58172025 |
| ZBTB37 | 0.43702691 | −0.1139302 | −0.2354581 | 0.58165794 |
| ABCC1 | 0.18408949 | −0.4076036 | −0.3436022 | 0.58159665 |
| KIAA1462 | −0.0369722 | −0.2957843 | −0.3958515 | −0.581181 |
| KLC1 | 0.12045277 | 0.19334898 | 0.07592528 | 0.580911 |
| TNFAIP2 | 0.58082109 | −0.1179663 | −0.1069083 | 0.14039616 |
| C11orf73 | −0.1772893 | 0.18157432 | −0.0987016 | −0.5808104 |
| MRPS7 | −0.1540971 | 0.14995503 | 0.08995564 | −0.5803809 |
| GOLGA4 | 0.11484779 | −0.0270357 | 0.0465447 | 0.58004838 |
| VPS26A | 0.21370065 | −0.5207642 | −0.5799409 | −0.0120287 |
| HAX1 | −0.3162913 | 0.36316754 | 0.09355769 | −0.5798084 |
| AMOT | 0.57969835 | −0.0704498 | −0.1051322 | 0.19246419 |
| FN1 | 0.02926367 | −0.0252838 | −0.3393865 | 0.57945992 |
| STOML2 | −0.2016475 | 0.12723347 | 0.01645552 | −0.5790051 |
| PIN1 | −0.0579407 | 0.24127051 | 0.04936464 | −0.5780146 |
| MTERFD1 | −0.2388666 | 0.08309974 | 0.11054767 | −0.5779766 |
| RPS12 | −0.1367366 | 0.21244554 | 0.09407296 | −0.5778193 |
| TXNDC17 | 0.01672647 | 0.14875685 | 0.17880615 | −0.5774703 |
| CYR61 | 0.01951561 | −0.577428 | −0.1953133 | −0.4436776 |
| UBAP2L | 0.09107439 | −0.1220571 | −0.0303203 | 0.57729377 |
| EIF4G3 | 0.07291953 | 0.01702326 | −0.3976598 | 0.57666025 |
| MTOR | 0.2685828 | −0.0683595 | −0.1885263 | 0.5765338 |
| SEPN1 | 0.13660324 | −0.3024581 | −0.5765135 | 0.465927 |
| MYLK | 0.27921701 | −0.079535 | −0.2195108 | 0.57649644 |
| TMEM101 | 0.10155348 | 0.21010762 | 0.02120692 | −0.5758062 |
| BCL2L11 | 0.21834069 | 0.27879988 | −0.575641 | 0.2806781 |
| LUC7L3 | −0.2715495 | −0.0124251 | 0.57551263 | 0.46473987 |
| ADH5 | −0.0884563 | 0.088362 | 0.21255541 | −0.575314 |
| GCN1L1 | 0.29922506 | −0.1576355 | −0.1022072 | 0.57487343 |
| FAU | −0.0500859 | 0.31894667 | 0.07807538 | −0.5745164 |
| TLN1 | 0.29698121 | −0.1488732 | −0.1582954 | 0.5741382 |
| PRKACA | 0.13962128 | 0.09447358 | −0.4928556 | 0.57372766 |
| TIMP2 | 0.14055961 | −0.2407639 | −0.4450665 | 0.57318889 |
| NSMCE1 | −0.1532289 | 0.2044891 | −0.0851212 | −0.5730993 |
| GALNS | −0.0108121 | −0.0157572 | 0.21085516 | 0.57305797 |
| SERTAD1 | −0.3327589 | 0.28644617 | 0.02202144 | −0.5729135 |
| NOTCH2NL | 0.57286845 | −0.5299901 | −0.1938303 | 0.4941777 |
| PSMC1 | −0.0735354 | −0.0133338 | 0.07395138 | −0.5726789 |
| EXOSC4 | −0.1250863 | 0.26597249 | 0.18334276 | −0.5721212 |
| ZSCAN30 | 0.19336421 | −0.002084 | 0.07963653 | 0.57199151 |
| ADORA2B | −0.128825 | 0.36304567 | 0.02468456 | −0.5716074 |
| SETD5 | 0.16133136 | −0.2320933 | −0.4820513 | 0.57159898 |
| USP34 | 0.3031619 | −0.1979102 | −0.571417 | 0.39670192 |
| MOV10 | 0.28747989 | 0.08448284 | −0.2278989 | 0.5708904 |
| SS18L2 | −0.2598961 | −0.2386609 | 0.24995563 | −0.570576 |
| NFIX | 0.4487442 | −0.4499578 | −0.2783189 | 0.57057276 |
| FRMD4A | 0.06759762 | −0.3506559 | −0.3275229 | 0.57050485 |
| AKIRIN2 | 0.0659957 | −0.2158195 | −0.5699803 | 0.04094788 |
| HAUS1 | −0.0803022 | 0.05839543 | 0.34763095 | −0.5698397 |
| NCOR1 | 0.18146096 | −0.1365149 | −0.3522181 | 0.56977585 |
| RBBP6 | 0.07361438 | 0.0296485 | −0.3997748 | 0.56957711 |
| SSR4 | −0.1612098 | 0.13625687 | 0.09518211 | −0.5690535 |
| ACTL6A | −0.1166318 | 0.07240063 | 0.33716007 | −0.5690428 |
| SOD1 | 0.02576813 | 0.18437878 | 0.1240553 | −0.5688333 |
| NDUFB3 | −0.0387811 | 0.29664904 | 0.31291233 | −0.5680716 |
| HERC2 | 0.56801277 | −0.253225 | −0.5294764 | 0.42908941 |
| ATP5I | −0.0562656 | 0.30105298 | 0.14927512 | −0.5680001 |
| FAM120B | 0.02145747 | −0.567595 | −0.4581603 | 0.21523379 |
| COX6A1 | −0.0553017 | 0.18235164 | −0.0115008 | −0.5675486 |
| PBX1 | 0.46050841 | 0.01243548 | −0.2266404 | −0.5675264 |
| IGFBP6 | −0.1252464 | 0.27561675 | 0.48165647 | −0.5673845 |
| AKAP9 | 0.21581127 | −0.087094 | −0.1098759 | 0.56734411 |
| PLAU | −0.5673287 | −0.1208923 | −0.209237 | 0.11024304 |
| ANKFY1 | 0.28728414 | −0.5670507 | −0.489924 | 0.47855987 |
| CPSF1 | 0.01858501 | −0.105163 | 0.05693289 | 0.56636831 |
| RNF181 | 0.05372649 | 0.18268907 | −0.0024656 | −0.5663306 |
| TOMM7 | −0.0883211 | 0.21282568 | 0.24889375 | −0.5658542 |
| LSM4 | −0.0618379 | 0.25836229 | 0.06550459 | −0.5656159 |
| ATG5 | −0.0195318 | −0.0583603 | 0.01209598 | −0.5650826 |
| RPL5 | −0.1386788 | 0.15779469 | 0.12552444 | −0.565028 |
| STARD3NL | −0.0727079 | 0.12386452 | 0.20176155 | −0.5649802 |
| RBM14 | −0.0684095 | 0.05433799 | −0.3342708 | 0.56492846 |
| AMPD2 | −0.0410706 | 0.09101496 | 0.08424238 | 0.56492526 |
| MAST2 | 0.17742984 | −0.3716446 | −0.1087907 | 0.56463898 |
| FAM174A | −0.1242805 | −0.1416328 | 0.32441573 | −0.5646133 |
| RPS27L | −0.1006892 | 0.20633141 | 0.14669635 | −0.5645059 |
| RHOG | −0.0318366 | 0.00239613 | 0.0910649 | 0.56439051 |
| PSMB4 | −0.0038407 | 0.2127347 | 0.11048191 | −0.5637491 |
| SRRM1 | 0.08148667 | −0.1316648 | −0.5635081 | 0.56330303 |
| NDUFB6 | −0.1085849 | 0.30670713 | 0.13013016 | −0.5634041 |
| PPP3CA | 0.21905449 | −0.2709168 | −0.5630928 | 0.17145656 |
| IKZF5 | −0.1056305 | −0.4574443 | −0.563053 | 0.48427223 |
| MED24 | 0.14316795 | 0.03896264 | 0.05937926 | 0.5630384 |
| ZNF142 | 0.18373347 | −0.1949545 | −0.5426388 | 0.56296111 |
| PCDHGC3 | −0.0964456 | −0.1887165 | −0.562752 | 0.40247203 |
| TMEM214 | 0.24889452 | −0.1851012 | −0.0511774 | 0.56263044 |
| TMEM14C | −0.0545916 | 0.12380815 | 0.12934648 | −0.5625395 |
| RBM3 | −0.0796274 | 0.04732422 | 0.13892706 | −0.5619069 |
| RHBDF2 | −0.0435509 | −0.3445649 | −0.0452669 | 0.56181949 |
| FRG1 | −0.0290216 | 0.14024825 | 0.23229854 | −0.5616458 |
| COQ5 | −0.1576225 | 0.05519564 | 0.00620944 | −0.5605841 |
| DCXR | 0.01410179 | 0.23453438 | −0.0696864 | −0.5604387 |
| PPP1R9B | 0.10378623 | −0.0465876 | 0.18280109 | 0.55994745 |
| MRPL17 | −0.1359594 | 0.16672228 | 0.06861519 | −0.5589964 |
| CTGF | −0.2132757 | −0.558971 | −0.5088502 | −0.3837502 |
| RAB7A | 0.02676518 | −0.5588245 | −0.4001748 | 0.28268406 |
| SREBF1 | −0.0373682 | −0.0154967 | −0.0132332 | 0.55850969 |
| ATP5H | −0.1837906 | 0.18001758 | 0.13515163 | −0.5583839 |
| TMEM208 | 0.03783463 | 0.22830451 | 0.47662752 | −0.5582157 |
| TIMMDC1 | −0.1056647 | 0.23450937 | 0.12585896 | −0.5579571 |
| SLC50A1 | 0.09271278 | 0.22447123 | −0.0383386 | −0.5578085 |
| CCDC57 | 0.03049013 | −0.230491 | 0.04398965 | 0.55768048 |
| ZNF462 | 0.51044446 | −0.245407 | −0.5574908 | 0.53701934 |
| PBX3 | 0.06672902 | −0.088653 | −0.2325674 | 0.55697265 |
| CELF2 | 0.46442181 | 0.05689647 | −0.415574 | 0.55695957 |
| C5orf28 | 0.01973129 | −0.0719692 | 0.28731329 | −0.556229 |
| RPTOR | 0.18549565 | −0.0356531 | −0.2628679 | 0.5561621 |
| TET3 | 0.24804048 | −0.2404849 | −0.5559853 | 0.52136739 |
| SELK | 0.02544383 | −0.0686865 | 0.17587077 | −0.5559827 |
| RAI1 | 0.3198781 | −0.4547992 | −0.5554582 | 0.15012235 |
| PPA1 | −0.0944888 | −0.0017129 | 0.15944897 | −0.5552173 |
| MPG | −0.1111622 | 0.22837236 | −0.0366842 | −0.5548056 |
| PRDX5 | −0.2081414 | 0.18055277 | 0.22167361 | −0.5547389 |
| TANC1 | 0.26441955 | −0.3583628 | −0.3847149 | 0.55448115 |
| PRR14L | 0.38125177 | −0.1660961 | −0.5153809 | 0.55447216 |
| RNFT1 | 0.08160982 | 0.01388201 | 0.2251117 | −0.5543453 |
| ZFAND2A | −0.089749 | 0.12900685 | 0.53579734 | −0.5541725 |
| HIST1H3B | −0.174013 | 0.02844044 | −0.0423323 | 0.5539376 |
| PSMC5 | 0.05542158 | −0.5536641 | −0.3241225 | 0.03492764 |
| TMEM218 | −0.1024553 | 0.14234129 | 0.11674077 | −0.5534551 |
| FAM214A | −0.1502468 | −0.5534291 | −0.5398987 | 0.2721808 |
| ANKRD17 | 0.27227484 | −0.165448 | −0.3911186 | 0.55332563 |
| SF1 | 0.02049894 | −0.2413227 | −0.1700993 | 0.55330612 |
| CSNK2A2 | 0.0978229 | −0.4124502 | −0.5532296 | 0.30332224 |
| APOL2 | 0.12380709 | 0.55295396 | 0.10435444 | 0.06757095 |
| C9orf89 | −0.0965402 | 0.37362362 | 0.10521518 | −0.5529308 |
| MARVELD1 | 0.0920998 | −0.0396936 | −0.1416973 | 0.55279349 |
| UBE4B | 0.07952726 | 0.00368896 | −0.2495437 | 0.55278567 |
| ADCY1 | −0.4292595 | −0.4413571 | −0.2695221 | 0.55260388 |
| PLXNB2 | 0.414472 | −0.422416 | −0.2276244 | 0.55258383 |
| SPRY4 | 0.00105236 | −0.1846097 | 0.55240283 | 0.45101859 |
| TCEAL3 | 0.06046035 | 0.55213377 | 0.04806349 | −0.1352728 |
| AOX1 | 0.38574024 | −0.0141461 | −0.5520031 | 0.17468445 |
| RELA | 0.12621618 | 0.07646938 | −0.0806561 | 0.55198731 |
| TEFM | 0.02192639 | 0.15355753 | 0.03058851 | −0.551747 |
| RPL27A | −0.1062605 | 0.08405093 | 0.11159807 | −0.5516193 |
| CCDC12 | 0.22683203 | 0.55152437 | 0.26953684 | −0.431155 |
| IL27RA | −0.5507006 | −0.0117677 | 0.00344222 | 0.28382383 |
| MAN2B2 | 0.22170079 | −0.2233025 | −0.0761453 | 0.55062616 |
| TSPO | −0.1622391 | 0.22312761 | 0.05241577 | −0.5503484 |
| TXN | −0.0495886 | 0.19978955 | 0.15784693 | −0.5501175 |
| LAMTOR5 | −0.0173054 | 0.0032075 | 0.33499889 | −0.5500412 |
| DYNLT1 | −0.0804737 | 0.10839169 | 0.1390772 | −0.5498241 |
| CDK10 | 0.03159956 | −0.0602099 | 0.54977639 | −0.1762513 |
| INTS3 | 0.01980387 | −0.2066687 | −0.1889085 | 0.54934334 |
| PDS5B | 0.14666468 | −0.3991796 | −0.5492778 | 0.13234049 |
| B2M | −0.1402403 | 0.09059853 | 0.25307709 | −0.5492138 |
| TAB2 | 0.09696458 | −0.3469202 | −0.5485589 | 0.41452399 |
| SRPR | 0.16851295 | −0.5479903 | −0.4459725 | 0.15583633 |
| CEP170 | 0.12952091 | −0.3773164 | −0.183294 | 0.54792944 |
| ZFYVE26 | 0.28225027 | −0.0820584 | −0.0754109 | 0.54768926 |
| CLUH | 0.14882095 | −0.2047762 | 0.00691356 | 0.54767119 |
| NCK1 | 0.01424151 | 0.03681804 | 0.18131492 | −0.547378 |
| ACACA | 0.3182275 | −0.4282022 | −0.5470147 | 0.20322552 |
| PLXNA3 | 0.06274598 | −0.32651 | −0.0916999 | 0.54688184 |
| DCTN3 | −0.0005519 | 0.1771386 | 0.20963098 | −0.5467645 |
| IPO9 | 0.10751791 | −0.1619177 | −0.2272642 | 0.54639746 |
| ATXN10 | −0.0656014 | −0.3070797 | −0.5461319 | −0.4144326 |
| RPSA | −0.0528037 | 0.15902745 | 0.11785869 | −0.5458575 |
| CD82 | −0.5453555 | −0.0907558 | −0.2513622 | −0.0355855 |
| HNRNPA2B1 | −0.0847303 | 0.05491734 | 0.12889554 | 0.54534867 |
| PCNXL3 | 0.23940964 | −0.028154 | −0.1407714 | 0.54526122 |
| DLG5 | 0.22482403 | −0.2941573 | −0.1685403 | 0.54484573 |
| NDUFB2 | −0.0179394 | 0.22836765 | 0.11489402 | −0.543856 |
| SNX1 | 0.21318915 | −0.54385 | −0.3033339 | 0.00804938 |
| ADAMTS1 | −0.0442496 | −0.2417127 | 0.09185611 | 0.54381249 |
| CELSR1 | 0.3241773 | −0.3784632 | −0.5437458 | 0.46467174 |
| RGP1 | 0.20668867 | −0.1737492 | −0.0446742 | 0.54358465 |
| DDX6 | 0.27596183 | −0.3474973 | −0.5433117 | 0.29654869 |
| ITGB1BP1 | −0.070396 | 0.00284986 | 0.05871082 | −0.5433111 |
| NPTX1 | 0.54328732 | −0.2173608 | 0.10778803 | 0.49260505 |
| RUSC2 | 0.1782357 | 0.01625714 | 0.01430451 | 0.54313659 |
| BOD1 | −0.2378745 | 0.08792207 | 0.18434588 | −0.5427684 |
| ZFP36L2 | 0.53214408 | −0.2837423 | −0.2625826 | 0.54265881 |
| GOLGB1 | 0.40524156 | −0.1512733 | −0.1536096 | 0.54226669 |
| KIAA2026 | 0.3004757 | −0.1614181 | −0.2670171 | 0.54225858 |
| TEAD1 | 0.15987569 | −0.1167364 | −0.3723834 | 0.54223578 |
| STAT1 | 0.14527698 | 0.54217847 | 0.04550296 | 0.31588701 |
| MED16 | 0.13214685 | 0.00767986 | −0.3100873 | 0.54215918 |
| NCKAP1 | 0.04849628 | −0.0851515 | −0.2493605 | 0.5415064 |
| SNX11 | −0.2828527 | −0.122489 | 0.08286002 | −0.5414856 |
| ITPA | −0.1581527 | 0.21845963 | 0.01916987 | −0.5409746 |
| SNRPF | −0.0199678 | 0.24925543 | 0.33992809 | −0.540838 |
| RPL11 | −0.0533179 | 0.1614374 | 0.1648822 | −0.540793 |
| IFNGR1 | −0.1297576 | 0.00932483 | 0.35143084 | −0.5404665 |
| ZBTB8OS | −0.0251358 | 0.06818483 | 0.10923555 | −0.5402949 |
| COPS3 | −0.1355724 | −0.0115668 | 0.07531204 | −0.5401716 |
| GTF3A | 0.02215305 | −0.0054405 | 0.08591066 | −0.5400328 |
| MYH10 | 0.33795632 | −0.2004441 | −0.0885091 | 0.53967835 |
| FLRT2 | 0.01350983 | −0.1837515 | −0.2305458 | 0.53937713 |
| C19orf10 | −0.1224246 | 0.0216805 | 0.02129544 | −0.5390284 |
| FLNB | 0.4647784 | −0.1939721 | −0.2126771 | 0.53848793 |
| FOSL2 | 0.43563544 | 0.09849434 | 0.10220321 | 0.538395 |
| MRPL23 | −0.0115564 | 0.17133515 | 0.02101825 | −0.5383191 |
| PSMD10 | 0.03504185 | 0.20728358 | 0.17560179 | −0.5381196 |
| TMED3 | −0.1536267 | 0.31326279 | 0.02922971 | −0.537901 |
| SLC5A6 | −0.3003709 | 0.07750865 | 0.03959711 | 0.53768723 |
| SH2B3 | −0.1163054 | −0.1450827 | −0.2825038 | 0.53757918 |
| HMGCS1 | −0.3305917 | −0.5370143 | 0.00832256 | −0.2637056 |
| MICAL2 | 0.11437893 | −0.3012273 | −0.5369197 | 0.5109278 |
| MIB2 | 0.12512166 | 0.05208757 | 0.4430477 | 0.5367477 |
| KIAA0922 | 0.00416956 | −0.0041655 | −0.1504559 | 0.53643396 |
| GLIS3 | 0.00203391 | −0.1055821 | −0.5210895 | 0.53608693 |
| OAZ1 | −0.1245782 | 0.19175499 | 0.05039872 | −0.53585 |
| HTATIP2 | 0.02643847 | 0.07907669 | 0.29516068 | −0.5354689 |
| SYNJ1 | 0.13651481 | −0.3894577 | −0.5354501 | 0.52322086 |
| DLG4 | −0.0561056 | −0.1397818 | 0.39153087 | 0.53478171 |
| AMPD3 | 0.02660482 | −0.0768976 | −0.5346955 | 0.37069721 |
| C1orf52 | −0.1551285 | 0.17863385 | 0.17099136 | −0.5339771 |
| RPL27 | −0.0950535 | 0.20910506 | 0.00249085 | −0.5339142 |
| CCDC90B | −0.0203532 | 0.08365205 | −0.0031126 | −0.5335047 |
| TAF12 | −0.2037725 | 0.09053668 | 0.44392418 | −0.5333095 |
| PTMA | −0.0263855 | 0.20299441 | 0.11506632 | −0.5330454 |
| DCP1A | 0.0959493 | 0.12701547 | −0.2292176 | 0.53301059 |
| ZNF611 | 0.33941082 | −0.338503 | −0.3026404 | 0.53295984 |
| PDCD5 | −0.1816878 | 0.14341064 | 0.16735679 | −0.5329234 |
| SENP3 | −0.049522 | 0.0405658 | −0.0059901 | −0.5326796 |
| MYH9 | 0.31089449 | −0.2766289 | −0.0849269 | 0.53243457 |
| UBB | −0.1275473 | 0.34276553 | 0.25013562 | −0.5322683 |
| FOXJ2 | 0.0943295 | −0.1707981 | −0.532134 | 0.35974137 |
| UBXN7 | 0.34426854 | −0.4220562 | −0.5320176 | 0.21032465 |
| HIST1H2BC | −0.2395357 | 0.09547506 | 0.10785155 | −0.5318357 |
| ZNFX1 | 0.28774959 | 0.23208658 | −0.0871684 | 0.53148038 |
| FNDC3B | 0.13549415 | −0.0913198 | −0.3291899 | 0.53141716 |
| FAM208B | 0.23285088 | −0.1084359 | −0.1633975 | 0.53132134 |
| HSPA5 | −0.3371878 | −0.5312859 | −0.2252102 | 0.19921754 |
| AFF4 | 0.31745645 | −0.1968303 | −0.530761 | 0.50764876 |
| HIST1H2AE | −0.2764649 | 0.10425154 | −0.0134419 | −0.5307397 |
| FAM60A | −0.1079917 | −0.0895979 | 0.13742678 | −0.5301617 |
| 42628 | −0.1533024 | 0.05561799 | 0.18131649 | −0.5299712 |
| CLIC1 | −0.1923872 | 0.16483548 | 0.13549438 | −0.5295827 |
| AGRN | 0.27558339 | −0.0579213 | 0.076167 | 0.52925189 |
| APTX | 0.04982703 | 0.09702217 | 0.18911084 | −0.5292267 |
| PSMB3 | −0.1170493 | 0.22811528 | 0.02866022 | −0.5289281 |
| SASH1 | 0.23063043 | −0.1508981 | −0.3022163 | 0.52843822 |
| CLTA | −0.0160922 | 0.19449866 | 0.0284277 | −0.5283923 |
| RHEB | −0.0854633 | 0.11298084 | 0.14423935 | −0.5280287 |
| DNAJA2 | −0.0290591 | 0.11076994 | 0.22349002 | −0.5279776 |
| UBA2 | −0.121009 | −0.0177761 | 0.07974037 | −0.5278594 |
| VPS13B | 0.31899421 | −0.2888357 | −0.5278486 | 0.39657989 |
| DPAGT1 | −0.0092699 | −0.0179682 | 0.00097294 | 0.52759332 |
| NCOA1 | 0.11922704 | −0.0770116 | −0.3367521 | 0.52759176 |
| COA6 | −0.2018072 | 0.01181063 | 0.20666232 | −0.5273021 |
| UTRN | 0.5272641 | −0.1507467 | −0.3697148 | 0.41744473 |
| NMB | −0.0803696 | 0.52653001 | 0.25605111 | −0.386272 |
| GSKIP | −0.0953593 | 0.06274327 | 0.15604186 | −0.5265042 |
| CBX5 | 0.40516196 | −0.3362878 | −0.5255397 | 0.52631335 |
| B4GALT6 | 0.14903867 | −0.2551047 | −0.5258619 | −0.2885695 |
| PIEZO1 | 0.26726074 | −0.0839819 | 0.10616602 | 0.5257401 |
| TACC2 | 0.07785822 | 0.05357652 | −0.3045174 | 0.52568547 |
| USP2 | 0.18524173 | −0.2238737 | −0.1428463 | 0.5256606 |
| NFU1 | 0.02382332 | −0.1171561 | 0.09135458 | −0.5256517 |
| MAPK1IP1L | 0.08474335 | −0.049168 | −0.439918 | 0.5255741 |
| PUM2 | 0.14354758 | −0.525253 | −0.5039825 | 0.30639196 |
| BCL9L | 0.43148049 | −0.5247745 | −0.382059 | 0.2162332 |
| SSR1 | −0.1341022 | −0.0152326 | −0.5245806 | 0.1665891 |
| ITPK1 | −0.1475098 | 0.02564897 | −0.2163489 | 0.52443003 |
| MAPKBP1 | 0.35639774 | −0.1954318 | −0.09164 | 0.52421017 |
| ZNF318 | 0.16221073 | −0.1305823 | −0.4172037 | 0.52397477 |
| ETHE1 | −0.1236209 | 0.12825111 | −0.0149539 | −0.5239603 |
| ZCCHC10 | −0.0469071 | 0.17484015 | 0.20658526 | −0.5235749 |
| SPRED2 | 0.29759305 | −0.2433991 | −0.4984676 | 0.52357348 |
| PRKCA | 0.47320811 | −0.3568228 | −0.5161944 | 0.52322305 |
| CHD7 | 0.04625625 | −0.5171732 | −0.3726806 | 0.52319348 |
| TFAP2A | 0.07020193 | −0.0046544 | −0.5081897 | 0.52290499 |
| MEA1 | −0.1140631 | 0.25307496 | 0.13728377 | −0.5228735 |
| RPL8 | −0.0691117 | 0.19718839 | 0.11204859 | −0.5228686 |
| ATP5G1 | −0.0987907 | 0.25777759 | 0.05680114 | −0.5228324 |
| RALGAPB | 0.16790084 | −0.1272312 | −0.2349644 | 0.52243123 |
| ATP6V1F | −0.1494279 | 0.23550716 | 0.02011351 | −0.5224178 |
| SMIM7 | 0.03026485 | 0.16906032 | 0.10619775 | −0.5222803 |
| C1orf50 | −0.0074135 | −0.1316025 | −0.0585286 | −0.5217887 |
| ATXN1L | 0.20851962 | −0.0546624 | −0.0138853 | 0.52166478 |
| TAOK3 | 0.0538397 | −0.2896611 | −0.3525882 | 0.52161088 |
| HIVEP2 | 0.50606996 | −0.2700093 | −0.3116946 | 0.52112415 |
| STS | 0.16837591 | 0.0330668 | −0.5211023 | 0.10023032 |
| WDR26 | 0.21678475 | −0.2614728 | −0.5205466 | 0.25525346 |
| TOP2A | 0.23197873 | −0.0157788 | −0.0004755 | 0.52031806 |
| CCDC6 | 0.00012011 | 0.02476587 | −0.5199046 | 0.34146741 |
| VPS13D | 0.51979295 | −0.1525629 | −0.3394081 | 0.44907785 |
| RNF38 | 0.10221754 | −0.0880927 | −0.4384741 | 0.51959276 |
| KDM2A | 0.20039343 | −0.1247406 | −0.1651819 | 0.5194527 |
| ASNS | −0.5193598 | 0.1240141 | −0.0700706 | −0.1460477 |
| CKAP5 | 0.21754962 | 0.01758074 | −0.0924651 | 0.51925607 |
| VPS29 | 0.03956266 | 0.01277438 | 0.12846054 | −0.5191764 |
| HNRNPA0 | 0.16706893 | −0.5189423 | 0.01159142 | 0.21449568 |
| NUDT14 | −0.0876665 | −0.3162849 | −0.2012516 | −0.5185342 |
| SLC35F6 | 0.05645232 | −0.1211496 | −0.3064036 | 0.51820084 |
| CDK5RAP3 | −0.1167549 | 0.32194979 | 0.51819039 | 0.2764768 |
| RGS10 | −0.3211278 | −0.0010253 | 0.04414248 | −0.5181831 |
| CNIH1 | 0.02619286 | −0.0627346 | −0.0793689 | −0.517949 |
| NAE1 | −0.0675087 | 0.01645091 | 0.19791498 | −0.5177712 |
| MPHOSPH8 | 0.03397299 | 0.04108847 | −0.1430365 | 0.51749399 |
| KHDRBS1 | 0.27910105 | −0.5172959 | −0.5092018 | 0.05568034 |
| TRIM41 | −0.1116196 | 0.35823232 | 0.2769105 | 0.51727731 |
| UBE2B | 0.11653441 | −0.5172532 | −0.2155054 | −0.2752584 |
| RAP1A | 0.13294147 | −0.516972 | −0.2720791 | −0.232872 |
| WASF2 | 0.34014255 | −0.3643959 | −0.4821383 | 0.51616254 |
| RBM15 | 0.22202537 | −0.055309 | −0.0579706 | 0.51605167 |
| UBQLN2 | −0.1277609 | −0.3052531 | −0.5159802 | 0.47426771 |
| EIF4G1 | 0.1182776 | −0.0449694 | −0.0198472 | 0.5157993 |
| NDST1 | 0.24911668 | −0.0661302 | −0.1304274 | 0.51573738 |
| PRDM10 | 0.1337423 | −0.2275514 | −0.0212149 | 0.51568876 |
| TNRC6A | 0.19319192 | −0.2209476 | −0.0754911 | 0.51556923 |
| NDUFB8 | −0.0163846 | −0.0082133 | 0.17564266 | −0.5154261 |
| ISOC2 | −0.0271062 | 0.3686245 | 0.05562535 | −0.5150801 |
| HOOK3 | 0.256545 | −0.3867896 | −0.5149002 | 0.26530616 |
| PLEKHJ1 | −0.2399147 | 0.24455101 | 0.2686366 | −0.5141846 |
| LGALS1 | −0.1069833 | 0.29501308 | 0.34698272 | −0.5140236 |
| VPS25 | −0.1516772 | 0.17746878 | −0.035588 | −0.513589 |
| ATF7IP | 0.23155413 | −0.3138314 | −0.4273379 | 0.51355202 |
| CARD9 | −0.2608557 | 0.24247539 | 0.22937184 | 0.51328723 |
| GOLGA3 | 0.30791259 | −0.0979988 | −0.0860339 | 0.51305286 |
| SYNJ2 | 0.16974842 | −0.1488112 | −0.0425671 | 0.51292699 |
| ITGB8 | 0.26196209 | 0.27879816 | −0.5128835 | 0.50419115 |
| PPARD | −0.1151066 | −0.1069367 | −0.239489 | 0.51276537 |
| H6PD | 0.33567325 | −0.2253079 | −0.2978887 | 0.51213646 |
| CBS | −0.5120212 | 0.14999835 | 0.14907323 | 0.20563307 |
| TATDN1 | −0.1475095 | −0.0219852 | 0.17354358 | −0.5116292 |
| POMK | 0.04008045 | −0.094102 | −0.2373362 | 0.51160621 |
| TNKS1BP1 | 0.35298111 | 0.00989539 | 0.19019798 | 0.51156449 |
| ATP2B4 | 0.47159024 | −0.2346006 | −0.5115096 | 0.36158347 |
| DHX38 | 0.20496812 | 0.10325314 | 0.08395173 | 0.51141946 |
| TAOK1 | 0.38993145 | −0.1713866 | −0.2876863 | 0.51130902 |
| PSMD13 | −0.097676 | 0.15388899 | 0.10577048 | −0.5112565 |
| DOPEY2 | 0.51019407 | −0.0884928 | −0.308042 | 0.3938517 |
| IGF2R | 0.47067548 | −0.4318273 | −0.3895396 | 0.5095359 |
| HIVEP1 | 0.22127371 | −0.0720493 | −0.5089846 | 0.40328838 |
| TIAM2 | 0.50879968 | −0.0060125 | −0.3983448 | 0.28149008 |
| DDX39B | −0.5087881 | −0.2655738 | 0.27205764 | 0.34306313 |
| TTYH3 | 0.46459361 | −0.2733159 | −0.234031 | 0.50845309 |
| TXNDC9 | 0.03090648 | 0.16579991 | 0.21894476 | −0.5082878 |
| KIF13A | 0.29781575 | −0.0189662 | −0.0808723 | 0.50824021 |
| GLRX3 | −0.069425 | 0.1251562 | 0.05036684 | −0.5078272 |
| TIMM17B | −0.1101356 | 0.2747208 | −0.0117336 | −0.5077371 |
| ALKBH2 | −0.4539901 | 0.24612219 | 0.50765447 | −0.1551417 |
| SCAND1 | −0.0432603 | 0.2866841 | −0.0676189 | −0.5071335 |
| ELP5 | −0.02016 | 0.05505045 | 0.00174334 | −0.5069586 |
| DOPEY1 | 0.3066845 | −0.0577204 | −0.0144031 | 0.50691517 |
| TCOF1 | 0.14378755 | −0.0077061 | −0.0176247 | 0.50680879 |
| MGST3 | −0.0970302 | 0.17860739 | 0.13488892 | −0.5066877 |
| NFRKB | −0.0193744 | 0.10390296 | 0.03949668 | 0.50644244 |
| PNN | −0.004587 | 0.07446417 | 0.39988232 | 0.50634605 |
| CDC123 | −0.106382 | 0.07694548 | 0.14202872 | −0.5060748 |
| PPP2R5E | 0.07359214 | −0.2740628 | −0.5056397 | 0.12025767 |
| TCERG1 | −0.0593964 | 0.00679815 | 0.00402833 | 0.50559988 |
| SPHK1 | −0.5048876 | 0.1666443 | 0.50147085 | −0.1904178 |
| TBC1D16 | 0.22961629 | −0.0954713 | 0.0054563 | 0.50466407 |
| RPL29 | −0.0382206 | 0.24659113 | 0.03319595 | −0.5046262 |
| ATP7A | 0.33109761 | −0.0442711 | −0.1145455 | 0.50462524 |
| TNKS | 0.14055398 | −0.1143356 | −0.3073172 | 0.50439103 |
| PRTG | −0.0127114 | 0.00552363 | −0.5040723 | 0.35503648 |
| GFOD1 | 0.06292123 | −0.0310921 | 0.03732134 | 0.50400566 |
| SMC4 | 0.10694852 | −0.2129709 | −0.1281865 | 0.50369359 |
| MYCBP2 | 0.50364064 | −0.1013739 | −0.4058507 | 0.48207889 |
| ACADVL | −0.1957301 | 0.12070285 | 0.50350563 | 0.14778155 |
| HMGA1 | 0.13590961 | 0.01151469 | −0.1122424 | 0.50338802 |
| HNRNPM | 0.14995869 | −0.1989986 | −0.1602786 | 0.50328508 |
| C1orf54 | 0.09571617 | 0.1917904 | 0.16669633 | −0.5026295 |
| NDUFS5 | −0.0432345 | 0.19605896 | 0.0790887 | −0.5026206 |
| ADSL | −0.1888454 | 0.14906963 | 0.12194149 | −0.5022232 |
| AP3D1 | 0.11118791 | −0.0357219 | 0.04693049 | 0.50209159 |
| SETD1A | 0.2709323 | −0.5015214 | −0.4543843 | 0.31215405 |
| TNFAIP3 | 0.18669904 | −0.5015093 | −0.2886948 | 0.39744213 |
| RYBP | 0.07093389 | −0.2771175 | −0.500935 | 0.12758249 |
| RPLP1 | −0.1198657 | 0.16404093 | −0.0647548 | −0.5009318 |
| NDUFC1 | −0.1030795 | 0.0440726 | 0.09811742 | −0.5008456 |
| TEP1 | 0.10532 | −0.2414393 | 0.08085992 | 0.5005537 |
| WDR7 | 0.34077546 | −0.0437672 | −0.2479611 | 0.50048656 |
| AGAP1 | 0.37761605 | −0.0898255 | −0.0848244 | 0.50045323 |
| RFK | 0.00601232 | 0.00793012 | 0.05656493 | −0.500388 |
| PCNT | 0.46283256 | −0.2965203 | −0.133546 | 0.50035115 |
| MOB3A | −0.0307228 | −0.2325328 | −0.1942487 | 0.50034547 |

### REFERENCES

### Example 2

[1]. Hajra A, Bandyopadhyay D, Hajra S K. 2016. Zika Virus: A Global Threat to Humanity: A Comprehensive Review and Current Developments. N Am J Med Sci 8:123-128; [2]. Plourde A R, Bloch E M. 2016. A Literature Review of Zika Virus. Emerg Infect Dis 22; [3]. D'Ortenzio E, Matheron S, de Lamballerie X, Hubert B, Piorkowski G, Maquart M, Descamps D, Damond F, Yazdanpanah Y, Leparc-Goffart I. 2016. Evidence of Sexual Transmission of Zika Virus. N Engl J Med doi:10.1056/NEJMc1604449; [4]. Moreira J, Lamas C C, Siqueira A. 2016. Sexual Transmission of Zika Virus: Implications for Clinical Care and Public Health Policy. Clin Infect Dis doi:10.1093/cid/ciw211; [5]. Dick G W, Kitchen S F, Haddow A J. 1952. Zika virus. I. Isolations and serological specificity. Trans R Soc Trop Med Hyg 46:509-520; [6]. Macnamara F N. 1954. Zika virus: a report on three cases of human infection during an epidemic of jaundice in Nigeria. Trans R Soc Trop Med Hyg 48:139-145; [7]. Wang Z, Wang P, An J. 2016. Zika virus and Zika fever. Virol Sin 31:103-109; [8]. Cordeiro M T, Pena L J, Brito C A, Gil L H, Marques E T. 2016. Positive IgM for Zika virus in the cerebrospinal fluid of 30 neonates with microcephaly in Brazil. Lancet doi:10.1016/50140-6736(16)30253-7; [9]. de Carvalho N S, de Carvalho B F, Fugaca C A, Doris B, Biscaia E S. 2016. Zika virus infection during pregnancy and microcephaly occurrence: a review of literature and Brazilian data. Braz J Infect Dis doi:10.1016/j.bjid.2016.02.006; [10]. Nourollahpour Shiadeh M, Rostami A, Danesh M, Sajedi A A. 2016. Zika virus as new emerging global health threat for pregnancy and child birth. J Matern Fetal Neonatal Med doi:10.1080/14767058.2016.1177820:1; [11]. Brasil P, Sequeira P C, Freitas A D, Zogbi H E, Calvet G A, de Souza R V, Siqueira A M, de Mendonca M C, Nogueira R M, de Filippis A M, Solomon T. 2016. Guillain-Barre syndrome associated with Zika virus infection. Lancet 387:1482; [12]. Hamel R, Dejarnac O, Wichit S, Ekchariyawat P, Neyret A, Luplertlop N, Perera-Lecoin M, Surasombatpattana P, Talignani L, Thomas F, Cao-Lormeau V M, Choumet V, Briant L, Despres P, Amara A, Yssel H, Misse D. 2015. Biology of Zika Virus Infection in Human Skin Cells. J Virol 89:8880-8896; [13]. Briant L, Despres P, Choumet V, Misse D. 2014. Role of skin immune cells on the host susceptibility to mosquito-borne viruses. Virology 464-465:26-32; [14]. Tang H, Hammack C, Ogden S C, Wen Z, Qian X, Li Y, Yao B, Shin J, Zhang F, Lee E M, Christian K M, Didier R A, Jin P, Song H, Ming G L. 2016. Zika Virus Infects Human Cortical Neural Progenitors and Attenuates Their Growth. Cell Stem Cell 18:587-590; [15]. Bayer A, Lennemann N J, Ouyang Y, Bramley J C, Morosky S, Marques E T, Jr., Cherry S, Sadovsky Y, Coyne C B. 2016. Type III Interferons Produced by Human Placental Trophoblasts Confer Protection against Zika Virus Infection. Cell Host Microbe doi:10.1016/j.chom.2016.03.008; [16]. Dang J, Tiwari S, Lichinchi G, Qin Y, Patil V, Eroshkin A, Rana T. 2016. Zika Virus Depletes Neural Progenitors in Human Cerebral Organoids through Activation of the Innate Immune Receptor TLR3. Cell Stem Cell, 19, 258-65; [17]. Lazear H M, Govero J, Smith A M, Platt D J, Fernandez E, Miner J J, Diamond M S. 2016. A Mouse Model of Zika Virus Pathogenesis. Cell Host Microbe doi:10.1016/j.chom.2016.03.010:10.1016/j.chom.2016.1003.1010; [18]. Rossi S L, Tesh R B, Azar S R, Muruato A E, Hanley K A, Auguste A J, Langsjoen R M, Paessler S, Vasilakis N, Weaver S C. 2016. Characterization of a Novel Murine Model to Study Zika Virus. Am J Trop Med Hyg doi:10.4269/ajtmh.16-0111; [19]. Aliota M T, Caine E A, Walker E C, Larkin K E, Camacho E, Osorio J E. 2016. Characterization of Lethal Zika Virus Infection in AG129 Mice. PLoS Negl Trop Dis 10:e0004682; [20]. Lambert C, Doring T, Prange R. 2007. Hepatitis B virus maturation is sensitive to functional inhibition of ESCRT-III, Vps4, and gamma 2-adaptin. J Virol 81:9050-9060; [21]. Nowakowski Tomasz J, Pollen Alex A, Di Lullo E, Sandoval-Espinosa C, Bershteyn M, Kriegstein Arnold R. 2016. Expression Analysis Highlights AXL as a Candidate Zika Virus Entry Receptor in Neural Stem Cells. Cell Stem Cell doi:10.1016/j.stem.2016.03.012; [22]. Sanchez E L, Lagunoff M. 2015. Viral activation of cellular metabolism. Virology 479-480:609-618; [23]. Goodwin C M, Xu S, Munger J. 2015. Stealing the Keys to the Kitchen: Viral Manipulation of the Host Cell Metabolic Network. Trends Microbiol 23:789-798; [24]. Maynard N D, Gutschow M V, Birch E W, Covert M W. 2010. The virus as metabolic engineer. Biotechnol J 5:686-694; [25]. Brecher M, Chen H, Liu B, Banavali N K, Jones S A, Zhang J, Li Z, Kramer L D, Li H. 2015. Novel Broad Spectrum Inhibitors Targeting the Flavivirus Methyltransferase. PLoS One 10:e0130062; [26]. Fischer M A, Smith J L, Shum D, Stein D A, Parkins C, Bhinder B, Radu C, Hirsch A J, Djaballah H, Nelson J A, Fruh K. 2013. Flaviviruses are sensitive to inhibition of thymidine synthesis pathways. J Virol 87:9411-9419; [27]. Chen Y L, Yin Z, Duraiswamy J, Schul W, Lim C C, Liu B, Xu H Y, Qing M, Yip A, Wang G, Chan W L, Tan H P, Lo M, Liung S, Kondreddi R R, Rao R, Gu H, He H, Keller T H, Shi P Y. 2010 Inhibition of dengue virus RNA synthesis by an adenosine nucleoside. Antimicrob Agents Chemother 54:2932-2939; [28]. Chan J F, Yip C C, Tsang J O, Tee K M, Cai J P, Chik K K, Zhu Z, Chan C C, Choi G K, Sridhar S, Zhang A J, Lu G, Chiu K, Lo A C, Tsao S W, Kok K H, Jin D Y, Chan K H, Yuen K Y. 2016. Differential cell line susceptibility to the emerging Zika virus: implications for disease pathogenesis, non-vector-borne human transmission and animal reservoirs. Emerg Microbes Infect 5:e93; [29]. Torres S, Hernandez J C, Giraldo D, Arboleda M, Rojas M, Smit J M, Urcuqui-Inchima S. 2013. Differential expression of Toll-like receptors in dendritic cells of patients with dengue during early and late acute phases of the disease. PLoS Negl Trop Dis 7:e2060; [30]. Wang J P, Liu P, Latz E, Golenbock D T, Finberg R W, Libraty D H. 2006. Flavivirus Activation of Plasmacytoid Dendritic Cells Delineates Key Elements of TLR7 Signaling beyond Endosomal Recognition. The Journal of Immunology 177:7114-7121; [31]. Diamond M. 2003. Evasion of innate and adaptive immunity by flaviviruses. Immunology and Cell Biology 81:196-206; [32]. Ye J, Zhu B, Fu Z F, Chen H, Cao S. 2013. Immune evasion strategies of flaviviruses. Vaccine 31:461-471; [33]. Xiang J, McLinden J H, Rydze R A, Chang Q, Kaufman T M, Klinzman D, Stapleton J T. 2009. Viruses within the Flaviviridae decrease CD4 expression and inhibit HIV replication in human CD4+ cells. J Immunol 183:7860-7869; [34]. Rolfe A J, Bosco D B, Wang J, Nowakowski R S, Fan J, Ren Y. 2016. Bioinformatic analysis reveals the expression of unique transcriptomic signatures in Zika virus infected human neural stem cells. Cell Biosci 6:42; [35]. Ramos H J, Lanteri M C, Blahnik G, Negash A, Suthar M S, Brassil M M, Sodhi K, Treuting P M, Busch M P, Norris P J, Gale M, Jr. 2012. IL-1beta signaling promotes CNS-intrinsic immune control of West Nile virus infection. PLoS Pathog 8:e1003039; [36]. Liu P, Woda M, Ennis F A, Libraty D H. 2009. Dengue virus infection differentially regulates endothelial barrier function over time through type I interferon effects. J Infect Dis 200:191-201; [37]. Karimi O, Goorhuis A, Schinkel J, Codrington J, Vreden S G, Vermaat J S, Stijnis C, Grobusch M P. 2016. Thrombocytopenia and subcutaneous bleedings in a patient with Zika virus infection. Lancet 387:939-940; [38]. Heaton N, Perera R, Berger K, Khadka S, LaCount D, Kuhn R, Randall G. 2010. Dengue virus nonstructural protein 3 redistributes fatty acid synthase to sites of viral replication and increases cellular fatty acid synthesis. PNAS 107:17345-17350; [39]. Thai M, Graham N A, Braas D, Nehil M, Komisopoulou E, Kurdistani S K, McCormick F, Graeber T G, Christofk H R. 2014. Adenovirus E4ORF1-induced MYC activation promotes host cell anabolic glucose metabolism and virus replication. Cell Metab 19:694-701; [40]. Wang L, Huang J, Jiang M. 2011. CREB5 computational regulation network construction and analysis between frontal cortex of HIV encephalitis (HIVE) and HIVE-control patients. Cell Biochem Biophys 60:199-207; [41]. Domingo-Gil E, Gonzalez J M, Esteban M. 2010. Identification of cellular genes induced in human cells after activation of the OAS/RNaseL pathway by vaccinia virus recombinants expressing these antiviral enzymes. J Interferon Cytokine Res 30:171-188; [42]. Huang da W, Sherman B T, Lempicki R A. 2009. Systematic and integrative analysis of large gene lists using DAVID bioinformatics resources. Nat Protoc 4:44-57; [43]. Bindea G, Mlecnik B, Hackl H, Charoentong P, Tosolini M, Kirilovsky A, Fridman W H, Pages F, Trajanoski Z, Galon J. 2009. ClueGO: a Cytoscape plug-in to decipher functionally grouped gene ontology and pathway annotation networks. Bioinformatics 25:1091-1093; [44]. Shannon P, Markiel A, Ozier O, Baliga N S, Wang J T, Ramage D, Amin N, Schwikowski B, Ideker T. 2003. Cytoscape: a software environment for integrated models of biomolecular interaction networks. Genome Res 13:2498-2504.

### Embodiments

Embodiment 1: A method of treating a Zika viral infection, said method comprising administering to a subject in need thereof an effective amount of a compound as set forth in any of FIG. 1A, 1B, 2, 3, 6, 8, or 9.

Embodiment 2: The method of embodiment 1, wherein said compound is Ganciclovir, Procaine hydrochloride. Zidovudine. Acyclovir. Drostanolone Propionate, Dapivirine (TMC120), Tilorone hydrochloride, or Docosanol.

Embodiment 3: A method of treating a Zika viral infection, said method comprising administering to a subject in need thereof an effective amount of an inhibitor of NS5 polymerase.

Embodiment 4: The method of embodiment 3, wherein said inhibitor of NS5 polymerase is beclabuvir, dasabuvir, deleobuvir, filibuvir, radalbuvir, setrobuvir, or sofosbuvir.

Embodiment 5: The method of embodiment 3 or 4, wherein said inhibitor of NS5 polymerase is sofosbuvir.

Embodiment 6: A method of treating a Zika viral infection, said method comprising administering to a subject in need thereof an effective amount of an HIV protease inhibitor.

Embodiment 7: The method of embodiment 6, wherein said HIV protease inhibitor is nelfinavir.

Embodiment 8: A method of treating a Zika viral infection, said method comprising administering to a subject in need thereof an effective amount of a calcium channel blocker.

Embodiment 9: The method of embodiment 8, wherein said calcium channel blocker is manidipine, cilnidipine, or benidipine.

Embodiment 10: A method of treating a Zika viral infection, said method comprising administering to a subject in need thereof an effective amount of a combination therapeutic composition comprising an NS5 polymerase inhibitor and a HIV protease inhibitor, according to any one of embodiments 1 to 7.

Embodiment 11: The method of any one of embodiments 1 to 7, or 10, wherein said combination therapeutic composition comprises Suramin, Ganciclovir, Procaine hydrochloride, Zidovudine, Acyclovir, Drostanolone Propionate, Dapivirine (TMC120), Tilorone hydrochloride, Docosanol, clomiphene, amphotericin B, toremifene, mycophenolic acid, fluoxetine, niclosamide, or polyhydroxyalkanoate (PHA).

### Further Embodiments

Embodiment 1. A method of treating a Zika viral infection in a subject in need thereof, said method comprising administering to said subject an effective amount of an NS5 polymerase inhibitor.

Embodiment 2. The method of embodiment 1, wherein said NS5 polymerase inhibitor is beclabuvir, dasabuvir, deleobuvir, filibuvir, radalbuvir, setrobuvir, or sofosbuvir.

Embodiment 3. The method of embodiment 1 or 2, wherein said NS5 polymerase inhibitor is sofosbuvir.

Embodiment 4. A method of treating a Zika viral infection in a subject in need thereof, said method comprising administering to said subject an effective amount of an HIV protease inhibitor.

Embodiment 5. The method of embodiment 4, wherein said HIV protease inhibitor is amprenavir, atazanavir, darunavir, fosamprenavir, indinavir, lopinavir, nelfinavir, ritonavir, saquinavir, tipranavir, asunaprevir, boceprevir, grazoprevir, paritaprevir, simeprevir, or telaprevir.

Embodiment 6. The method of embodiment 4 or 5, wherein said HIV protease inhibitor is nelfinavir.

Embodiment 7. A method of treating a Zika viral infection in a subject in need thereof, said method comprising administering to said subject a combined effective amount of an NS5 polymerase inhibitor and an HIV protease inhibitor, according to any one of embodiments 1 to 6.

Embodiment 8. The method of any one of embodiments 1 to 7, wherein said NS5 polymerase inhibitor is beclabuvir, dasabuvir, deleobuvir, filibuvir, radalbuvir, setrobuvir, or sofosbuvir.

Embodiment 9. The method of any one of embodiments 1 to 7, wherein said HIV protease inhibitor is amprenavir, atazanavir, darunavir, fosamprenavir, indinavir, lopinavir, nelfinavir, ritonavir, saquinavir, tipranavir, asunaprevir, boceprevir, grazoprevir, paritaprevir, simeprevir, or telaprevir.

Embodiment 10. A method of treating a Zika viral infection in a subject in need thereof, said method comprising administering to said subject an effective amount of a protein or a gene encoding the protein, wherein the protein is a ZIKV non-structural (NS) protein, ZIKV protease, or ZIKV RNA polymerase.

Embodiment 11. The method of embodiment 10, wherein the ZIKV NS protein is NS5.

Embodiment 12. The method of embodiment 10 or 11, wherein the ZIKV NS protein is NS2B-NS3.

Embodiment 13. The method of any one of embodiments 10 to 12, wherein the ZIKV protein is NS5 RNA polymerase.

Embodiment 14. The method of any one of embodiments 10 to 13, wherein the ZIKV protein is NS2B-NS3 protease.

Embodiment 15. A method of treating a Zika viral infection in a subject in need thereof, said method comprising administering to said subject an effective amount of an inhibitor, wherein the inhibitor is suramin, ganciclovir, procaine hydrochloride, zidovudine, acyclovir, drostanolone propionate, dapivirine, tilorone hydrochloride, docosanol, clomiphene, amphotericin B, toremiphine, mycophenolic acid, fluoxetine, niclosamide, polyhydroxyalkanoate, or a combination thereof, according to any one of embodiments 1 to 14.

What is claimed is:

1. A method of treating a Zika viral infection in a subject in need thereof, said method comprising administering to said subject an effective amount of an NS5 polymerase inhibitor.

2. The method according to claim 1, wherein said NS5 polymerase inhibitor is beclabuvir, dasabuvir, deleobuvir, filibuvir, radalbuvir, setrobuvir, or sofosbuvir.

3. The method according to claim 2, wherein said NS5 polymerase inhibitor is sofosbuvir.

4. A method of treating a Zika viral infection in a subject in need thereof, said method comprising administering to said subject an effective amount of an HIV protease inhibitor.

5. The method according to claim 4, wherein said HIV protease inhibitor is amprenavir, atazanavir, darunavir, fosamprenavir, indinavir, lopinavir, nelfinavir, ritonavir, saquinavir, tipranavir, asunaprevir, boceprevir, grazoprevir, paritaprevir, simeprevir, or telaprevir.

6. The method according to claim 5, wherein said HIV protease inhibitor is nelfinavir.

7. A method of treating a Zika viral infection in a subject in need thereof, said method comprising administering to said subject a combined effective amount of an NS5 polymerase inhibitor and an HIV protease inhibitor.

8. The method according to claim 7, wherein said NS5 polymerase inhibitor is beclabuvir, dasabuvir, deleobuvir, filibuvir, radalbuvir, setrobuvir, or sofosbuvir.

9. The method according to claim 7, wherein said HIV protease inhibitor is amprenavir, atazanavir, darunavir, fosamprenavir, indinavir, lopinavir, nelfinavir, ritonavir, saquinavir, tipranavir, asunaprevir, boceprevir, grazoprevir, paritaprevir, simeprevir, or telaprevir.

10. A method of treating a Zika viral infection in a subject in need thereof, said method comprising administering to said subject an effective amount of a protein or a gene encoding the protein, wherein the protein is a ZIKV non-structural (NS) protein, ZIKV protease, or ZIKV RNA polymerase.

11. The method of claim 10, wherein the ZIKV NS protein is NS5.

12. The method of claim 10, wherein the ZIKV NS protein is NS2B-NS3.

13. The method of claim 10, wherein the ZIKV protein is NS5 RNA polymerase.

14. The method of claim 10, wherein the ZIKV protein is NS2B-NS3 protease.

15. A method of treating a Zika viral infection in a subject in need thereof, said method comprising administering to said subject an effective amount of an inhibitor, wherein the inhibitor is suramin, ganciclovir, procaine hydrochloride, zidovudine, acyclovir, drostanolone propionate, dapivirine, tilorone hydrochloride, docosanol, clomiphene, amphotericin B, toremiphine, mycophenolic acid, fluoxetine, niclosamide, polyhydroxyalkanoate, or a combination thereof.
